SEQUENCE LISTING (43 sequences)

SEQ ID NO: 1 - protein, 112 aa, Homo sapiens
  source 1..112: note = MXI1 biomarker
MPSPRLQHSKPPRRLSRAQKHSSGSSNTSTANRRAHLRLCLERLKVLIPLGPDCTRHTTLGLLNKAKAHIKKLEEAERKSQHQLENLEREQRFLKWRLEQLQGPQEMERIRM

SEQ ID NO: 2 - DNA, 3047 bp, Homo sapiens
  source 1..3047: mol_type = other DNA; note = MXI1 biomarker
catcgcccgaggcgtccgccctgcaaatctgccagcccccgagaggggtttggaaccttagcacccaagtctaagtcaacttcatagtgtgaacatggctacgcctcttcattcccgtccatgccgagcccccgactgcagcattcaaagcccccacggaggttgagccgggcacagaaacacagcagcgggagcagcaacaccagcactgccaacagacgagctcatctgcgcctttgtttagaacgcttaaaagttctgattccactaggaccagactgcacccggcacacaacacttggtttgctcaacaaagccaaagcacacatcaagaaacttgaagaagctgaaagaaaaagccagcaccagctcgagaatttggaacgagaacagagatttttaaagtggcgactggaacagctgcagggtcctcaggagatggaacgaatacgaatggacagcattggatcaactatttcttcagatcgttctgattcagagcgagaggagattgaagtggatgttgaaagcacagagttctcccatggagaagtggacaatataagtaccaccagcatcagtgacattgatgaccacagcagcctgccgagtattgggagtgacgagggttactccagtgccagtgtcaaactttcattcacttcatagaacccagcatgacataacagtgcagggcaaaatattcactgggccaattcaatacaaacaatctcttaaattgggttcatgatgcagtctcctctttaaaacaaaacaaaacaaaacaaaactatacttgaacaaaagggtcagaggacctgtatttaagcaaatacttagcaaaaagtggggcagagcctcccaaggagaacaaatattcagaatattcatattggaaaaatcacaatttttaatggcagcagaaaacttgtgtgaaattttcttgatttgagttgattgagaagaggacattggagatgccatcctctttctcttttctagtttgctcatactacattgagtagacacatttaaggatggggttatgaacccttcctgagctttatggtcctaaaagcaaaataaaaactattcgaatgaaaagacaagaaaatcaggtattaatcttggatagctaataatgagctattaaaactcagcctgggacagtttatcatgaagcctgtggatgatcaatcctttattattattttttttttttgaaaaaagctcatttcatgctctgcaaaaggagagactcccatgaagccttttgaaagggatcatcatgcagctcaactttctgttggattccatgctaagcaagctaaccttatcctgcattgttagcactaggcacccagctgccacctctccatcctgctgcccttaggccacatgggagcagtccatgcatgacagcctctatcctacaaggcctatgagtatggattgggggggccaaaaggaaaaagctccatgtgcctctttgtctgcgtgggtcagaagagttgtgcacgcagattagcaggccaaggtctgagccacagcagcatttttatttcagattttgataactgtttatatgtgttgaaaaccaaaatgacatctttttaaagcttatccataaaaaaaaatagatgtcttttatagtggaaaaacacatggggaaaaaaatcatctattttgatgcagcatttgataatgataaaacacctcacacctcactctttatagtgcacaaaatgaatgaggtctgggctaggtagaaaaagggtcaatgctatttttgtttttagaatcattaccttttaccagcttttaaccatctgatatctatagtagacacactatcatagttaacatagtaagttcagcacttgtctcattttaatgtaaagatttgcttccattttcctacaggcagtctctctcttcctcacagtcccactgtgcaggtgctattgttactcttacgaatattttcagtaatgttattttcttctaagtgaaatttctagcctgcactttgatgtcatgtgttccctttgtctttcaaactccaaggttcccttgtggccctctcccttaccctgggaaggcctcttggagaccttacccctggctgtttggactttgtatactttaaataatttaactacccttaattacttaaaaaaaaaaaaaagctttatgattttcataacttattgctgattttaatggattgttaatttcagtcctgtagttttattttatgtttagatagggctgggcaaggaaaaagaaaataaagacaaccatatttagcagtgcagttgagttgtgtgttaatgttagactatccctttgtgagtgacactttaacagcattcactgcttctatatatagtgtaccatcttggtcatacattacgcctcaacatatacttgtgctcttcctttgcctccagaagaagtttttccttgattgtgctatgtttcagtggaagaaattctttgaagtagatgtgagtgaaaaactgcatgcctttagaagcccagtatcagaacttgctacgtttcaggtgctagggacttaatgaaaaacaggacaaaacaattcctttttgtggcccaggtaaattatttctggtttcacttataattactaatggctgagtcaagatgttgtctctgtgtttgcttactcttgatcaagtgtgagacagtttgaagactgtgctaccatacaaagtgaatgaagccagtgactaagcttctgtttgttttgttattctcatggccttcgcttgcattatttgggccttcattcagatgaacttgaggtgccattttgttgcatatgtacaggattatgggctggaaagcatttgttataaacctatagtgcacattttaactgccccctaaattacccttccctgggtttgttttccttggggtggtgtagattgtatgagtaagaagtattaattttttaaaagacaaatcaactttgaagacacaaaagttaattggaagaaataaaaactgtgaacgaagaa

SEQ ID NO: 3 - protein, 513 aa, Homo sapiens
  source 1..513: note = ZNF395 biomarker
MASVLSRRLGKRSLLGARVLGPSASEGPSAAPPSEPLLEGAAPQPFTTSDDTPCQEQPKEVLKAPSTSGLQQVAFQPGQKVYVWYGGQECTGLVEQHSWMEGQVTVWLLEQKLQVCCRVEEVWLAELQGPCPQAPPLEPGAQALAYRPVSRNIDVPKRKSDAVEMDEMMAAMVLTSLSCSPVVQSPPGTEANFSASRAACDPWKESGDISDSGSSTTSGHWSGSSGVSTPSPPHPQASPKYLGDAFGSPQTDHGFETDPDPFLLDEPAPRKRKNSVKVMYKCLWPNCGKVLRSIVGIKRHVKALHLGDTVDSDQFKREEDFYYTEVQLKEESAAAAAAAAAGTPVPGTPTSEPAPTPSMTGLPLSALPPPLHKAQSSGPEHPGPESSLPSGALSKSAPGSFWHIQADHAYQALPSFQIPVSPHIYTSVSWAAAPSAACSLSPVRSRSLSFSEPQQPAPAMKSHLIVTSPPRAQSGARKARGEAKKCRKVYGIEHRDQWCTACRWKKACQRFLD

SEQ ID NO: 4 - DNA, 4797 bp, Homo sapiens
  source 1..4797: mol_type = other DNA; note = ZNF395 biomarker
aagtgcgcatgtgcgcgaggagtcgctcgggcacttattgagcgccgactgtctacgggcggccgggggtgatgggcagaggcttcagtgtccccttcgcctccgcaggagaggagaggcagcagcatggcgagtgtcctgtcccgacgccttggaaagcggtccctcctgggagcccgggtgttgggacccagtgcctcggaggggccctcggctgccccaccctcggagccactgctagaaggggccgctccccagcctttcaccacctctgatgacaccccctgccaggagcagcccaaggaagtccttaaggctcccagcacctcgggccttcagcaggtggcctttcagcctgggcagaaggtttatgtgtggtacgggggtcaagagtgcacaggactggtggagcagcacagctggatggagggtcaggtgaccgtctggctgctggagcagaagctgcaggtctgctgcagggtggaggaggtgtggctggcagagctgcagggcccctgtccccaggcaccacccctggagcccggagcccaggccctggcctacaggcccgtctccaggaacatcgatgtcccaaagaggaagtcggacgcagtggaaatggatgagatgatggcggccatggtgctgacgtccctgtcctgcagccctgttgtacagagtcctcccgggaccgaggccaacttctctgcttcccgtgcggcctgcgacccatggaaggagagtggtgacatctcggacagcggcagcagcactaccagcggtcactggagtgggagcagtggtgtctccaccccctcgcccccccacccccaggccagccccaagtatttgggggatgcttttggttctccccaaactgatcatggctttgagaccgatcctgaccctttcctgctggacgaaccagctccacgaaaaagaaagaactctgtgaaggtgatgtacaagtgcctgtggccaaactgtggcaaagttctgcgctccattgtgggcatcaaacgacacgtcaaagccctccatctgggggacacagtggactctgatcagttcaagcgggaggaggatttctactacacagaggtgcagctgaaggaggaatctgctgctgctgctgctgctgctgccgcaggcaccccagtccctgggactcccacctccgagccagctcccacccccagcatgactggcctgcctctgtctgctcttccaccacctctgcacaaagcccagtcctccggcccagaacatcctggcccggagtcctccctgccctcaggggctctcagcaagtcagctcctgggtccttctggcacattcaggcagatcatgcataccaggctctgccatccttccagatcccagtctcaccacacatctacaccagtgtcagctgggctgctgccccctccgccgcctgctctctctctccggtccggagccggtcgctaagcttcagcgagccccagcagccagcacctgcgatgaaatctcatctgatcgtcacttctccaccccgggcccagagtggtgccaggaaagcccgaggggaggctaagaagtgccgcaaggtgtatggcatcgagcaccgggaccagtggtgcacggcctgccggtggaagaaggcctgccagcgctttctggactgagctgtgctgcaggttctactctgttcctggccctgccggcagccactgacaagaggccagtgtgtcaccagccctcagcagaaaccgaaagagaaagaacggaaacacggagtttgggctctgttggctaaggtgtaacacttaaagcaattttctcccattgtgcgaacattttattttttaaaaaaaagaaacaaaaatatttttccccctaaaataggagagagccaaaactgaccaaggctattcagcagtgaaccagtgaccaaagaattaattaccctccgtttcccacatccccactctctaggggattagcttgtgcgtgtcaaaagaaggaacagctcgttctgcttcctgctgagtcggtgaattctttgctttctaaactcttccagaaaggactgtgagcaagatgaatttacttttcttaaaaaaaaaaaaaaaaaaaaaaaaaaagagtttctggctgatgggtgactcagagtgcaggactgcctggccgtggggcagaggggtttgcccttctcggagggtacctcctgttccctgtctgagcatcctgcatggaagtcaaaggaaatccctttcttggtgacgacttaaatctgggttccctcagacattgggttgcaccccaacaaatattaaatggcttcttcttaaagcccagagaaagaggttttttaaaagactgtcgccaaatagctgagccaaaaggctgatcagaattcactttttggaatgtggcagttaaacactaccttgatcattctctcctctttcctcgaggaactcctggagggtttgagcgtctggaaactctctgctctgacccgaggaagcaccctcctgacgccgccttcctccggttattgaaaggacgcctcagaaatgctttgttttcttttacgatgtattcagaagcctttactgattaaagttttcttttatttgggtggccgggagagacccagggaggttctggaggttcctttctgtctcctggccccaccagggatttccccatttctgtttgctgcctgaaagcaggatgaggaaggccaaggagagtccttgcacccgtgagcgtcaggatgaggaaatgacaggaggaagacgtgggtttgggttagtggctgctggcgttttggcccttggtgtttctggagcctccagggatctaggggagcctgggctgcgtgcatgtcgataagcagagctgttcttggggagaaggagggaggtctcgggagtgtagcaccatgccaaccagccctgcgcgaagacagagtgagccacgcccggatggcagggcatgtttctgttttggtgtctcactttcctcccagcgtgacttatttggggattcctcagggcctactggaatgtgactgcccactgcccagctgcctcgggtacaagtcctggccctatgtcccagctgtcaggggctcagggaatcctacccagccacctgtcctgggatggagtgtcagcatccaccccttggttgtcatcgaggccgccctcccagtcctgggtgaagatatttgggccaccagggctcccttggccccttcacgtaggaaatagacacgtgctttttaatgcaggacactttgagtgttacaaaatctgtagacctggcagtagggtcatgatgttgggaagggtgtagtgccctaggttggtgacagaagggacagacacttgtgcacaggtgtctttggtgatggggtttttttttttataacttagtaaaaaaaaaaaaatgtatgtggaattctgtctcttggtaaagctcaaagccaggctagcctgaggtggcgcagggctctccttcctgtcccttcgatctccttgagaattaagagctggcagctgctgatggtgtttcccaacccccctcacttcccaagacaacccccagcttcaggtcctcatggggaggggagggcacgttcttgacacatgggaacttcgctcaggagggcctccccttcccctctccctcagagttttcactgccgtctcgtctttagaaagctgtttgaattccccccgcccccagtttggaccgtgtagatataactggatatacggatttttctctttgtgcaggcttcttatgccgttggtatacagggcaggaaagagaggaataaagggagagagcagtgtggaaaccacggtggttttgctttgttcttactaggttttggtgccaccttccctgcctgcgcttgtgccccctctcctccttggcactggcggcctccttgcctcccttccacccgtgctgccatcccgtgcctgtcgtgttggttcttcacacgtgctctgttctcggggttgttccattcatgccttcttggagggtgagggtggcttgggaaccgacccagtgatcatgcctactttcttctttgtatctccctccttcccagcccacccgggcagcagactctgatggaaggaaggtgccgtaggtgggcttttagaaactaacgggactggttttcaaagcagttatcttgggaaactgtttattccagcgatgtgacttttttcagaatatttcttggaatcatattcagagtctggggctgtgtgttgagcagccttaaggatgctagacactcatttagtgcccagggagtccagcgaatgacgtctgtggccaagcgaggtctcaggtgcaaagcaaaaggaccatttaaagtaaaatagcttggattcaatcatgtgacttttaaattggctcagaaagcaattttgtaatttcagagagtgttttgagccatggccacgttgtcattgtgagtctatagcttgactccttggagaacaatattcatttggttgtggagactgatttgctgggagaaatctgtcctgttactttctggtcatcccaggttctgacttttaccaggggcaaaaaaaaaaaaagcaagagggagataaatcccatctgtgagtttgtcttattggcgcctttttcctcagctgtcttccaagtattatttttactgttaaaaaattttttaaaaatgtgaaatgtaatgtttttacagcaacaatatgaaatatattttataaggaataaaatggtaccttgtctga

SEQ ID NO: 5 - protein, 940 aa, Homo sapiens
  source 1..940: note = XPC biomarker
MARKRAAGGEPRGRELRSQKSKAKSKARREEEEEDAFEDEKPPKKSLLSKVSQGKRKRGCSHPGGSADGPAKKKVAKVTVKSENLKVIKDEALSDGDDLRDFPSDLKKAHHLKRGATMNEDSNEEEEESENDWEEVEELSEPVLGDVRESTAFSRSLLPVKPVEIEIETPEQAKTRERSEKIKLEFETYLRRAMKRFNKGVHEDTHKVHLLCLLANGFYRNNICSQPDLHAIGLSIIPARFTRVLPRDVDTYYLSNLVKWFIGTFTVNAELSASEQDNLQTTLERRFAIYSARDDEELVHIFLLILRALQLLTRLVLSLQPIPLKSATAKGKKPSKERLTADPGGSSETSSQVLENHTKPKTSKGTKQEETFAKGTCRPSAKGKRNKGGRKKRSKPSSSEEDEGPGDKQEKATQRRPHGRERRVASRVSYKEESGSDEAGSGSDFELSSGEASDPSDEDSEPGPPKQRKAPAPQRTKAGSKSASRTHRGSHRKDPSLPAASSSSSSSKRGKKMCSDGEKAEKRSIAGIDQWLEVFCEQEEKWVCVDCVHGVVGQPLTCYKYATKPMTYVVGIDSDGWVRDVTQRYDPVWMTVTRKCRVDAEWWAETLRPYQSPFMDREKKEDLEFQAKHMDQPLPTAIGLYKNHPLYALKRHLLKYEAIYPETAAILGYCRGEAVYSRDCVHTLHSRDTWLKKARVVRLGEVPYKMVKGFSNRARKARLAEPQLREENDLGLFGYWQTEEYQPPVAVDGKVPRNEFGNVYLFLPSMMPIGCVQLNLPNLHRVARKLDIDCVQAITGFDFHGGYSHPVTDGYIVCEEFKDVLLTAWENEQAVIERKEKEKKEKRALGNWKLLAKGLLIRERLKRRYGPKSEAAAPHTDAGGGLSSDEEEGTSSQAEAARILAASWPQNREDEEKQKLKGGPKKTKREKKAAASHLFPFEQL

SEQ ID NO: 6 - DNA, 3650 bp, Homo sapiens
  source 1..3650: mol_type = other DNA; note = XPC biomarker
actcgcgaagtggaatttgcccagacaagcaacatggctcggaaacgcgcggccggcggggagccgcggggacgcgaactgcgcagccagaaatccaaggccaagagcaaggcccggcgtgaggaggaggaggaggatgcctttgaagatgagaaacccccaaagaagagccttctctccaaagtttcacaaggaaagaggaaaagaggctgcagtcatcctgggggttcagcagatggtccagcaaaaaagaaagtggccaaggtgactgttaaatctgaaaacctcaaggttataaaggatgaagccctcagcgatggggatgacctcagggactttccaagtgacctcaagaaggcacaccatctgaagagaggggctaccatgaatgaagacagcaatgaagaagaggaagaaagtgaaaatgattgggaagaggttgaagaacttagtgagcctgtgctgggtgacgtgagagaaagtacagccttctctcgatctcttctgcctgtgaagccagtggagatagagattgaaacgccagagcaggcgaagacaagagaaagaagtgaaaagataaaactggagtttgagacatatcttcggagggcgatgaaacgtttcaataaaggggtccatgaggacacacacaaggttcaccttctctgcctgctagcaaatggcttctatcgaaataacatctgcagccagccagatctgcatgctattggcctgtccatcatcccagcccgctttaccagagtgctgcctcgagatgtggacacctactacctctcaaacctggtgaagtggttcattggaacatttacagttaatgcagaactttcagccagtgaacaagataacctgcagactacattggaaaggagatttgctatttactctgctcgagatgatgaggaattggtccatatattcttactgattctccgggctctgcagctcttgacccggctggtattgtctctacagccaattcctctgaagtcagcaacagcaaagggaaagaaaccttccaaggaaagattgactgcggatccaggaggctcctcagaaacttccagccaagttctagaaaaccacaccaaaccaaagaccagcaaaggaaccaaacaagaggaaacctttgctaagggcacctgcaggccaagtgccaaagggaagaggaacaagggaggcagaaagaaacggagcaagccctcctccagcgaggaagatgagggcccaggagacaagcaggagaaggcaacccagcgacgtccgcatggccgggagcggcgggtggcctccagggtgtcttataaagaggagagtgggagtgatgaggctggcagcggctctgattttgagctctccagtggagaagcctctgatccctctgatgaggattccgaacctggccctccaaagcagaggaaagcccccgctcctcagaggacaaaggctgggtccaagagtgcctccaggacccatcgtgggagccatcgtaaggacccaagcttgccagcggcatcctcaagctcttcaagcagtaaaagaggcaagaaaatgtgcagcgatggtgagaaggcagaaaaaagaagcatagctggtatagaccagtggctagaggtgttctgtgagcaggaggaaaagtgggtatgtgtagactgtgtgcacggtgtggtgggccagcctctgacctgttacaagtacgccaccaagcccatgacctatgtggtgggcattgacagtgacggctgggtccgagatgtcacacagaggtacgacccagtctggatgacagtgacccgcaagtgccgggttgatgctgagtggtgggccgagaccttgagaccataccagagcccatttatggacagggagaagaaagaagacttggagtttcaggctaaacacatggaccagcctttgcccactgccattggcttatataagaaccaccctctgtatgccctgaagcggcatctcctgaaatatgaggccatctatcccgagacagctgccatccttgggtattgtcgtggagaagcggtctactccagggattgtgtgcacactctgcattccagggacacgtggctgaagaaagcaagagtggtgaggcttggagaagtaccctacaagatggtgaaaggcttttctaaccgtgctcggaaagcccgacttgctgagccccagctgcgggaagaaaatgacctgggcctgtttggctactggcagacagaggagtatcagcccccagtggccgtggacgggaaggtgccccggaacgagtttgggaatgtgtacctcttcctgcccagcatgatgcctattggctgtgtccagctgaacctgcccaatctacaccgcgtggcccgcaagctggacatcgactgtgtccaggccatcactggctttgatttccatggcggctactcccatcccgtgactgatggatacatcgtctgcgaggaattcaaagacgtgctcctgactgcctgggaaaatgagcaggcagtcattgaaaggaaggagaaggagaaaaaggagaagcgggctctagggaactggaagttgctggccaaaggtctgctcatcagggagaggctgaagcgtcgctacgggcccaagagtgaggcagcagctccccacacagatgcaggaggtggactctcttctgatgaagaggaggggaccagctctcaagcagaagcggccaggatactggctgcctcctggcctcaaaaccgagaagatgaagaaaagcagaagctgaagggtgggcccaagaagaccaaaagggaaaagaaagcagcagcttcccacctgttcccatttgagcagctgtgagctgagcgcccactagaggggcacccaccagttgctgctgccccactacaggccccacacctgccctgggcatgcccagcccctggtggtgggggcttctctgctgagaaggcaaactgaggcagcatgcacggaggcggggtcaggggagacgaggccaagctgaggaggtgctgcaggtcccgtctggctccagcccttgtcagattcacccagggtgaagccttcaaagctttttgctaccaaagcccactcaccctttgagctacagaacactttgctaggagatactcttctgcctcctagacctgttctttccatctttagaaacatcagtttttgtatggaagccaccgggagatttctggatggtggtgcatccgtgaatgcgctgatcgtttcttccagttagagtcttcatctgtccgacaagttcactcgcctcggttgcggacctaggaccatttctctgcaggccacttaccttcccctgagtcaggcttactaatgctgccctcactgcctctttgcagtaggggagagagcagagaagtacaggtcatctgctgggatctagttttccaagtaacattttgtggtgacagaagcctaaaaaaagctaaaatcaggaaagaaaaggaaaaatacgaattgaaaattaaggaaatgttagtaaaatagatgagtgttaaactagattgtattcattactagataaaatgtataaagctctctgtactaaggagaaatgacttttataacattttgagaaaataataaagcatttatctaaa

SEQ ID NO: 7 - protein, 350 aa, Homo sapiens
  source 1..350: note = MTHFD2 biomarker
MAATSLMSALAARLLQPAHSCSLRLRPFHLAAVRNEAVVISGRKLAQQIKQEVRQEVEEWVASGNKRPHLSVILVGENPASHSYVLNKTRAAAVVGINSETIMKPASISEEELLNLINKLNNDDNVDGLLVQLPLPEHIDERRICNAVSPDKDVDGFHVINVGRMCLDQYSMLPATPWGVWEIIKRTGIPTLGKNVVVAGRSKNVGMPIAMLLHTDGAHERPGGDATVTISHRYTPKEQLKKHTILADIVISAAGIPNLITADMIKEGAAVIDVGINRVHDPVTAKPKLVGDVDFEGVRQKAGYITPVPGGVGPMTVAMLMKNTIIAAKKVLRLEEREVLKSKELGVATN

SEQ ID NO: 8 - DNA, 4402 bp, Homo sapiens
  source 1..4402: mol_type = other DNA; note = MTHFD2 biomarker
tcccggcgcagtcaccggcgcggtctatggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccaaaccttttgagttcaactgatcaaaccaaaggaaaagtgttgctagagaaaattagggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtatggtatgaatttctgaaattctgtcttcctatgactcctagttgtgacctaaccagtttttttctcatttaatcaatgtagctattcctatattcagtaacacttacttctatagccttaaatagataattttttttcttctatttttttttttttttttgtagagatggggtcttgctatgttgcctgggctggtattgaactcctggcctcaagcaatcaatcctcttgcctcagcttcccagagggctgggattacaggcctgggccacccacacccaaacagaggtttaaagagctttggagaaaccagcctcgtcctccagtctctttgtttgtttgttttgagactgagtcttgctctgttgcccaggctggagtgcagtggcatgatctcgggtcactgcaacctctgcctctcgggttcaaacaattcttgtgcctcagcctcctgagtagctgggattacaggtgcatgccaccatgcctggctaatttttgtgtttttagtagagatggggtttcatcacattggccaggctggtctcaaactcctgacctcaagtgatccacctgcctcggcctcccaaaatgctaggattacaggcgtgagccactgcacctggcctgtcctacagtcttatatcacaacaggttgtgccaagctaggagcttaatctgtaaaatacttgaggcagggataggtttgtctgttttggcttttggctttcatgcgagcagaagtagctcctgctctaggcagcttgaggcagaaagagaagaaacaattgagttgacctacctccgtcctcgttcttctgccagctggcgttattcttttgatagttctctggagtttaaagaagtttagatagtttgctggagatgtttttgtaagtgacgtatagcttatgtctctaatccatgcctgcttacctagattattaaatgtggagcaagatgaccagacaggattaaattttattgaattacagttacaagtggaaaaaatgactgacttactgcatggagaagtattatgtcaacctgttaaaagctatggaaattaaaatgttcatgtatatacaaaatataaatgacatgtaaggtaaaattacagtgatcaatttacaacaaagaaattaacagtagttttccagcttgtttgattaatcaagcaggtttactcttgaatccatagcatttctacaatttaatttggacaaagttctattccagatcagccaggttttaaaactgaaatgtgttaagggtggattgttagcttccccaggattggtgtggatgataaatactttttttaaaaattaattattattgtttataagacatggattccagctgtctccctggctggagtgcagtggcatgattatagctcactgcagccttgaactcctggacttaaacgatcctcctgcatcagcctctgaaatggctgggactacaggtgtgtgccagcatgcctagctaatttttaaacatttgtttagagatgggtctcactatgttgcccaggctggtcttgacctcctgacctctgctgattccccccacctctgcctcccaagtagcgggattacaggtgtgagccactgtgtctgacaagaatttatacttaagcataggagatggttctggaaattctaagaaattctgctctcagtaagagtagaggtttggagctttacctcttggcagtatcccttggaagggagctctgagattaaatgtaatcaaacacttattgagagctttctatggaccaggcactacactagacgctgggaattacagaggcaattacctgtcctatactcaaggtgttaatgatctagtgtcatttttctttaaaaaagaaagcacctgcatttgtcatcttgagcaaatatagtaactctaacctcaaggttcaagtgatttactcaaggtttaaatcatagttgttttggagaaagtaagtgtcagatgcactgtggctgtcagggtcatagtaggcattaaatcaacatggttggtgttcattaaatgtggattgaagataaaatgactgatcttaagtttcatgaggaataggtttgcttcattgaaactggtttttatcttcccaaatagttttcaatcattcctttaaagtactaggttgattaatattgcaaagttgtaatgtaaaatgtgaccttacctctgtctaacctgtattgttcacatgaatgggagtttgactttgggaataaacattgaaaatttgcagggaggaccca

SEQ ID NO: 9 - protein, 239 aa, Homo sapiens
  source 1..239: note = PSME2 biomarker
MAKPCGVRLSGEARKQVEVFRQNLFQEAEEFLYRFLPQKIIYLNQLLQEDSLNVADLTSLRAPLDIPIPDPPPKDDEMETDKQEKKEVHKCGFLPGNEKVLSLLALVKPEVWTLKEKCILVITWIQHLIPKIEDGNDFGVAIQEKVLERVNAVKTKVEAFQTTISKYFSERGDAVAKASKETHVMDYRALVHERDEAAYGELRAMVLDLRAFYAELYHIISSNLEKIVNPKGEEKPSMY

SEQ ID NO: 10 - DNA, 793 bp, Homo sapiens
  source 1..793: mol_type = other DNA; note = PSME2 biomarker
gagaccagagatctagcgactgaagcagcatggccaagccgtgtggggtgcgcctgagcggggaagcccgcaaacaggtggaggtcttcagacagaatcttttccaggaggctgaggaattcctctacagattcttgccacagaaaatcatatacctgaatcagctcttgcaagaggactccctcaatgtggctgacttgacttccctccgggccccactggacatccccatcccagaccctccacccaaggatgatgagatggaaacagataagcaggagaagaaagaagtccataagtgtggatttctccctgggaatgagaaagtcctgtccctgcttgccctggttaagccagaagtctggactctcaaagagaaatgcattctggtgattacatggatccaacacctgatccccaagattgaagatggaaatgattttggggtagcaatccaggagaaggtgctggagagggtgaatgccgtcaagaccaaagtggaagctttccagacaaccatttccaagtacttctcagaacgtggggatgctgtggccaaggcctccaaggagactcatgtaatggattaccgggccttggtgcatgagcgagatgaggcagcctatggggagctcagggccatggtgctggacctgagggccttctatgctgagctttatcatatcatcagcagcaacctggagaaaattgtcaacccaaagggtgaagaaaagccatctatgtactgaacccgggactagaaggaaaataaatgatctatatgttgtgtgga

SEQ ID NO: 11 - protein, 1132 aa, Homo sapiens
  source 1..1132: note = JAK2 biomarker
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 12 - DNA, 7023 bp, Homo sapiens
  source 1..7023: mol_type = other DNA; note = JAK2 biomarker
agaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgtcccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctggggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtcagttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaattaagtataaggggttgttcgttgttgtcatttgttatagtgctactccactttagacaccatagctaaaataaaatatggtgggttttgtgtgtgtgtgtgtgtgtgtgtgtgtgtgtgtgtgtgtgttatttatacaaaacttaaaatacttgctgttttgattaaaaagaaaatagtttcttactttatttttactggtatgttctacttttttgaaagttgtactgaagacttctgattttgggttgaagggaaggaaaaggaagaaatgttttttacattcattattatacttaaagcatttttaaagcattttaatagttctggatgcagaaatcatctaaaatgacagtgaattaggttttaaaaagattttagatttttttgaaagtttaatttttatttgtaaagactcctcaaggatttgtatatgcaacacagtaaggagatcttccattttactacctttcaagtgaaaaatagcctatcatacaatatgcttgatttcagattttcatactaaaacttaactacatacttaaaagtaggttcttatcaagggtctctaacattgctttttaaaacaagatgtgaactaacttttcttaaacatttttttaaatgcttcatcttttagttttatataaagaatcccacatgtacattcttgtttttagaatggggtgactaccttattataaaattccaagtttccaagagacttcttttcattgaggcttcgtaaagttttccattttgattctgactacacataaaaataagataaccctgtagttattaagttggttctgtacaagaaacaggtaagtaattattgtaccagttaatgccaaaatatttttcacgttaatattcttcagaaacaagggtaaaggtattcttagaattatgtaattctgtaactattccagtatattaagttatacaatctttatataaatgactttttccatgggtacttgtttggaaaatagtcactttttcacgctatttatatatgctgccagtaacactataatttgctatggaagagtgttcttttaacctaaggcttctagtttacagtcaagtgtaattttcatcacaactataacttctggtatttaaattttatttaaacagctacaaaagagttagcaaatacctacagttctgtatctatagagccaatcttgatggtgggtgtggcattatgtgctcactttattgagcctatgttaatttctttagcatgctccccctaaattgaaatagtgatgtagtaaatattcagaagcgattttcttttgcatttttacctaaccaaggaaacgggccacacaccttggtttagggatgttgtgatagcttaccttccagtttttaagaaatgcttcctacaactgctgtcaaccactgtattgtctttaatgaacactgttgtatcccatcctaattcttgtactgaaattatttctcatgaaagtttctctaatatttctaatgaaagtttctctaatttgggggcataatgtactaagaatcagtttgctgtatattagaataaatagtaacagtaagtcagcaggattatccaaacaaaagactaggttttatgagataagcttgatttaagaaaaaaacaattaaagtatgaatatcagaaatactgtgtgtttactctcagattttagttggttggatttaatatcaagataactagctgctaagcgtttcataattctcacagtgatattagatttcaaaatgacactgagagaactgaaaaactacatcagtcaaattcatgtatgtatatcatatagcctttaactttttacattaatcagattcttagtaaaatgcagactgtatacctaaatattaaaatatttacttttataatcttaccttttatttcaatataaataaaattcttcttaggttaaaaaa

SEQ ID NO: 13 - protein, 1204 aa, Homo sapiens
  source 1..1204: note = INTS2 biomarke
MKDQQTVIMTECTSLQFVSPFAFEAMQKVDVVCLASLSDPELRLLLPCLVRMALCAPADQSQSWAQDKKLILRLLSGVEAVNSIVALLSVDFHALEQDASKEQQLRHKLGGGSGESILVSQLQHGLTLEFEHSDSPRRLRLVLSELLAIMNKVSESNGEFFFKSSELFESPVYLEEAADVLCILQAELPSLLPIVDVAEALLHVRNGAWFLCLLVANVPDSFNEVCRGLIKNGERQDEESLGGRRRTDALRFLCKMNPSQALKVRGMVVEECHLPGLGVALTLDHTKNEACEDGVSDLVCFVSGLLLGTNAKVRTWFGTFIRNGQQRKRETSSSVLWQMRRQLLLELMGILPTVRSTRIVEEADVDMEPNVSVYSGLKEEHVVKASALLRLYCALMGIAGLKPTEEEAEQLLQLMTSRPPATPAGVRFVSLSFCMLLAFSTLVSTPEQEQLMVVWLSWMIKEEAYFESTSGVSASFGEMLLLVAMYFHSNQLSAIIDLVCSTLGMKIVIKPSSLSRMKTIFTQEIFTEQVVTAHAVRVPVTSNLSANITGFLPIHCIYQLLRSRSFTKHKVSIKDWIYRQLCETSTPLHPQLLPLIDVYINSILTPASKSNPEATNQPVTEQEILNIFQGVIGGDNIRLNQRFSITAQLLVLYYILSYEEALLANTKTLAAMQRKPKSYSSSLMDQIPIKFLIRQAQGLQQELGGLHSALLRLLATNYPHLCIVDDWICEEEITGTDALLRRMLLTNNAKNHSPKQLQEAFSAVPVNNTQVMQIIEHLTLLSASELIPYAEVLTSNMSQLLNSGVPRRILQTVNKLWMVLNTVMPRRLWVMTVNALQPSIKFVRQQKYTQNDLMIDPLIVLRCDQRVHRCPPLMDITLHMLNGYLLASKAYLSAHLKETEQDRPSQNNTIGLVGQTDAPEVTREELKNALLAAQDSAAVQILLEICLPTEEEKANGVNPDSLLRNVQSVITTSAPNKGMEEGEDNLLCNLREVQCLICCLLHQMYIADPNIAKLVHFQGYPCELLPLTVAGIPSMHICLDFIPELIAQPELEKQIFAIQLLSHLCIQYALPKSLSVARLAVNVMGTLLTVLTQAKRYAFFMPTLPSLVSFCRAFPPLYEDIMSLLIQIGQVCASDVATQTRDIDPIITRLQQIKEKPSGWSQICKDSSYKNGSRDTGSMDPDVQLCHCIERTVIEIINMSVSGI

SEQ ID NO: 14 - DNA, 5847 bp, Homo sapiens
  source 1..5847: mol_type = other DNA; note = INTS2 biomarker
agaccagtacggtggccgacgggagtcagacgctggggatgaatgaaggatcaacaaacagtaataatgactgaatgtacaagtcttcagtttgtcagcccttttgcttttgaggcaatgcagaaggtggatgttgtttgcctggcatctttaagtgatccagaattaagacttcttctgccctgtttggtacggatggcactttgtgcacctgctgaccagagccaaagctgggctcaggataagaaactcatccttcgccttctttctggagtggaagctgtcaactccattgttgcattgttgtccgtggactttcatgctttagaacaagatgccagcaaagaacagcagcttaggcataaacttggaggaggcagtggagagagcatcctggtatcacagcttcagcatggactgacgttagagtttgaacacagtgattcacctcgtcgattgcgtcttgtgcttagtgaactgttggcaattatgaacaaggtgtctgagtccaacggagaattttttttcaagtcttctgaactttttgagagtccagtatatttggaggaagctgcagatgtactttgtattttacaagcagagctcccttccttgctccctatagttgatgtagctgaagctttgctacatgttagaaatggtgcctggttcttgtgtctcttggtggccaatgttcctgatagttttaatgaagtttgtaggggcctgataaaaaatggagaacgacaagatgaagaaagtcttggaggaaggcgcaggacagatgccttacgcttcttgtgtaaaatgaatccttctcaggccctcaaggtccgaggcatggtggtggaagaatgtcacttgccaggccttggtgtggctttgacattggatcatactaaaaatgaagcttgtgaggatggagtgagtgacttggtttgttttgtaagtggtttgcttcttggaacaaatgcgaaagtccggacttggtttggaacttttatccgaaatggacagcagagaaaaagagagaccagcagttctgtcctttggcagatgagaaggcagcttcttctggagttgatgggcattcttcccacagtaagaagcacccgaattgtggaagaagctgatgtggatatggagcccaatgtgtctgtgtattcggggctgaaagaagagcatgttgtgaaagccagtgcactcttacgtctgtactgtgctttgatggggatcgctggactcaaaccaactgaagaagaagctgagcaattactgcagttgatgacgagccgtcctcctgctacgccagctggggttcgctttgtttcactttccttttgtatgctactggccttttctacacttgtcagtacacctgaacaggagcagctgatggtggtgtggctaagttggatgataaaagaagaagcgtattttgagagtacttcaggcgtctctgcttcttttggggagatgttattattggtggctatgtactttcacagcaaccagcttagtgctatcattgacttggtctgttccactttggggatgaagattgtaattaagccaagctccttgagcaggatgaagacaatcttcacacaggaaatttttactgagcaggttgtcacagctcatgcagttcgggtccctgtcaccagcaacctgagtgccaacattactggatttttgcctattcattgtatttaccagcttctcaggagccgttcctttaccaagcacaaagtgtcaataaaagattggatttatagacagctgtgtgaaacctctactccacttcatcctcaattacttcctttgattgatgtgtacataaattctatacttactcctgcgtcgaaatctaatccagaagccacaaatcagccagtcacagaacaggagatactcaatattttccaaggagtcattgggggtgacaacatccgccttaatcagcgtttcagtatcacagcacagcttttggtgctctactatatactgtcttatgaagaggctcttctagcaaacacgaagactttagctgccatgcaaagaaagcccaaatcatattcttcttctttaatggatcagattcctatcaaattccttattcgacaggctcaagggctgcagcaggagttgggagggttgcattcagctttactacgtctccttgctactaactacccacatttatgtattgtggatgactggatttgtgaagaagaaatcacagggactgatgccctgctacggcgaatgctcctgactaataatgctaaaaatcattctcccaaacaactccaagaagcattttcagctgtcccagtaaataacacacaagtgatgcagattatagaacacttgactctactctctgccagtgaacttataccatatgcggaagtgttaacatccaatatgagccagctattgaattcaggggttccacggagaattctgcaaacagtcaataaactatggatggttcttaatactgtgatgcctagaaggctatgggtaatgacggttaatgcacttcagccttcaataaagtttgtacgacaacaaaagtatactcagaatgacctgatgatagatcctctcattgtcctaaggtgtgatcagagggttcacagatgccccccactgatggatattaccctacacatgttgaatggatatcttcttgcatctaaagcctaccttagtgctcatctgaaggaaacagagcaagataggccttcccagaataatacaattggtttagttggacaaactgatgctccggaagttaccagggaagaattgaaaaatgcattactggccgctcaggatagtgcagctgtccagattctcttagagatttgcctacctactgaagaggagaaagcaaatggtgtcaatccagatagcttgttaagaaatgttcaaagtgttattaccaccagcgctccaaataagggaatggaggaaggagaagacaatttgctctgtaaccttcgagaagttcagtgccttatctgttgtctcttgcaccaaatgtacattgcagatcccaacattgctaagcttgttcactttcagggttatccatgtgaacttttgcctctgacggtcgcaggtattccatctatgcacatctgtctagatttcatacctgagcttattgcacagccagaacttgagaaacagatatttgctatccagttgctttctcacttgtgtatacaatatgcattaccaaagtcacttagtgtggctcgtttagctgtcaatgtcatgggaactttgttaacagttttaacacaggctaagcggtatgctttttttatgccaactctgccaagtttggtctctttttgtcgagcatttcctccattgtatgaggatattatgtctttgctgatccaaatagggcaagtttgtgcctctgatgttgccactcagacaagagacattgatccaattattacacgtcttcaacaaataaaggagaaaccaagtggatggtctcaaatctgtaaagattcatcttataaaaatggatccagggacactggaagcatggatcctgatgtacagctctgtcactgtattgaaagaacagtaattgaaataataaatatgagtgttagtggaatttaaaacaaaatttaaaacaacaaaaagttgtttgctgcatatacccaacatgaatctgcatattagtaacaactctaaactgaatgggaacagtaaagtattgtcttggaatcactaaaacaattcaattcaacatgagtatagtttagaactttatgagaattatgcttgcttgtttctgattggcacatctttggatctactttgctgatatgtttctattgtagcagctgagctttttttttttccactgggaacacatgtaagaaactcattattggaaagggaatttggccttgtatttagcttttgaagtgaagactgccatgcctttaatttcttataaaaatgagtctgtgggtagccctagtgtttattttaactgtgagcttgtaacagaatgtgacaaagatgcaaagatgggagaggaaaaaagggtaaagggaaaggagaattaaggaaataataggagttaaaaacacaagtagaaatctcaaagatttgcagtgcaagtaatagtaatgcaagttggaattctagttctcaagaaagagtattgagaagacttttaaaaaggcaagtagcttttgtaaatgatttctgtggaaatacagatgaggatttaaagatttcacatatttgcttcaatttttattaatatatgaagccatatgtttaaagagatacttgaataatttggaattttaagatactggtgtaaaagtgtttacagaaacatctttgttcaaagaagaacctgagagatctcatttagttttatgttttaaatttatttttataatgctttattaacttacctaatgctcagaggggggaaatatgtatcaaattaaatgaaggtagagcaataaaacccactggattaaagagctcttggtttgtcatcaggattataattcatatcttactttgagaagatctttgagtaagaaaatgcagtgtttgaacctgaggaaaagttaaagtgtagaaaatattgtcttgccgaaggattttgcagtcctctgtcagtaacttccattgattaggcagacatattcaggtaaaccctaatcattaaaaaaaaattatcaatgtagaaagtaattcccttttttctctctgagatatacctcaatcacacacttccccacccccacttgaaacagacctcttcacttgtgttttttttttttttttcctgaggtggagtcttcccctgttgcccaggctggagtgcagtgggatgatcttggctcactgcaacttctgccacctgggttcaagggattctcgtgcctcaacctcctgagtagctgggactgcaggcacgcgccacctgtatttttgtatttttagtaaagacgggggtttgccatgttgcccagactggttttgaactcctggcctcaggtgatctgcccaccttggcctcccaaagtgctgggattacaggtgtgagccaccgcacctggccagaccgcttcacttgtaaaagaaattaggctaataagaaggtgtagtttttgagaaatgaaatttaactttagccttttcactagtaaatagtcacatctcattttcttcctttgtaaaatggggttactactggccctacctcatattctatgagaatgagtttgtagctgtttcaaatcatgaagtgcatagtatcacatgtgatagaatatttataactttttattagatgcttaatgttcaattaagtaattttgatgtgaaaaataaaagtaataaaagtatcttaaaaatagcataagaattttcatatttttaaacaaggcagttttgtagtcccttaagattaaatacaactgctccttttttttttaaactgaggccttgcgatattttgtgtgaatagatatgccctaggagttcagaaaaagttaaaagtatgttttctaattaaatgcagtgcacattcctggatcaatattcaaagactggtcataacctgctgtgttaaaataatcacatatgctctttttcatcagatttgttgatgatgtaaataaaatgtgtaaatatattagtaaatgttaatattcatgtattttaagttaaggttataaaatttgtcacaatgtgtttttttattcaagtgaaaacagatgtgtgcagctattttgaatattggtttataaacattcatattctttatcaaacgaa

SEQ ID NO: 15 - protein, 277 aa, Homo sapiens
  source 1..277: note = PSMB7 biomarker
MAAVSVYAPPVGGFSFDNCRRNAVLEADFAKRGYKLPKVRKTGTTIAGVVYKDGIVLGADTRATEGMVVADKNCSKIHFISPNIYCCGAGTAADTDMTTQLISSNLELHSLSTGRLPRVVTANRMLKQMLFRYQGYIGAALVLGGVDVTGPHLYSIYPHGSTDKLPYVTMGSGSLAAMAVFEDKFRPDMEEEEAKNLVSEAIAAGIFNDLGSGSNIDLCVISKNKLDFLRPYTVPNKKGTRLGRYRCEKGTTAVLTEKITPLEIEVLEETVQTMDTS

SEQ ID NO: 16 - DNA, 984 bp, Homo sapiens
  source 1..984: mol_type = other DNA; note = PSMB7 biomarker
actgctttcttgggaagatggcggctgtgtcggtgtatgctccaccagttggaggcttctcttttgataactgccgcaggaatgccgtcttggaagccgattttgcaaagaggggatacaagcttccaaaggtccggaaaactggcacgaccatcgctggggtggtctataaggatggcatagttcttggagcagatacaagagcaactgaagggatggttgttgctgacaagaactgttcaaaaatacacttcatatctcctaatatttattgttgtggtgctgggacagctgcagacacagacatgacaacccagctcatttcttccaacctggagctccactccctctccactggccgtcttcccagagttgtgacagccaatcggatgctgaagcagatgcttttcaggtatcaaggttacattggtgcagccctagttttagggggagtagatgttactggacctcacctctacagcatctatcctcatggatcaactgataagttgccttatgtcaccatgggttctggctccttggcagcaatggctgtatttgaagataagtttaggccagacatggaggaggaggaagccaagaatctggtgagcgaagccatcgcagctggcatcttcaacgacctgggctccggaagcaacattgacctctgcgtcatcagcaagaacaagctggattttctccgcccatacacagtgcccaacaagaaggggaccaggcttggccggtacaggtgtgagaaagggactactgcagtcctcactgagaaaatcactcctctggagattgaggtgctggaagaaacagtccaaacaatggacacttcctgaatggcatcagtgggtggctggccgcggttctggaaggtggtgagcattgaggcccagtaagacactcatgtggctagtgtttgccgaatgaaactcaactcaataaaaaacaaaaaccaaattgggcagctga

SEQ ID NO: 17 - protein, 266 aa, Homo sapiens
  source 1..266: note = RBM7 biomarker
MGAAAAEADRTLFVGNLETKVTEELLFELFHQAGPVIKVKIPKDKDGKPKQFAFVNFKHEVSVPYAMNLLNGIKLYGRPIKIQFRSGSSHAPQDVSLSYPQHHVGNSSPTSTSPSRYERTMDNMTSSAQIIQRSFSSPENFQRQAVMNSALRQMSYGGKFGSSPLDQSGFSPSVQSHSHSFNQSSSSQWRQGTPSSQRKVRMNSYPYLADRHYSREQRYTDHGSDHHYRGKRDDFFYEDRNHDDWSHDYDNRRDSSRDGKWRSSRH

SEQ ID NO: 18 - DNA, 3607 bp, Homo sapiens
  source 1..3607: mol_type = other DNA; note = RBM7 biomarker
gctgagatgggggcggcggcggcggaagcggatcgcactctctttgtgggcaaccttgaaacgaaagtgaccgaggagctccttttcgagcttttccaccaggctgggccagtaataaaggtgaaaattccaaaagataaggatggtaaaccaaagcagtttgcgtttgtgaatttcaaacatgaagtgtctgttccttatgcaatgaatctacttaatggaatcaaactttatggaaggcctatcaaaattcaatttagatcaggaagtagtcatgccccacaagatgtcagtttgtcatatccccaacatcatgttggaaattcaagccctacctccacatctcctagcaggtacgaaaggactatggataacatgacttcatcagcacagataattcagagatctttctcttctccagaaaattttcagagacaagcagtgatgaacagtgctttgagacaaatgtcatatggtggaaaatttggttcttcacctctggatcaatcaggattttcaccatcagttcaatcacacagtcatagtttcaatcagtcttcaagctcccagtggcgccaaggtacaccatcatcacagcgtaaagtcagaatgaattcttatccctacctagcagatagacattatagccgggaacagcgttacactgatcatgggtctgaccatcattacagaggaaagagagatgatttcttctatgaagacaggaatcatgatgactggagccatgactatgataacagaagagacagtagtagagatggaaaatggcgctcatctcgacactaacacatgttaaaaggacattgtttttatagggtcattttaggccctttgactaagttgatatggaaatattttgttgaaaaactgtacagagcagctttacaagttgtcacatttctttataaatttttttaaagctacagtttaatacaaaatgaattgcggttttattacattaataacctttcacctcagggttttatgaagaggaaagggttttatgcaaaagaaagtgctacaattcctaatcattttagacactttaggagggggtgaagttgtatgataaagcagatattttaattatttgttatctttttgtattgcaagaaatttcttgctagtgaatcaagaaaacatccaggttgacagtctaaaatggctactggtattttagttaattcaaaaatgaaacttttcagtgattcactttactaacattctatttgagaaggcttattggtaaagtttggggataaaggcattgcttaacttcttatataatttaggtataaattctgtgacatgctcttgagctttaccctagttgaacatacatgtgtagatttacacatactgtttcattctaaaatttagaaattgttcattaaatcccatttgaggtataagtcactcaggaagttaaaatatctctacacgtatatttttacattaaaaatacagtgttagcataaatccccttttcaggaagaacaaaaatgtcagtgcatagttagataaaatggtaaaatgttttactgaaagcatacttttttggaaaatagattcatgaagcctttaagtgctgcttctgtcagtcaaacgttaaaaactttaacattttcaaagtgcccagactgtgtacaaagacacatgtaatggagattgtacaggttgtttttttgtttgaacctttgaaagagtttaatcttaacgttttctaattttaaaattttaaaatcttgtttaacaaaagcttgtattaagatactgttttcatttcattacagaattgtttataaaagttcatttgttgaaaaataaggatcctttttaataccacagcatttgtactgttcctttttaatatactgaaaatataaaaggaagggtgtgtgttatttttttttttttatgtcactgacttcagagacattgtacacaaagaattaacatacttttattcactgattgcctgctgtttagaatatcagttctttaattttgagcatcctgaaatacatcttttgtacatatagagcatgatgtttcacaatcagattttcagtgacccctcgattatagcttggagtactttagttaccctcagtggtctctaggagttaaagtacacttagatttcctgtttataaatgaattcagtttttctgtttctaagtaaatgtagctattcaactactcactatagaattaagataaacaatttgtttgcttaagcattttcacagaatttgtttccaatgagattattatttttgggtatcttatccatatgaaaacttagtaggtttgtagatgttaacgaagattaatactgcatgtttgtttttgttttttttttgagacagagtcttgctacgacacccaggctagagtgcaatggcgggatctcaggtcactgcaacctccatatcccgggttcaagtgattctcctgccttagcctcccgagtagctgggattacaggcacccaccaccacgcctggctagtttttgtatttttagtagagatggggtttcaccacattggccaggctggtctcaaactcctgacctcaagtgatccacctaccttggcctaccgaggtgctggaattacaggtgtgagccaccgcgcctggcctaatactgctttattacaacgttatctgtgggtcggatccttttatattggttaacagatgaccctgactcagaataatctttttcaatggctttttgagggaagcttgtgaagttctggtgaatcttctttttcacttcactttcagtgagctgaaagtaaccaaactaaatacatgtattgtgtaaagggacaggacaagacagccttaaaaaattgaatatagttggtgagacaactcagaagtacaggtttgagcatcccttattcaaaatgcttgagaagtgttttgggttctggaatatttgcattaatgcttgccagttgagcatcccaggtccggaaatccacagtgctccaatgagcctttcccctgagtgtcacatctgtattggcactcaaaaagtttcatattttggagcatttcagatttcagatttgggatgcttcatctatattgacagctgcaagaacagaaaggaagaagagattatttttgtgggagaacagtttctcccatagtgtttcctgtggaatgctagtgtctcataaagtcttctaaaaaaaagaaaaaaaaaatcaaatgtttggaagccattttgtgttactgtgtgactttcttttactcaaaaacagcaccataaaatttctgacaagtactataggtaaagaaatccctttatacttaacctagtattttctacctttccccatctaaaataaaatttttataccactttctaattatgttgtgtgtgcccactttccccatacctactgctaccatcttagagtaggcaattttctcaagaggattattccaagaacaccctaatgttttcctgcttccagtcttctttcctcaaatttattccacactcttaagaattgaagatgtaaatctgtcactcgcctgaataaagcttcagtggcctctga

SEQ ID NO: 19 - protein, 635 aa, Homo sapiens
  source 1..635: note = SLC6A8 biomarker
MAKKSAENGIYSVSGDEKKGPLIAPGPDGAPAKGDGPVGLGTPGGRLAVPPRETWTRQMDFIMSCVGFAVGLGNVWRFPYLCYKNGGGVFLIPYVLIALVGGIPIFFLEISLGQFMKAGSINVWNICPLFKGLGYASMVIVFYCNTYYIMVLAWGFYYLVKSFTTTLPWATCGHTWNTPDCVEIFRHEDCANASLANLTCDQLADRRSPVIEFWENKVLRLSGGLEVPGALNWEVTLCLLACWVLVYFCVWKGVKSTGKIVYFTATFPYVVLVVLLVRGVLLPGALDGIIYYLKPDWSKLGSPQVWIDAGTQIFFSYAIGLGALTALGSYNRFNNNCYKDAIILALINSGTSFFAGFVVFSILGFMAAEQGVHISKVAESGPGLAFIAYPRAVTLMPVAPLWAALFFFMLLLLGLDSQFVGVEGFITGLLDLLPASYYFRFQREISVALCCALCFVIDLSMVTDGGMYVFQLFDYYSASGTTLLWQAFWECVVVAWVYGADRFMDDIACMIGYRPCPWMKWCWSFFTPLVCMGIFIFNVVYYEPLVYNNTYVYPWWGEAMGWAFALSSMLCVPLHLLGCLLRAKGTMAERWQHLTQPIWGLHHLEYRAQDADVRGLTTLTPVSESSKVVVVESVM

SEQ ID NO: 20 - DNA, 3931 bp, Homo sapiens
  source 1..3931: mol_type = other DNA; note = SLC6A8 biomarker
gaagagagggatagtcggagcgaggtggcgagtcgctgagcccgccgcggccccgagagcggctgcagccgccgccgccgggaaggagagggcgaggcgcgcccgagccgccgccgccgccgccaccgccgccgccgccaccaccgccaccggagtcgcgggccagccgggcagcctccgcgggccccggccggggcggggggcgcgggccacaggcccctgctccggccgccgcttgcagaccgcgggcgccgatgtcgcccgcgccccgctaggctgagcctcgggtcgggcgaggagccgccgcagccgccgccgcccgagccgcgggcaggagcctcgggagccgccgccgccgccgccgccgcccggccgggccccgccgccgcccgcgcgcccccgggcccccgacacacatgagattcttcaggctcactttcaagtgcttcgtggactgcttctgactgcgccgcccgcgccccgcaccccgccgcccgcccgccgccccgtcccccggcccggccgccccccggcccccggccggcccgcgccctcggggccctccccggtgccgccggtgccccccgcctgaccgccgccccccgtgaggcgccgcgaccccggcccggccgtgcggcccgccgaggccatggcgaagaagagcgccgagaacggcatctatagcgtgtccggcgacgagaagaagggccccctcatcgcgcccgggcccgacggggccccggccaagggcgacggccccgtgggcctggggacacccggcggccgcctggccgtgccgccgcgcgagacctggacgcgccagatggacttcatcatgtcgtgcgtgggcttcgccgtgggcttgggcaacgtgtggcgcttcccctacctgtgctacaagaacggcggaggtgtgttccttattccctacgtcctgatcgccctggttggaggaatccccattttcttcttagagatctcgctgggccagttcatgaaggccggcagcatcaatgtctggaacatctgtcccctgttcaaaggcctgggctacgcctccatggtgatcgtcttctactgcaacacctactacatcatggtgctggcctggggcttctattacctggtcaagtcctttaccaccacgctgccctgggccacatgtggccacacctggaacactcccgactgcgtggagatcttccgccatgaagactgtgccaatgccagcctggccaacctcacctgtgaccagcttgctgaccgccggtcccctgtcatcgagttctgggagaacaaagtcttgaggctgtctgggggactggaggtgccaggggccctcaactgggaggtgaccctttgtctgctggcctgctgggtgctggtctacttctgtgtctggaagggggtcaaatccacgggaaagatcgtgtacttcactgctacattcccctacgtggtcctggtcgtgctgctggtgcgtggagtgctgctgcctggcgccctggatggcatcatttactatctcaagcctgactggtcaaagctggggtcccctcaggtgtggatagatgcggggacccagattttcttttcttacgccattggcctgggggccctcacagccctgggcagctacaaccgcttcaacaacaactgctacaaggacgccatcatcctggctctcatcaacagtgggaccagcttctttgctggcttcgtggtcttctccatcctgggcttcatggctgcagagcagggcgtgcacatctccaaggtggcagagtcagggccgggcctggccttcatcgcctacccgcgggctgtcacgctgatgccagtggccccactctgggctgccctgttcttcttcatgctgttgctgcttggtctcgacagccagtttgtaggtgtggagggcttcatcaccggcctcctcgacctcctcccggcctcctactacttccgtttccaaagggagatctctgtggccctctgttgtgccctctgctttgtcatcgatctctccatggtgactgatggcgggatgtacgtcttccagctgtttgactactactcggccagcggcaccaccctgctctggcaggccttttgggagtgcgtggtggtggcctgggtgtacggagctgaccgcttcatggacgacattgcctgtatgatcgggtaccgaccttgcccctggatgaaatggtgctggtccttcttcaccccgctggtctgcatgggcatcttcatcttcaacgttgtgtactacgagccgctggtctacaacaacacctacgtgtacccgtggtggggtgaggccatgggctgggccttcgccctgtcctccatgctgtgcgtgccgctgcacctcctgggctgcctcctcagggccaagggcaccatggctgagcgctggcagcacctgacccagcccatctggggcctccaccacttggagtaccgagctcaggacgcagatgtcaggggcctgaccaccctgaccccagtgtccgagagcagcaaggtcgtcgtggtggagagtgtcatgtgacaactcagctcacatcaccagctcacctctggtagccatagcagcccctgcttcagccccaccgcacccctccagggggcctgcctttccctgacacttttggggtctgcctgggggaggaggggagaaagcaccatgagtgctcactaaaacaactttttccatttttaataaaacgccaaaaatatcacaacccaccaaaaatagatgcctctccccctccagccctagccgagctggtcctaggccccgcctagtgccccacccccacccacagtgctgcactcctcctgcccctgccacgcccaccccctgcccacctctccaggctctgctctgcagcacacccgtgggtgacccctcaccccagaagcagcagtggcagcttgggaaatgtgaggaagggaaggagggagagacgggagggaggagagagaggagaagggaggcaggggaggggcagcagaaccaaggcaaatatttcagctgggctatacccctctccccatccctgttatagaagcttagagagccagccagcaatggaaccttctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggtgtctgggctgctaacctggcctgctcaggcttcccaccctgtgcggggcacacccccaggaagggaccctggacacggctcccacgtccaggcttaaggtggatgcacttcccgcacctccagtcttctgtgtagcagctttaacccacgtttgtctgtcacgtccagtcccgagacggctgagtgaccccaagaaaggcttccccgacacccagacagaggctgcagggctggggctgggtgagggtggcgggcctgcggggacattctactgtgctaaaaagccactgcagacatagcaataaaaacatgtcattttccaaa

SEQ ID NO: 21 - protein, 453 aa, Homo sapiens
  source 1..453: note = SERINC1 biomarker
MGSVLGLCSMASWIPCLCGSAPCLLCRCCPSGNNSTVTRLIYALFLLVGVCVACVMLIPGMEEQLNKIPGFCENEKGVVPCNILVGYKAVYRLCFGLAMFYLLLSLLMIKVKSSSDPRAAVHNGFWFFKFAAAIAIIIGAFFIPEGTFTTVWFYVGMAGAFCFILIQLVLLIDFAHSWNESWVEKMEEGNSRCWYAALLSATALNYLLSLVAIVLFFVYYTHPASCSENKAFISVNMLLCVGASVMSILPKIQESQPRSGLLQSSVITVYTMYLTWSAMTNEPETNCNPSLLSIIGYNTTSTVPKEGQSVQWWHAQGIIGLILFLLCVFYSSIRTSNNSQVNKLTLTSDESTLIEDGGARSDGSLEDGDDVHRAVDNERDGVTYSYSFFHFMLFLASLYIMMTLTNWYRYEPSREMKSQWTAVWVKISSSWIGIVLYVWTLVAPLVLTNRDFD

SEQ ID NO: 22 - DNA, 3125 bp, Homo sapiens
  source 1..3125: mol_type = genomic DNA; note = SERINC1 biomarker
aatctcggaaaggcgagaaagaagctgtctccatcttgtctgtatccgctgctcttgtgacgttgtggagatggggagcgtcctggggctgtgctccatggcgagctggataccatgtttgtgtggaagtgccccgtgtttgctatgccgatgctgtcctagtggaaacaactccactgtaactagattgatctatgcacttttcttgcttgttggagtatgtgtagcttgtgtaatgttgataccaggaatggaagaacaactgaataagattcctggattttgtgagaatgagaaaggtgttgtcccttgtaacattttggttggctataaagctgtatatcgtttgtgctttggtttggctatgttctatcttcttctctctttactaatgatcaaagtgaagagtagcagtgatcctagagctgcagtgcacaatggattttggttctttaaatttgctgcagcaattgcaattattattggggcattcttcattccagaaggaacttttacaactgtgtggttttatgtaggcatggcaggtgccttttgtttcatcctcatacaactagtcttacttattgattttgcacattcatggaatgaatcgtgggttgaaaaaatggaagaagggaactcgagatgttggtatgcagccttgttatcagctacagctctgaattatctgctgtctttagttgctatcgtcctgttctttgtctactacactcatccagccagttgttcagaaaacaaggcgttcatcagtgtcaacatgctcctctgcgttggtgcttctgtaatgtctatactgccaaaaatccaagaatcacaaccaagatctggtttgttacagtcttcagtaattacagtctacacaatgtatttgacatggtcagctatgaccaatgaaccagaaacaaattgcaacccaagtctactaagcataattggctacaatacaacaagcactgtcccaaaggaagggcagtcagtccagtggtggcatgctcaaggaattataggactaattctctttttgttgtgtgtattttattccagcatccgtacttcaaacaatagtcaggttaataaactgactctaacaagtgatgaatctacattaatagaagatggtggagctagaagtgatggatcactggaggatggggacgatgttcaccgagctgtagataatgaaagggatggtgtcacttacagttattccttctttcacttcatgcttttcctggcttcactttatatcatgatgacccttaccaactggtacaggtatgaaccctctcgtgagatgaaaagtcagtggacagctgtctgggtgaaaatctcttccagttggattggcatcgtgctgtatgtttggacactcgtggcaccacttgttcttacaaatcgtgattttgactgagtgagacttctagcatgaaagtcccactttgattattgcttatttgaaaacagtattcccaacttttgtaaagttgtgtatgtttttgcttcccatgtaacttctccagtgttctggcatgaattagattttactgcttgtcattttgttattttcttaccaagtgcattgatatgtgaagtagaatgaattgcagaggaaagttttatgaatatggtgatgagttagtaaaagtggccattattgggcttattctctgctctatagttgtgaaatgaagagtaaaaacaaatttgtttgactattttaaaattatattagaccttaagctgttttagcaagcattaaagcaaatgtatggctgccttttgaaatatttgatgtgttgcctggcaggatactgcaaagaacatggtttattttaaaatttataaacaagtcacttaaatgccagttgtctgaaaaatcttataaggttttacccttgatacggaatttacacaggtagggagtgtttagtggacaatagtgtaggttatggatggaggtgtcggtactaaattgaataacgagtaaataatcttacttgggtagagatggcctttgccaacaaagtgaactgttttggttgttttaaactcatgaagtatgggttcagtggaaatgtttggaactctgaaggatttagacaaggttttgaaaaggataatcatgggttagaaggaagtgtttgaaagtcactttgaaagttagttttgggccagcacggtagctcacccttgtaatcccagcactttgggaggctgaggtgggtagattacttgagcccaggaattcaagaccagcctgggcaacatggtgaaaccctgtttctataaaaaataatctgggctttgtagcatatgcctgtggtcccagctactgaggaggctgaggtgggaggattgcttgagcccaggaggcagaggttgcagtgagccaaggtcacgtcactgcactctagcctgggcaacagagtaagacaaaaaaatatatatatattgaaaatcaaaggaggcaaaattttgacagggaaggaagtaactgcaaaacactaggctttagtaggtacttatataaaatctagtccagttctctcatttaaaaaaatgaagacactgaaatacagacttaaatagctcagatagctaattaggaaatttcaagttggccaataatagcattctctctgacatttaaaaataatttctattcaaaatacatgcataattgattttacacctcattactggtggataatttatgtgatgtggattgctggtgtccagcatgacccataaacaggtcagaagaatgatggaatgttttagaataaactcctgcttatagtatactacacagttcaaaagatgtttaaaatgcttttgtatttactgccatgtaattgaaatatatagattattgtaacctttcaacctgaaaatcaagcagtatgagagtttagttatttgtatgtgtcactagtgtctaatgaagcttttaaaatctacaatttcttctttaaaaatatttattaatgtgaatggaatataacaattcagcttaattccccaaccttattctgtgtgtagacattgtattccacaattttgaatggctgtgttttacctctaaataaatgaattcagagaaa

SEQ ID NO: 23 - protein, 181 aa, Homo sapiens
  source 1..181: note = ARL1 biomarker
MGGFFSSIFSSLFGTREMRILILGLDGAGKTTILYRLQVGEVVTTIPTIGFNVETVTYKNLKFQVWDLGGQTSIRPYWRCYYSNTDAVIYVVDSCDRDRIGISKSELVAMLEEEELRKAILVVFANKQDMEQAMTSSEMANSLGLPALKDRKWQIFKTSATKGTGLDEAMEWLVETLKSRQ

SEQ ID NO: 24 - DNA, 3197 bp, Homo sapiens
  source 1..3197: mol_type = other DNA; note = ARL1 biomarker
cccgcccccgcctggagtccgacgtggaagttgctggctgactgggcttgcgaggaaaccgcctcggagctgcagccgaaggccaaggaatcactgaagatcggcgagggaggacagggggttcatcatgggtggctttttctcaagtatattttccagtctgtttggaactcgggaaatgagaattttaattttgggattagatggagcaggaaaaaccacaattttgtacagattacaagtgggagaagttgttactactatacctaccattggatttaatgtagagacggtgacgtacaaaaaccttaaattccaagtctgggatttaggaggacagacaagtatcaggccatactggagatgttactattcaaacacagatgcagtcatttatgtagtagacagttgtgaccgagaccgaattggcatttccaaatcagagttagttgccatgttggaggaagaagagctgagaaaagccattttagtggtgtttgcaaataaacaggacatggaacaggccatgacttcctcagagatggcaaattcacttgggttacctgccttgaaggaccgaaaatggcagatattcaaaacgtcagcaaccaaaggcaccggccttgatgaggcaatggaatggttagttgaaacattaaaaagcagacagtaattcagtccattcttctcccctgaaatgaagactacatcacctctctccctttggaaacagtcaagtgtacttcacactactagatgttaaaactatatgattattggcatatactgactgactgcaatatttgtagtaaatagggaaaataagtatttagttggagggataatttgatcgaatcacctgaatgttctatgtaatgtaaaatattcttttcttgctttcttgtgttaaggtatatattctatttgtatggaattcttattcaaatacagttctattaaagagtatactcctattggatgaaaaaaacctatttttgaatagtagttgcagcttgtttgtataaatactaaaaaatatcttaacagatttttttccccttaaaatgaattatctttccaaagattagactcagtatgagagtaggtgaggaacatagtttgtgtaatataatctttatttgcttacctgaccactttttattaagctgttttgtttaaaagcttttggttataggcatgaatattgttttgatggttaacttcactgacttatctgtaagtgtcattttatagtactttacagttgtcaaaaaaaatcttacatatattgatctagttgatcggtagaggaagcttgtgaagtaggtaggacaggttatgatgtcttcactgtatggacgtgaacccagggttctaataggttggcttgcccaaggtcacctgctagcttgtaaggtaggaatagggctcaaagtaaaacttttggttcagaagcttgactcataaactatactatatattcccaaaaatgtcagtattgacaacacagtctaactcttaaataaaattatgctaattagttacattcaaaataatgtttataaatttatttgtctatactagtagaagtgatcagtgacatactataaataatcctaaatggttatatttgatagaattcataacacattaaagagaaaatttcacattaaaggaaggtttaaaaatctattttcagattataaaaggtgtctttactggttttcattccttttatctacatgtaattggacatgtaaacttgttaaaaatcaaaatcttactgattgtacagggagaaattaagcattctgttttaaaaattaggcttattgctagactgtaagtttaaaaacaattcatgttcaaaggagcttttcaaattacccagtccaccacagaatcagctaatgtgacagaagtaaatatagtgcgtcataattgctaaaaacagggtgaatttctttcacttaagttggaaaaccagcctctaaaaaattaggtgctggtaatgagagcttaataccatggttccctgcaggtctcagaaacattagtagttcctttcttggccctcactgtccttccagtgttggcctctggcctcttcccctcacatttatgttcattctccgggcttatattcattctctgctgctttctcccttcacccgtgggactctcacccttcttgctcattctccagcacccattcctactttagtctctttgaaatcttttttggagattttccttcagctacaaatgttccagtacaaccaatattactcctgaggggcaaagactttttcatatttatgtccctagtatctggtatggcgcctggcatatggcatttcagaatatgttcatagttgaaacagtaggatagatatttgtcatcttgacaagtagccctttgcaatttatacttgagttcactcctggtcaatggcacatggctggaaaatgcagaaagcaagttcacttacagcctgaggcttataaagcttgtacagctgcctcccggcagtccaggaaaactgcaaaaaatgggaagtaaggtcctggcaggcagacccggcatgtagccgctggccatcttgaatcctagggcatgaagttgccccaaagttcagcacttggttaagcctgatccctctggtttatcacaaagaataggatgggataaagaaagtggacacttaaataagctataaattatatggtccttgtctagcaggagacaactgcacaggtatactaccagcgtttggatattgatatatacatggcaaatctgaatattgttttatgagaggcctggcaatgggtaaaacaccaaacttgaaatcagaacataggtgcaaatggccactcctgtagtttcttttctgtaaaatgggatctggacgaatgataccaaacgtttctttctaacattgtattttataactctggcctatgattgtgttgtgtcttgcattaaaaaaaaaaatttgagagtggtagaattacttctgttatctgaaatacctgagatgcactttaaactgttgaaatgtaagctctgagggcagggaccgtatcttatttactgttagtatccgttgcatctagtgtggtgcacctggcacacagtaggcactcaataaatatgtattgattaaaaacaaagacgtcttcaatatgccaga

SEQ ID NO: 25 - protein, 443 aa, Homo sapiens
  source 1..443: note = COPS2 biomarker
MSDMEDDFMCDDEEDYDLEYSEDSNSEPNVDLENQYYNSKALKEDDPKAALSSFQKVLELEGEKGEWGFKALKQMIKINFKLTNFPEMMNRYKQLLTYIRSAVTRNYSEKSINSILDYISTSKQMDLLQEFYETTLEALKDAKNDRLWFKTNTKLGKLYLEREEYGKLQKILRQLHQSCQTDDGEDDLKKGTQLLEIYALEIQMYTAQKNNKKLKALYEQSLHIKSAIPHPLIMGVIRECGGKMHLREGEFEKAHTDFFEAFKNYDESGSPRRTTCLKYLVLANMLMKSGINPFDSQEAKPYKNDPEILAMTNLVSAYQNNDITEFEKILKTNHSNIMDDPFIREHIEELLRNIRTQVLIKLIKPYTRIHIPFISKELNIDVADVESLLVQCILDNTIHGRIDQVNQLLELDHQKRGGARYTALDKWTNQLNSLNQAVVSKLA

SEQ ID NO: 26 - DNA, 6576 bp, Homo sapiens
  source 1..6576: mol_type = other DNA; note = COPS2 biomarker
ctcctccccctcccggccaagatgtctgacatggaggatgatttcatgtgcgatgatgaggaggactacgacctggaatactctgaagatagtaactccgagccaaatgtggatttggaaaatcagtactataattccaaagcattaaaagaagatgacccaaaagcggcattaagcagtttccaaaaggttttggaacttgaaggtgaaaaaggagaatggggatttaaagcactgaaacaaatgattaagattaacttcaagttgacaaactttccagaaatgatgaatagatataagcagctattgacctatattcggagtgcagtcacaagaaattattctgaaaaatccattaattctattcttgattatatctctacttctaaacagatggatttactgcaggaattctatgaaacaacactggaagctttgaaagatgctaagaatgatagactgtggtttaagacaaacacaaagcttggaaaattatatttagaacgagaggaatatggaaagcttcaaaaaattttacgccagttacatcagtcgtgccagactgatgatggagaagatgatctgaaaaaaggtacacagttattagaaatatatgctttggaaattcaaatgtacacagcacagaaaaataacaaaaaacttaaagcactctatgaacagtcacttcacatcaagtctgccatccctcatccactgattatgggagttatcagagaatgtggtggtaaaatgcacttgagggaaggtgaatttgaaaaggcacacactgatttttttgaagccttcaagaattatgatgaatctggaagtccaagacgaaccacttgcttaaaatatttggtcttagcaaatatgcttatgaaatcgggaataaatccatttgactcacaggaggccaagccgtacaaaaatgatccagaaattttagcaatgacgaatttagtaagtgcctatcagaataatgacatcactgaatttgaaaagattctaaaaacaaatcacagcaacatcatggatgatcctttcataagagaacacattgaagagcttttgcgaaacatcagaacacaagtgcttataaaattaattaagccttacacaagaatacatattccttttatttctaaggagttaaacatagatgtagctgatgtggagagcttgctggtgcagtgcatattggataacactattcatggccgaattgatcaagtcaaccaactccttgaactggatcatcagaagaggggtggtgcacgatatactgcactagataaatggaccaaccaactaaattctctcaaccaggctgtagtcagtaaactggcttaacagagaacaagcttttacagacgtccttaaggcaacagtgcagagatgtaatccttaaaagaactgggaatggcaaaactactgtcggttgatgtgtcctgaaaattattggagttatggcagaagtgcttttttgatcaactggtttgtgttttgctgctgcatttatcccaagaaaaacagctttaatctccagaagaaaaccaaaataccatgggatttatgctgtattgacatcttgccctaaacgtacaacatcatagtaatttgtcatgggcaacatgaccagagagaagatttttgtcatgattttaaatacactgacacgctactgttggttaaatttaaacatgttttacctgcagaaattctctcacaaataacctgcaataacttgaaatgcatacccttttgaacacttccttttctcatgtataaattaaaatgtttgctgcattttgcaaaatgtcaattctctaaaaatgtgtccgtatatttctgtacctgcagtgtagtaaaggtttagacgaaaccccataattatagtggcatactgtcacttaggtttcaagcagcaaaataaacagtgcagctcagaaattgtagtttggttcttgatgtgtttttattacatttggagttgttttgttttttagtaccttcgaaatttcaaattattttatcttcagttaatgattttaaaaagcctgggggcaaataagttggttatttgctttcaagtttttaaaagtagtctttattgatagagtaaggagaactactttctaacaaaacacgtgcatagttatgacagtgatgctttaaaggaataaaattctttttttttaaagagtgatattccttttcaaaagaatactaactctcagaatgttcactttaaacgaatatgccagaacatagacagctaaatgaatgttactctgcatagtgatcatgctggaaggttatttcctaatgccagcaatctaccattgcccaaaacctgctgagtttactcttttagaattgcattcaaagttaatttgtcacacacactaaactttatgattatacattgttttaaaaaatatagtattaggaagcttgattatttttagttaccattacttggcaccaaatgaaagtttccaaaacttccacctaactttgaggtaatgcagaaagtatataactggctttgaaggcaatcccaaaagagttttaaaggttttttgagcagtggcagtatacttaggagaatgaactgtggccttccaaggtaactaccttaaaggaactcagctcatttgaatgtattgagttttggatgtatttgtttcattttttaaaaagttcacattattttatagtgtcgaaaggaagaactaggattaacataatttctttggtttttctattgcttgttattattatgtaaaaactgggtggcagttcagaaggaagattgtggttacagaagagtgacaaccaagaattttttgatcattaaatcagattttataaacagtggaaggagcatggacttaaaacaaggcatgcttattcggttttgtcaaaattttacgaaaatatgtgatatatatttatactaaaaatatataatccttagatttagaaaagcaatcagttaatgtctttagcagactaaagcagtattaaacacaggtacaagttggaaattgtagaaaacggaaagaaaacaaaagacaaaatgtctatggtagggaataaaagtttaagatattataaaattatgtgtattttctcttttacataaatcatttgtgaaaagtgtgctaaactttttttacaagagtgatattaattaggatttatttttcaatataatttggagaccctttgttatccaaataaaaatgatgagtttttgtgcctgtattcaaatatgtatgcatgtgataacccttgaaagctaaagcccttcttaacttttgagttgatggaattagaattcaaagatttgaatgaaatgatttaacctttatcctccaattcttacagtgcccagttctcctgtgctatctttgctttgtacaatagtgcatcttccactttctagagagaaagcatgcacttgttatttggaaaactgggctaaatatataacagtatccaaagttataccataataatttattgtaattgtgtattacatagctttgtttacccagatataggtgcgttcttttttttctgttagtcatctgtgacttttgttctggaatacaggtttttaaatatatcttaacagtctgactaacttaaaataatttattcttcccttaaaacatttttctgtgtttttgtgcatcaaatattgtagagttgaaatcttagagattgcttatcgaaatataaatttaggggaagttaaaaatcgattggcaaatttgtagcatttattcactgattaaatcttttccacttttgtgaaaaccataccagtggtttacatcatattgtaatgtgttcatctcattcttctttttatccctaaacctagctaaaagttactgcaaagaaatctttggctgccacaagtagatgctctctactacaagagctggatttccattactcactcttgctcttacattaaagttgttgattaaatactttttctctacatcttaatgtaacataactgatttctttttaaagatgagtatatatgtactaagtaatgctctcattacatgagtcccttctaactacaggctgatacatctttcaggaaaactgagaacctaatatttaagtgaggcctattttaaatgagctatactgtgggtggtagtgtttgttaccttatgtaagacttaggtttgtcttagactgaaaaactcttattcttaatatttaaaatatccatgatggtagaaattttgtgggaaattttcttttttaatcaatagttacagttcttcagtaaacacaggtatgtatgcttttgtggtgtcagtgtcagtctaaatctatgggtgaagcagttagtccataagacaaaaaattatttgcctagtggattccctttacctctttttaagaagctcagagatctgtaatcattaatttactaaggattttttaaaatattattttagtcacttttaaacctgaaaatttctcttttgatttagatttttgaagtgaggcttgagcatacacaagtaaagctgggagtttcattcttcatatgggatttatgactaaatatttctagcattttgaagaagataaacattttgaactgaagtatataacaaaaaaaggaatttgacatttaactattttgtatcaaagtgtttaagaacactatccagaacaagagatggagtatcaggtttcagaatgtgacgcaaaaggcatttggtcttcatttatgtgaccacaattaaaattttcctctttttttctttttctggaggttttcttttagttttcattgttttaggtttttttttcccccaaatcagcatcctatttatatttgtttgatacggtttttttgtttttgtttttgttttgctttttttggaaacagtttcgctcttgtcacccaggctggagtgcagtggcgcaatctcagttcactgccacctccacctcccgggttcaagcgattctcctgcctcaatctccggagtagctgggattacagacacccgccaccacgcccagctaatttttgtgtttttagtagagacaagatttcaccatgttggccaggctggtctcaaactcctgacctcaggcgatccaccgctttggcctcccatagtgctgggattacaggcgtgagccaccacgcccagcctgatacagttttaagcaggaatttggcagaaatttcgtaacgaagacaaattggaggttctctttaactcgagattattaaatttatagtaacttcttgggccattaaaatgtgagtttaaatgccatttggaggatttgtgttatattaagcaccacaaatttaaactgtcaactagtgattctacatgtttctcaaaatgctaaagtttagcatcacttacttgcttttgaattttaaggagattcgtcttaggtaatgtctcagaagaaaccagtgtgtggttctctaaaattttacttttttactgtcacgtataataatatctaaactacctgtagtagttaaaaatattacggcatcaaaggtatatgccaagtgtaccttaaaaccctgtgacaggaaaaaagaaaaaattatttcttaagtacttactattcgggggcaactttgttccgtgttggcaaaattatgaactttgctcagaatttccacatgaatagctgaccaaaacttttttgtgtgcatatgtatacatatgtaataagtgaaacatacttgtgggagtacaaaattcatcaccagtttatcacagtaattttccagttttctattttacttaatatcacttatttctgacacaggcaaatgtcatatgccatgaaaagaattaccagtgtaccagtaacttgcgtgttatgactaggtttcatgtggaagactgtccaaatttacataatctagagtaattttttaattctgaagtaagggcattttaaaattaattgctaaaggttgttttattgtacatccattttatgggagtttagagtttagaaaaagtaatctctagcgtcatcccatgaagttgtcaaaagttttggcatgcttattagcaacatattaatgccaagtgttgagtatctaatttctgttcaaatgtttgcctatcccttactgtttacgacagtaataaatttttatgcatctattttaaataacactgaagaaaattaaaatatatttcaagaaatggagcaatgacatggaaaatagaaagttgtttggttttgcaatgtgttacataattggttgcattataaattttctccattttgtttatgataagctaatataaaaggtatcaaattccttgagctaacactaggtgggaaggaagtagtaggtgattaggaatgtaacaggatgtctgcctgaaatggtcagttccaccaattaataattgagaactgattatatgtgcaggatgtgtcataagatgctgtaaggcttaaatactcaagggtttttttatttatctggaagtgcacattccttattggtaataaaaaatacgtaatcta

SEQ ID NO: 27 - protein, 442 aa, Homo sapiens [sequence omitted]

SEQ ID NO: 28 - DNA, 2187 bp, Homo sapiens
  source 1..2187: mol_type = other DNA; note = CRBN biomarker
agacatggccggcgaaggagatcagcaggacgctgcgcacaacatgggcaaccacctgccgctcctgcctgcagagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaaacagatgtgatagagataaagttatctaacaaattggttatattctaagatctgctttggaaattattgcctctgatacatacctaagtaaacataacattaatacctaagtaaacataacattacttggagggttgcagtttctaagtgaaactgtatttgaaacttttaagtatactttaggaaacaagcatgaacggcagtctagaataccagaaacatctacttgggtagcttggtgccattatcctgtggaatctgatatgtctggtagcatgtcattgatgggacatgaagacatctttggaaatgatgagattatttcctgtgttaaaaaaaaaaaaaatcttaaattcctacaatgtgaaactgaaactaataatttgatcctgatgtatgggacagcgtatctgtaccagtgctctaaataacaaaagctagggtgacaagtacatgttccttttggaaagaagcaaggcaatgtatattaattattctaaaagggctttgttcctttccattttctttaacttctctgagatactgatttgtaaattttgaaaattagttaaaatatgcagttttttgagcccacgaatagttgtcatttcctttatgtgcctgttagtaaaaagtagtattgtgtatttgctcagtatctgaactataagcccatttatactgttccatacaaaagctatttttcaaaaattaatttgaaccaaaactactactatagggaaaagatgccaaaacatgtcccctcacccagactaaacttgatactgtattattttgttcaatgtaaattgaagaaaatctgtaagtaagtaaaccttaagtgtgaaactaaa

SEQ ID NO: 29 - protein, 400 aa, Homo sapiens [sequence omitted]

SEQ ID NO: 30 - DNA, 1727 bp, Homo sapiens
  source 1..1727: mol_type = other DNA; note = MAP2K2 biomarker
ctctcggactcgggctgcggcgtcagccttcttcgggcctcggcagcggtagcggctcgctcgcctcagccccagcgcccctcggctaccctcggcccaggcccgcagcgccgcccgccctcggccgccccgacgccggcctgggccgcggccgcagccccgggctcgcgtaggcgccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacaggctggcgcctccacccaccctcctgcctcacccctgcggagagcaccgtggcggggcgacagcgcatgcaggaacgggggtctcctctcctgcccgtcctggccggggtgcctctggggacgggcgacgctgctgtgtgtggtctcagaggctctgcttccttaggttacaaaacaaaacagggagagaaaaagcaaa

SEQ ID NO: 31 - protein, 204 aa, Homo sapiens
  source 1..204: note = ARHGDIA biomarker
MAEQEPTAEQLAQIAAENEEDEHSVNYKPPAQKSIQEIQELDKDDESLRKYKEALLGRVAVSADPNVPNVVVTGLTLVCSSAPGPLELDLTGDLESFKKQSFVLKEGVEYRIKISFRVNREIVSGMKYIQHTYRKGVKIDKTDYMVGSYGPRAEEYEFLTPVEEAPKGMLARGSYSIKSRFTDDDKTDHLSWEWNLTIKKDWKD

SEQ ID NO: 32 - DNA, 1837 bp, Homo sapiens
  source 1..1837: mol_type = other DNA; note = ARHGDIA biomarker
agtgcgggagggatcctgaaccgcgcggccgaaccctccggtgtcccgacccaggctaagcttgagcatggctgagcaggagcccacagccgagcagctggcccagattgcagcggagaacgaggaggatgagcactcggtcaactacaagcccccggcccagaagagcatccaggagatccaggagctggacaaggacgacgagagcctgcgaaagtacaaggaggccctgctgggccgcgtggccgtttccgcagaccccaacgtccccaacgtcgtggtgactggcctgaccctggtgtgcagctcggccccgggccccctggagctggacctgacgggcgacctggagagcttcaagaagcagtcgtttgtgctgaaggagggtgtggagtaccggataaaaatctctttccgggttaaccgagagatagtgtccggcatgaagtacatccagcatacgtacaggaaaggcgtcaagattgacaagactgactacatggtaggcagctatgggccccgggccgaggagtacgagttcctgacccccgtggaggaggcacccaagggtatgctggcccggggcagctacagcatcaagtcccgcttcacagacgacgacaagaccgaccacctgtcctgggagtggaatctcaccatcaagaaggactggaaggactgagcccagccagaggcgggcagggcagactgacggacggacgacggacaggcggatgtgtcccccccagcccctcccctccccataccaaagtgctgacaggccctccgtgcccctcccaccctggtccgcctccctggcctggctcaaccgagtgcctccgacccccctcctcagccctcccccacccacaggcccagcctcctcggtctcctgtctcgttgctgcttctgcctgtgctgtgggggagagaggccgcagccaggcctctgctgccctttctgtgccccccaggttctatctccccgtcacacccgaggcctggcttcaggagggagcggagcagccattctccaggccccgtggttgcccctggacgtgtgcgtctgctgctccggggtggagctggggtgtgggatgcacggcctcgtgggggccgggccgtcctccagccccgctgctccctggccagcccccttgtcgctgtcggtcccgtctaaccatgatgccttaacatgtggagtgtaccgtggggcctcactagcctctaactccctgtgtctgcatgagcatgtggcctccccgtcccttccccggtggcgaacccagtgacccagggacacgtggggtgtgctgctgctgctccccagcccaccagtgcctggccagcctgcccccttccctggacagggctgtggagatggctccggcggcttggggaaagccaaattgccaaaactcaagtcacctcagtaccatccaggaggctgggtattgtcctgcctctgccttttctgtctcagcgggcagtgcccagagcccacacccccccaagagccctcgatggacagcctcactcaccccacctgggcccagccaggagccccgcctggccatcagtatttattgcctccgtccgtgccgtccctgggccactggcctggcgcctgttcccccaggctctcagtgccaccacccccggcaggccttccctgacccagccaggaacaaacaagggaccaagtgcacacattgctgagagccgtctcctgtgcctcccccgccccatccccggtcttcgtgttgtgtctgccaggctcaggcagaggcgcctgtccctgcttcttttctgaccgggaaataaatgcccctgaagga

SEQ ID NO: 33 - protein, 371 aa, Homo sapiens
  source 1..371: note = GRINA biomarker
MSHEKSFLVSGDNYPPPNPGYPGGPQPPMPPYAQPPYPGAPYPQPPFQPSPYGQPGYPHGPSPYPQGGYPQGPYPQGGYPQGPYPQEGYPQGPYPQGGYPQGPYPQSPFPPNPYGQPQVFPGQDPDSPQHGNYQEEGPPSYYDNQDFPATNWDDKSIRQAFIRKVFLVLTLQLSVTLSTVSVFTFVAEVKGFVRENVWTYYVSYAVFFISLIVLSCCGDFRRKHPWNLVALSVLTASLSYMVGMIASFYNTEAVIMAVGITTAVCFTVVIFSMQTRYDFTSCMGVLLVSMVVLFIFAILCIFIRNRILEIVYASLGALLFTCFLAVDTQLLLGNKQLSLSPEEYVFAALNLYTDIINIFLYILTIIGRAKE

SEQ ID NO: 34 - DNA, 1970 bp, Homo sapiens
  source 1..1970: mol_type = other DNA; note = GRINA biomarker
acaaggccccgctgcgtcttccgagccgcaggcgcaggcccagctgagcggccgccgagcgggtgcgggtgcgggcgcatcggccatcaccgcgcggccgcgcagcggacaccgtgcgtaccggcctgcggcgcccggccaccggtgagtccccggcccgagcccaggagcgcctctgacccgctgcgccgcgcggcctgccgcccccgcccccgcccccacgcggatcttgcgcatccgagcgtggccgcctcgggggcggaccgcggaacccgaggccatgtcccatgaaaagagttttttggtgtctggggacaactatcctccccccaaccctggatatccgggggggccccagccacccatgcccccctatgctcagcctccctaccctggggccccttacccacagccccctttccagccctccccctacggtcagccagggtacccccatggccccagcccctacccccaagggggctacccacagggtccctacccccaagggggctacccacagggcccctacccacaagagggctacccacagggcccctacccccaagggggctacccccaggggccatatccccagagccccttcccccccaacccctatggacagccacaggtcttcccaggacaagaccctgactcaccccagcatggaaactaccaggaggagggtcccccatcctactatgacaaccaggacttccctgccaccaactgggatgacaagagcatccgacaggccttcatccgcaaggtgttcctagtgctgaccttgcagctgtcggtgaccctgtccacggtgtctgtgttcacttttgttgcggaggtgaagggctttgtccgggagaatgtctggacctactatgtctcctatgctgtcttcttcatctctctcatcgtcctcagctgttgtggggacttccggcgaaagcacccctggaaccttgttgcactgtcggtcctgaccgccagcctgtcgtacatggtggggatgatcgccagcttctacaacaccgaggcagtcatcatggccgtgggcatcaccacagccgtctgcttcaccgtcgtcatcttctccatgcagacccgctacgacttcacctcatgcatgggcgtgctcctggtgagcatggtggtgctcttcatcttcgccattctctgcatcttcatccggaaccgcatcctggagatcgtgtacgcctcactgggcgctctgctcttcacctgcttcctcgcagtggacacccagctgctactggggaacaagcagctgtccctgagcccagaagagtatgtgtttgctgcgctgaacctgtacacagacatcatcaacatcttcctgtacatcctcaccatcattggccgcgccaaggagtagccgagctccagctcgctgtgcccgctcaggtggcacggctggcctggaccctgcccctggcacggcagtgccagctgtacttcccctctctcttgtccccaggcacagcctagggaaaaggatgcctctctccaaccctcctgtatgtacactgcagatacttccatttggacccgctgtggccacagcatggcccctttagtcctcccgcccccgccaaggggcaccaaggccacgtttccgtgccacctcctgtctactcattgttgcatgagccctgtctgccagcccaccccagggactgggggcagcaccaggtcccggggagagggattgagccaagaggtgagggtgcacgtcttccctcctgtcccagctccccagcctggcgtagagcacccctcccctcccccccacccccctggagtgctgccctctggggacatgcggagtgggggtcttatccctgtgctgagccctgagggcagagaggatggcatgtttcaggggagggggaagccttcctctcaatttgttgtcagtgaaattccaataaatgggatttgctctctgc

SEQ ID NO: 35 - protein, 593 aa, Homo sapiens
  source 1..593: note = STXBP2 biomarker
MAPSGLKAVVGEKILSGVIRSVKKDGEWKVLIMDHPSMRILSSCCKMSDILAEGITIVEDINKRREPIPSLEAIYLLSPTEKSVQALIKDFQGTPTFTYKAAHIFFTDTCPEPLFSELGRSRLAKVVKTLKEIHLAFLPYEAQVFSLDAPHSTYNLYCPFRAEERTRQLEVLAQQIATLCATLQEYPAIRYRKGPEDTAQLAHAVLAKLNAFKADTPSLGEGPEKTRSQLLIMDRAADPVSPLLHELTFQAMAYDLLDIEQDTYRYETTGLSEAREKAVLLDEDDDLWVELRHMHIADVSKKVTELLRTFCESKRLTTDKANIKDLSQILKKMPQYQKELNKYSTHLHLADDCMKHFKGSVEKLCSVEQDLAMGSDAEGEKIKDSMKLIVPVLLDAAVPAYDKIRVLLLYILLRNGVSEENLAKLIQHANVQAHSSLIRNLEQLGGTVTNPGGSGTSSRLEPRERMEPTYQLSRWTPVIKDVMEDAVEDRLDRNLWPFVSDPAPTASSQAAVSARFGHWHKNKAGIEARAGPRLIVYVMGGVAMSEMRAAYEVTRATEGKWEVLIGSSHILTPTRFLDDLKALDKKLEDIALP

SEQ ID NO: 36 - DNA, 1885 bp, Homo sapiens
  source 1..1885: mol_type = other DNA; note = STXBP2 biomarker
gggacacacccggaagcggcggcggcgcccctcggggaagatggcgccctcggggctgaaggcggtggtgggggaaaaaattctgagcggagttattcggagtgtcaagaaggatggggagtggaaggtgcttatcatggatcacccaagcatgcgcatcttgtcttcctgctgcaaaatgtcagatatcctggctgagggcatcaccattgttgaagacatcaacaaacggcgggaacccattcccagtctggaggccatttatttgctgagccccacggagaagtcggttcaggccctgatcaaagacttccaggggaccccgactttcacctacaaagcggcccatatcttcttcaccgacacctgccccgagcccctgttcagtgagctaggccgctctcgtctggcaaaggtggtgaagacgttgaaggagattcaccttgccttcctcccctacgaggcccaggtgttctccctcgatgctccccacagcacctacaacctctactgccccttccgggcagaggagcgcacgcggcagctcgaggtgctggcccagcagattgccacgctgtgcgccaccctgcaggagtacccggccatccgctaccgcaagggcccagaggacacagcccagttggcccacgccgtcctggccaagctgaacgccttcaaggcagacactcccagtctgggcgagggcccagagaaaacccgctcccagctgctgataatggaccgggcagctgaccccgtgtccccactactgcatgagctcacgttccaggccatggcgtatgatctgctggacatagagcaggacacatacaggtatgagaccaccgggctgagcgaggcgcgggagaaggccgtcttgctggacgaggacgatgacttgtgggtggagcttcgccacatgcatatcgcagatgtgtccaagaaggtcacggagctcctgaggaccttctgtgagagcaagaggctgaccacggacaaggcgaacatcaaagacctatcccagatcctgaaaaagatgccgcagtaccagaaggagctgaataagtattctacgcacctgcatctagcagatgattgtatgaagcacttcaagggctcggtggagaagctgtgtagtgtggagcaggacctggccatgggctccgacgcagagggggagaagatcaaggactccatgaagctgatcgttccggtgctgctggacgcggcggtgcccgcctacgacaagatccgggtcctgctgctctacatcctccttcggaatggtgtgagtgaggagaacctggccaagctgatccagcatgccaatgtacaggcgcacagcagcctcatccgtaacctggagcagctgggaggcactgtcaccaaccccgggggctcggggacctccagccggctggagccgagagaacgcatggagcccacctatcagctgtcccgctggaccccggtcatcaaggatgtaatggaggacgccgtggaggaccggctggacaggaacctgtggcccttcgtatccgaccccgcccccacggccagctcccaggccgctgtcagtgcccgcttcggtcactggcacaagaacaaggctggcatagaagcccgggcgggcccccggctcatcgtgtatgtcatgggcggtgtggccatgtcagagatgagggccgcctacgaggtgaccagggccaccgagggcaagtgggaggtgctcattggctcctcacacatcctcaccccgacccgcttcctggatgacctgaaggcactggacaagaagctggaggacattgccctgccctgacccctggccccgccccctacccctccctttccagagaaataaactcttcccgtcgctctgcca

SEQ ID NO: 37 - DNA, 25 bp, synthetic construct
  source 1..25: mol_type = other DNA; note = sg-Arhgdia-#1
caccgtgagttcctgacacccatgg

SEQ ID NO: 38 - DNA, 25 bp, synthetic construct
  source 1..25: mol_type = other DNA; note = sg-Arhgdia-#2
caccgtgagttcctgacacccatgg

SEQ ID NO: 39 - DNA, 25 bp, synthetic construct
  source 1..25: mol_type = other DNA; note = non-targeting sgRNA control
caccgacggaggctaagcgtcgcaa

SEQ ID NO: 40 - DNA, 24 bp, synthetic construct
  source 1..24: mol_type = other DNA; note = influenza virus M2 gene forward
catggaatggctaaagacaagacc

SEQ ID NO: 41 - DNA, 21 bp, synthetic construct
  source 1..21: mol_type = other DNA; note = influenza virus M2 gene reverse
ccattaagggcattttggaca

SEQ ID NO: 42 - DNA, 20 bp, synthetic construct
  source 1..20: mol_type = other DNA; note = GAPDH forward
ggcaaattcaacggcacagt

SEQ ID NO: 43 - DNA, 19 bp, synthetic construct
  source 1..19: mol_type = other DNA; note = GAPDH reverse
agatggtgatgggcttccc

(19) United States
(12) Patent Application Publication
(10) Pub. No.: US 2024/0353397 A1
(43) Pub. Date: Oct. 24, 2024

(54) MARKERS OF RESISTANCE AND DISEASE TOLERANCE AND USES THEREOF

(71) Applicant: Ramot at Tel-Aviv University Ltd., Tel Aviv (IL)

(72) Inventors: Irit GAT-VIKS, Tel Aviv (IL); Eran BACHARACH, Tel Aviv (IL); Gal YANKOVITZ, Tel Aviv (IL); Ofir COHN, Tel Aviv (IL); Naama PESHES-YALOZ, Tel Aviv (IL)

(21) Appl. No.: 18/683,168

(22) PCT Filed: Aug. 11, 2022
(86) PCT No.: PCT/IL2022/050880

§ 371 Date: Feb. 12, 2024

Related U.S. Application Data
(60) Provisional application No. 63/232,460, filed on Aug. 12, 2021.

(51) Int. Cl. G01N 33/50; A61K 31/473; A61K 31/496

(52) U.S. Cl. CPC G01N 33/5091; A61K 31/473; A61K 31/496; G01N 33/5023

(57) ABSTRACT

The present disclosure provides methods for evaluating the immunological state in a subject by determining the levels of resistance and/or disease tolerance of the subject, using specific signatory biomarkers for disease tolerance and resistance. The present disclosure further provides diagnostic compositions and kits, as well as therapeutic methods based on manipulating the expression of the disclosed resistance and/or disease-tolerance biomarkers to modulate the immunological state and disease outcome.

### TECHNOLOGICAL FIELD

The present disclosure relates to the field of personalized medicine. More specifically, the invention provides compositions, kits and methods for the diagnosis, prognosis of various diseases as well as tailoring personalized treatments based on determining and modulating disease tolerance and resistance state of subjects in need.

### BACKGROUND ART

References considered to be relevant as background to the presently disclosed subject matter are listed below:
[1] Martins, R., Carlos, A. R., Braza, F., Thompson, J. A., Bastos-Amador, P., Ramos, S., and Soares, M. P. (2019). Disease Tolerance as an Inherent Component of Immunity. Annu. Rev. Immunol. 37, 405-437.
[2] Soares, M. P., Gozzelino, R., and Weis, S. (2014). Tissue damage control in disease tolerance. Trends in Immunology 35, 483-494.
[3] Soares, M. P., Teixeira, L., and Moita, L. F. (2017). Disease tolerance and immunity in host protection against infection. Nature Reviews Immunology 17, 83-96.
[4] Best, K. T., Nichols, A. E. C., Knapp, E., Hammert, W. C., Ketonis, C., Jonason, J. H., Awad, H. A., and Loiselle, A. E. (2020). NF-κB activation persists into the remodeling phase of tendon healing and promotes myofibroblast survival. Sci. Signal. 13, eabb7209.
[5] Park, Y. R., Sultan, Md.T., Park, H. J., Lee, J. M., Ju, H. W., Lee, O. J., Lee, D. J., Kaplan, D. L., and Park, C. H. (2018). NF-κB signaling is key in the wound healing processes of silk fibroin. Acta Biomaterialia 67, 183-195.
[6] Chovatiya, R., and Medzhitov, R. (2014). Stress, inflammation, and defense of homeostasis. Mol Cell 54, 281-288.
[7] Medzhitov, R., Schneider, D. S., and Soares, M. P. (2012). Disease tolerance as a defense strategy. Science 335, 936-941.
Torti, M. F., Giovannoni, F., Quintana, F. J., and García, C. C. (2021). The Aryl Hydrocarbon Receptor as a Modulator of Anti-viral Immunity. Frontiers in Immunology 12, 440.
Horng, H.-C., Chang, W.-H., Yeh, C.-C., Huang, B.-S., Chang, C.-P., Chen, Y.-J., Tsui, K.-H., and Wang, P.-H. (2017). Estrogen Effects on Wound Healing. International Journal of Molecular Sciences 18.

Acknowledgement of the above references herein is not to be inferred as meaning that these are in any way relevant to the patentability of the presently disclosed subject matter.

### BACKGROUND OF THE INVENTION

The host response to infection has two main arms of immune defense. One is the arm of resistance that detects, neutralizes, and eliminates the invading pathogen. The other is the arm of disease tolerance that limits stress and collateral tissue damage caused by resistance and by the pathogen; this arm does not have a direct effect on the pathogen [1-3]. Particularly, disease tolerance functions to suppress resistance and to restore homeostasis through tissue repair and renewal mechanisms. This broad concept of disease tolerance should not be confused with immune ‘tolerance’ used in the immunological literature to specifically describe the lack of responsiveness to particular antigens. The present disclosure refers to disease tolerance as either ‘disease tolerance’ or ‘tolerance’ interchangeably.

The resistance-tolerance balance is of particular significance in pathobiology of infectious diseases: failure of host defenses can result from either a failure of resistance or from failure of tolerance. In line with this, therapeutic interventions may target immunodeficiencies or deficiencies in tolerance and resistance [3]. Thus, gaining a molecular understanding of resistance and tolerance can facilitate development of effective therapeutic interventions.

While many functional properties of resistance and tolerance have been demonstrated [1], this concept raises a fundamental question of whether distinct cell states for resistance and tolerance exist at the molecular level, how are they characterized, and how do they relate to human disease. A major challenge in dissecting distinct resistance and tolerance cell states is that these two arms of defense are tightly linked: resistance and tolerance are jointly activated during infection, and the two processes have multiple shared genes and functionalities. For instance, both the pro-inflammatory response (resistance) and maintenance of homeostasis and tissue repair (tolerance) require NF-κB signaling [4-5]. Thus, uncoupling between resistance and tolerance cell states poses a significant challenge.

### SUMMARY OF THE INVENTION

A first aspect of the present disclosure relates to a method for evaluating the immune and/or the immunological state in a subject by determining the levels of resistance and/or tolerance of the subject. More specifically, in some embodiments, the method disclosed herein comprises the following steps. The first step (a), involves determining in at least one biological sample of the subject the expression level of at least three biomarkers of at least one of: (i) at least one biomarker of resistance, to obtain an expression value for each of the at least one biomarker/s. More specifically, the at least one biomarker/s of resistance is at least one of: MAX Interactor 1 (MXI1), Zinc Finger Protein 395 (ZNF395), Xeroderma Pigmentosum, Complementation group C (XPC), Methylenetetrahydrofolate Dehydrogenase 2 (MTHFD2), Proteasome Activator Subunit 2 (PSME2), Janus Kinase 2 (JAK2), Integrator Complex Subunit 12 (INTS12), Proteasome 20S Subunit Beta 7 (PSMB7), RNA Binding Motif Protein 7 (RBM7), Solute Carrier Family 6 Member 8 (SLC6A8) or any combination thereof; and (ii) at least one biomarker of tolerance, to obtain an expression value for each of the at least one biomarker/s. More specifically, the at least one biomarker/s of tolerance is at last one of: Serine Incorporator 1 (SERINC1), ADP Ribosylation Factor Like GTPase 1 (ARL1), COP9 Signalosome Subunit 2 (COPS2), Cereblon (CRBN), Mitogen-Activated Protein Kinase Kinase 2 (MAP2K2), Rho GDP Dissociation Inhibitor Alpha (ARHGDIA), Glutamate Ionotropic Receptor NMDA Type Subunit Associated Protein 1 (GRINA), Syntaxin Binding Protein 2 (STXBP2), RNA Binding Motif Protein 7 (RBM7), Solute Carrier Family 6 Member 8 (SLC6A8) or any combination thereof. The next step (b), of the disclosed method involves determining if the expression values obtained in steps (a) (i) and/or (a) (ii) for each of the at least three biomarkers is positive or negative with respect to a predetermined standard expression value or to an expression value of the biomarker/s in at least one control sample. Thus, wherein at least one of: (I) a positive expression value of at least one of the resistance biomarkers MTHFD2, PSME2, JAK2, INTS12, PSMB7 and RBM7 biomarker/s in said sample, and/or a negative expression value of at least one of the MXI1, ZNF395, XPC and SLC6A8 biomarker/s in the sample, indicate(s) that the resistance level is elevated in the subject; and/or (II) a positive expression value of at least one of the tolerance biomarkers MAP2K2, ARHGDIA, GRINA, STXBP2 and SLC6A8 biomarker/s in the sample, and/or a negative expression value of at least one of the SERINCI, ARLI, COPS2, CRBN and RBM7 biomarker/s in the sample indicate(s) that the tolerance level is elevated in the subject, thereby determining the immune/immunological state in the subject.

A further aspect of the present disclosure relates to a prognostic method for determining the susceptibility of a subject to at least one pathologic disorder, and/or predicting the outcome of the at least one pathological disorder in the subject. More specifically, the method comprising the following steps: First in step (a), determining the level/s of resistance and/or tolerance of the subject. The next step (b), involves classifying the subject as a subject susceptible to the pathologic disorder and/or to develop a negative outcome of the pathological disorder, based on the resistance and tolerance levels of he subject and the levels of resistance and tolerance that characterize the particular disorder. More specifically, the subject is determined susceptible if the level of resistance and/or tolerance determined in step (a) is at least one of: (i) elevated resistance and/or reduced tolerance, in a disorder where a reduced susceptibility and/or positive outcome in a subject is characterized with reduced resistance and/or elevated tolerance; and (ii) reduced resistance and/or elevated tolerance, in a disorder where a reduced susceptibility and/or positive outcome in a subject is characterized with elevated resistance and/or reduced tolerance, thereby determining the susceptibility of said subject and/or predicting the outcome of the pathological disorder in the subject.

A further aspect of the present disclosure relates to a prognostic method for predicting and assessing responsiveness of a subject suffering from a pathologic disorder to at least one compound or to a treatment regimen comprising this specific compound. Optionally, or additionally, the disclosed method may be also applicable for monitoring disease progression. In some embodiments, the method disclosed herein may comprise the following steps. First in step (a), determining the levels of resistance and/or tolerance of the subject. The next step (b), involves classifying the subject as: (I) a responder to the at least one compound or a treatment regimen comprising the compound, if at least one sample obtained after the initiation of the treatment regimen and/or a sample of the subject contacted with the compound displays at least one of: (i) an elevated resistance and/or reduced tolerance, in a disorder where responsiveness and/or positive outcome in a subject is characterized with elevated resistance and/or reduced tolerance; and (ii) reduced resistance and/or elevated tolerance, in a disorder where responsiveness and/or positive outcome is characterized with reduced resistance and/or elevated tolerance. Alternatively, the subject may be classified as (II), a non-responder to the at least one compound or a treatment regimen comprising the compound, if at least one sample obtained after the initiation of the treatment regimen and/or a sample of the subject contacted with the compound displays at least one of: (i) elevated resistance and/or reduced tolerance, in a disorder where a responsiveness and/or positive outcome is characterized with reduced resistance and/or elevated tolerance; and (ii) reduced resistance and/or elevated tolerance, in a disorder where a responsiveness and/or positive outcome in a subject is characterized with elevated resistance and/or reduced tolerance. The method thereby enables predicting and assessing responsiveness of the subject to the treatment regimen.

A further aspect of the present disclosure relates to a method for determining a personalized treatment regimen for a subject suffering from a pathologic disorder. The therapeutic method disclosed herein is personally adapted for each patient and may further provide a continuous and monitored treatment regimen. This therapeutic method therefore combines diagnostic steps for determining the immunological state of the treated subject, specifically, the resistance and/or tolerance levels of the treated subject. More specifically, in some embodiments, the method comprising the following steps. First in step (a), determining the level/s of resistance and/or tolerance of the subject. The next step (b), involves selecting a treatment regimen determined as modifying the levels of resistance and/or tolerance in the subject. More specifically, where reduction of resistance and/or elevation of tolerance is required, an appropriate treatment regimen selected is a treatment that reduces resistance and/or elevates tolerance. In some other embodiments, where elevation of resistance and/or reduction of tolerance is required, an appropriate treatment regimen selected is a treatment that elevates resistance and/or reduces tolerance. It should be further understood that an appropriate treatment regimen may affect only one of, resistance or tolerance. A treatment regimen is selected if at least one of: (i) the treatment regimen elevates resistance and/or reduces tolerance, in at least one sample of the subject, wherein the subject is suffering from a disorder where a positive outcome is characterized with elevated resistance and/or reduced tolerance; and (ii) the treatment regimen reduces resistance and/or elevated tolerance, in at least one sample of said subject, wherein the subject is suffering from a disorder where positive outcome is characterized with reduced resistance and/or elevated tolerance. A further aspect of the present disclosure relates to a method for treating, preventing, inhibiting, reducing, eliminating, protecting or delaying the onset at least one pathological disorder in a subject in need thereof. The therapeutic methods disclosed herein provide tailored and monitored treatment as discussed above, by combining a diagnostic step that allows determination of the specific state of the subject and evaluation of the effect of a particular therapeutic compound on each treated subject. In some embodiments, the method comprises the following steps. First in the diagnostic step (a), determining the levels of resistance and/or tolerance of the subject. The next step (b), involves classifying the subject as a responder or non-responder to a candidate compound or a treatment regimen comprising the compound. The next step (c) concerns administering a specific compound or subjecting the subject to a treatment regime comprising the compound, if at least one of: (i) the compound or a treatment regimen comprising the compound elevates resistance and/or reduces tolerance, in at least one sample of the subject. In case the subject is suffering from a disorder where a positive outcome is characterized with elevated resistance and/or reduced tolerance; and (ii) the compound or a treatment regimen comprising the compound reduces resistance and/or elevated tolerance, in at least one sample of the subject. In case the subject is suffering from a disorder where positive outcome is characterized with reduced resistance and/or elevated tolerance.

Another aspect of the present disclosure relates to a method for manipulating the immunological state of a subject suffering from a pathologic condition by modulating the levels of resistance and/or tolerance of the subject. In some embodiments, the method comprising administering to the subject a therapeutically effective amount of at least one of:

In some embodiments (a), at least one compound or a procedure that leads to an increase in the level of at least one of MTHFD2, PSME2, INTS12, PSMB7 and RBM7, and optionally, JAK2, biomarker/s and/or at least one compound or a procedure that leads to a decrease in the level of at least one of MXI1, ZNF395, XPC and SLC6A8 biomarker/s, and/or at least one compound or a procedure that leads to a decrease in the level of at least one of MAP2K2, ARHGDIA, GRINA and STXBP2 biomarker/s and/or at least one compound or a procedure that leads to an increase in the level of at least one of SERINC1, ARL1, COPS2 and CRBN biomarker/s, for a subject suffering from a disorder where a positive outcome is characterized with elevated resistance and/or reduced tolerance.

In yet some further embodiments (b), at least one compound or a procedure that leads to a decrease in the level of at least one of MTHFD2, PSME2, INTS12, PSMB7 and RBM7, and optionally, JAK2, biomarker/s and/or at least one compound or a procedure that leads to an increase in the level of at least one of MXI1, ZNF395, XPC and SLC6A8 biomarker/s, and/or at least one compound or a procedure that leads to an increase in the level of at least one of MAP2K2, ARHGDIA, GRINA and STXBP2 biomarker/s and/or at least one compound or a procedure that leads to a decrease in the level of at least one of SERINC1, ARLI, COPS2 and CRBN biomarker/s, for a subject suffering from a disorder where positive outcome is characterized with reduced resistance and/or elevated tolerance.

A further aspect of the present disclosure relates to a screening method for identifying (and or evaluating) at least one therapeutic compound for the treatment of a pathologic disorder. It should be noted that a selected compound modifies the level of resistance and/or tolerance in at least one subject suffering from the pathologic disorder. In some embodiments, the method comprising the steps of:

First (a), determining the levels of resistance and/or tolerance of at least one biological sample contacted with the candidate compound. The sample is of a subject suffering from the specific pathologic disorder. The next step (b), involves determining that the candidate compound is a therapeutic compound for the disorder if: (i) the candidate compound elevates resistance and/or reduces tolerance, in a sample of the subject contacted with the candidate compound, as compared to a control sample, in case the subject is suffering from a disorder where a positive outcome is characterized with elevated resistance and/or reduced tolerance; and (ii) the compound reduces resistance and/or elevates tolerance, in a sample of the subject contacted with the candidate compound, as compared to a control sample, in case subject is suffering from a disorder where positive outcome is characterized with reduced resistance and/or elevated tolerance.

A further aspect of the present disclosure relates to a diagnostic composition comprising at least one detecting molecule or any combination or mixture of plurality of detecting molecules specific for determining the level of expression of at least three biomarkers of at least one of: (i) at least one biomarker of resistance, such at least one biomarker is at least one of MXII, ZNF395, XPC, MTHFD2, PSME2, JAK2, INTS12, PSMB7, RBM7, SLC6A8 or any combination thereof; and (ii) at least one biomarker of tolerance, such at least one biomarker is at least one of SERINC1, ARLI, COPS2, CRBN, MAP2K2, ARHGDIA, GRINA, STXBP2, RBM7, SLC6A8 or any combination thereof. It should be noted that each of the detecting molecules is specific for one of the biomarker/s. A further aspect of the present disclosure relates to a kit comprising: (a) at least one detecting molecule specific for determining the level of expression of at least three biomarkers of at least one of: (i) at least one biomarker of resistance, the at least one biomarker is at least one of MXI1, ZNF395, XPC, MTHFD2, PSME2, JAK2, INTS12, PSMB7, RBM7, SLC6A8 or any combination thereof; and (ii) at least one biomarker of tolerance, the at least one biomarker is at least one of SERINC1, ARL1, COPS2, CRBN, MAP2K2, ARHGDIA, GRINA, STXBP2, RBM7, SLC6A8 or any combination thereof in a biological sample. It should be noted that each of the detecting molecule/s is specific for one of the biomarkers. In some embodiment the kit may optionally further comprises at least one of: (b) pre-determined calibration curve/s or predetermined standard/s providing standard expression values of said at least one biomarker/s; and (c) at least one control sample.

These and other aspects of the invention will become apparent by the hand of the following disclosure.

### BRIEF DESCRIPTION OF THE DRA WINGS

In order to better understand the subject matter that is disclosed herein and to exemplify how it may be carried out in practice, embodiments will now be described, by way of non-limiting example only, with reference to the accompanying drawings, in which:

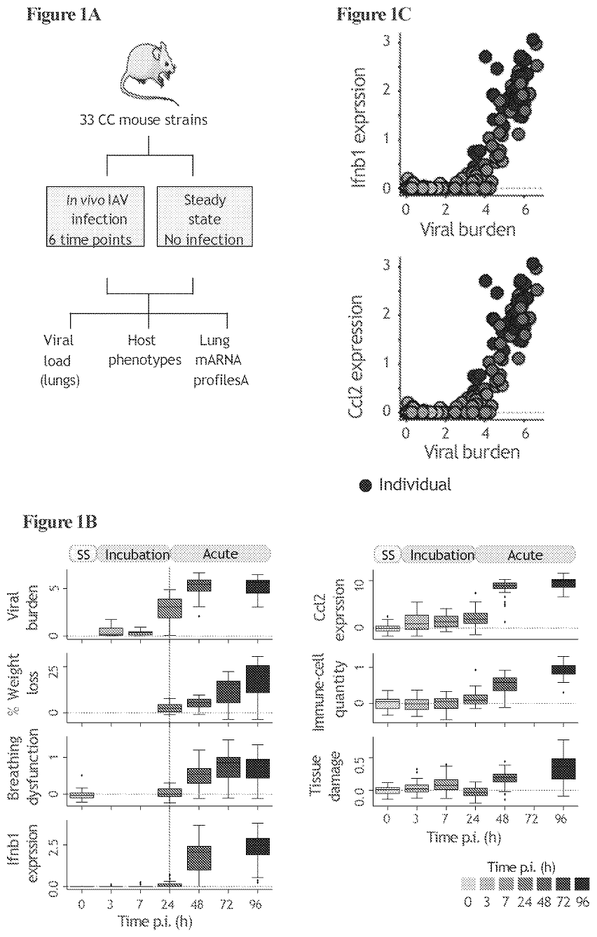

FIG. 1A-1C. Diversity in the host response to IAV infection

FIG. 1A. Study design. Before and during IAV infection, 33 CC mouse strains were phenotyped and analyzed by mRNA profiling of their lungs.

FIG. 1B. Phenotypic diversity. Shown are different phenotypes (panels) at each time point. For each time point, the box plots represent phenotypic distribution across 33 independent animals of different mouse strains. SS, steady state.

FIG. 1C. Relations among disease phenotypes. Relationship between Ifnbl and Ccl2 mRNA expression (log-scaled, y axes) and viral burden (log scaled, x axis) in 33 animals of different strains (dots) over time (color coded as in B).

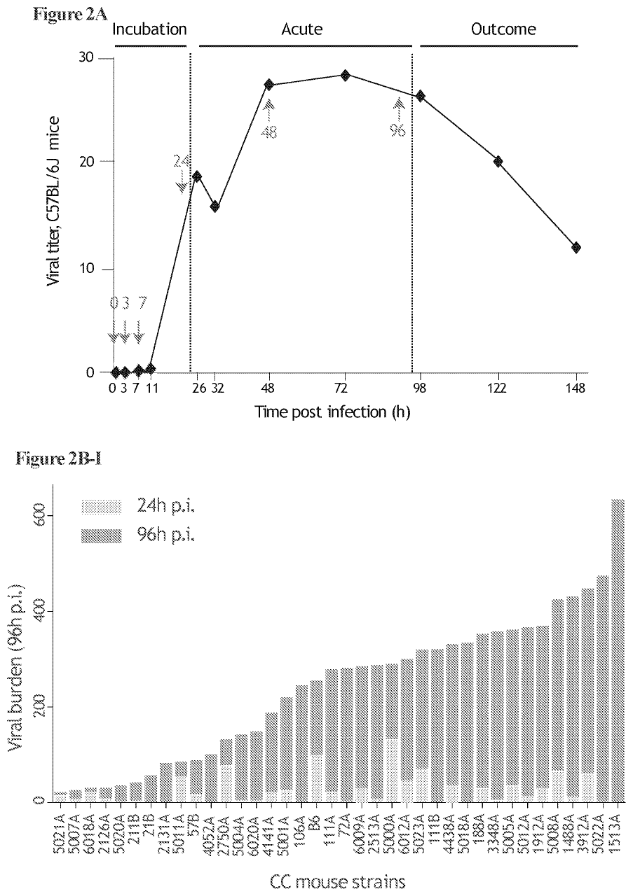

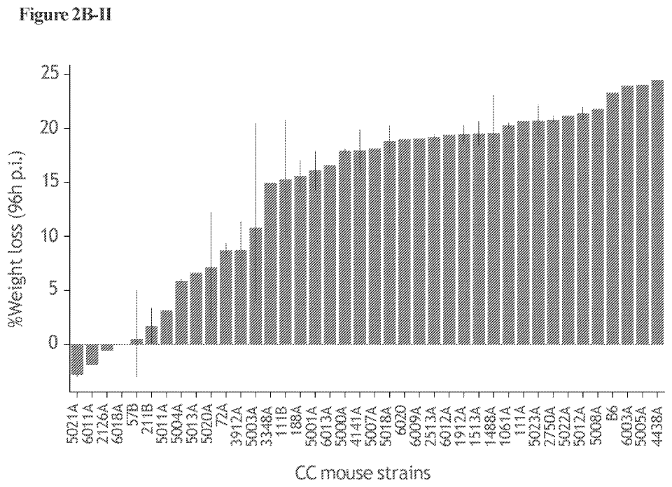

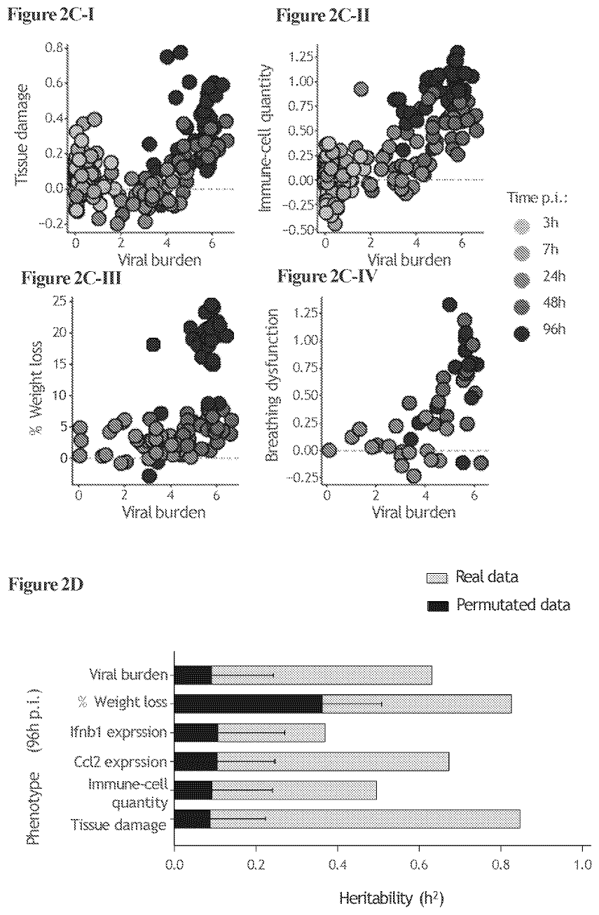

FIG. 2A-2D. Diversity of IAV infection severity across mouse strains

FIG. 2A. Viral titer during IAV infection of the C57BL/6J mouse strain (data from Altboum et al., 2014).

FIG. 2B-I-2B-II. Viral burden (expression levels of viral mRNA; 2B-I) and percentage of whole-body weight loss (2B-II) at 96h p.i. (y axis) across the CC mouse strains (x axis).

FIG. 2C-I-2C-IV. Relations between disease phenotypes and viral burden. The plots are shown as in FIG. 1C, tissue damage (2C-I), immune cell quantity (2C-II), weight loss (2C-III), breathing disfunction (2C-IV).

FIG. 2D. Heritability (h², x axis) of different disease phenotypes at 96h p.i. (y axis), either in real data (gray) or permuted data (black).

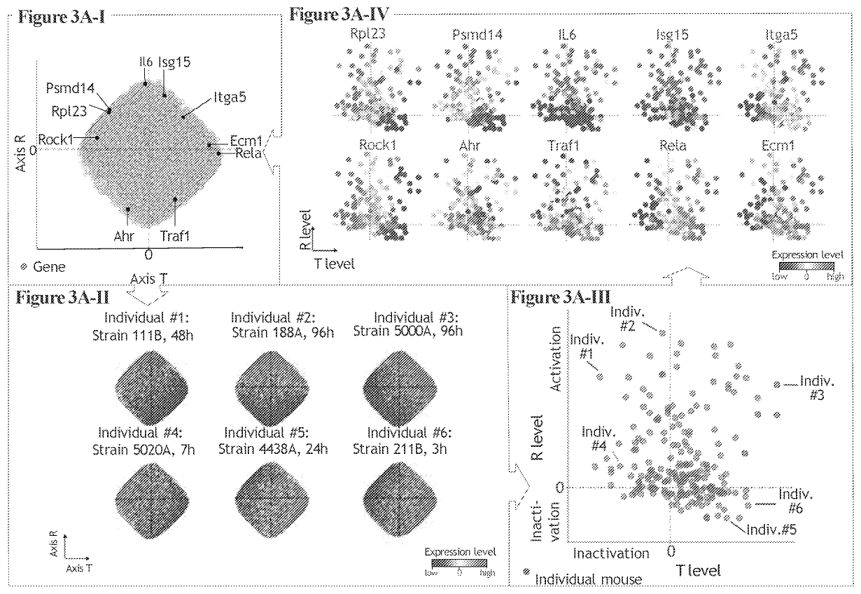

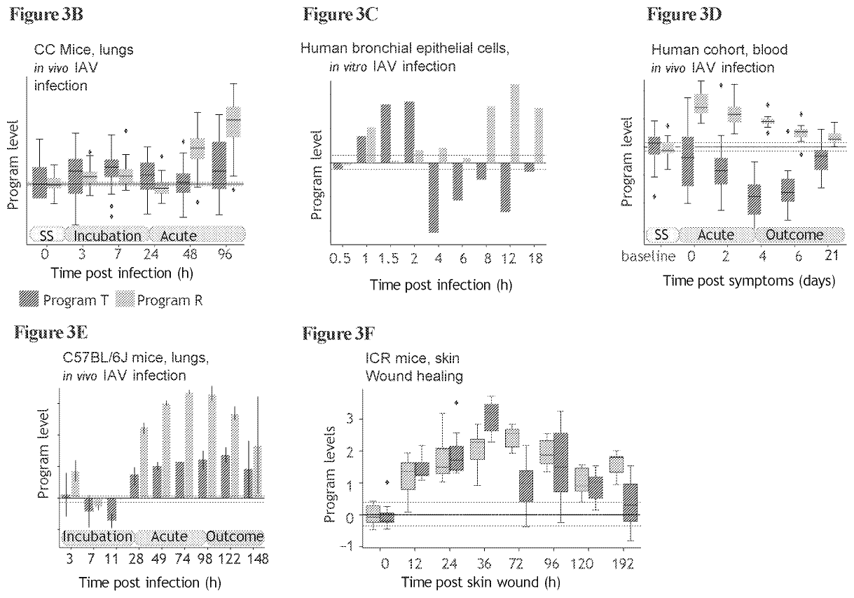

FIG. 3A-3F. Two generic programs capture the wide diversity of host transcriptional responses to IAV infection

FIG. 3AI-3AIV. Definition of programs and their levels. (3A-I) A gene map. A two-dimensional space in which each gene (a dot) is located in a particular coordinate, such that nearby genes have similar transcriptional patterns across all mouse strains and all time points. T/R: the horizontal/vertical axes. (3A-II) Gene expression in specific individuals. In each panel, the same map (from 3A-I) is colored by gene expression from one specific individual (indicated on top). The blue/red scale indicates low/high expression levels (no data smoothing). The gradient along the R and T axes allows compression of the overall state of each individual into two numbers, referred to as the ‘levels’ of program R and program T. (3A-III) A two-dimensional representation of the overall expression state of each individual. Presented are the levels of programs R and T for each individual (a dot) across all mice (all individuals from each time point). Individuals from A-II are indicated. (3A-IV) State-specificity of individual genes. All panels show the same landscape from 3A-III, where each panel is colored according to the expression of one specific gene (indicated on top)—that is, each dot (an individual mouse) is colored by the expression of the relevant gene in this individual (coloring as in 3A-II). Genes are those indicated in panel 3A-I, demonstrating that the state-specificity of genes (3A-IV) is encoded by their positions in the map (3A-I).

FIGS. 3B-3F. R and T levels during IAV infection. Shown are R and T levels (color coded) as a function of time across (FIG. 3B) lung samples from CC strains, (FIG. 3C) in vitro infection of primary human bronchial epithelial cells, (FIG. 3D) blood samples from human subjects (time post symptoms onset), and (FIG. 3E) the C57BL/6J mouse strain. (FIG. 3F) levels during skin response to wound. Dashed lines: empirical p<0.001 for panel B and p<0.05 for panels 3C−3E.

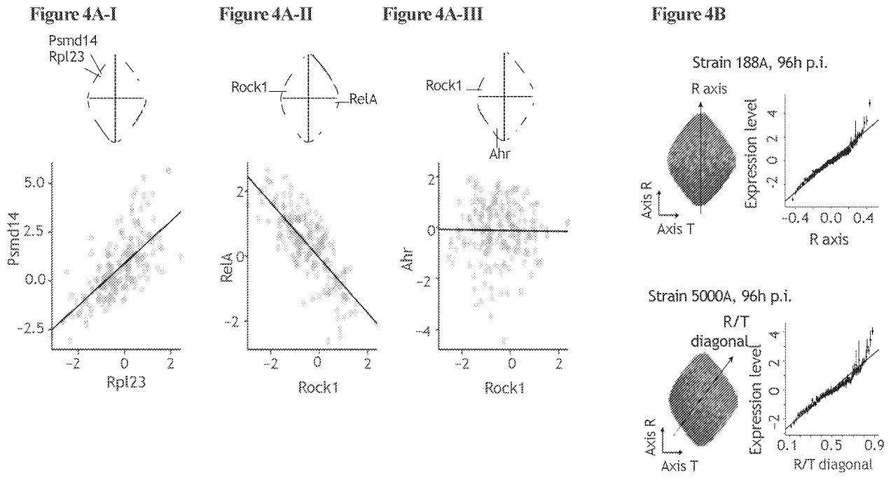

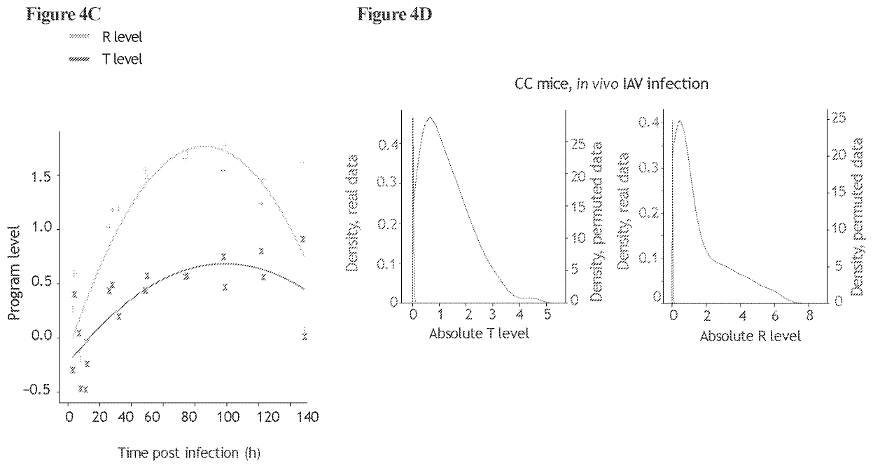

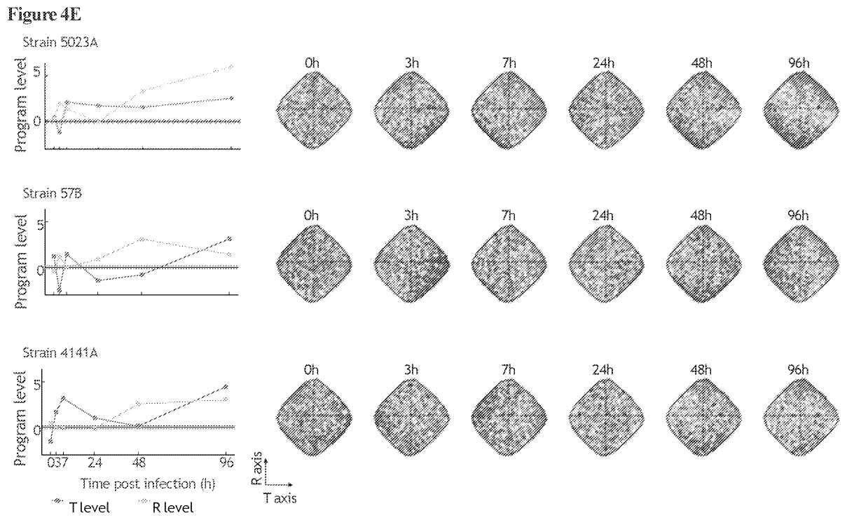

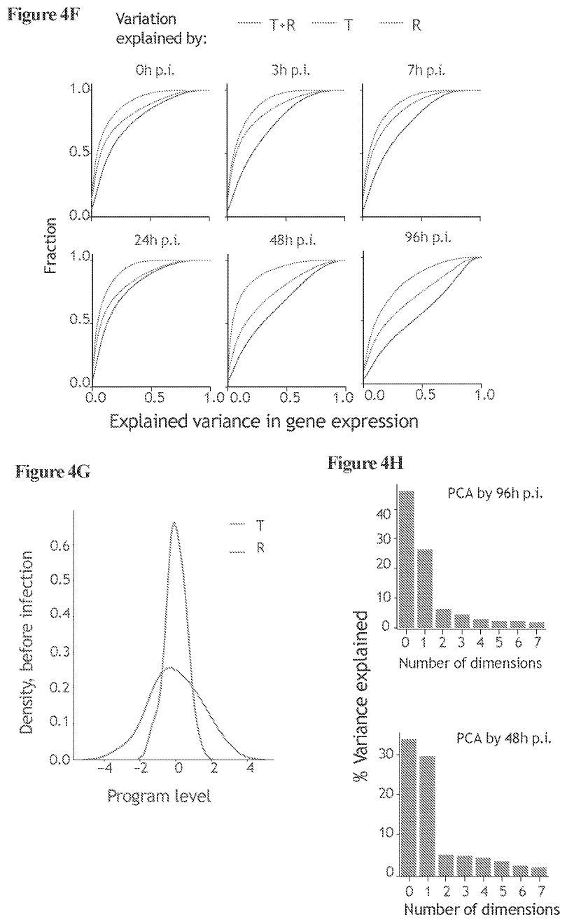

FIG. 4A-4H. Additional characterization of the model

FIG. 4A-1-4AII-I. The similarity rule. Scatter plots for the relations between pairs of genes across all individual mice (all strains and time points). The positions of genes in the gene map (the map from FIG. 4A-I) are indicated on top. The plots demonstrate the organization of the map: (i) nearby genes in the map are positively correlated (e.g., Rpl23, Psmd14, FIG. 4A-I), (ii) genes in opposite positions in the map are negatively correlated (e.g., Rockl, RelA, FIG. 4A-II) and (iii) other pairs of genes are either uncorrelated or weakly correlated (e.g., Ahr, Rockl, FIG. 4A-III).

FIG. 4B. A nearly linear change in the expression of genes along the gradient. A detailed analysis of two individual mice at 96h p.i.: one from strain 188A (top panels) and the other from strain 5000A (bottom panels). For each individual, the left panel shows the gene map, color coded with its gene expression. The direction of the gene expression's gradient is indicated as an arrow on top of the map. For each individual, the right panel presents a sliding window of expression levels along the direction of the gradient.

FIG. 4C. Reproducibility of R and T levels. R and T levels (color coded) in different individuals (dots) of the C57BL/6J strain. Individuals were measured at different time points during IAV infection (x axis). Measurements are two independent individuals per time point; data from Altboum et al. (2014).

FIG. 4D. R and T levels are robust and reliable. Distribution of absolute T and R levels (left and right, respectively) in the CC mice during in vivo IAV infection. T and R levels were calculated using the measured data (green) and permuted data (purple). The empirical p-value cutoff in FIG. 3B and in panel E is based on this analysis.

FIG. 4E. Demonstration of R and T dynamics along the course of IAV infection, for three CC mouse strains (top, middle, and bottom). R and T levels are shown either by the calculated levels (left) or by coloring the gene map with the expression levels of each gene (right). Left: dashed lines indicate empirical p<0.001 based on the analysis in panel D.

FIG. 4F. The R and T scores explain substantial fractions of the variation in gene expression. Cumulative distributions of the explained variation in gene expression, calculated for different time points (plots, indicated on top), when using only T levels (orange), only R levels (green), or both (blue), to explain variation in gene expression.

FIG. 4G. Distribution of baseline T and R levels in steady state (before infection) across the CC mice. The plot indicates that the R and T levels are centered around zero and that nearly-zero levels are prevalent.

FIG. 4H. Principal component analysis of the CC's gene expression data at 96h p.i. (top) and 48h p.i. (bottom). For each principal component, shown is the total variation explained by the component.

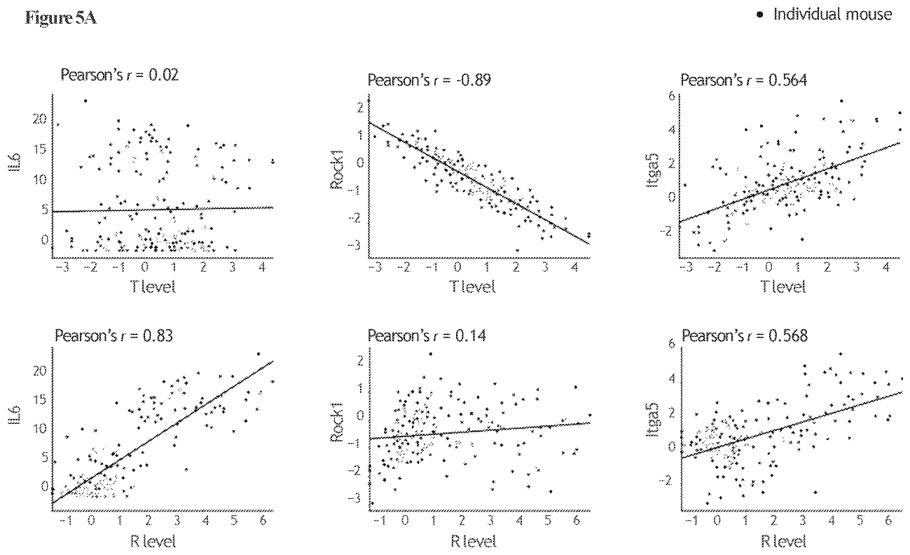

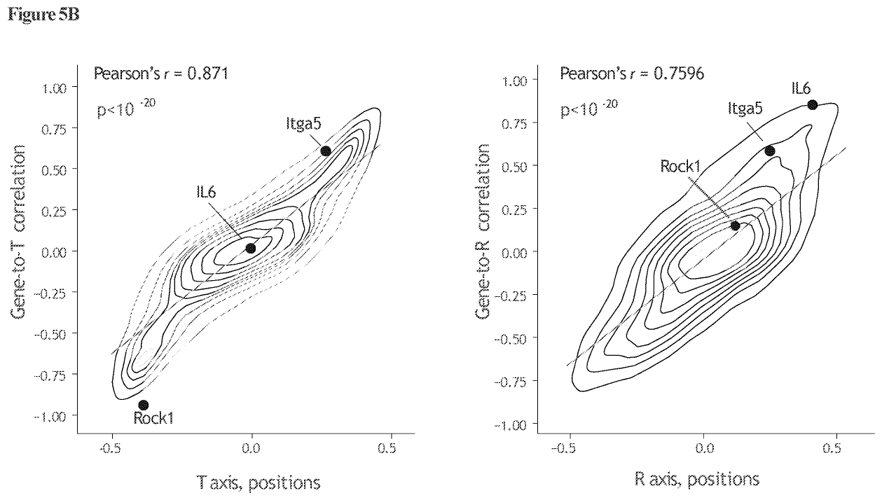

FIG. 5A-5C. Characterization of the map

FIG. 5A. Examples of relations between the position of genes in the map and their correlations with program levels. For each gene (a sub-panel), shown is a scatter plot of its expression level (y axis) and the levels of T (top) and R (bottom) (x axis) across all individuals (dots), including individuals of all time points and strains. The correlations, referred to as ‘gene-to-program correlations’, are indicated.

FIG. 5B. A global view of the relations between the position of genes in the map and their correlations with program levels. Relations between the position of genes in the map (x axis) and their gene-to-program correlations (y axis), visualized using two-dimensional kernel density estimates (KDE) plots. The red lines indicate the fitted linear regression line. Specific genes from panel A are indicated. The plots show that genes closer to the end of an axis in the map are better correlated with the respective program.

FIG. 5C. Color coding of the gene map with the explained gene-expression variation. Each gene (a dot in the gene maps) is color coded by its explained variation (white to pink scale). In each panel, the explained variation was calculated for a particular time point (left to right panels) using both R and T (I), T levels (II), or R levels (III). The plots indicate that genes closer to the boundary of the map are better correlated with the R/T state.

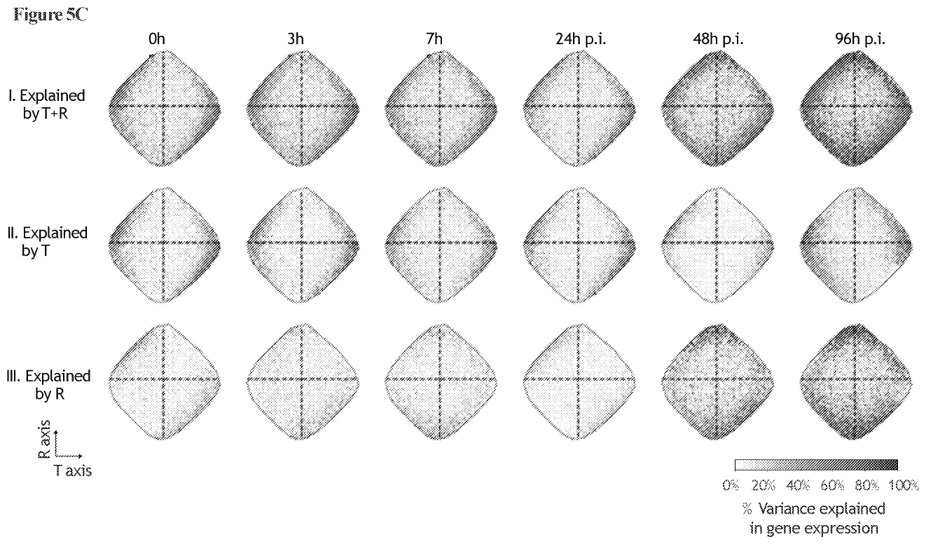

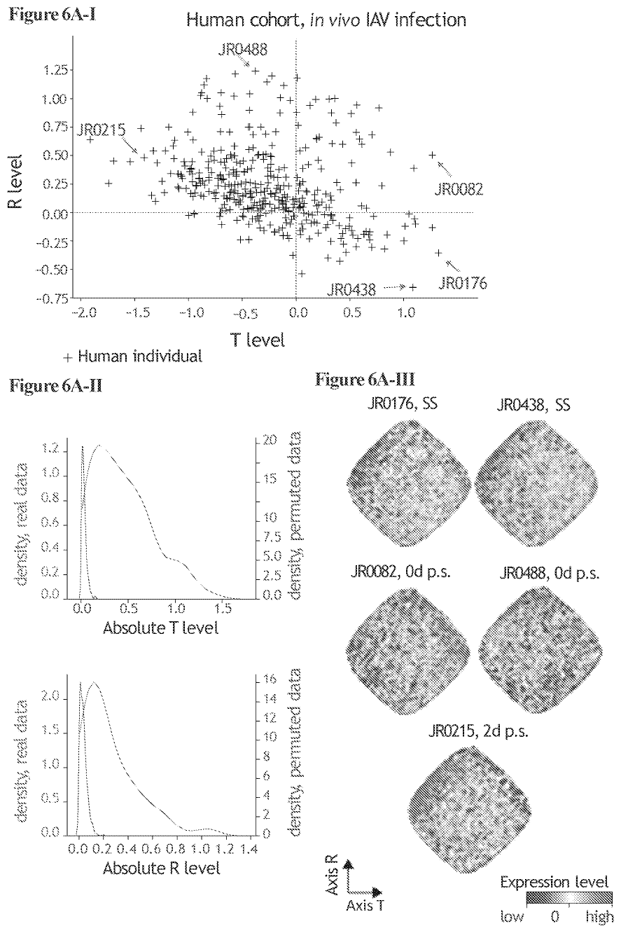

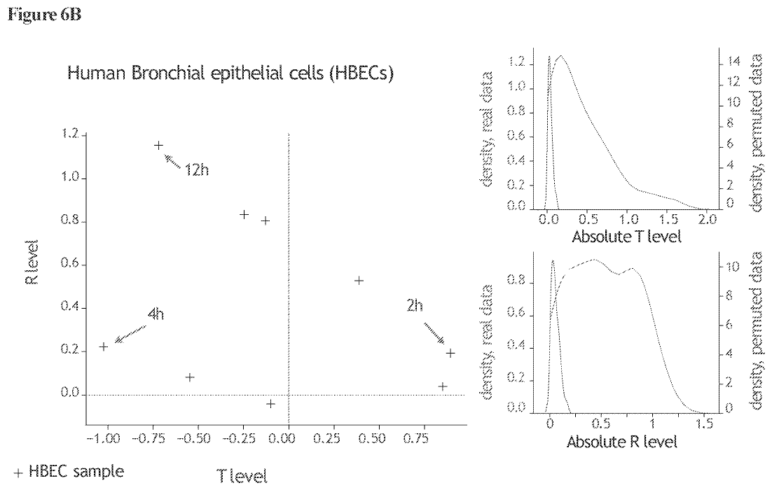

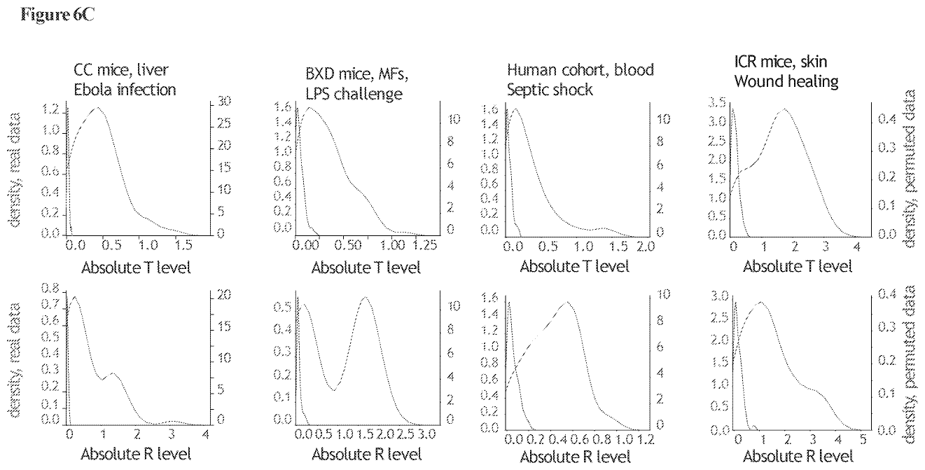

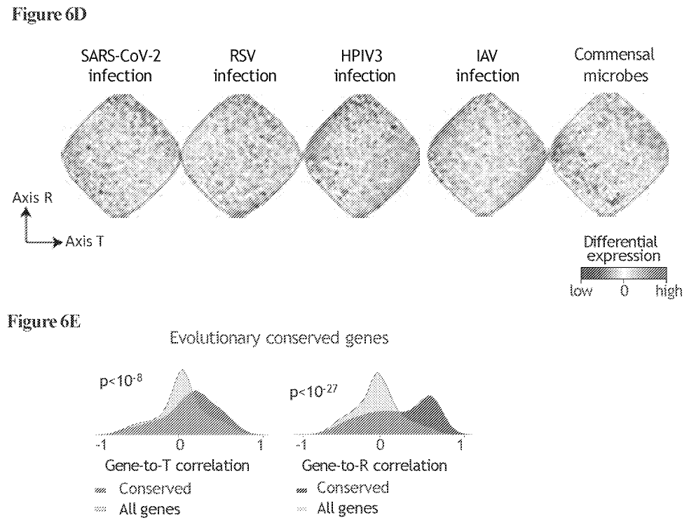

FIG. 6A-6E. Generality and contribution of the R and T programs

FIG. 6A-I-6A-III. Blood samples before and during in vivo IAV infection in a human cohort. Shown are the R and T levels across the samples (6A-I and 6A-II) as well as specific examples (6A-III) (data from Zhai et al. (2015)).

FIG. 6B. Samples of human bronchial epithelial cells during IAV infection (data from Shapira et al. (2009)). Shown are the R and T levels across the samples.

FIG. 6C. Variation in program levels (y axis) of the T and R programs (x axis), for permuted data (gray) and original data (red) from various datasets (panels).

FIG. 6D. Response to viral infections of human epithelial cells. Each panel represents the transcriptional response to a different pathogen. Response to infection (i.e., differential expression of infected versus uninfected cells) is represented in colors (blue to red scale). Data from Daamen et al. (2021) (viral infections) and Schaupp et al. (2020) (commensal microbes).

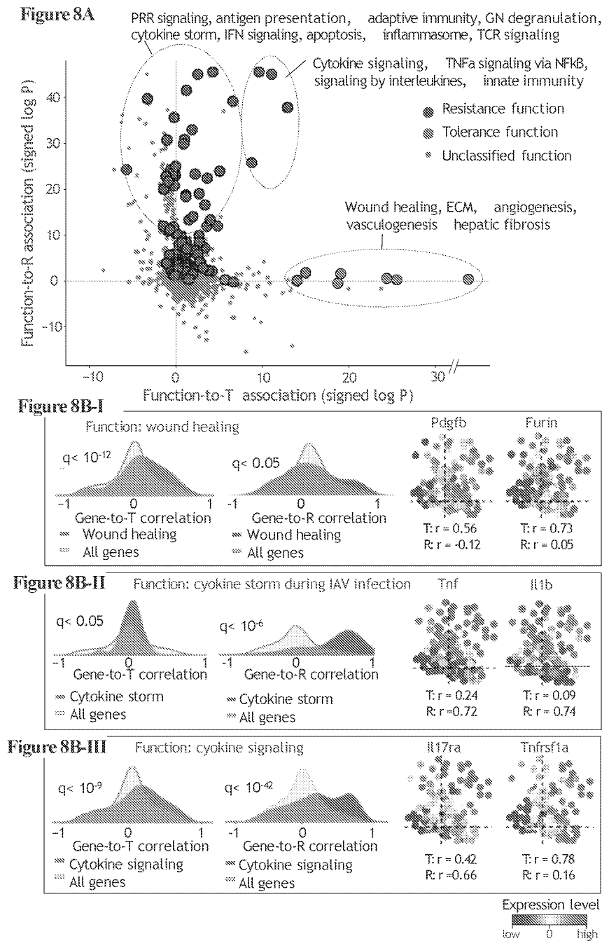

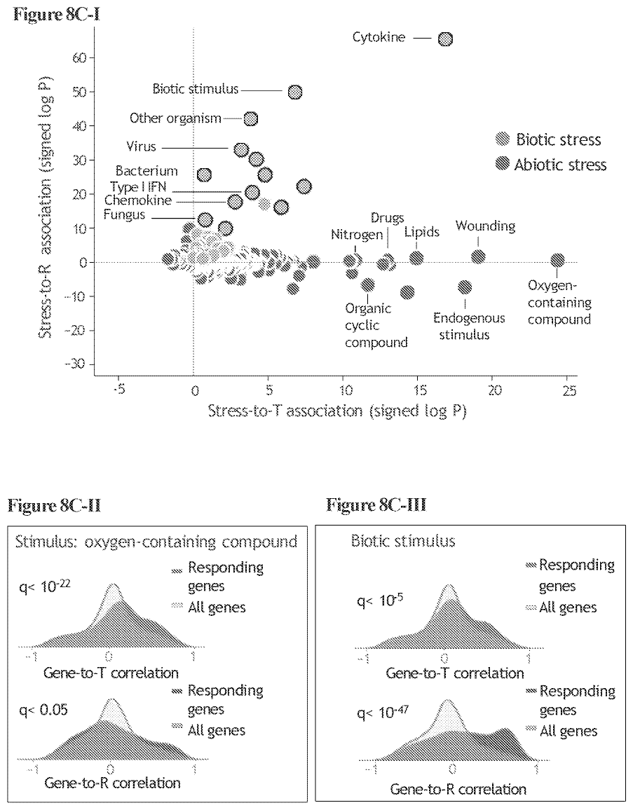

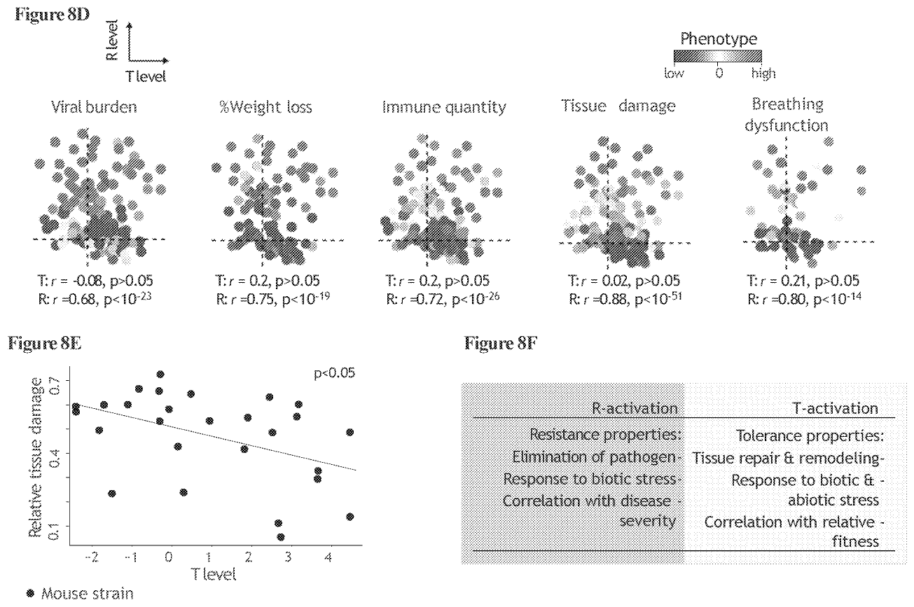

FIG. 6E: Analysis of evolutionarily-conserved genes. Shown are associations of a gene set for evolutionarily conserved genes (data from Hagai et al. (2018), conserved in human, macaque, rat, and mouse), demonstrated as in FIG. 8.

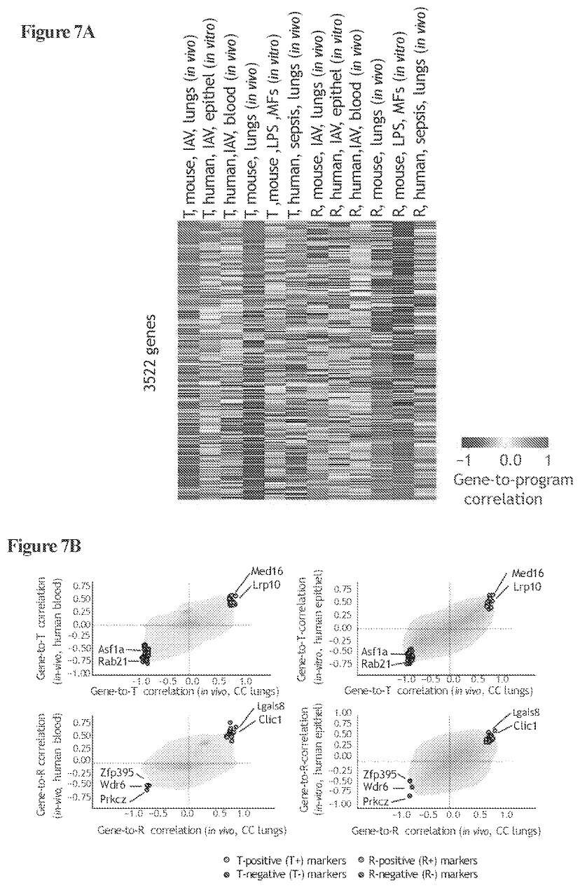

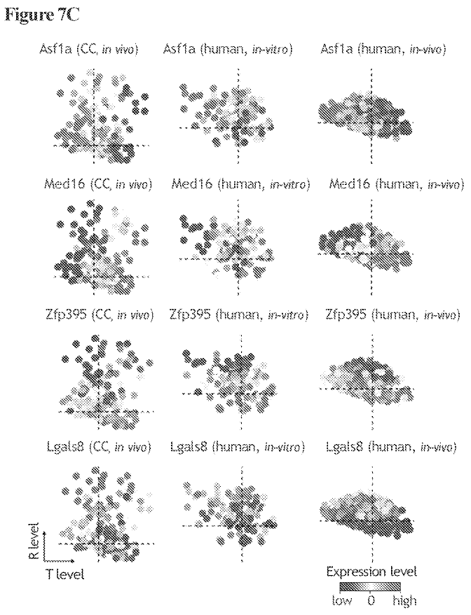

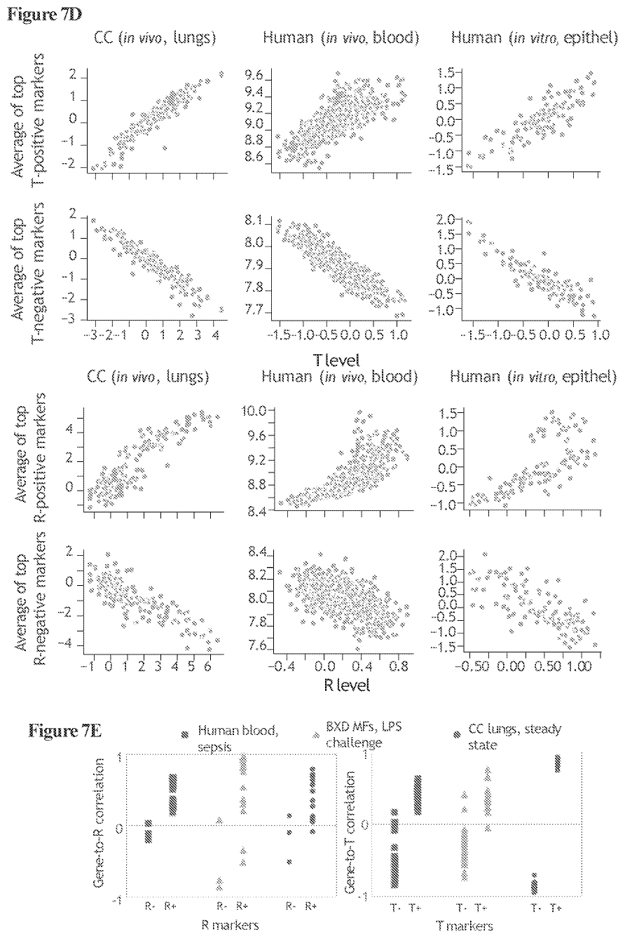

FIG. 7A-7E. Gene markers for the tissue-immune state of resistance and disease tolerance

FIG. 7A. Consistency of gene-to-program correlations across datasets. The heatmap presents correlations (color coded) between each gene (row) and the R or T level, using data in various independent datasets (columns). The heatmap highlights consistency of relations between genes and the R/T state, and a general anti-correlation between R and T levels.

FIG. 7B. Examples for the consistency of gene-to-program correlations. Comparisons of gene-to-program correlations in mouse (x axis) and human (y axis), for program T (top panels) and R (bottom panels), using human blood samples (in vivo study, left panels) and human bronchial epithelial cells (in vitro study, right panels). Selected markers of R and T levels (both positive and negative markers) are highlighted in color.

FIG. 7C. For several selected markers from B, detailed visualization of gene expression (color coding) across individuals (dots) of different R and T levels (axes). Coloring is based on expression data in the CC cohort and the human in vitro and in vivo studies (left to right panels).

FIG. 7D. Comparison of R and T levels that were calculated using all genes (using a deconvolution approach, x axis) versus R and T levels that were calculated as the average of marker genes (y axis). Positive and negative markers (top and bottom, respectively) are from B.

FIG. 7E. For the selected markers from B (R+, R-, T+ and T-groups), shown are gene-to-program correlations in several datasets.

FIG. 8A-8F. The identified transcriptional programs form the molecular basis of resistance and disease tolerance

FIG. 8A. Functional analysis. For each gene set of a certain functional category (a dot), indicated are the associations of the genes in the set with the levels of T (x axis) and R (y axis) (positive/negative values for associations with activation/inactivation of these programs). Established resistance/tolerance functions are color coded.

FIG. 8B-I-8B-III. Selected functions. For each function (i.e., a gene set), specifically, 8B-I (wound healing), 8B-II (cytokine storm), 8B-III (cytokine signaling) shown are the distributions of gene-to-program correlations for genes in this gene set compared to all genes (left: T, middle: R). Indicated are function-to-program association q-values (Wilcoxon test q-values for the bias in the correlations of the gene set). Right: Plots of two representative genes from each gene set (shown as in FIG. 3A-IV), highlighting gene-to-program correlations.

FIG. 8C-1-8C-III. Response to stress. 8C-I: Associations of genes induced following a certain stress (a dot) with the levels of T (x axis) and R (y axis). Biotic and abiotic stresses are color coded. 8C-II, 8C-III, Selected stress responses, using the same visualization as in FIG. 8B.

FIG. 8D. Disease severity is linked to program R. For each phenotype (a panel, indicated on top), shown is the landscape of R/T levels across individuals (as in FIG. 3A-IV) with individuals (dots) colored by their measured levels of the phenotype (blue/red indicating low/high phenotype levels; for each individual, R/T levels and phenotypes are at the same time point).

FIG. 8E. The relative damage is linked to program T. T levels (x axis) vs. relative tissue damage (y axis) across mouse strains (dots). The relative tissue damage is defined as the tissue damage relative to the IAV load in each CC strain.

FIG. 8F. Summary of the demonstrated links between known resistance/tolerance properties and the R/T programs.

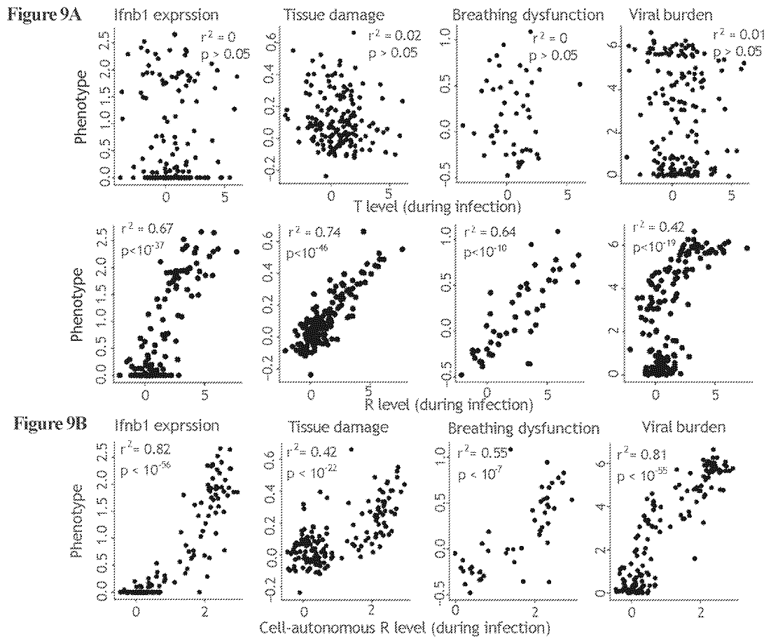

FIG. 9A-9B. Additional evidence that programs R and T act at the cell autonomous level FIG. 9A-9B. Comparisons of program levels with various phenotypes, using either the T levels (FIG. 9A, top), R levels (FIG. 9A, bottom), or the cell-autonomous R levels (FIG. 9B).

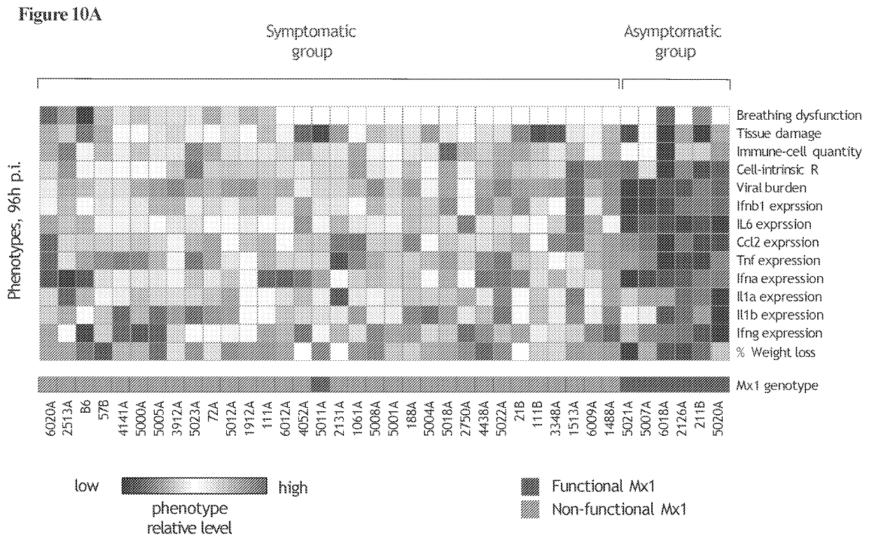

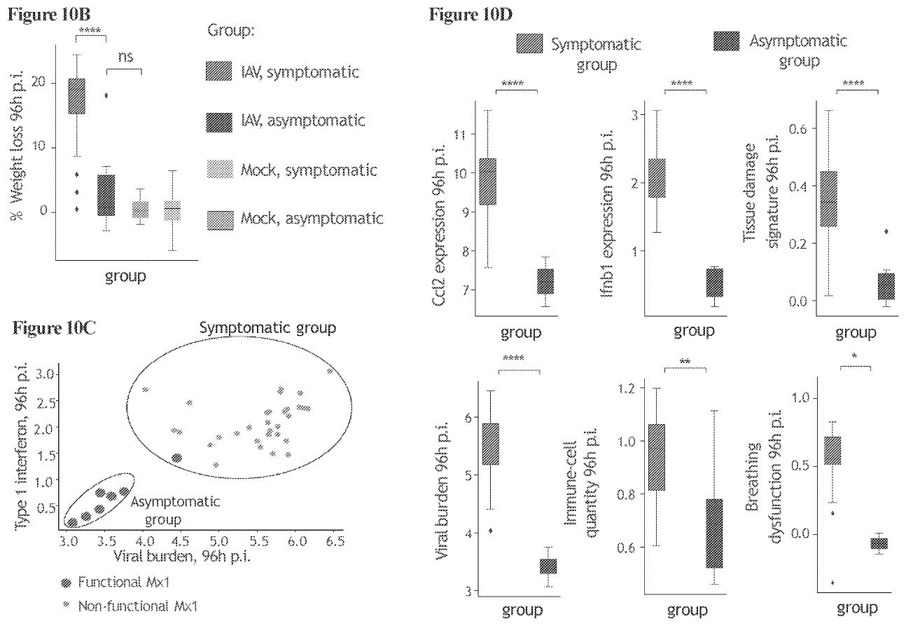

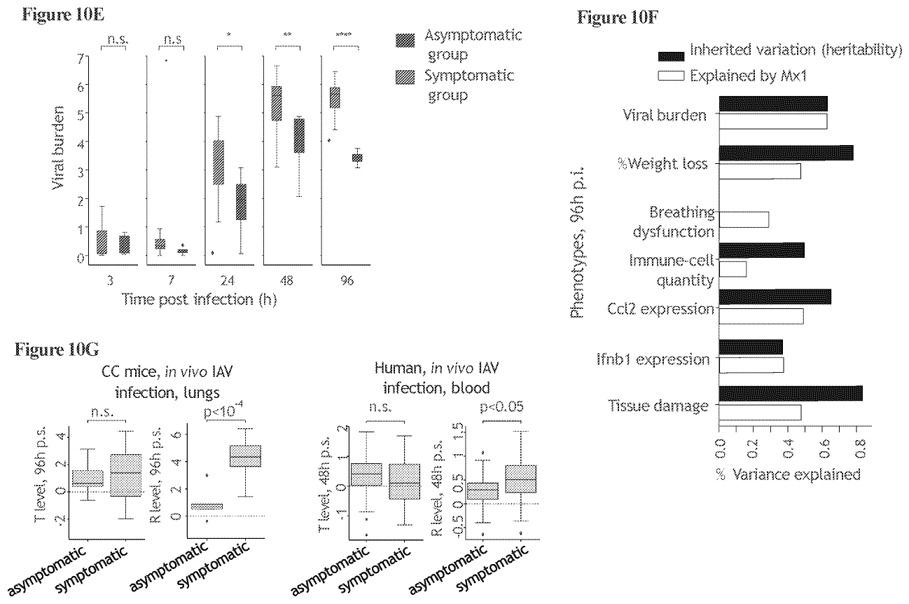

FIG. 10A-10G. Characterization of symptomatic and asymptomatic CC strains, and the relations to genetic variation in the Mxl gene

FIG. 10A. For each phenotype at 96h p.i. (rows) and each CC mouse strain (columns), shown is the measured phenotype (color coded, relative to the average across strains). The heatmap highlights a partition of individuals into two groups (symptomatic and asymptomatic). The two groups are used in plots B-E, G. Variation in the Mx1 gene, whose function has a known influence on susceptibility to IAV infection (Ferris et al., 2013), is indicated (bottom).

FIG. 10B. Comparison of weight loss (either IA V infection or mock treatment at 96h p.i) between the symptomatic and asymptomatic groups.

FIG. 10C. A scatter plot of viral burden (x axis) and type 1 interferon (y axis) at 96h p.i., across strains (dots). Circles indicate the symptomatic and asymptomatic groups. Color coding indicates strains that carry a functional (blue) or a non-functional (orange) Mx1 gene.

FIG. 10D. Comparison of disease severity phenotypes at 96h p.i. (panels) between the symptomatic and asymptomatic groups.

FIG. 10E. Comparison of viral burden between the symptomatic and asymptomatic groups, across different time points post infections.

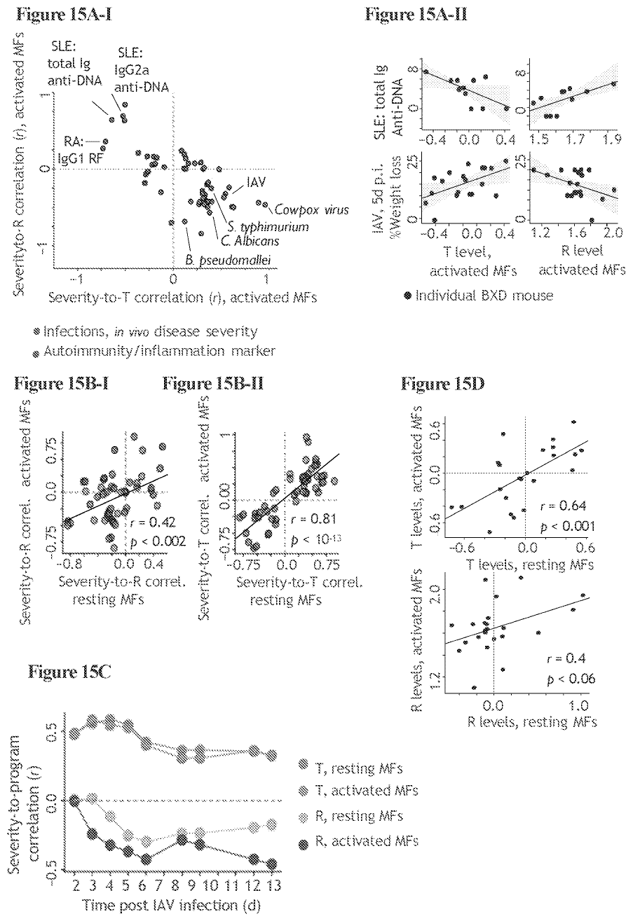

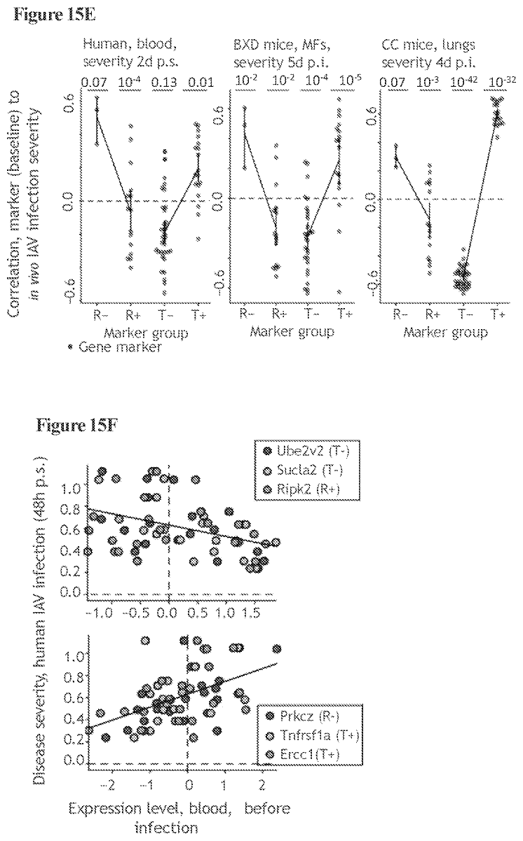

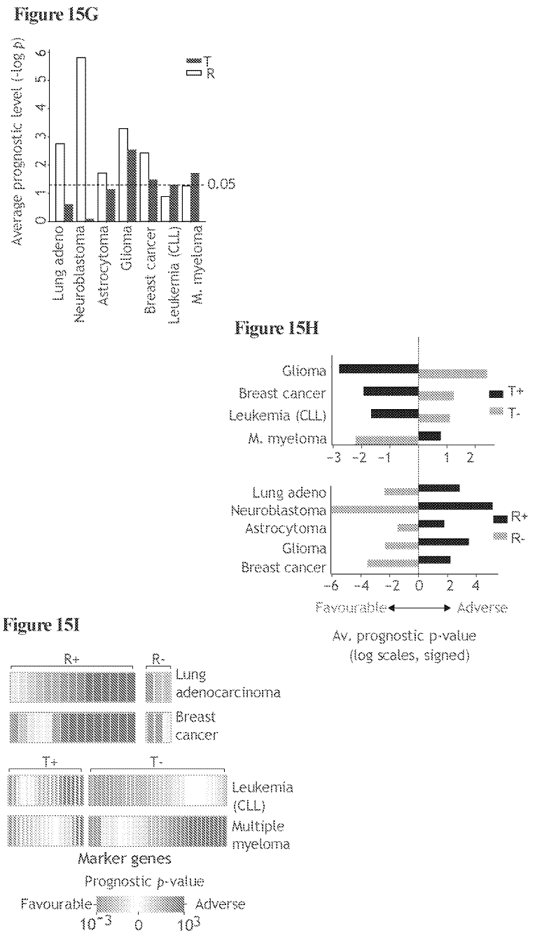

FIG. 10F. Percentage of explained phenotypic diversity in the IAV-infected individuals from the CC cohort. Y axis: phenotypes at 96h p.i. X axis: the percentage of phenotypic diversity that is explained by genetic background (i.e., the ‘inherited variation’, also referred to as ‘heritability’ (h²), black) and the percentage of phenotypic diversity that is explained by genetic variation in the Mx1 gene (white). In breathing functions, heritability was not measured. Overall, plots A-F, emphasize the presence of two phenotypic groups (symptomatic and asymptomatic strains), and further indicate that genetic variation in Mx1 explains much of the variation between these groups. The variation within the symptomatic group is further explored in FIG. 15.

FIG. 10G. Program R is linked to disease symptoms. Comparisons of T and R levels between symptomatic and asymptomatic individuals, either in the CC cohort at 96h p.i. (left) or the human cohort at 48h post symptoms (p.s., right).

FIG. 11A-11D. A variety of tissue-immune states are shaped by the combined contribution of the resistance and disease tolerance programs

FIG. 11A. Antagonistic relations between resistance and tolerance. Top: Average R levels (dotted line) and T levels (solid line). Bottom: Correlations (Pearson's r) between R and T levels at each time point. Data across CC mice (left) and human subjects (right) during IAV infection.

FIG. 11B. The spectrum of resistance and tolerance in health and disease. Scatter plots of R and T levels in healthy individuals (green dots) and IAV-infected symptomatic individuals (red dots) using murine lungs (left) and human blood (right). Symptomatic individuals were measured at 48h post symptom onset (p.s., human) or 96h p.i. (mice).

FIG. 11C: Scatter plots of R and T levels in normal and inflammatory conditions. Presented are individuals with Ebola infection (murine liver), SARS-COV-2 infection (human blood), septic shock (human blood), and LPS-activated murine peritoneal MFs. Each plot includes control (unstimulated) samples.

FIG. 11D: Changes in R and T levels in inflammatory conditions compared to normal conditions. Bar plots of R levels (left) and T levels (right) for each of the datasets in B and C. Indicated are t-test p-values.

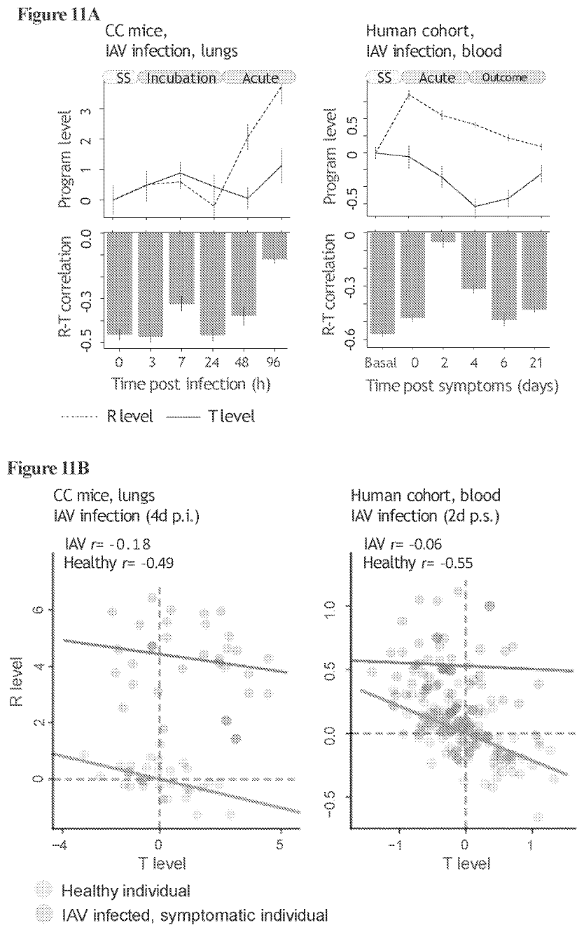

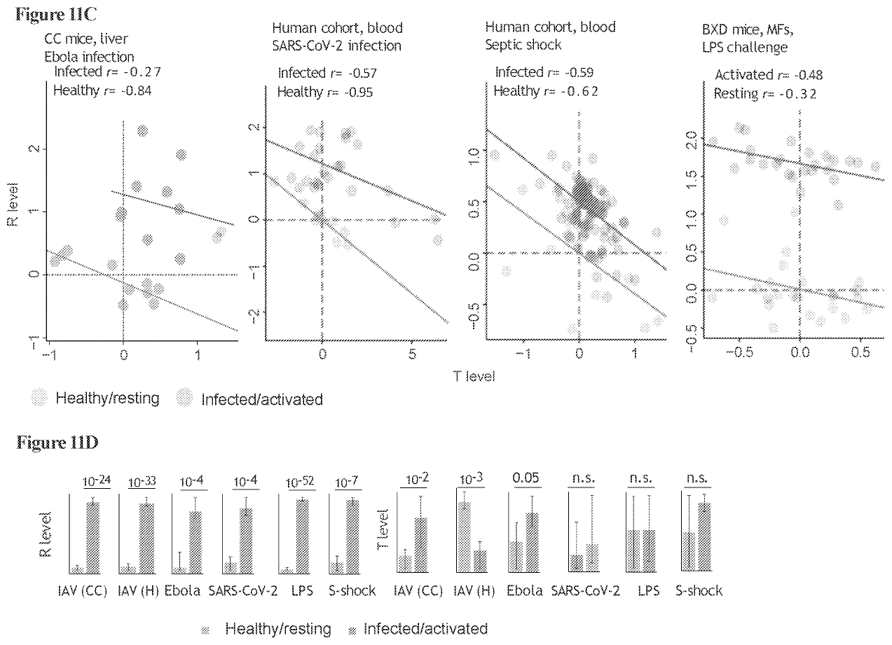

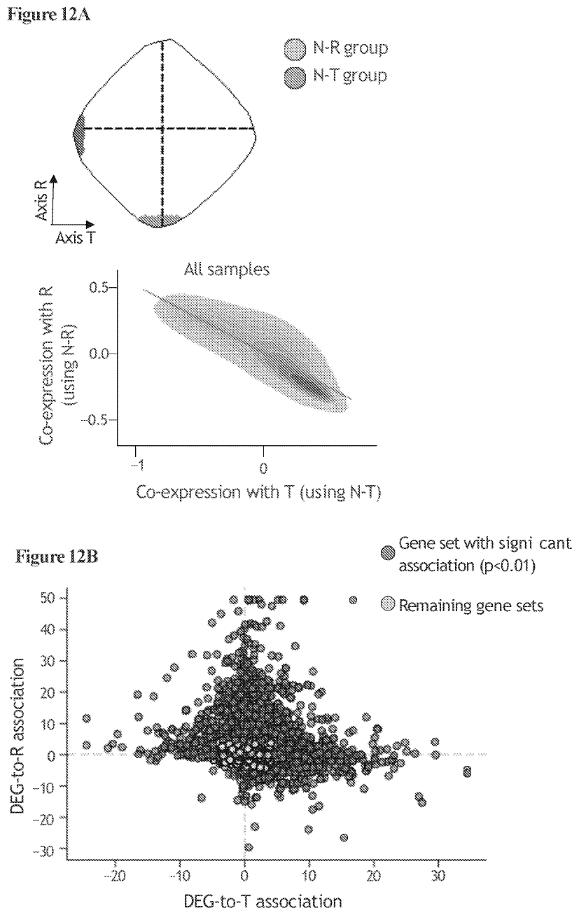

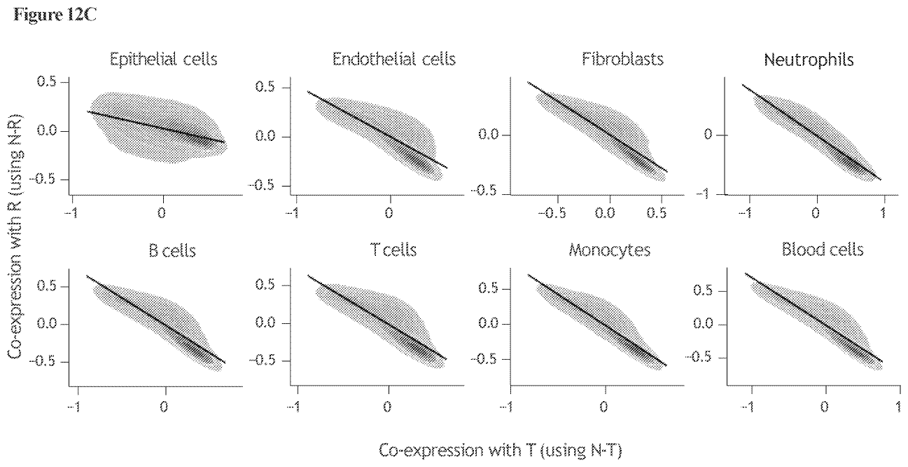

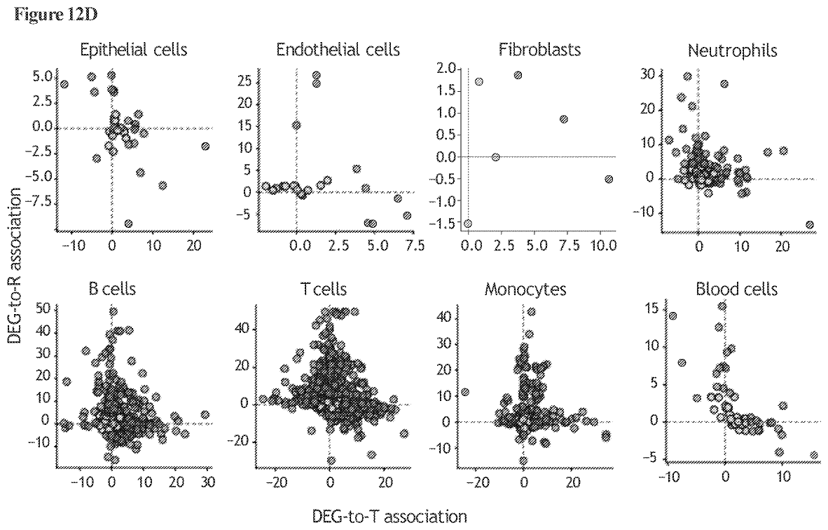

FIG. 12A-12D. Systematic analyses of transcriptome datasets

FIG. 12A, 12C. Co-expression with R and T levels in human data. The analysis relies on the SEEK algorithm (Zhu et al., 2015) and involves a systematic analysis of 3405 datasets from the GEO repository. (FIG. 12A) Top: the query genes of SEEK are indicated. Bottom: The plot presents the SEEK's co-expression of each human gene with the query set across a large collection of human transcriptome datasets (x and y axes: co-expression with the N-T and N-R query, respectively). (FIG. 12C) The same plots as in A but using human datasets of each specific cell type independently (indicated on top).

FIG. 12B, 12D. Analysis of a comprehensive collection of 4872 immunological signatures from the C7 MSigDB repository. (FIG. 12B) A scatter plot of DEG-to-T associations (x axis) against DEG-to-R associations (y axis), calculated over the collection of immunological signatures from the MSigDB repository (using all 4872 signatures). Red/gray indicate significant/insignificant associations, respectively (p=0.01 cutoff). (FIG. 12D) The same plots as in B but using signatures of specific cell types (indicated on top).

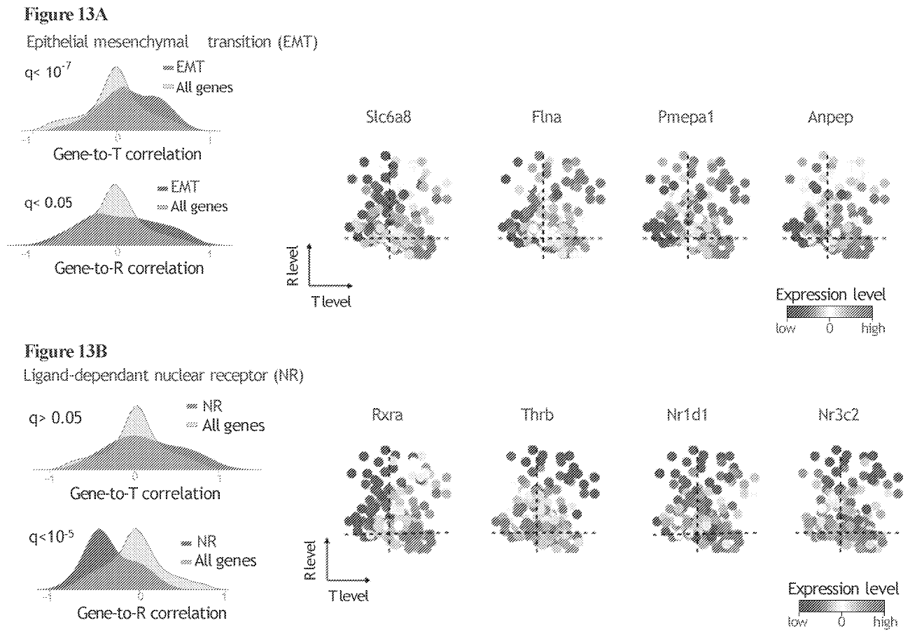

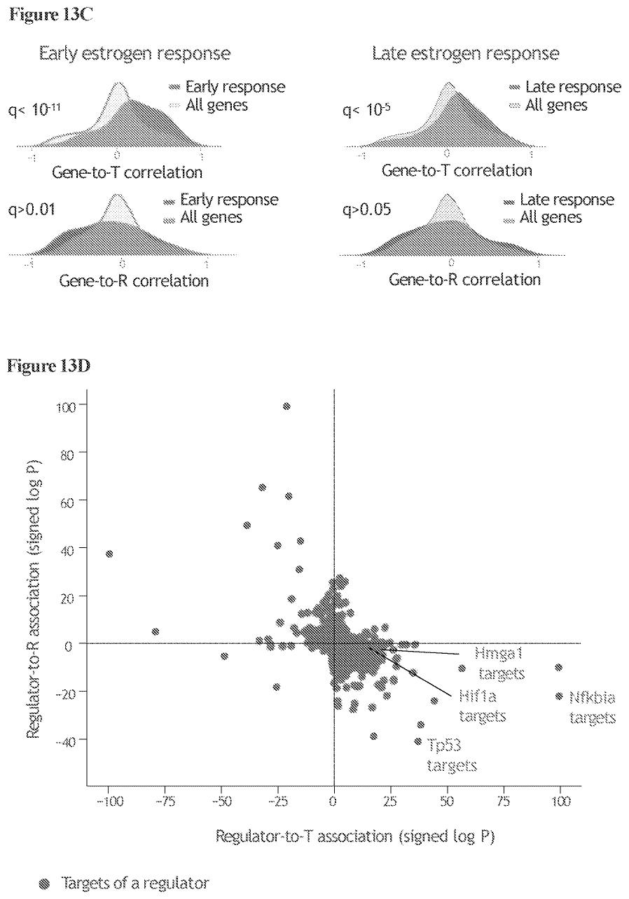

FIG. 13A-13E. Relations of programs R and T with selected functions and regulators

FIG. 13A-13C. For three functional categories (FIG. 13A: epithelial-mesenchymal transition, FIG. 13B: ligand-dependent nuclear receptors, FIG. 13C: early and late response to estrogen), the plots represent the associations with resistance and tolerance. Plots are presented as in FIG. 8B.

FIG. 13D-13E. Response to regulators. (FIG. 13D) For each set of target genes that are controlled by a certain regulator (a dot), indicated are the associations of these genes with the levels of T (x axis) and R (y axis). (FIG. 13E) Gene sets of selected regulators are demonstrated, using the same visualization as in FIG. 8B.

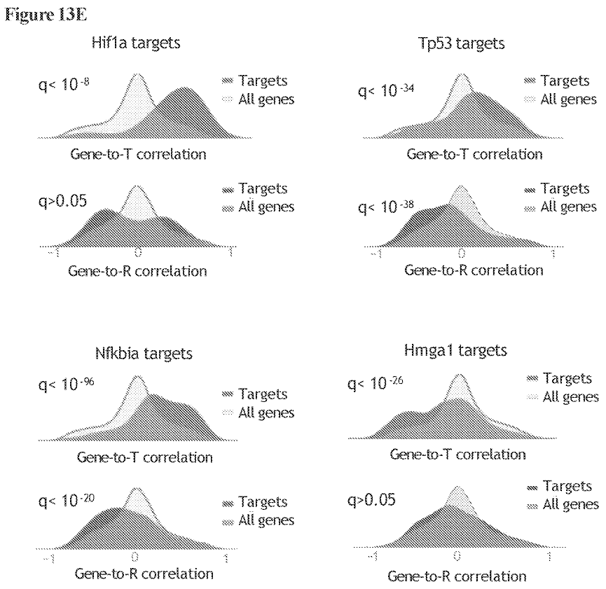

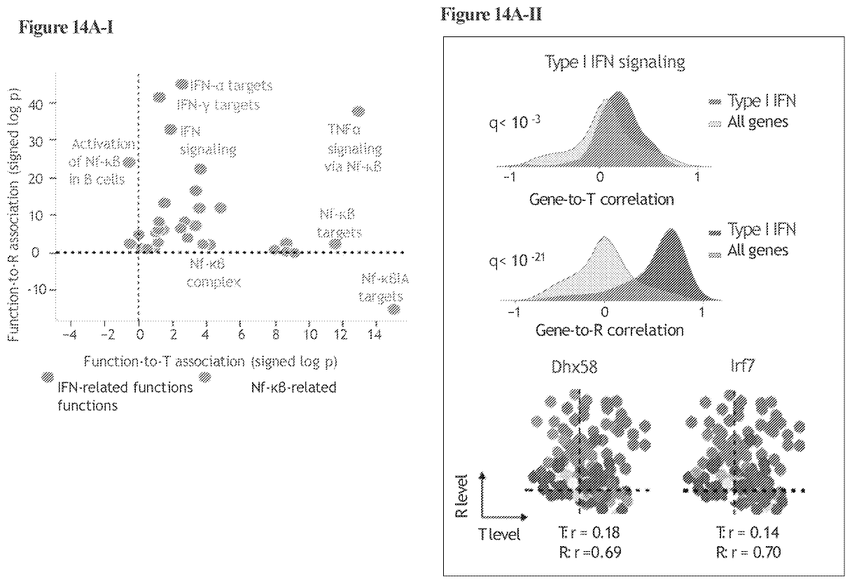

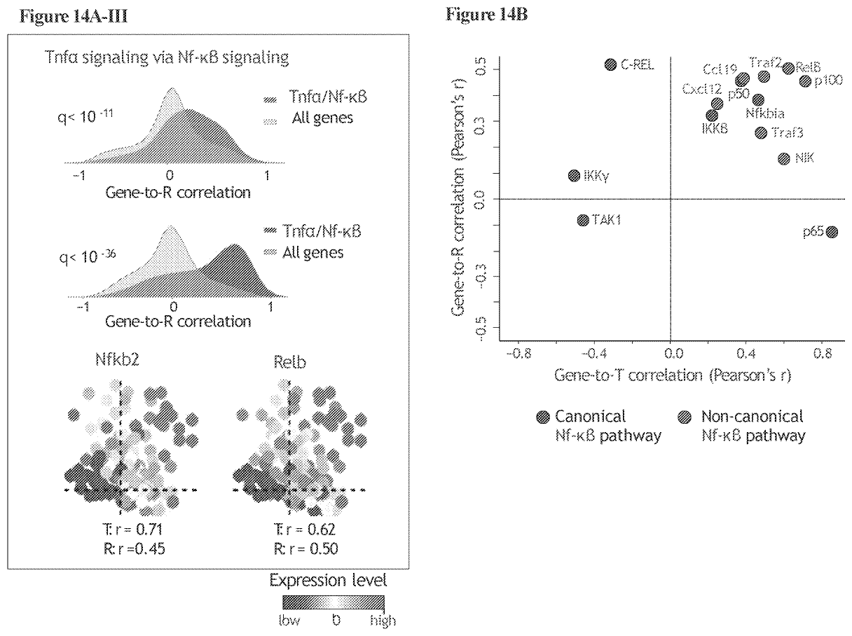

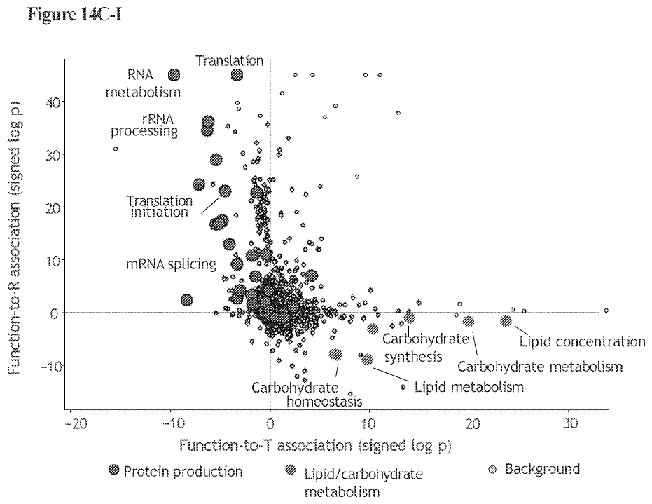

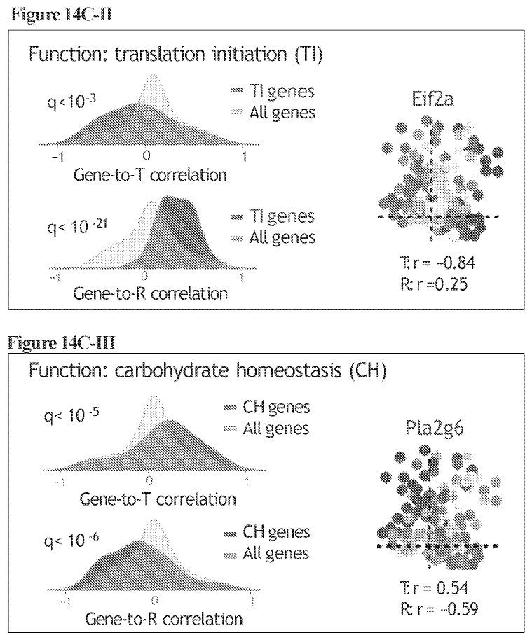

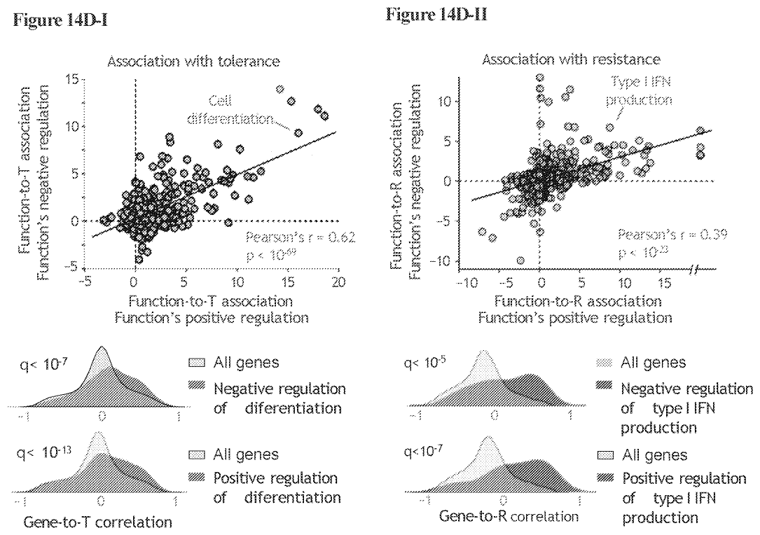

FIG. 14A-14D. Novel functions of resistance and disease tolerance

FIG. 14A-1-4A-III. Associations of NFκB-related functions (14A-I, 14A-III orange, e.g., ‘TNF-mediated NF-κB signaling’) and interferon-related functions (14A-I, 14A-II light blue, e.g., ‘type I IFN signaling’) with R and T levels, presented as in FIG. 8A, 8B.

FIG. 14B. Correlations (Pearson's r) of genes (dots) in the canonical (blue) and non-canonical (red) NFκB signaling pathways with T levels (x axis) and R levels (y axis) (shared genes were excluded). FIG. 14C-I-14C-III. Associations of ‘protein production’ (14C-I, 14C-II blue) and ‘lipid and carbohydrate metabolism’ (14C-1, 14C-III, red) functions with R and T levels, presented as in FIG. 8A, 8B.

FIG. 14D-1-14D-II. Coordination of positive and negative factors. 14D-I, 14D-14D-II: associations of positive regulators (x axis) and negative regulators (y axis) of each functional category (dots) with T levels (14D-I top panel) and R levels (14D-II top panel). Bottom: 14D-I, II Examples of specific associations, presented as in panel 14A.

FIG. 15A-151. Resistance and disease tolerance are predictive and prognostic for autoimmunity, infectious diseases and cancer survival in validation cohorts

FIG. 15A-1-15A-II. Resistance and tolerance in activated MFs are central to autoimmune and infectious diseases. 15A-1: The correlations between each disease (a dot) with T levels (x axis) and R levels (y axis) in activated MFs, across the BXD strains. Included are disease phenotypes of severity to infectious diseases (blue) and levels of autoimmune/inflammatory markers (red). 15A-11: Examples of these correlations across the BXD strains (dots), for two specific diseases. FIG. 15B-15D. R and T levels in both resting and activated MFs are predictive.

FIG. 15B-1-15B-II. The correlations of each disease (a dot) with R or T levels (15B-I or 15B-II panels) in resident MFs (x axis) versus activated MFs (y axis). Included are all disease phenotypes from A.

FIG. 15C. Pearson's correlations between program levels in peritoneal MFs and IAV-infection severity (across BXD strains, y axis). Time points of disease severity are indicated (x axis). Resting/activated MFs and programs are color coded.

FIG. 15D. The baseline R and T levels in resting MFs (x axis) can predict the R and T response in activated MFs (y axis), across BXD mouse strains (dots).

FIG. 15E. Corroboration using gene markers. Correlations between the baseline expression of markers and the late severity of IAV infection (y axis) in human (left), BXD mice (middle) and CC mice (right). Disease severity was measured at 2d post symptom onset (p.s., human), 5d p.i. (BXD) 4d p.i. (CC). Markers were measured in blood (human), resting peritoneal MFs (BXD) and lungs (CCs). R-, R+, T-, T+marker groups are for R-inactivation, R-activation, T-inactivation, and T-activation, respectively. Top: Fisher's combined p-values.

FIG. 15F. The relationships between the baseline expression of markers with disease severity at 48h p.s. (in human blood, color coded; marker groups are indicated).

FIG. 15G-15H. R/T markers are prognostic for cancer survival. Average of prognostic p-values for different human cancers. Averaging of −log p-values across all markers (FIG. 15G) or a separate averaging of the R+, R-, T+ and T-groups (FIG. 15H). Included are tumors for which either R or T levels are significant predictors (p<10⁻⁷).

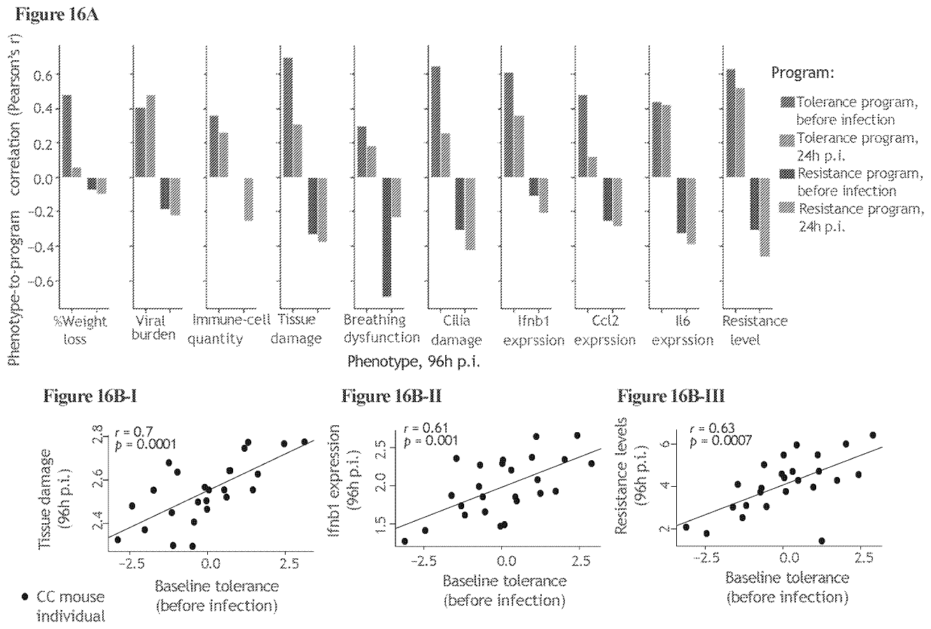

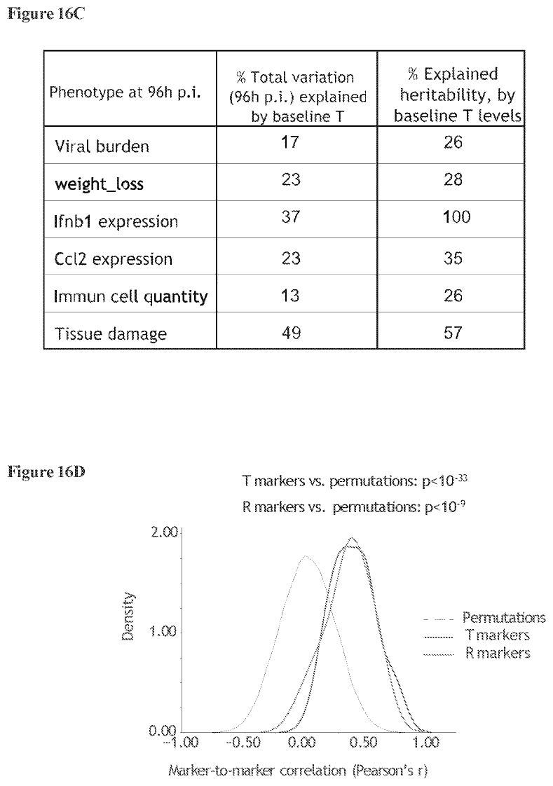

FIG. 151. The prognostic p-values of R and T markers in four cancer types are shown as heatmaps. FIG. 16A-16E. The baseline resistance/disease-tolerance state is correlated with severity of IAV infection

FIG. 16A. Pearson's correlations between baseline/early program levels and late disease severity phenotypes (across the 27 symptomatic strains, y axis). Disease phenotypes (at 96h p.i.) are indicated (x axis). Program levels are for tolerance and cell-intrinsic resistance, before infection or at 24h p.i. (color coded).

FIG. 16B-I-16B-III. Examples of the relationships between baseline T and disease severity at 96h p.i. across symptomatic strains (dots), 16B-I (tissue damage), 16B-II (IFN expression), 16B-III (resistance level).

FIG. 16C. Percentage of inherited variation in IAV infection severity that is explained by the baseline level of tolerance. For each phenotype at 96h p.i. (column 1), reported are: (i) the percentage of total variation that is explained by variation in baseline (before infection) T levels (column 2), and (ii) the percentage of inherited variation that is explained by variation in baseline (before infection) T levels (column 3). Calculations are based on inherited variation (heritability) values that are reported in FIG. 2D.

FIGS. 16D-16E. R and T levels persist for a relatively long period of time in healthy individuals. Shown is the correlation between measurements (i) at the beginning of the winter and (ii) at least several weeks afterwards (mid-winter).

FIG. 16D. Distribution of correlations across the T and R markers, showing consistency over time.

FIG. 16E. Correlations (color coded) of all T-negative markers (columns, beginning of the winter) against all T-negative (top) and T-positive (bottom) markers (rows, mid-winter). T and R markers are from Table 1.

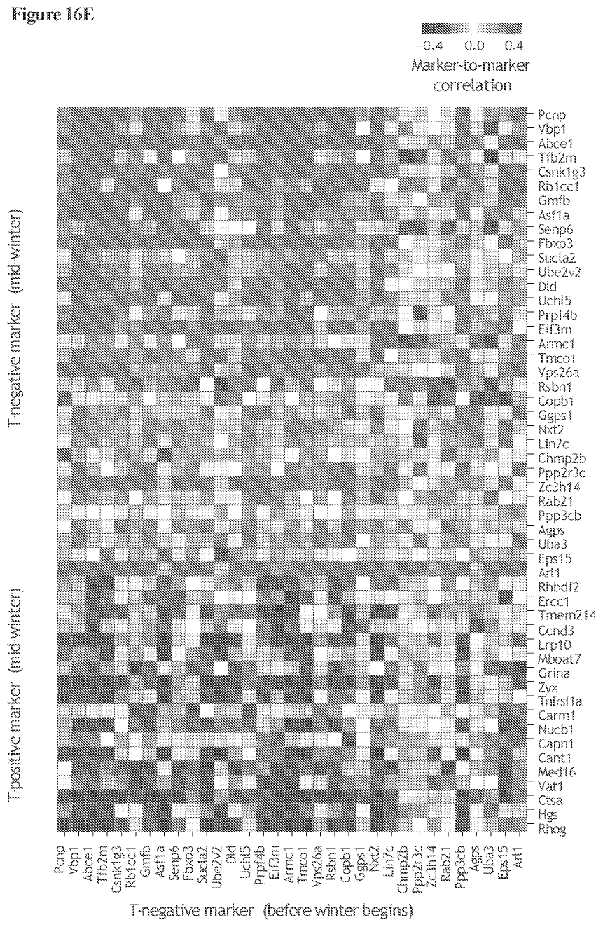

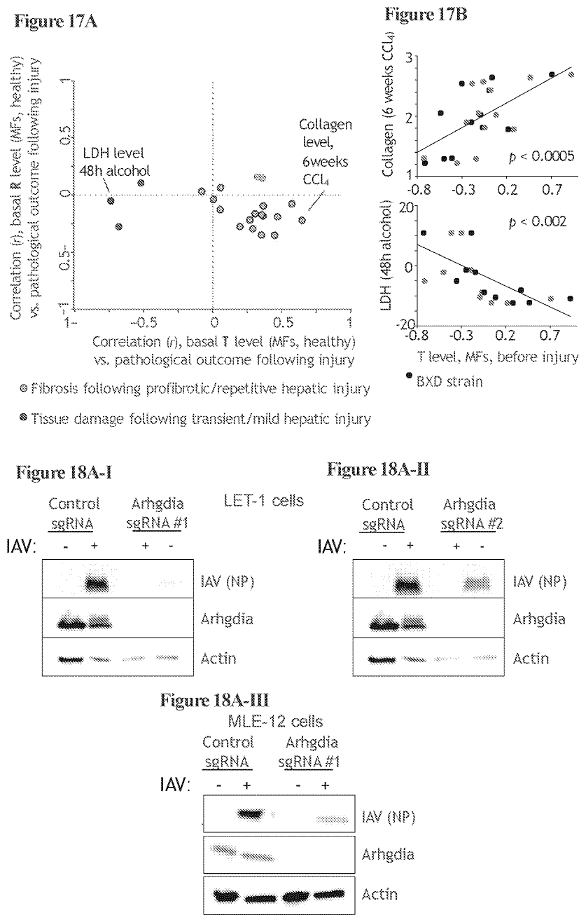

FIGS. 17A-17B. Baseline T and R states in peritoneal MFs are linked to the in vivo response to stimuli

Response to injury.

FIG. 17A. For various phenotypes (dots), shown are correlations between the phenotype and the baseline T (x axis) or R (y axis) levels. Correlations were calculated across the BXD strains. T and R levels were measured in resting peritoneal MFs from healthy BXD mice. Phenotypes included are fibrosis biomarkers following profibrotic/repetitive injury (yellow) and tissue damage markers following mild/transient injury (dark gray).

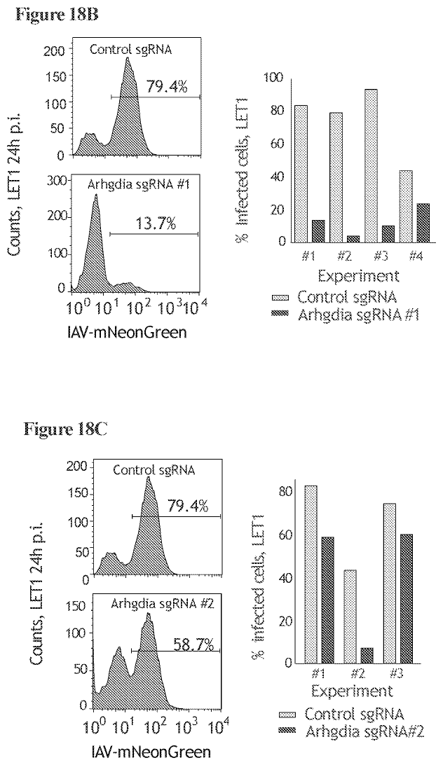

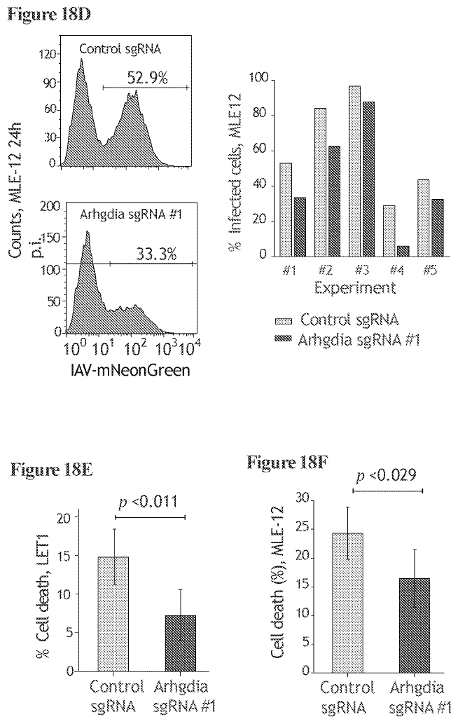

FIG. 17B. selected examples, demonstrating baseline T levels (x axis) versus phenotypes (y axis) across mouse strains (dots), using T levels in resting (gray) and activated (black) MFs. FIG. 18A-18F. Reduced IAV infection and cell death in Arhgdia-depleted cells

FIG. 18A-I-18A-III. Immunoblot analysis of Arhgdia and IAV nucleoprotein (NP) expression in parental and Arhgdia-depleted LET1 (FIG. 18A-I and FIG. 18A-II: sgRNA #1 and sgRNA #2, SEQ ID NOs: 37 and 38, respectively) and MLE-12 (FIG. 18A-III) cells, 24h post infection.

FIGS. 18B-18D. Representative flow cytometry data of Arhgdia-depleted LET1 in two independent clonal lines (denoted as sgRNA #1 and sgRNA #2) (FIG. 18B, FIG. 18C) or MLE-12 (FIG. 18D) cells versus control cells, infected with PR8-mNeonGreen virus for 24 h. Bar graph (right), represents percentage infected cells in indicated independent experiments.

FIGS. 18E, 18F. Shown is the percentage of dead cells, determined by flow cytometry and the Live/Dead assay in Arhgdia-depleted LET1 (18E) or MLE-12 (18F) cells, versus control cells. The data consist of at least three independent experiments. In all experiments, cells were infected at a multiplicity of infection (MOI) of 5 and quantified at 24h post infection.

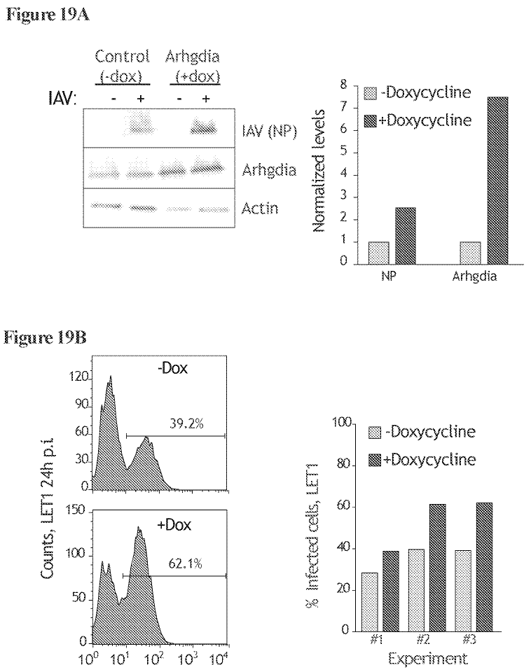

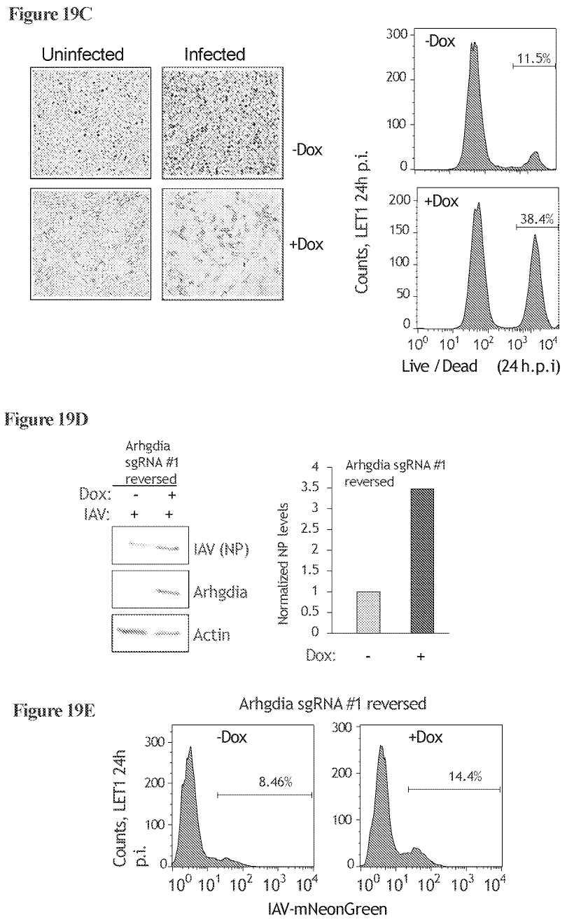

FIG. 19A-19E. Arhgdia expression modulates IAV infection and the subsequent cell death FIG. 19A. Western blot analysis of NP and Arhgdia protein expression in Arhgdia/Tet-on LET1 cell line (pool) with (+) or without (−) doxycycline (dox) treatment. Right: Quantification analysis of band density with (dark grey) or without (light grey) doxycycline.

FIG. 19B. Left: Representative flow cytometry data of inducible Arhgdia LET1 cells infected with IAV-mNeonGreen virus (PR8 strain) for 24 hr. Right: Bar graph, represents % infected cells in three independent experiments.

FIG. 19C. Left: bright field images of Arhgdia/Tet-on LET1 cells, exposed (or not) to doxycycline, and infected (or not) with PR8 for 24 hr. Right: cell death after IAV infection was analyzed by flow cytometry using the Live/Dead assay in tetracycline-inducible Arhgdia stable cell line with (+) or without (−) doxycycline (dox) treatment.

FIG. 19D. Western blot analysis of NP and Arhgdia protein expression in Arhgdia sgRNA #1 reversed Let1 cells, with and without doxycycline (Dox), infected with IAV to assay viral gene expression. Right: Quantification analysis of band density with (dark grey) or without (light grey) doxycycline (Dox).

FIG. 19E. Representative flow cytometry data of Arhgdia sgRNA #1 reversed Let1 cells, with and without doxycycline (Dox), infected with IAV-mNeonGreen virus (PR8 strain) for 24 hr.

In all experiments, cells were induced with doxycycline for 72 hr before IAV infection and were infected at MOI=5.

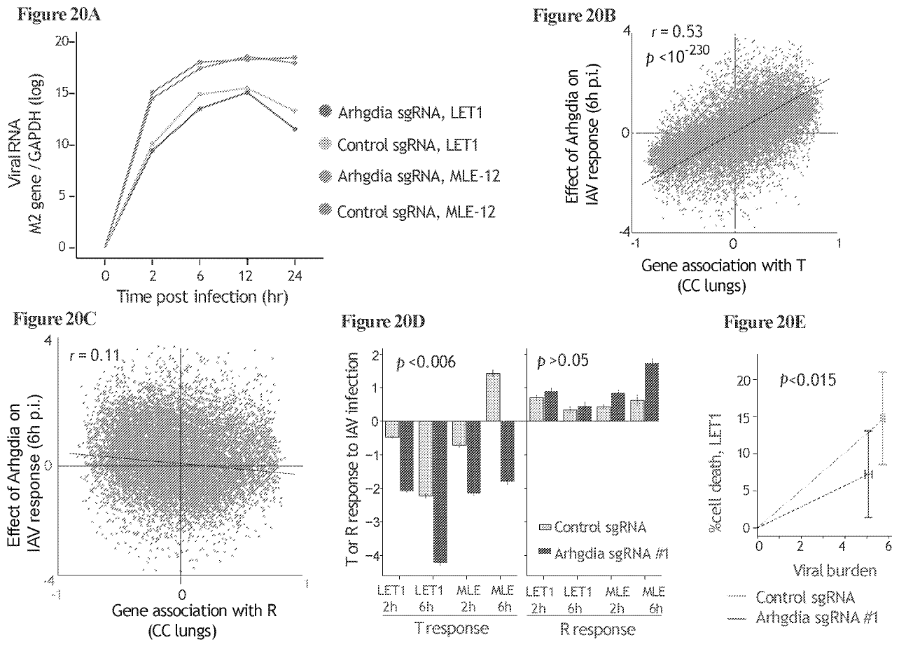

FIG. 20A-20E. Arhgdia affects disease-tolerance responses in IAV-infected epithelial cells

FIG. 20A. Viral RNA levels in cells expressing, or not, Arhgdia. The viral RNA levels were determined by qRT-PCR, using primers specific for the viral M2 gene and cellular GAPDH, respectively.

FIG. 20B. For each gene (a dot), shown is its association with T levels (using lung data across CC mice; x axis) compared to the effect of Arhgdia on this gene (differential expression in LET1 cells: response at 6h post infection. of control versus Arhgdia-depleted cells, y axis).

FIG. 20C.Comparison between the signatures of program R and Arhgdia. For each gene (a dot), shown is its association with R levels (using lung data across CC mice; x axis) compared to the effect of Arhgdia on this gene (differential expression in LET1 cells: control 6h at post infection. versus Arhgdia-depletion at 6h post infection., y axis).

FIG. 20D. Shown are T and R responses to IAV infection in Arhgdia-depleted and control MLE-12 (MLE) or LET1 cells, at the indicated time points (2-6h post infection). (T: p<0.006, R: p >0.05, paired t-test). Confidence intervals were calculated using bootstrapping of genes.

FIG. 20E. Comparison between the ‘relative tissue damage’ in Arhgdia-deleted LET1 cells versus control LET1 cells at 24h p.i. The relative tissue damage is defined as the slope of cell-death against the viral burden.

In all plots, MOI=5 of PR8 virus, titrated on MDCK cells.

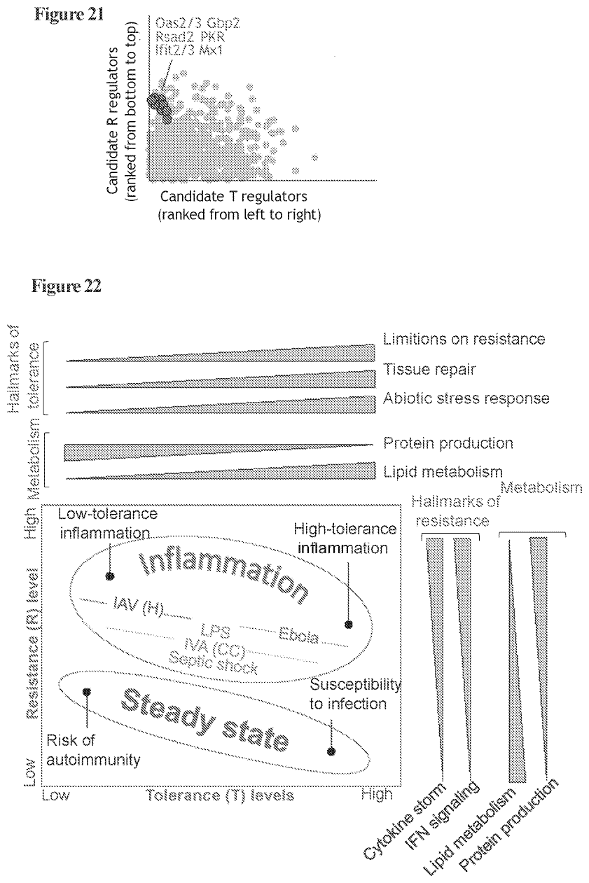

FIG. 21. Identification of resistance regulators

Ranking of regulator candidates by their associations with the disease tolerance and resistance programs. Validated IAV restriction factors (purple) obtained high ranking for the resistance program.

FIG. 22. The functions of resistance and disease-tolerance in health and disease

The molecular programs of resistance and tolerance generate a wide spectrum of molecular states, both during inflammation and in a healthy steady state. The programs involve a variety of functions and are predictive to a broad range of diseases. The methodology developed here for the assessment of resistance and tolerance could be used in clinical settings.

### DETAILED DESCRIPTION OF THE INVENTION

In the present disclosure, the inventors explored gene signatures that are linked to the two main defense strategies: disease tolerance and resistance. A gene program was identified for the disease tolerance strategy (T) that is separable from the gene program of the resistance strategy (R). The present disclosure further provided refinement for the current gene signature of resistance by allowing its precise definition that is uncoupled from the gene signature of disease tolerance. These definitions allowed the inventors to study, for the first time, the relations between the two defense strategies at the molecular level. The inventors found that both T and R states are robustly detected at the gene-expression level in multiple cell types, including non-immune, innate and adaptive immune cell types. The analysis revealed generic signatures for the identified T and R states across the various cell types (both mouse and human cells), allowing the development of personalized and quantitative metrics for the molecular level of each program. The two programs explain a roughly equal percentage of the molecular and phenotypic variation, consistent with previous studies that have shown a roughly equal number of disease-tolerance and resistance mutations that influence the progression of infections [Ayres, J. S., Freitag, N. & Schneider, D. S. Genetics 178, 1807-1815 (2008)].

A tight link was found herein between the baseline T levels in peritoneal macrophages (MFs) (before stimulus) and the in vivo pathological response following stimulations. While excessive baseline T activity in MFs is correlated with elevated pathology in case of chronic hepatic injury and infections, the baseline MF state of poor T levels is correlated with elevated pathology in response to transient hepatic injury. The inventors speculate that the homeostatic intermediate levels of program T may benefit from the compromise between opposing forces. In line with this, there is a high prevalence of the intermediate T level in healthy subjects. Characterizing the associations of programs T and R with additional disease outcomes and therapeutic responses is highly valuable. For example, one additional physiological condition in which the T program could be very important is pregnancy: it has been earlier shown that a strong pro-inflammatory phenotype may lead to a spontaneous miscarriage [Christiansen, O. B., Nielsen, H. S. & Kolte, A. M. Seminars in Fetal and Neonatal Medicine 11, 302-308 (2006); van Dunné, F. M., et al., Genes & Immunity 7, 688-692 (2006)], and a counterbalancing T program is potentially needed for a successful pregnancy. In an additional example, another physiological condition in which the T program could be very important is the pathological response to hepatitis B and hepatitis C viruses, such as the effect of T on the long-term progression from acute to chronic infection, the development of fibrosis, cirrhosis and hepatocarcinogenesis.

The data of the present disclosure suggest that a high-T/low-R state before infection is associated with a greater severity of future infections, implying a central role of the baseline T-R balance in disease progression. These findings further suggest a ‘hit hard, hit quickly’ model of the host defense against pathogens: a greater severity of infection could be a consequence of an early inhibition of resistance by disease-tolerance functions. It has been long argued whether death resulting from dysregulation of inflammation in sepsis is due to a poor initial immune response followed by high pathogen burden and secondary hyper-inflammation or, alternatively, is due to a disproportionately strong initial immune response against a low pathogen burden that leads to inadequate systemic responses. The findings disclosed herein argue for the former, in line with studies of bacterial sepsis [Westendorp, R. G. et al. The Lancet 349, 170-173 (1997)] and respiratory infections [Bradley, K. C. et al. Cell Reports 28, 245-256.e4 (2019); Graham, J. B. et al. PLOS Pathog 17, e1009287-e1009287 (2021); Lee, J. S. & Shin, E.-C. Nature Reviews Immunology 20, 585-586 (2020)], and consistent with the efficacy of approaches that boost the baseline/early resistance such as vaccines [O'Neill, L. A. J. & Netea, M. G. Nat Rev Immunol 20, 335-337 (2020); Sánchez-Ramón, S. et al. Frontiers in Immunology 9, 2936 (2018)].

Using independent cohorts, the inventors show that the baseline resistance and tolerance levels are significant predictors, with opposing directions, of clinical susceptibility to invading pathogens, abnormalities related to autoimmune diseases, and cancer survival. These results highlight the central role of the balance between the molecular state of resistance and tolerance in keeping the inflammatory status in check, with potential implications to clinical diagnosis and disease treatment. Thus, a first aspect of the present disclosure relates to a method for evaluating the immune and/or the immunological state of a subject by determining the levels of resistance and/or tolerance of the subject. In some embodiments, determination of the state of resistance and/or tolerance is determined using particular sets of biomarkers specific for each one of resistance and tolerance. It should be understood that determination of the biomarker signature for each of the resistance and/or tolerance is based in some embodiments on the expression level of each of the biomarkers. More specifically, in some embodiments, determination of the expression level of the biomarkers disclosed herein may be performed at the nucleic acid level (e.g., the mRNA level) and/or at the protein level. Thus, in some embodiments, the term biomarker/s as used herein relates to a measurable substance or molecule in an organism whose presence corelates, and thus indicative of a specific phenomenon, state, condition or process (e.g., disease. or any other physiological state). In some embodiments biomarkers of the present disclosure relate to biomarker gene/s or biomarker gene product/s (e.g., biomarker proteins and/or biomarker mRNA). In some embodiments of the present disclosure, the biomarkers disclosed herein reflect the tolerance and the resistance state of the examined subject. More specifically, in some embodiments, the method disclosed herein comprises the following steps: The first step (a), involves determining in at least one biological sample of the subject the expression level of at least one of:

In some embodiments (i), the expression level of at least one biomarker of resistance is determined, to obtain an expression value for each of the at least one biomarker/s. More specifically, the at least one biomarker/s of resistance is at least one of: MAX Interactor 1 (MXI1), Zinc Finger Protein 395 (ZNF395), Xeroderma Pigmentosum, Complementation group C (XPC), Methylenetetrahydrofolate Dehydrogenase 2 (MTHFD2), Proteasome Activator Subunit 2 (PSME2), Integrator Complex Subunit 12 (INTS12), Proteasome 20S Subunit Beta 7 (PSMB7), RNA Binding Motif Protein 7 (RBM7), Solute Carrier Family 6 Member 8 (SLC6A8), and optionally, of Janus Kinase 2 (JAK2), or any combination thereof; and

In some additional or alternative embodiments (ii), the expression level of at least one biomarker of tolerance is determined, to obtain an expression value for each of the at least one biomarker/s. More specifically, the at least one biomarker/s of tolerance is at last one of: Serine Incorporator 1 (SERINC1), ADP Ribosylation Factor Like GTPase 1 (ARLI), COP9 Signalosome Subunit 2 (COPS2), Cereblon (CRBN), Mitogen-Activated Protein Kinase Kinase 2 (MAP2K2), Rho GDP

Dissociation Inhibitor Alpha (ARHGDIA), Glutamate Ionotropic Receptor NMDA Type Subunit Associated Protein 1 (GRINA), Syntaxin Binding Protein 2 (STXBP2), RNA Binding Motif Protein 7 (RBM7), Solute Carrier Family 6 Member 8 (SLC6A8) or any combination thereof.

The next step (b), of the disclosed methods involves determining if the expression values obtained in steps (a) (i) and/or (a) (ii) for each of the at least one biomarker/s, is positive or negative with respect to a predetermined standard expression value or to an expression value of the biomarker/s in at least one control sample.

Thus, wherein at least one of:
In some embodiments (I), a positive expression value of at least one of the resistance biomarkers MTHFD2, PSME2, INTS12, PSMB7 and RBM7, and optionally of JAK2, biomarker/s in the sample, and/or a negative expression value of at least one of the MXI1, ZNF395, XPC and SLC6A8 biomarker/s in the sample, indicate(s) that the resistance level is elevated in the subject. In yet some further alternative or additional embodiments (II), a positive expression value of at least one of the tolerance biomarkers MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample indicate(s) that the tolerance level is elevated in the subject. Thus, the disclosure thereby provides the determination of the immune and/or immunological state in and of the subject.

In some alternative embodiments of the disclosed methods, the first step (a), involves determining in at least one biological sample of the subject the expression level of at least three biomarkers of at least one of: In some embodiments (i), the expression level of at least one biomarker of resistance is determined, to obtain an expression value for each of the at least one biomarker/s. More specifically, the at least one biomarker/s of resistance is at least one of: MXI1, ZNF395, Xeroderma XPC, MTHFD2, PSME2, INTS12, PSMB7, RBM7, SLC6A8, and optionally, of JAK2, or any combination thereof; and

In some additional or alternative embodiments (ii), the expression level of at least one biomarker of tolerance is determined, to obtain an expression value for each of the at least one biomarker/s. More specifically, the at least one biomarker/s of tolerance is at last one of: SERINC1, ARLI, COPS2, CRBN, MAP2K2, ARHGDIA, GRINA, STXBP2, RBM7, SLC6A8 or any combination thereof.

It should be understood the at least three biomarkers may be in some embodiments, at least three or more of the resistance biomarkers (i), in some other embodiments, at least three or more of the tolerance biomarkers (ii), or in some other embodiments, at least three or more of the resistance (i), and the tolerance biomarkers (ii). The next step (b), of the disclosed methods involves determining if the expression values obtained in steps (a) (i) and/or (a) (ii) for each of the at least three biomarkers, is positive or negative with respect to a predetermined standard expression value or to an expression value of the biomarker/s in at least one control sample.

Thus, wherein at least one of:
In some embodiments (I), a positive expression value of at least one of the resistance biomarkers MTHFD2, PSME2, INTS12, PSMB7 and RBM7, and optionally of JAK2, biomarker/s in the sample, and/or a negative expression value of at least one of the MXI1, ZNF395, XPC and SLC6A8 biomarker/s in the sample, indicate(s) that the resistance level is elevated in the subject.

In yet some further alternative or additional embodiments (II), a positive expression value of at least one of the tolerance biomarkers MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample indicate(s) that the tolerance level is elevated in the subject. Thus, the disclosure thereby provides the determination of the immune and/or immunological state in and of the subject.

Thus, the disclosed methods involve in the first step determination of the expression level of specific biomarkers to obtain an expression value for each, as will be elaborated herein after. The second step involves determination if the expression value is positive or negative. It should be understood that determination of a “positive” or alternatively “negative” expression value with respect to a standard value or a control value may involve in some embodiments comparison of the expression value of the examined sample as obtained in steps (a) (i) and/or (a) (ii), with the expression value obtained for a control sample, or from any established or predetermined expression value (e.g., a standard value) obtained from a known control (either healthy controls or of subjects suffering from a pathological disorder). Thus, in some embodiments, “positive” is meant an expression value that is higher, increased, elevated, overexpressed in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the expression value of a healthy control, any other suitable control or any other predetermined standard. Still further, a “negative” expression value in some embodiments may be a reduced, low, non-existing or lack of expression of a biomarker in about 5% to 100% or more, specifically, 5%, 10%, 15%, 20%, 25%, 30%, 35%, 40%, 45%, 50%, 55%, 60%, 65%, 70%, 75%, 80%, 85%, 90%, 95%, 100%, when compared to the expression value of a healthy control, any other suitable control or any other predetermined standard.

As used herein, “healthy controls” or “healthy population” may refer to a population of subjects that does not suffer from a disease of interest or refer to a population before appearance of a disease of interest. In some embodiments, the expression value of a control population refers to a baseline level of resistance and/or tolerance of a healthy population or to a baseline level of resistance and/or tolerance before appearance of a disease of interest in a studied population. In some other embodiment, a “healthy control” or “control” may refer to the to a baseline level of resistance and/or tolerance before appearance of a disease of interest in a specific patient.

Thus, in some embodiments, step (b) of the methods of the invention may involves comparing the expression value obtained in steps (a) (i) and/or (a) (ii) with the expression value of an appropriate control or standard. Wherein the expression value obtained in the examined sample for at least one of MTHFD2, PSME2, INTS12, PSMB7 and RBM7, and optionally, JAK2, resistance biomarkers, is “positive”, specifically, higher, overexpressed, elevated when compared to a control, the subject is classified as a subject that has high levels of resistance, also referred to herein as “elevated resistance”. It should be noted that in case of biomarkers that are overexpressed at high levels of resistance, a “positive” expression value should be in the range of the expression value of a control patient determined with high levels of resistance, or any other cut off value obtained for a population of patients known to have high levels of resistance.

Still further, similarly and/or additionally, when the expression value obtained in the examined sample for at least one of MXI1, ZNF395, XPC and SLC6A8 resistance biomarkers, is determined as “negative”, specifically, reduced, low or non-existing expression when compared to a control, the subject is classified as a subject that has high levels of resistance. It should be noted that in case of biomarkers such as MXI1, ZNF395, XPC and SLC6A8, that display reduced, low or non-existing expression at high levels of resistance, a “negative” expression value should be in the range of the expression value of a control patient diagnosed with high levels of resistance, or any other cut off value obtained for a population of patients known to have high levels of resistance.

However, wherein the expression value obtained in the examined sample for at least one of MTHFD2, PSME2, INTS12, PSMB7 and RBM7, and optionally, JAK2, resistance biomarkers, is “negative”, specifically, reduced, low or non-existing expression when compared to a control, the subject is classified as a subject that has low levels of resistance, also referred to herein as “reduced resistance”. It should be noted that in case of biomarkers that are overexpressed at high levels of resistance, a “negative” expression value should be in the range of the expression value of a control patient determined with low levels of resistance, or any other cut off value obtained for a population of patients known to have low levels of resistance.

Similarly and/or additionally, when the expression value obtained in the examined sample for at least one of MXI1, ZNF395, XPC and SLC6A8 resistance biomarkers, is determined as “positive”, specifically, higher, overexpressed, elevated, when compared to a control, the subject is classified as a subject that has low levels of resistance. It should be noted that in case of biomarkers such as MXI1, ZNF395, XPC and SLC6A8, that display reduced, low or non-existing expression at high levels of resistance, a “positive” expression value should be in the range of the expression value of a control patient diagnosed with low levels of resistance, or any other cut off value obtained for a population of patients known to have low levels of resistance.

The same applies to positive and/or negative tolerance markers mentioned in step (iv).

More specifically, wherein the expression value obtained in the examined sample for at least one of MAP2K2, ARHGDIA, GRINA, STXBP2 and SLC6A8, tolerance biomarkers, is “positive”, specifically, higher, overexpressed, elevated when compared to a control, the subject is classified as a subject that has high levels of tolerance, also referred to herein as “elevated tolerance “. It should be noted that in case of biomarkers that are overexpressed at high levels of resistance, a “positive” expression value should be in the range of the expression value of a control patient determined with high levels of tolerance, or any other cut off value obtained for a population of patients known to have high levels of tolerance.

Still further, similarly and/or additionally, when the expression value obtained in the examined sample for at least one of SERINC1, ARLI, COPS2, CRBN and RBM7 tolerance biomarkers, is determined as “negative”, specifically, reduced, low or non-existing expression when compared to a control, the subject is classified as a subject that has high levels of tolerance. It should be noted that in case of biomarkers such as SERINC1, ARLI, COPS2, CRBN and RBM7, that display reduced, low or non-existing expression at high levels of tolerance, a “negative” expression value should be in the range of the expression value of a control patient diagnosed with high levels of tolerance, or any other cut off value obtained for a population of patients known to have high levels of tolerance. However, wherein the expression value obtained in the examined sample for at least one of MAP2K2, ARHGDIA, GRINA, STXBP2 and SLC6A8, tolerance biomarkers, is “negative”, specifically, reduced, low or non-existing expression when compared to a control, the subject is classified as a subject that has low levels of tolerance, also referred to herein as “reduced tolerance”. It should be noted that in case of biomarkers that are overexpressed at high levels of tolerance, a “negative” expression value should be in the range of the expression value of a control patient determined with low levels of tolerance, or any other cut off value obtained for a population of patients known to have low levels of tolerance.

Similarly and/or additionally, when the expression value obtained in the examined sample for at least one of SERINCI, ARLI, COPS2, CRBN and RBM7 tolerance biomarkers, is determined as “positive”, specifically, higher, overexpressed, elevated, when compared to a control, the subject is classified as a subject that has low levels of tolerance. It should be noted that in case of biomarkers such as SERINC1, ARLI, COPS2, CRBN and RBM7, that display reduced, low or non-existing expression at high levels of tolerance, a “positive” expression value should be in the range of the expression value of a control patient diagnosed with low levels of tolerance, or any other cut off value obtained for a population of patients known to have low levels of tolerance.

It should be understood that the term tolerance is used interchangeably with the term “disease tolerance” in the present disclosure and refers to the capacity to bear, endure, or tolerate a state of disease, by limiting the negative impact of infection on host health and fitness without exerting a direct impact on pathogens. Disease tolerance is a physiological term, referring to the relations between the level of health and the pathogen. The physiological status of disease tolerance is defined herein as relative tissue damage. More specifically, the relative tissue damage as used herein, is defined through reaction norms plot the level of damage for an individual at each pathogen burden. This term reflects the slope of the damage-to-pathogen regression in this plot, such that a shallower slope indicates a better ability to tolerate the pathogen.

The tolerance process may involve in some embodiments, controlled and attenuated responsiveness of the immune system to biotic or a biotic stimulus, for example, any biotic or abiotic pathogenic entity, in a manner that maintains vital homeostasis of the subject, tissue recovery. Tolerance as used herein involves the induction of processes mediating wound healing, tissue repair and renewal, production of anti-inflammatory mediators (e.g., anti-inflammatory cytokines). Tolerance involves tissue damage control mechanisms that adjust the metabolic output of host tissues to different forms of stress and damage associated with biotic or a biotic stimulus. More specifically, tissue damage control mechanisms that adjust the metabolic output of host tissues to different forms of stress and damage associated with pathogens. Disease tolerance is the term used to define this defense strategy. which does not exert a direct impact on pathogens but is essential to limit the health and fitness costs of infection.

Thus, the phrase “tolerance level is elevated” reflects decrease, reduction and attenuation in relative tissue damage as defined herein, and in some embodiments, in other specific parameters and symptoms that may include in some embodiments weight loss, breathing disfunction, disfunction of other organs, and the like, in about 5%, 10%, 15%, 20%, 25%, 30%, 35%, 40%, 45%, 50%, 55%, 60%, 65%, 70%, 75%, 80%, 85%, 90%, 95%, 100%, as compared to baseline levels in steady state. Optionally, an elevation, induction, increase, enlargement in the level of tissue repair mechanism, tissue damage control, wound healing, anti-inflammatory mediators, in about 5%, 10%, 15%, 20%, 25%, 30%, 35%, 40%, 45%, 50%, 55%, 60%, 65%, 70%, 75%, 80%, 85%, 90%, 95%, 100%, as compared to baseline levels in steady state.

The term resistance as referred to herein, relates to the ability to eliminate or restrict the replication of an invading pathogen. For instance, in the case of the influenza virus, infection induces a unique spectrum of host defense genes, including interferon-stimulated genes (ISGs) and genes encoding other proteins with antiviral potential. Cellular proteins with putative antiviral activity (hereafter referred to as “restriction factors”) can target various steps in the virus life-cycle. In the context of influenza virus infection, restriction factors are those that target virus entry, genomic replication, translation and virus release. Thus, in some embodiments, the phrase resistance level is elevated reflects an elevation, induction, increase, enlargement in the level of at least one of the level and/or activity of at least one of the specified restriction factors, for example, in about 5%, 10%, 15%, 20%, 25%, 30%, 35%, 40%, 45%, 50%, 55%, 60%, 65%, 70%, 75%, 80%, 85%, 90%, 95%, 100%, as compared to baseline levels in steady state. It should be further appreciated that additional parameters that may be elevated when the resistance is increased may also include an increase in the immune cell quantity, and specifically, distribution thereof in the diseased tissue.

As indicated herein, the present disclosure provides methods for determining the immune-state, or immunological-state of a subject. In some embodiments, immunological-state reflects the state of the immune system of a subject. The immune system acts to protect the host from pathogenic agents, biotic and non-biotic stimuli, in the environment (bacteria, viruses, fungi, parasites, toxins, and chemical entities). It serves to distinguish “nonself” from “self.” In addition, the immune system plays an important role in the identification and elimination of tumor cells or other diseased and aging cells and in the response to injury and trauma. Thus, an effective and efficient immune system is central to host defense against infectious diseases and cancer. The immune system responds to challenge (e.g., a pathogenic infection) in a manner that is reflected by the resistance level, and maintain overall integrity of the infected tissue, as reflected by the level of tolerance. As shown in the present disclosure, the immunological state of a subject as determined by the methods disclosed herein is the level of resistance and disease tolerance in a subject.

In some specific embodiments, the resistance biomarker of the invention may be the MAX Interactor 1 (MXI1) protein. Thus, in certain embodiments, the methods, compositions and kits of the invention may use as a diagnostic tool the expression value of this biomarker either alone or in any combination with any of the biomarker/s disclosed by the invention. MXI1 as described herein refers to the human MXI1 (UNITPROT ID: P50539-1, P50539-3, or P50539-4, gene accession number: NM_005962.4, NM_130439.3 or NM_001008541.1 respectively). This protein is a Transcriptional repressor and binds with MAX to form a sequence-specific DNA-binding protein complex thereby antagonizing MYC transcriptional activity by competing for MAX. In more specific embodiments, the MXI1 protein as used herein may comprise the amino acid sequence as denoted by SEQ ID NO. 1, or any derivatives and homologs thereof, and may be encoded by the nucleic acid sequence as denoted by SEQ ID NO. 2, and any variants, homologs and orthologs thereof. It should be understood that in some embodiments, “negative”, reduced, low, decreased levels of MXII reflect elevated resistance. In some specific embodiments, the resistance biomarker of the invention may be the Zinc Finger Protein 395 (ZNF395) protein. Thus, in certain embodiments, the methods, compositions and kits of the invention may use as a diagnostic tool the expression value of this biomarker either alone or in any combination with any of the biomarker/s disclosed by the invention. ZNF395 as described herein refers to the human ZNF395 (UNITPROT ID: Q9H8N7-1, gene accession number: NM_018660.3). This protein plays a role in papillomavirus genes transcription. In more specific embodiments, the ZNF395 protein as used herein may comprise the amino acid sequence as denoted by SEQ ID NO. 3, or any derivatives and homologs thereof, and may be encoded by the nucleic acid sequence as denoted by SEQ ID NO. 4, and any variants, homologs and orthologs thereof. It should be understood that in some embodiments, “negative”, reduced, low, decreased levels of ZNF395 reflect elevated resistance.

In some specific embodiments, the resistance biomarker of the invention may be the Xeroderma Pigmentosum, Complementation group C (XPC) protein. Thus, in certain embodiments, the methods, compositions and kits of the invention may use as a diagnostic tool the expression value of this biomarker either alone or in any combination with any of the biomarker/s disclosed by the invention. XPC as described herein refers to the human XPC (UNITPROT ID: Q01831-1, gene accession number: NM_004628.5). This protein is involved in global genome nucleotide excision repair (GG-NER) by acting as damage sensing and DNA-binding factor component of the XPC complex. In absence of DNA repair, the XPC complex also acts as a transcription coactivator: XPC interacts with the DNA-binding transcription factor E2F1 at a subset of promoters to recruit KAT2A and histone acetyltransferase complexes (HAT). In more specific embodiments, the XPC protein as used herein may comprise the amino acid sequence as denoted by SEQ ID NO. 5, or any derivatives and homologs thereof, and may be encoded by the nucleic acid sequence as denoted by SEQ ID NO. 6, and any variants, homologs and orthologs thereof. It should be understood that in some embodiments, “negative”, reduced, low, decreased levels of ZNF395 reflect elevated resistance. In some specific embodiments, the resistance biomarker of the invention may be the Methylenetetrahydrofolate Dehydrogenase 2 (MTHFD2) protein. Thus, in certain embodiments, the methods, compositions and kits of the invention may use as a diagnostic tool the expression value of this biomarker either alone or in any combination with any of the biomarker/s disclosed by the invention. MTHFD2 as described herein refers to the human MTHFD2 (UNITPROT ID: P13995-1, gene accession number: NM_006636.4). This protein which has dehydrogenase activity is NAD-specific can also utilize NADP at a reduced efficiency.

In more specific embodiments, the MTHFD2 protein as used herein may comprise the amino acid sequence as denoted by SEQ ID NO. 7, or any derivatives and homologs thereof, and may be encoded by the nucleic acid sequence as denoted by SEQ ID NO. 8, and any variants, homologs and orthologs thereof. It should be understood that in some embodiments, “positive”, increased, high, elevated levels of MTHFD2 reflect elevated resistance.

In some specific embodiments, the resistance biomarker of the invention may be the Proteasome Activator Subunit 2 (PSME2) protein. Thus, in certain embodiments, the methods, compositions and kits of the invention may use as a diagnostic tool the expression value of this biomarker either alone or in any combination with any of the biomarker/s disclosed by the invention. PSME2 as described herein refers to the human PSME2 (UNITPROT ID: Q9UL46-1, gene accession number: NM_002818.3). This protein is implicated in immunoproteasome assembly and required for efficient antigen processing. The PA28 activator complex enhances the generation of class I binding peptides by altering the cleavage pattern of the proteasome.
In more specific embodiments, the PSME2 protein as used herein may comprise the amino acid sequence as denoted by SEQ ID NO. 9, or any derivatives and homologs thereof, and may be encoded by the nucleic acid sequence as denoted by SEQ ID NO. 10, and any variants, homologs and orthologs thereof. It should be understood that in some embodiments, “positive”, increased, high, elevated levels of PSME2 reflect elevated resistance.

In some specific embodiments, an optional resistance biomarker of the invention may be the Janus Kinase 2 (JAK2) protein. Thus, in certain embodiments, the methods, compositions and kits of the invention may use as a diagnostic tool the expression value of this biomarker either alone or in any combination with any of the biomarker/s disclosed by the invention. JAK2 as described herein refers to the human JAK2 (UNITPROT ID: 060674-1, gene accession number: NM_004972.4). This protein is a non-receptor tyrosine kinase involved in various processes such as cell growth, development, differentiation or histone modifications. Mediates essential signaling events in both innate and adaptive immunity. In the cytoplasm, plays a pivotal role in signal transduction via its association with type I receptors such as growth hormone (GHR), prolactin (PRLR), leptin (LEPR), erythropoietin (EPOR), thrombopoietin (THPO); or type II receptors including IFN-alpha, IFN-beta, IFN-gamma and multiple interleukins. In more specific embodiments, the JAK2 protein as used herein may comprise the amino acid sequence as denoted by SEQ ID NO. 11, or any derivatives and homologs thereof, and may be encoded by the nucleic acid sequence as denoted by SEQ ID NO. 12. and any variants, homologs and orthologs thereof. It should be understood that in some embodiments, “positive”, increased, high, elevated levels of JAK2 reflect elevated resistance.

In some specific embodiments, the resistance biomarker of the invention may be the Integrator Complex Subunit 12 (INTS12) protein. Thus, in certain embodiments, the methods, compositions and kits of the invention may use as a diagnostic tool the expression value of this biomarker either alone or in any combination with any of the biomarker/s disclosed by the invention. INTS12 as described herein refers to the human INTS12 (UNITPROT ID: Q9H0H0-1, gene accession number: NM_020748.4). This protein is a component of the Integrator (INT) complex, a complex involved in the small nuclear RNAs (snRNA) U1 and U2 transcription and in their 3′-box-dependent processing. The Integrator complex is associated with the C-terminal domain (CTD) of RNA polymerase II largest subunit (POLR2A) and is recruited to the U1 and U2 snRNAs genes (Probable). Mediates recruitment of cytoplasmic dynein to the nuclear envelope, probably as component of the INT complex. In more specific embodiments, the INTS12 protein as used herein may comprise the amino acid sequence as denoted by SEQ ID NO. 13, or any derivatives and homologs thereof, and may be encoded by the nucleic acid sequence as denoted by SEQ ID NO. 14, and any variants, homologs and orthologs thereof. It should be understood that in some embodiments, “positive”, increased, high, elevated levels of INTS12 reflect elevated resistance.

In some specific embodiments, the resistance biomarker of the invention may be the Proteasome 20S Subunit Beta 7 (PSMB7) protein. Thus, in certain embodiments, the methods, compositions and kits of the invention may use as a diagnostic tool the expression value of this biomarker either alone or in any combination with any of the biomarker/s disclosed by the invention. PSMB7 as described herein refers to the human PSMB7 (UNITPROT ID: Q99436-1, gene accession number: NM_002799.4).

This protein is a component of the 20S core proteasome complex involved in the proteolytic degradation of most intracellular proteins. This complex plays numerous essential roles within the cell by associating with different regulatory particles. Associated with two 19S regulatory particles, forms the 26S proteasome and thus participates in the ATP-dependent degradation of ubiquitinated proteins. The 26S proteasome plays a key role in the maintenance of protein homeostasis by removing misfolded or damaged proteins that could impair cellular functions, and by removing proteins whose functions are no longer required. Associated with the PA200 or PA28, the 20S proteasome mediates ubiquitin-independent protein degradation. This type of proteolysis is required in several pathways including spermatogenesis (20S-PA200 complex) or generation of a subset of MHC class I-presented antigenic peptides (20S-PA28 complex). Within the 20S core complex, PSMB7 displays a trypsin-like activity. In more specific embodiments, the PSMB7 protein as used herein may comprise the amino acid sequence as denoted by SEQ ID NO. 15, or any derivatives and homologs thereof, and may be encoded by the nucleic acid sequence as denoted by SEQ ID NO. 16, and any variants, homologs and orthologs thereof. It should be understood that in some embodiments, “positive”. increased, high, elevated levels of PSMB7 reflect elevated resistance.

In some specific embodiments, the resistance or tolerance biomarker of the invention may be the RNA Binding Motif Protein 7 (RBM7) protein. Thus, in certain embodiments, the methods, compositions and kits of the invention may use as a diagnostic tool the expression value of this biomarker either alone or in any combination with any of the biomarker/s disclosed by the invention. RBM7 as described herein refers to the human RBM7 (UNITPROT ID: Q9Y580-1, gene accession number: NM_016090.4). This protein is an RNA-binding subunit of the trimeric nuclear exosome targeting (NEXT) complex, a complex that functions as an RNA exosome cofactor that directs a subset of non-coding short-lived RNAs for exosomal degradation. In more specific embodiments, the RBM7 protein as used herein may comprise the amino acid sequence as denoted by SEQ ID NO. 17, or any derivatives and homologs thereof, and may be encoded by the nucleic acid sequence as denoted by SEQ ID NO. 18, and any variants, homologs and orthologs thereof. It should be understood that in some embodiments, “positive”, increased, high, elevated levels of RBM7 reflect elevated resistance. Still further, in some embodiments, “negative”, reduced, low, decreased levels of RBM7 reflect elevated tolerance

In some specific embodiments, the resistance or tolerance biomarker of the invention may be the Solute Carrier Family 6 Member 8 (SLC6A8) protein. Thus, in certain embodiments, the methods, compositions and kits of the invention may use as a diagnostic tool the expression value of this biomarker either alone or in any combination with any of the biomarker/s disclosed by the invention. SLC6A8 as described herein refers to the human SLC6A8 (UNITPROT ID: P48029-1, P48029-4, gene accession number: NM_005629.4, NM_001142806.1 respectively). This protein is required for the uptake of creatine in muscles and brain. In more specific embodiments, the SLC6A8 protein as used herein may comprise the amino acid sequence as denoted by SEQ ID NO. 19, or any derivatives and homologs thereof, and may be encoded by the nucleic acid sequence as denoted by SEQ ID NO. 20, and any variants, homologs and orthologs thereof. It should be understood that in some embodiments, “negative”, reduced, low, decreased levels of SLC6A8 reflect elevated resistance. Still further, in some embodiments, “positive”, increased, high, elevated levels of SLC6A8 reflect elevated tolerance.

In some specific embodiments, the tolerance biomarker of the invention may be the Serine Incorporator 1 (SERINC1) protein. Thus, in certain embodiments, the methods, compositions and kits of the invention may use as a diagnostic tool the expression value of this biomarker either alone or in any combination with any of the biomarker/s disclosed by the invention. SERINC1 as described herein refers to the human SERINC1 (UNITPROT ID: Q9NRX5-1, gene accession number: NM_020755.4). This protein enhances the incorporation of serine into phosphatidylserine and sphingolipids. In more specific embodiments, the SERINC1 protein as used herein may comprise the amino acid sequence as denoted by SEQ ID NO. 21, or any derivatives and homologs thereof, and may be encoded by the nucleic acid sequence as denoted by SEQ ID NO. 22, and any variants, homologs and orthologs thereof. It should be understood that in some embodiments, “negative”, reduced, low, decreased levels of SERINC1 reflect elevated tolerance.

In some specific embodiments, the tolerance biomarker of the invention may be the ADP Ribosylation Factor Like GTPase 1 (ARL1) protein. Thus, in certain embodiments, the methods, compositions and kits of the invention may use as a diagnostic tool the expression value of this biomarker either alone or in any combination with any of the biomarker/s disclosed by the invention. ARL1 as described herein refers to the human ARL1 (UNITPROT ID: P40616-1, gene accession number: NM_001177.6). This protein is a GTP-binding protein that recruits several effectors, such as golgins, arfaptins and Arf-GEFs to the trans-Golgi network and modulates their functions at the Golgi complex. Plays a role in fundamental cellular processes, including cell polarity, innate immunity, or protein secretion mediated by arfaptins, which were shown to play a role in maintaining insulin secretion from pancreatic beta cells. In more specific embodiments, the ARL1 protein as used herein may comprise the amino acid sequence as denoted by SEQ ID NO. 23, or any derivatives and homologs thereof, and may be encoded by the nucleic acid sequence as denoted by SEQ ID NO. 24, and any variants, homologs and orthologs thereof. It should be understood that in some embodiments. “negative”, reduced, low, decreased levels of ARLI reflect elevated tolerance.

In some specific embodiments, the tolerance biomarker of the invention may be the COP9 Signalosome Subunit 2 (COPS2) protein. Thus, in certain embodiments, the methods, compositions and kits of the invention may use as a diagnostic tool the expression value of this biomarker either alone or in any combination with any of the biomarker/s disclosed by the invention. COPS2 as described herein refers to the human COPS2 (UNITPROT ID: P61201-1, P61201-2, gene accession number: NM_004236.4. NM_001143887.2 respectively). This protein is an essential component of the COP9 signalosome complex (CSN), a complex involved in various cellular and developmental processes. The CSN complex is an essential regulator of the ubiquitin (Ubl) conjugation pathway by mediating the deneddylation of the cullin subunits of SCF-type E3 ligase complexes, leading to decrease the Ubl ligase activity of SCF-type complexes such as SCF, CSA or DDB2. Involved in early stage of neuronal differentiation via its interaction with NIF3L1. In more specific embodiments, the COPS2 protein as used herein may comprise the amino acid sequence as denoted by SEQ ID NO. 25, or any derivatives and homologs thereof, and may be encoded by the nucleic acid sequence as denoted by SEQ ID NO. 26, and any variants, homologs and orthologs thereof. It should be understood that in some embodiments, “negative”, reduced, low, decreased levels of COPS2 reflect elevated tolerance.

In some specific embodiments, the tolerance biomarker of the invention may be the Cereblon (CRBN) protein. Thus, in certain embodiments, the methods, compositions and kits of the invention may use as a diagnostic tool the expression value of this biomarker either alone or in any combination with any of the biomarker/s disclosed by the invention. CRBN as described herein refers to the human CRBN (UNITPROT ID: Q96SW2-1, Q96SW2-2, gene accession number: NM_016302.4, NM_001173482.1 respectively). This protein is a substrate recognition component of a DCX (DDB1-CUL4-X-box) E3 protein ligase complex that mediates the ubiquitination and subsequent proteasomal degradation of target proteins, such as MEIS2 (Probable). In more specific embodiments, the CRBN protein as used herein may comprise the amino acid sequence as denoted by SEQ ID NO. 27, or any derivatives and homologs thereof, and may be encoded by the nucleic acid sequence as denoted by SEQ ID NO. 28, and any variants, homologs and orthologs thereof. It should be understood that in some embodiments, “negative”, reduced, low, decreased levels of CRBN reflect elevated tolerance.

In some specific embodiments, the tolerance biomarker of the invention may be the Mitogen-Activated Protein Kinase Kinase 2 (MAP2K2) protein. Thus, in certain embodiments, the methods, compositions and kits of the invention may use as a diagnostic tool the expression value of this biomarker either alone or in any combination with any of the biomarker/s disclosed by the invention. MAP2K2 as described herein refers to the human MAP2K2 (UNITPROT ID: P36507-1, gene accession number: NM_030662.4). This protein catalyzes the concomitant phosphorylation of a threonine and a tyrosine residue in a Thr-Glu-Tyr sequence located in MAP kinases. In more specific embodiments, the MAP2K2 protein as used herein may comprise the amino acid sequence as denoted by SEQ ID NO. 29, or any derivatives and homologs thereof, and may be encoded by the nucleic acid sequence as denoted by SEQ ID NO. 30, and any variants, homologs and orthologs thereof. It should be understood that in some embodiments, “positive”, increased, high, elevated levels of MAP2K2 reflect elevated tolerance.

In some specific embodiments, the tolerance biomarker of the invention may be the Rho GDP Dissociation Inhibitor Alpha (ARHGDIA) protein. Thus, in certain embodiments, the methods, compositions and kits of the invention may use as a diagnostic tool the expression value of this biomarker either alone or in any combination with any of the biomarker/s disclosed by the invention. ARHGDIA as described herein refers to the human ARHGDIA (UNITPROT ID: P52565-1, P52565-2, gene accession number: NM_004309.6, NM_001185078.3 respectively). This protein controls Rho proteins homeostasis. Regulates the GDP/GTP exchange reaction of the Rho proteins by inhibiting the dissociation of GDP from them, and the subsequent binding of GTP to them. In more specific embodiments, the ARHGDIA protein as used herein may comprise the amino acid sequence as denoted by SEQ ID NO. 31, or any derivatives and homologs thereof, and may be encoded by the nucleic acid sequence as denoted by SEQ ID NO. 32, and any variants, homologs and orthologs thereof. It should be understood that in some embodiments, “positive”, increased, high, elevated levels of ARHGDIA reflect elevated tolerance.

In some specific embodiments, the tolerance biomarker of the invention may be the Glutamate Ionotropic Receptor NMDA Type Subunit Associated Protein 1 (GRINA) protein. Thus, in certain embodiments, the methods, compositions and kits of the invention may use as a diagnostic tool the expression value of this biomarker either alone or in any combination with any of the biomarker/s disclosed by the invention. GRINA as described herein refers to the human GRINA (UNITPROT ID: Q7Z429-1, gene accession number: NM_000837.2). This protein is potential apoptotic regulator. In more specific embodiments, the GRINA protein as used herein may comprise the amino acid sequence as denoted by SEQ ID NO. 33, or any derivatives and homologs thereof, and may be encoded by the nucleic acid sequence as denoted by SEQ ID NO. 34, and any variants, homologs and orthologs thereof. It should be understood that in some embodiments, “positive”. increased, high, elevated levels of GRINA reflect elevated tolerance.

In some specific embodiments, the tolerance biomarker of the invention may be the Syntaxin Binding Protein 2 (STXBP2) protein. Thus, in certain embodiments, the methods, compositions and kits of the invention may use as a diagnostic tool the expression value of this biomarker either alone or in any combination with any of the biomarker/s disclosed by the invention. STXBP2 as described herein refers to the human STXBP2 (UNITPROT ID: Q15833-1, Q15833-2, Q15833-3, gene accession number: NM_006949.4. NM_001272034.2, NM_001127396.3 respectively). This protein is involved in intracellular vesicle trafficking and vesicle fusion with membranes. Contributes to the granule exocytosis machinery through interaction with soluble N-ethylmaleimide-sensitive factor attachment protein receptor (SNARE) proteins that regulate membrane fusion. Regulates cytotoxic granule exocytosis in natural killer (NK) cells. In more specific embodiments, the STXBP2 protein as used herein may comprise the amino acid sequence as denoted by SEQ ID NO. 35, or any derivatives and homologs thereof, and may be encoded by the nucleic acid sequence as denoted by SEQ ID NO. 36, and any variants, homologs and orthologs thereof. It should be understood that in some embodiments, “positive”, increased, high, elevated levels of STXBP2 reflect elevated tolerance. It should be understood that the present disclosure encompasses the use of at least one of each and every biomarker disclosed by the present disclosure, as well as any of the specified biomarkers as denoted by any of the amino acid sequences as denoted by SEQ ID NO: 1, 3, 5, 7, 9, 11, 13, 15, 17, 19, 21. 23, 25, 27, 29, 31, 33 and 35, or any derivatives and homologs thereof, as well as to any of the disclosed biomarker that comprise the nucleic acid sequence as denoted by any one of SEQ ID NO: 2, 4, 6, 8, 20, 12, 14, 16, 18, 20, 22, 24, 26, 28, 30, 32, 34 and 36, and any variants, homologs and orthologs thereof.

Variants of the polynucleotides ad polypeptides of the biomarkers disclosed herein, may have at least 80% sequence similarity to the entire sequence, often at least 85% sequence similarity, 90% sequence similarity, or at least 95%, 96%, 97%, 98%, or 99% sequence similarity or identity to the entire sequence at the nucleic acid level, with the nucleic acid sequence of the specific biomarkers, such as the various polynucleotides of the invention, as denoted by any one of SEQ ID NO: 2, 4, 6, 8, 20, 12, 14, 16, 18, 20, 22, 24, 26, 28, 30, 32, 34 and 36. Alternatively, when protein biomarkers are used, the disclosed sequence similarity or identity to the entire sequence at the amino acid level, with the amino acid sequence of the specific biomarkers, such as the various polypeptides of the invention as denoted by any one of SEQ ID NO: 1, 3, 5, 7, 9, 11, 13, 15, 17, 19, 21, 23, 25, 27, 29, 31, 33 and 35. The term “derivative” is used to define nucleic acid sequence or amino acid sequence variants, and covalent modifications of a polynucleotide or polypeptide made use of in the present invention, e.g. of a specified sequence. The functional derivatives of any of the polynucleotides or polypeptide utilized according to the present invention, e.g. of a specified sequence of any one of the polynucleotides of SEQ ID NO: 2, 4, 6, 8, 20, 12, 14, 16, 18, 20, 22, 24, 26, 28, 30, 32, 34 and 36, or the polypeptides of SEQ ID NO: 1, 3, 5, 7, 9, 11, 13, 15, 17, 19, 21, 23, 25, 27, 29, 31, 33 and 35, preferably have at least about 65%, more preferably at least about 75%, even more preferably at least about 85%, most preferably at least about 95% overall sequence homology with the nucleic acid sequence of the polynucleotide, or the amino acid sequence of the polypeptide, as structurally defined above, e.g. of a specified sequence, more specifically, the entire nucleic acid sequence of the polynucleotides as denoted by any one of 2, 4, 6, 8, 20, 12, 14, 16, 18, 20, 22, 24, 26, 28, 30, 32, 34 and 36, or the polypeptides of SEQ ID NO: 1, 3, 5, 7, 9, 11, 13, 15, 17, 19, 21, 23, 25, 27, 29, 31, 33, specifically, any homolog that retains association with the T and/or the R level, as discussed by the present disclosure. “Homology” with respect to a native polynucleotide or polypeptide and its functional derivative is defined herein as the percentage of nucleic acid bases or amino acids in the sequence that are identical with the bases of a corresponding polynucleotide or the amino acids of a corresponding polypeptide. Methods and computer programs for the alignment are well known. Still further, in some embodiments, the variants or derivatives disclosed herein may further comprise or include any insertions, deletions to the disclosed sequences of any one of SEQ ID NO: 1 to 36. It should be appreciated that by the term “insertions” or “deletions”, as used herein it is meant any addition or deletion, respectively, of nucleic acid bases to the polynucleotides used by the invention, of between 1 to 50 nucleic acid bases, between 20 to 1 nucleic acid bases, and specifically, between 1 to 10 nucleic acid bases. More particularly, insertions or deletions may be of any one of 1, 2, 3, 4, 5, 6, 7, 8, 9, or 10 nucleic acids, of any of the sequences disclosed herein, specifically, any one of SEQ ID NO: 2, 4, 6, 8, 20, 12, 14, 16, 18, 20, 22, 24, 26, 28, 30, 32, 34 and 36. Or alternatively, of amino acids to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acid residues, of any of the sequences disclosed herein, specifically, any of the amino acid sequences as denoted by SEQ ID NO: 1, 3, 5, 7, 9, 11, 13, 15, 17, 19, 21, 23, 25, 27, 29, 31, 33 and 35. The terms “identical”, “substantial identity”, “substantial homology” or percent “identity”, in the context of two or more nucleic acids or polynucleotide sequences, or alternatively, amino acid residues or polypeptide sequences, refer to two or more sequences or subsequences that are the same or have a specified percentage of nucleic acid bases or nucleotides or alternatively, amino acid residues, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, specifically, over the whole sequence.

In some embodiments, the expression value of at least one resistance biomarker, at times at least two proteins, at times at least three biomarkers, at times at least four biomarkers, at times at least five biomarkers, at times at least six biomarker, at times at least seven biomarkers, at times at least eight biomarkers, at times at least nine biomarkers, at times at least ten biomarkers, of any one of MXI1, ZNF395, XPC, MTHFD2, PSME2, INTS12, PSMB7, RBM7, SLC6A8, and optionally, JAK2, may be determined.

In certain embodiments, the methods of the invention may involve determination of the expression level of all MXI1, ZNF395, XPC, MTHFD2, PSME2INTS12, PSMB7, RBM7, SLC6A8, and optionally, JAK2, resistance biomarkers.

It should be noted that additional resistance biomarkers of the invention are disclosed in Table 1B herein after.

Thus, in some embodiments, at least one of the disclosed resistance biomarkers or specifically, at least 1, 2, 3, 4, 5, 6, 7, 8, 9 or 10 of MXI1, ZNF395, XPC, MTHFD2, PSME2INTS12, PSMB7, RBM7, SLC6A8, and optionally, JAK2, resistance biomarkers may be used in the disclosed methods, optionally, with at least one additional resistance biomarker as disclosed in Table 1B, specifically, at least 1, 2, 3, 4, 5, 6, 7, 8, 9, 10, 11, 12, 13, 14, 15, 16, 17, 18 of the resistance biomarkers of Table 1B, and any combinations thereof.

In some embodiments, the expression value of at least one tolerance biomarker, at times at least two biomarkers, at times at least three biomarkers, at times at least four biomarkers, at times at least five biomarkers, at times at least six biomarkers, at times at least seven biomarkers, at times at least eight biomarkers, at times at least nine biomarkers, at times at least ten, biomarkers of any one of SERINC1, ARL1, COPS2, CRBN, MAP2K2, ARHGDIA, GRINA, STXBP2, RBM7 and SLC6A8 may be determined.

In certain embodiments, the methods of the invention may involve determination of the expression level of all SERINC1, ARL1, COPS2, CRBN, MAP2K2, ARHGDIA, GRINA, STXBP2, RBM7 and SLC6A8 tolerance biomarkers.

It should be noted that additional resistance biomarkers of the invention are disclosed in Table 1A herein after.

More specifically, in some embodiments, at least one of the disclosed tolerance biomarkers or specifically, at least 1, 2, 3, 4, 5, 6, 7, 8, 9 or 10 of the SERINC1, ARLI, COPS2, CRBN, MAP2K2, ARHGDIA, GRINA, STXBP2, RBM7 and SLC6A8, tolerance biomarkers may be used in the disclosed methods, optionally, with at least one additional tolerance marker as disclosed n Table 1A, specifically, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, of the tolerance biomarkers of Table 1A, and any combinations thereof.

In yet some further specific embodiments, the biomarkers determined in accordance with the methods disclosed herein may be any of the biomarkers disclosed by Table 4 and/or Table 5, and any combinations thereof with any of the biomarkers disclosed in any one of Table 1A, Table 1B, and any of the resistance and/or tolerance biomarkers disclosed by the present disclosure.

In certain embodiments, the method as well as the compositions and kits of the invention involve determining the expression level of the disclosed biomarkers and thus provide and use detecting molecules specific for at least one, at least two, at least three, at least four, at least five, at least six, at least seven, at least eight, at least nine biomarkers, at least ten, at least eleven, at least twelve, at least thirteen, at least fourteen, at least fifteen, at least sixteen, at least seventeen, at least eighteen of any one of MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers or of all resistance and/or tolerance biomarkers mentioned in Tables 1A and 1B as well as Tables 4 and 5 and further, involve determining the expression level of the disclosed biomarkers and thus provide and use detecting molecule/s specific for at least one additional biomarker. It should be noted that each detecting molecule is specific for one biomarker. In some embodiments, the method as well as the kits of the invention described herein after, involve determining the expression level of the disclosed biomarkers and thus provide and use further detecting molecules specific for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200, 250, 300, 350, 400, 450 and 500 at the most, additional biomarkers. In some embodiments, the total number of biomarkers determined by the disclosed methods, compositions and kits is 500 at the most. It should be however understood that for each of the disclosed biomarker used, the disclosed methods, compositions and kits provide at least one detecting molecules or more, for example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more detecting molecules. In some embodiments, where about 500 or less biomarkers are used, the disclosed methods may provide and use one, three, four or five detecting molecules for each. In some specific and non-limiting embodiments, the methods, compositions and kits of the invention involve determining the expression level of the disclosed biomarkers and thus provide and use in addition to detecting molecules specific for at least one of the biomarkers disclosed in Tables 1A and 1B as well as Tables 4 and 5.

In some embodiments, the methods, as well as the compositions and kits of the invention involve determining the expression level of the disclosed biomarkers and thus provide and use detecting molecules specific for at least one additional biomarker and at most, 499 additional marker biomarker/s. In some specific embodiments, the methods and kit/s of the invention involve determining the expression level of the disclosed biomarkers and thus provide and use detecting molecules specific for at least one of the biomarkers of Tables 1A and 1B as well as Table 4, and detecting molecules specific for at least one additional biomarkers, provided that detecting molecules specific for 100, 150, 200, 250, 300, 350, 384, 400, 450 and 500 at the most biomarkers are used. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 100 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 150 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 200 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 250 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN. MAP2K2, ARHGDIA, GRINA, STXBP2, RBM7 and SLC6A8 tolerance biomarkers, and at least one additional biomarker, such that the total number of biomarkers determined by the present disclosure is 300 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 350 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARL1, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 400 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 450 at most. In some embodiments, at least 1, 2, 3, 4, 5, 6, 7, 8, 9, 10, 11, 12, 13, 14, 15, 16, 17 or 18 of the resistance and/or tolerance biomarkers of the present disclosure, specifically, MXI1, ZNF395, XPC, MTHFD2, PSME2, INTS12, PSMB7, RBM7, SLC6A8, and optionally, JAK2, resistance biomarkers and/or SERINC1, ARLI, COPS2, CRBN, MAP2K2, ARHGDIA, GRINA, STXBP2, RBM7 and SLC6A8 tolerance biomarkers, and at least one additional biomarker, such that the total number of biomarkers determined by the present disclosure is 500 at most.

In yet some further embodiments, it should be understood that the methods of the invention as well as the compositions and kits described herein after, may involve the determination of the expression levels of the biomarkers of the invention and/or the use of detecting molecules specific for said biomarkers. Specifically, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen of the biomarker/s of the invention that may further comprise any additional biomarkers or control reference protein provided that 500 at the most biomarkers and control reference proteins are used. In yet some further specific and non-limiting embodiments, the method of the invention (as well as any compositions and kits thereof) may use the at least one biomarker of the 18-signatory biomarkers of the invention and in addition, at least one of SERINCI ARLI, COPS2, CRBN, MAP2K2, ARHGDIA, GRINA, STXBP2, MXI1, ZNF395, XPC, MTHFD2, PSME2, JAK2, INTS12, PSMB7, RBM7 and SLC6A8. In some embodiments, the at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen of the biomarker/s of the invention may form at least about 10%, 15%, 20%, 25%, 30%, 35%, 40%, 45%, 50%, 55%, 60%, 65%, 70%, 75%, 80%, 85%, 90%, 95% or 100% of the biomarkers determined by the methods of the invention. In yet some further embodiments, the detecting molecules specific for at least one, at least two, at least three, at least four, at least five, at least six, at least seven, at least eight, at least nine at least ten, at least eleven, at least twelve, at least thirteen, at least fourteen, at least fifteen, at least sixteen, at least seventeen, at least eighteen of the biomarker/s of the invention, that are used by the methods of the invention and comprised within any of the compositions and kits of the invention may form at least 10%, 15%, 20%, 25%, 30%, 35%, 40%, 45%, 50%, 55%, 60%, 65%, 70%, 75%, 80%, 85%, 90%, 95% or 100% of detecting molecules used in accordance with the invention. It should be appreciated that for each of the selected biomarkers at least one detecting molecules may be used. In case more than one detecting molecule is used for a certain biomarker, such detecting molecules may be either identical or different.

Thus, in accordance with some embodiments, in the first steps (a) (i) and/or (a) (ii) of the method of the invention, the expression level of at least one of the biomarkers described herein is being determined. The terms “level of expression” or “expression level” are used interchangeably and generally refer to a numerical representation of the amount (quantity) of nucleic acid product or an amino acid product or polypeptide or protein in a biological sample. In yet some further embodiments, the “level of expression” or “expression level” refers to the numerical representation of the amount (quantity) of polynucleotide which may be gene in a biological sample.

“Expression” generally refers to the process by which gene-encoded information is converted into the structures present and operating in the cell. For example, the expression may be measured in the nucleic acid level, for example using Real-Time Polymerase Chain Reaction, sometimes also referred to as RT-PCR or quantitative PCR (qPCR). The luminosity in case of RT-PCR, or any other tag is captured by a detector that converts the signal intensity into a numerical representation which is said expression value, in terms of biomarker or a gene. Therefore, according to the invention “expression” of a gene, specifically, any gene encoding any of the biomarkers of the invention may refer to transcription into a polynucleotide and translation into a polypeptide. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after.

The expression level of the biomarkers, that may be biomarker proteins/genes (expression either at the nucleic acid, specifically, mRNA level or the protein level) of the invention is determined to obtain an expression value. The term “expression value” refers to the result of a calculation, that uses as an input the “level of expression” or “expression level” obtained experimentally. It should be appreciated that in some optional embodiments, determination of the expression value may further involves normalizing the “level of expression” or “expression level” by at least one normalization step as detailed herein, where the resulting calculated value termed herein “expression value” is obtained.

More specifically, as used herein, “normalized values” in some embodiments, are the quotient of raw expression values of biomarker/s, specifically, gene and any product thereof, e.g., mRNA, protein, divided by the expression value of a control reference biomarker from the same sample. Any assayed sample may contain more or less biological material than is intended, due to human error and equipment failures. Importantly, the same error or deviation applies to both the marker protein/gene of the invention and to the control reference gene and any product thereof, e.g., mRNA or protein, whose expression is essentially constant. Thus, division of the biomarker raw expression value by the control reference protein raw expression value yields a quotient which is essentially free from any technical failures or inaccuracies (except for major errors which destroy the sample for testing purposes) and constitutes a normalized expression value of the biomarker. This normalized expression value may then be compared with normalized cutoff values, i.e., cutoff values calculated from normalized expression values. In certain embodiments, the control reference biomarker, specifically, gene and any product thereof, e.g., mRNA or protein, may be a protein/gene that maintains stable in all samples analyzed.

Normalized biomarker expression (either the nucleic acid molecule, (mRNA) and/or the protein) level values that are higher (positive) or lower (negative) in comparison with a corresponding predetermined standard expression value or a cut-off value in a control sample predict to which population of subjects, either having high/low levels of resistance and/or high/low levels of tolerance, the tested sample belongs.

It should be appreciated that an important step in the method of the inventions is determining whether the expression value of any one of the biomarkers is changed or different when compared to a pre-determined cut off or is within the range of expression of such cutoff. Alternatively, or in addition, the expression value may be compared to the expression value of a control sample, for example, a sample obtained from a healthy population.

Thus, in yet more specific embodiments, the method of the invention involves comparing the expression values determined for the tested sample with predetermined standard values or cutoff values, or alternatively, with expression values of at least one control sample. As used herein the term “comparing” denotes any examination of the expression level and/or expression values obtained in the samples of the invention as detailed throughout in order to discover similarities or differences between at least two different samples. It should be noted that in some embodiments, comparing according to the present invention encompasses the possibility to use a computer-based approach. As described hereinabove, the method of the invention refers to a predetermined cutoff value/s. It should be noted that a “cutoff value”, sometimes referred to simply as “cutoff” herein, is a value that meets the requirements for both high diagnostic sensitivity (true positive rate) and high diagnostic specificity (true negative rate).

Simply put, “sensitivity” relates to the rate of identification of the patients having low/high levels of resistance and/or resistance (samples) as such, out of a group of samples, whereas “specificity” relates to the rate of correct identification of samples with low/high levels of resistance and/or resistance as such, out of a group of samples.

Cutoff values may be used as control sample/s or in addition to control sample/s, said cutoff values being the result of a statistical analysis of biomarker expression value/s (specifically the biomarker/s genes/proteins of the invention) differences in pre-established populations having known levels of resistance and/or tolerance. Pre-established populations as used herein refer to populations of patients with known levels of resistance and/or tolerance or alternatively, populations of healthy subjects. In yet some further embodiments, a negative or positive determination of the expression value as compared to the predetermined cutoff values, or the expression value of a control sample, also encompass values that are within the range of said cutoff. More specifically, in case the particular biomarker is found to be overexpressed in high levels of resistance and/or tolerance, an expression value that is determined by the method of the invention as “positive” when compared to a predetermined cutoff of population of patients having high levels of resistance and/or tolerance, or to the expression value of at least one, and preferably, more, known subject/s having high levels of resistance and/or tolerance, may indicate that the examined subject belongs to a population having high levels of resistance and/or tolerance, in case that the expression value is either higher (positive) or fall within the range (the average values of the cutoff predetermined for patient population having high levels of resistance and/or tolerance) of the control or standard value.

In a similar manner, a subject exhibiting an expression value that is “negative” (that is down-regulated) as compared to the cutoff patients, may be considered as belonging to population that do not have high levels of resistance and/or tolerance (e.g. having low levels of resistance and/or tolerance), in case the expression of the particular biomarker is associated with overexpression at high level of resistance and/tolerance.

In more specific embodiments, the expression value of such subject should fall within the range of the cutoff value predetermined for population having known high/low levels of resistance and/or tolerance. In some embodiments, “fall within the range” encompass values that differ from the cutoff value in about 1%, about 5%, about 10%, about 15%, about 20%, about 25%, about 30%, about 35%, about 40%, about 45%, about 50% or more.

Simply put, a “positive” expression value as used herein refers to high expression value that reflects overexpression, elevated expression, high expression and even in some embodiments, moderate expression value.

A “negative” expression value reflects a repressed, low, reduced, or non-existing expression (lack of expression). Thus, in some embodiments, when a specific biomarker is overexpressed at high levels of resistance or tolerance, a “positive” expression value of an examined sample may be a value that is higher or within the range of the expression value of a sample taken from a patient having known high levels of resistance and/or tolerance, or a standard cutoff value calculated for high levels of resistance and/or tolerance.

A “negative” value would be an expression value that is lower than the expression value of the patients (or standard value, or the value of a control sample) having high levels of resistance and/or tolerance. It should be emphasized that the nature of the invention is such that the accumulation of further patient data may improve the accuracy of the presently provided cutoff values, which are based on an ROC (Receiver Operating Characteristic) curve generated according to said patient data using analytical software program. The biomarker expression values are selected along the ROC curve for optimal combination of diagnostic sensitivity and diagnostic specificity which are as close to 100 percent as possible, and the resulting values are used as the cutoff values that distinguish between subjects having high or low levels of resistance and/or tolerance at a certain rate, and those who will not (with said given sensitivity and specificity). The ROC curve may evolve as more and more data and related biomarker gene expression values are recorded and taken into consideration, modifying the optimal cutoff values and improving sensitivity and specificity. Thus, it should be appreciated that the provided cutoff values should be viewed as a starting point that may shift as more data allows more accurate cutoff value calculation.

As noted above, the expression value determined for the examined sample (or alternatively, the normalized expression value) is compared with a predetermined cutoff or to a control sample. More specifically, in certain embodiments, the expression value obtained for the examined sample is compared with a predetermined standard or cutoff value.

In further embodiments, the predetermined standard expression value, or cutoff value has been pre-determined and calculated for a population having known levels of resistance and/or tolerance or in the context of the further disclosed methods of the invention, a population comprising at least one of healthy subjects, subjects susceptible to develop a disorder, subjects suffering from any disorder, subjects suffering from different stages of any disorder, subjects that respond to treatment, non-responder subjects, subjects in remission and subjects in relapse.

Still further, in certain alternative embodiments where a control sample is being used (instead of, or in addition to, pre-determined cutoff values), the expression value or the normalized expression values of the biomarkers used by the invention in the test sample are compared to the expression values in the control sample. In certain embodiments, such control sample may be obtained from at least one of a healthy subject, subjects susceptible to develop a disorder, a subject suffering from a disorder at a specific stage, a subject suffering from a disorder at a different specific stage a subject that responds to treatment, a non-responder subject, a subject in remission and a subject in relapse. It should be appreciated that “Standard”, or a “predetermined standard” as used herein, denotes either a single standard value or a plurality of standards with which the level of at least one of the biomarker expression from the tested sample is compared. The standards may be provided, for example, in the form of discrete numeric values or is calorimetric in the form of a chart with different colors or shadings for different levels of expression; or they may be provided in the form of a comparative curve prepared on the basis of such standards (standard curve).

In some specific and non-limiting embodiments, step (a) of the disclosed methods comprises determining in at least one biological sample of the subject the expression level of:

(i) the biomarkers of resistance to obtain an expression value for each of the biomarker/s, more specifically, for each of the following biomarkers of resistance MXI1, ZNF395, XPC, MTHFD2, PSME2, JAK2, INTS12, PSMB7, RBM7 and SLC6A8; and

(ii) the biomarkers of tolerance to obtain an expression value for each of the biomarker/s, more specifically, for each of the following biomarkers of tolerance are SERINC1, ARLI, COPS2, CRBN, MAP2K2, ARHGDIA, GRINA, STXBP2, RBM7 and SLC6A8.

In some embodiments, the step of determining the level of expression of at least one of the biomarker/s of resistance and/or at least one the biomarker/s of tolerance is performed by the step of contacting at least one detecting molecule or any combination or mixture of plurality of detecting molecules with a biological sample of the subject, or with any nucleic acid or protein product obtained therefrom. It should be understood that each of the detecting molecules is specific for one of the biomarkers. Still further, plurality of detecting molecules for each of the disclosed biomarker is meant at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more detecting molecules for each of the disclosed biomarkers.

In some specific embodiments, the at least one detecting molecule used for determining the level of expression may be any one of nucleic acid-based detecting molecules and/or amino acid-based detecting molecules.

More specifically, in some embodiments, nucleic acid detecting molecule/s useful in the methods disclosed herein may comprise at least one of: (a) at least one oligonucleotide/s, each oligonucleotide specifically hybridizes to a nucleic acid sequence encoding the at least one biomarker, or any parts or fragments thereof; and/or (b) at least one nucleic acid aptamer/s specific for the at least one of the biomarkers.

In some specific embodiments, when the expression level of the biomarkers discussed herein is determined at the nucleic acid level (e.g., mRNA), useful detecting molecules may be in some embodiments, nucleic acid detecting molecule/s. In yet some further specific embodiments, the nucleic acid detecting molecules may be at least one oligonucleotide/s that specifically hybridizes to a nucleic acid sequence encoding the at least one biomarker or any fragment/s, or mixture/s thereof. According to such embodiments, the determination of the expression level of the at least one biomarker/s may be performed by any nucleic acid-based method. Non-limiting examples for such procedures include, but are not limited to, Real-Time Polymerase Chain Reaction (RT-PCR) or quantitative PCR (qPCR).

It should be noted that for determining the expression value/s of at least one of the biomarkers of the invention, the methods of the invention may further comprise the step of providing at least one detecting molecule specific for determining the expression of at least on of the biomarkers of the invention. In some embodiments, such detecting molecules may be provided as a mixture, as a composition or as a kit. Thus, in some embodiments, the at least one detecting molecules may be provided as a mixture of detecting molecules, wherein each detecting molecule is specific for one biomarker. It should be appreciated however, that for each biomarker, one or several specific detecting molecules may be used and provided. In yet some further alternative embodiments, the detecting molecules may be provided separately for each biomarker, e.g., in specific tube, containers, slot/s, spot/s, well/s, dot/s, bead/s, particle/s, chip/s and the like. It further alternative embodiments, the detecting molecules may be attached or immobilized to a solid support, specifically, in recorded location.

The present disclosure further encompasses in some embodiments thereof any of the disclosed detecting molecules that may be provided either separated or mixed, either attached or immobilized to a solid support or provided unattached and not immobilized to a solid support.

Still further, in some embodiments, for determining the expression level of the specified biomarkers the sample or any nucleic acid molecules or proteins thereof is contacted with specific detecting molecules for each of the biomarkers.

The term “contacting” means to bring, put, incubates or mix together. As such, a first item is contacted with a second item when the two items are brought or put together, e.g., by touching them to each other or combining them. In the context of the present disclosure, the term “contacting” includes all measures or steps which allow interaction between the at least one of the detection molecules of at least one of the biomarkers, and optionally, for at least one suitable control reference mRNA/protein of the tested sample. The contacting is performed in a manner so that the at least one of detecting molecule of at least one of the biomarkers for example, can interact with or bind to the at least one of the biomarkers, in the tested sample. The binding will preferably be non-covalent, reversible binding, e.g., binding via salt bridges, hydrogen bonds, hydrophobic interactions or a combination thereof.

As indicated herein, the detecting molecules used in the disclosed methods, compositions and kits may be nucleic acid-based molecule. As used herein, “nucleic acid molecules” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

More specifically, in some other embodiments, the nucleic acid detecting molecules may comprise at least one isolated oligonucleotide/s, each oligonucleotide specifically hybridizes to a nucleic acid sequence encoding one of the at least one biomarker/s, or any parts or fragments of such encoding sequence/s. In an optional embodiment, where the expression levels of the biomarkers of the invention are normalized, the method of the invention may use nucleic acid detecting molecules specific for a nucleic acid sequence encoding the control reference protein/s.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly. Such oligonucleotide/s include aptamers, probes and/or primers. In yet some further specific embodiments, where the detecting molecules of the invention are nucleic acid based molecules, optional detecting molecule/s may be at least one nucleic acid aptamer specific for the at least one of the biomarker/s.

As used herein the term “aptamer” or “specific aptamers” denotes single-stranded nucleic acid (DNA or RNA) molecules which specifically recognizes and binds to a target molecule. The aptamers according to the invention may fold into a defined tertiary structure and can bind a specific target molecule with high specificities and affinities. Aptamers are usually obtained by selection from a large random sequence library, using methods well known in the art, such as SELEX and/or Molinex. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides.

According to the present invention and as appreciated in the art, the recognition between the aptamer and the antigen is specific and may be detected by the appearance of a detectable signal by using a colorimetric sensor or a fluorimetric/lumination sensor, radioactive sensor, or any appropriate means. The aptamers that may be used according to some aspects of the invention may be biotinylated. The aptamers may optionally include a chemically reactive group at the 3′ and/or 5′ termini. The term reactive group is used herein to denote any functional group comprising a group of atoms which is found in a molecule and is involved in chemical reactions. Some non-limiting examples for a reactive group include primary amines (NH2), thiol (SH), carboxy group (COOH), phosphates (PO4), Tosyl, and a photo-reactive group.

In some embodiments, the aptamer that may be applicable herein may optionally comprise a spacer between the nucleic acid sequence and the reactive group. The spacer may be an alkyl chain such as (CH₂) _{6/12}, namely comprising six to twelve carbon atoms.

In yet some other alternative embodiments, the detection molecule may be or may comprise at least one primer, at least one pair of primers, nucleotide probes and any combinations thereof. Thus, it should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more. In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available.

Still further, the detecting molecules according to some embodiments may be or may comprise at least one probe. As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe may be 5, 6, 7, 8, 9, 10, 11, 12, 13, 14, 15, 16, 17, 18, 19, 20, 21, 22, 23, 24, 25, 26, 27, 28, 29 and up to 30 or more nucleotides in length as long as it is less than the full length of the target mRNA or any gene encoding said mRNA. Probes can include oligonucleotides modified so as to have a tag which is detectable by fluorescence, chemiluminescence and the like. The probe can also be modified so as to have both a detectable tag and a quencher molecule, for example TaqMan (R) and Molecular Beacon (R) probes.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′O-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

According to the option of using nucleic acid-based detecting molecules for determining the expression level of the disclosed resistance and/or tolerance biomarkers, the expression level may be determined using amplification assay. The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction.

“Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers. Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan (R), MGB-TaqMan (R)), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan (R) and MGB-TaqMan (R) the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified biomarkers of the present disclosure or any control reference gene produced in a PCR amplification reaction. During PCR, the amplified products created using suitable primers hybridize to probe nucleic acids (TaqMan (R) probe, for example), which may be labeled according to some embodiments with both a reporter dye and a quencher dye. When these two dyes are in close proximity, i.e., both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan (R) (Perkin Elmer, Foster City, CA), is performed with a transcript-specific antisense probe. This probe is specific for the PCR product (e.g. a nucleic acid fragment derived from a gene) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured, and the PCR product is quantified. The PCR reactions can be performed in any solid support, for example, slides, microplates, 96 well plates, 384 well plates and the like so that samples derived from many individuals are processed and measured simultaneously. The TaqMan (R) system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia California). RT-PCR is performed using SYBR green as a fluorescent label which is incorporated into the PCR product during the PCR stage and produces fluorescence proportional to the amount of PCR product.

Both TaqMan (R) and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons (R) which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression.

According to this embodiment, the detecting molecule may be in the form of probe corresponding and thereby hybridizing to any region or at least one of the biomarker/s or any control reference protein. More particularly, it is important to choose regions which will permit hybridization to the target nucleic acids. Factors such as the Tm of the oligonucleotide, the percent GC content, the degree of secondary structure and the length of nucleic acid are important factors.

It should be noted however that a standard Northern blot assay or dot blot can also be used to ascertain an RNA transcript size and the relative amounts of the biomarkers of the invention or any control gene product, in accordance with conventional Northern hybridization techniques known to those persons of ordinary skill in the art.

In yet some alternative and/or additional embodiments, when the determination of the expression levels of the disclosed specific biomarkers is performed at the protein level, amino-acid based detecting molecules may be used. In yet some further specific embodiments, such amino-acid-based detecting molecule/s comprise at least one of: (a) at least one labeled or tagged biomarker/s or any fragment/s, peptide/s or mixture/s thereof; (b) at least one antibody specific for the at least one of the biomarkers; (c), at least one protein or peptide aptamer/s specific for the at least one of the biomarkers; and (d) any combination of (a), (b) and (c).

In yet some alternative embodiments, the determination of the expression level of the biomarkers used by the disclosed methods is performed at the protein level. Accordingly, in some embodiments, the detecting molecule/s may be amino-acid-based detecting molecule. The invention thus contemplates the use of amino acid-based molecules such as proteins or polypeptides as detecting molecules disclosed herein and would be known by a person skilled in the art to measure the level of the at least one biomarker disclosed herein. As used herein, the terms “protein” and “polypeptide” are used interchangeably to refer to a chain of amino acids linked together by peptide bonds. In a specific embodiment, a protein is composed of between at least 3 to at least 5000 or more amino acids linked together by peptide bonds. It should be noted that peptide bond as described herein is a covalent amid bond formed between two amino acid residues. In some embodiments, the detecting molecules used by the methods of the invention may be recombinantly expressed or synthetically prepared. In further embodiments, the recombinantly or synthetically expressed and prepared detecting molecules may be labeled or tagged. It should be noted that in some embodiments, these detecting molecules may be isolated detecting molecules. As used herein, “Recombinant proteins” denotes proteins encoded by a recombinant DNA which is a genetically engineered DNA formed by laboratory methods of genetic recombination to bring together genetic material from multiple sources and thus creating variable sequences. Techniques for detection and quantification known to persons skilled in the art (for example, Mass spectrometry (MS) or different immunological techniques such as Western Blotting, Immunoprecipitation, ELISAs, protein microarray analysis, Flow cytometry and the like) can then be used to measure the level of protein products corresponding to the biomarker of the invention. In specific embodiments, the detecting amino acid molecules applicable for the invention may be isolated antibodies, with specific binding selectively to at least one of the biomarker proteins. More specifically, the term “antibody” as used in this invention includes whole antibody molecules as well as functional fragments thereof, such as Fab, F (ab′) 2, and Fv that are capable of binding with antigenic portions of the target polypeptide, i.e. at least one of the biomarker protein/s. The antibody may be preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which, as used herein, refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, 1gM or subtypes thereof. The antibody can be conjugated to a labeling moiety as discussed above.

Still further, the antibodies used by the present invention may optionally be covalently or non-covalently linked to a detectable label or tag. In addition, the label and can also refer to indirect labeling of the protein by reactivity with another reagent that is directly labeled. Examples of indirect labeling include detection of at least one of the biomarker protein/s of the invention using a fluorescently labeled secondary antibody. More specifically, detectable labels suitable for such use include any composition detectable by spectroscopic, photochemical, biochemical, immunochemical, electrical, optical or chemical means.

In some other embodiments, the detecting molecules are peptide aptamers specific for the at least one of the biomarker proteins. “Peptide or protein aptamers” as used herein refers to small peptides with a single variable loop region tied to a protein scaffold on both ends that binds to a specific molecular target (e.g. protein), and which are bind to their targets only with said variable loop region and usually with high specificity properties.

It should be appreciated that in certain embodiments, the signature proteins, specifically, at least one, at least two, at least three, at least four, at least five, at least six, at least seven, or at least eight, at least nine, at least ten, at least eleven, at least twelve, at least thirteen, at least fourteen, at least fifteen, at least sixteen, at least seventeen, at least eighteen or all of the biomarkers of the invention (e.g., of Table 1A, 1B, 4 and 5) or any protein-fragments thereof may be also detected and quantified without the need for detection molecule/s. Detection can be based on MS approaches using non-targeted or targeted methods such as selected reaction monitoring (SRM) or parallel reaction monitoring (PRM). These analyses can be performed with or without a reference heavy standard and provide quantitative measure of the peptide/protein amount. The heavy reference can be a synthetic peptide, or a chemically labeled peptide/protein or metabolically labeled proteins. In the absence of a standard, the MS signal can provide the measure of peptide abundance.

Still further, in some embodiments, the biological sample analyzed by the disclosed methods may be any biological sample, for example, any body fluid sample (that may ither comprise cells or not), and/or any cell sample, cell fractions and/or cell organelles, and/or tissue sample, and/or organ sample of the examined subject.

As used herein, the term “sample” refers to cells, sub-cellular compartments thereof, tissue or organs. The tissue may be a whole tissue, or selected parts of a tissue. Tissue parts can be isolated by micro-dissection of a tissue, or by biopsy, or by enrichment of sub-cellular compartments. The term “sample” further refers to healthy as well as diseased or pathologically changed cells or tissues. Hence, the term further refers to a cell or a tissue associated with a disease, such a tumor, in particular cancer, and more specifically, breast cancer, MM, CLL, lung adenocarcinoma, Neuroblastoma and astrocytoma. Alternatively, a sample of an injured organ or tissue of a subject, for example, liver tissue of a subject suffering from liver injury. A sample can be cells that are placed in or adapted to tissue culture. A sample can additionally be a cell or tissue from any mammalian species, specifically, humans. A tissue sample can be further a fractionated or preselected sample, if desired, preselected or fractionated to contain or be enriched for particular cell types.

In some specific and non-limiting embodiments, the sample of the method of the invention may be a body fluid sample. More specifically, such sample may be any body fluid such as blood, plasma, lymph, urine, saliva, serum, cerebrospinal fluid, seminal plasma, pancreatic juice, breast milk, uterine, peritoneal cavity, lung lavage., or fluids collected from any organ or tissue cavity.

The sample can be fractionated or preselected by a number of known fractionation or pre selection techniques. A sample can also be any extract of the above. The term also encompasses protein fractions or alternatively, nucleic acid from cells or tissue. Thus, in some specific embodiments, the sample may be any one of a biological sample of organ/s, cell/s or tissue/s and a blood sample, including any blood or hematopoietic cells of any one of the erythroid, myeloid, lymphoid lineages. Specific embodiments relate to hematopoietic cells, for example, T cells, B cells and the like. In yet some other embodiments, the sample may be a primary tumor sample. In certain embodiments, the sample is obtained from a subject suffering from a disorder, or any biopsy of diseased tissue or organ. In some specific and non-limiting embodiments, the sample may be a blood sample. In yet some further embodiments, the sample analyzed by the disclosed methods may be a cell sample obtained from any tissue or organ of the examined subject. In some embodiments, the sample may be a skin cell sample. In yet some further embodiments, the sample may be any sample obtained by a biopsy from any tissue and/or organ. In some embodiments, the sample is a biopsy of a diseased tissue or organ. Still further in some embodiments, the sample may be a tumor biopsy.

It should be appreciated that the methods disclosed herein, for determining/evaluating the immunological state of a subject by determining the levels of resistance and/or tolerance of the subject based on particular signature disclosed herein, may be applicable for any subjects. More specifically, any healthy subject or any subject that suffers from a specific pathological condition. Therefore, in some embodiments, the subject evaluated by the methods of the present disclosure may be any one of: (a) a subject displaying healthy-homeostatic conditions, (b) a subject suffering from at least one pathologic disorder, and (c) a subject exposed to at least one biotic and/or at least one abiotic stimulus. In some specific embodiments, the method of the invention is used for determining the immunological state (e.g., resistance and/or tolerance) of a subject displaying any healthy, or non-diseased-homeostatic condition, for example, puberty, pregnancy, menstruation, menopause, aging, obesity, metabolic syndrome and the like. In such conditions, which are not defined as disease states, the immunological states determined by the method of invention can be used as risk factors for further pathologic conditions. For example, body wight changes as in obesity and/or metabolic syndrome. More specifically, the disclosed methods, compositions and kits may be applicable in some embodiments, as an indicator to assess the risk of future disease in subjects with a specific condition such as obesity or older adults. In some specific embodiments, the method of the invention is used for determining the immunological state (e.g., resistance and/or tolerance) of a subject suffering from a pathologic disorder. In some embodiments, the pathologic disorder may be at least one immune-related disorder. Still further, in some embodiments such disorder may be at least one of an infectious disease caused by at least one pathogen, an inflammatory disorder, an autoimmune disorder, a proliferative disorder, a neurodegenerative disorder (and/or any protein misfolding disorder), a metabolic disorder and a condition involving at least one wound in at least one tissue and/or organ of the subject. Still further, any of the disclosed conditions may be either a congenital or an acquired condition. It should be appreciated that in some embodiments, wound can be a chronic and/or acute wound and the condition involved is a wound healing. It should be understood that all conditions disclosed in connection with other aspects of the invention are also applicable in the present aspect. A further aspect of the present disclosure relates to a prognostic method for determining the susceptibility of a subject to at least one pathologic disorder, and/or predicting the outcome of the at least one pathological disorder in the subject. More specifically, the method comprising the following steps. First in step (a), determining the level/s of resistance and/or tolerance of the subject.

The next step (b), involves classifying the subject as a subject susceptible to the pathologic disorder and/or to develop a negative outcome of the pathological disorder, based on the resistance and tolerance levels of the subject and the levels of resistance and tolerance that characterize the particular disorder. More specifically, the subject is determined susceptible if the level of resistance and/or tolerance determined in step (a) is at least one of: (i) elevated resistance and/or reduced tolerance, in a disorder where a reduced susceptibility and/or positive outcome in a subject is characterized with reduced resistance and/or elevated tolerance; and (ii) reduced resistance and/or elevated tolerance, in a disorder where a reduced susceptibility and/or positive outcome in a subject is characterized with elevated resistance and/or reduced tolerance, thereby determining the susceptibility of said subject and/or predicting the outcome of the pathological disorder in the subject.

In yet some further embodiments, the prognostic methods in accordance with the present disclosure for determining the susceptibility of a subject to at least one pathologic disorder, and/or predicting the outcome of the at least one pathological disorder in the subject, the method comprising the following steps. First in step (a), determining the level/s of resistance and/or tolerance of the subject. In the next step (b), classifying the subject as: either (I) a subject susceptible to the pathologic disorder and/or to develop a negative outcome of the pathological disorder, if the level of resistance and/or tolerance determined in step (a) is at least one of: (i) elevated resistance and/or reduced tolerance, in a disorder where a reduced susceptibility and/or positive outcome in a subject is characterized with reduced resistance and/or elevated tolerance; (ii) reduced resistance and/or elevated tolerance, in a disorder where a reduced susceptibility and/or positive outcome in a subject is characterized with elevated resistance and/or reduced tolerance; (iii) reduced resistance and/or tolerance, in a disorder where a reduced susceptibility and/or positive outcome in a subject is characterized with elevated resistance and/or tolerance; and (iv) elevated resistance and/or tolerance, in a disorder where a reduced susceptibility and/or positive outcome in a subject is characterized with reduced resistance and tolerance. Alternatively, the subject may be classified as (II) a subject not susceptible to the pathologic disorder and/or as a subject having increased likelihood to develop (or capable of developing/displaying) a positive outcome of the disorder, if the level of resistance and/or tolerance determined in step (a) is at least one of: (i) elevated resistance and/or reduced tolerance, in a disorder where a reduced susceptibility and/or positive outcome in a subject is characterized with elevated resistance and/or reduced tolerance; (ii) reduced resistance and/or elevated tolerance, in a disorder where a reduced susceptibility and/or positive outcome is characterized with reduced resistance and/or elevated tolerance; (iii) reduced resistance and/or tolerance, in a disorder where a reduced susceptibility and/or positive outcome is characterized with reduced resistance and/or tolerance associated with a positive outcome; and (iv) elevated resistance and/or tolerance, in a disorder where a reduced susceptibility and/or positive outcome is characterized with elevated resistance and tolerance. Such methods thereby enable determining the susceptibility of the subject and/or predicting the outcome of the pathological disorder in the subject.

It should be appreciated that in some embodiments, a negative outcome as used herein refers to a severe form, relapse, any worsening of existing symptoms or conditions, deterioration of overall condition of the subject (e.g., body weight, physical and/or mental functioning, tissue and organ integrity and function), or even decreased survival and death of the subject etc. In yet some further embodiments, the positive outcome, as used herein, refers to a not severe form of the disease, a mild form, early stage or grade, enhanced chances for recovery, extended survival, remission, extended disease-free period and the like.

It should be noted that in some embodiments, of the prognostic methods disclosed herein, a positive or negative outcome in a subject is determined by determining for each disorder the standard levels of resistance and/or tolerance in a known and predetermined (control) population of subjects suffering from a particular disorder, and having developed a negative outcome of the pathological disorder and/or the standard levels of resistance and/or tolerance in a (control) population of subjects having developed a positive outcome for the disorder. These predetermined levels of resistance and/or tolerance are used in some embodiments as cutoff value/s for the determination of a specific expression cutoff that characterize or distinguish a population of patients having the same disorder, or healthy subjects, or subjects suffering from a different disorder, and displaying the relevant outcome. It should be noted that these cutoff values may be in some embodiment predetermined, and/or presented in calibration curves and/or provided by control samples (positive and/or negative) samples.

It should be understood that in some embodiments, determination of the resistance and/or tolerance in the subject, by the prognostic method disclosed herein is based on the determination of the expression level/s of at least one, at least two, at least three, at least four, at least five, at least six, at least seven, at least eight, at least nine biomarkers, at least ten, at least eleven, at least twelve, at least thirteen, at least fourteen, at least fifteen, at least sixteen, at least seventeen, at least eighteen of the disclosed biomarkers, and/or of any of the biomarkers disclosed by the present disclosure, specifically, any one of the biomarkers disclosed in Tables 1A, 1B, 4 and 5. It should be further understood that any additional biomarker and/or control protein/gene may be determined by the disclosed methods.

In some embodiments, of the disclosed prognostic method, step (a) that involves determination of the level of resistance and/or tolerance in the subject, may be performed by a method comprising the following steps:
First (a), determining in at least one biological sample of the subject the expression level of at least one of: (i) at least one biomarker of the resistance, to obtain an expression value for each of the at least one biomarker/s. More specifically, the at least one biomarker of resistance is at least one of MXI1, ZNF395, XPC, MTHFD2, PSME2, JAK2, INTS12, PSMB7, RBM7, SLC6A8 or any combination thereof; and
(ii) at least one biomarker of the tolerance, to obtain an expression value for each of the at least one biomarker/s. Specifically, the at least one biomarker of tolerance is at least one of SERINC1, ARLI, COPS2, CRBN, MAP2K2, ARHGDIA, GRINA, STXBP2, RBM7, SLC6A8 or any combination thereof.

The next step (b), concerns determining if the expression values obtained in steps (a) (i) and/or (a) (ii) for each of the at least one biomarker/s is positive or negative with respect to a predetermined standard expression value or to an expression value of the biomarker/s in at least one control sample. In should be noted that wherein at least one of: (I) a positive expression value of at least one of the MTHFD2, PSME2, JAK2, INTS12, PSMB7 and RBM7 biomarker/s in the sample, and/or a negative expression value of at least one of the MXI1, ZNF395, XPC and SLC6A8 biomarker/s in the sample indicate(s) that the resistance level is elevated in the subject; and/or (II) a positive expression value of at least one of the MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample indicate(s) that the tolerance level is elevated in the subject. In some embodiments, the level of expression of at least one of the tolerance and/or resistance biomarkers is determined in step (a), may be performed as defined by the present invention. More specifically, in some specific and non-limiting embodiments, step (a) of the disclosed prognostic methods comprises determining in at least one biological sample of the subject the expression level of: (i) the biomarkers of resistance to obtain an expression value for each of the biomarker/s, more specifically, for each of the following biomarkers of resistance MXI1, ZNF395, XPC, MTHFD2, PSME2, JAK2, INTS12, PSMB7, RBM7 and SLC6A8; and (ii) the biomarkers of tolerance to obtain an expression value for each of the biomarker/s, more specifically, for each of the following biomarkers of tolerance are SERINC1, ARLI, COPS2, CRBN, MAP2K2, ARHGDIA, GRINA, STXBP2, RBM7 and SLC6A8.

In some embodiments of the disclosed method, the first step (a), involves determining in at least one biological sample of the subject the expression level of at least three biomarkers of at least one of: (i) at least one biomarker of the resistance, to obtain an expression value for each of the at least one biomarker/s. More specifically, the at least one biomarker of resistance is at least one of MXI1, ZNF395, XPC, MTHFD2, PSME2, JAK2, INTS12, PSMB7, RBM7, SLC6A8 or any combination thereof; and
(ii) at least one biomarker of the tolerance, to obtain an expression value for each of the at least one biomarker/s. Specifically, the at least one biomarker of tolerance is at least one of SERINC1, ARLI, COPS2, CRBN, MAP2K2, ARHGDIA, GRINA, STXBP2, RBM7, SLC6A8 or any combination thereof.

The next step (b), concerns determining if the expression values obtained in steps (a) (i) and/or (a) (ii) for each of the at least three biomarkers is positive or negative with respect to a predetermined standard expression value or to an expression value of the biomarker/s in at least one control sample. In should be noted that wherein at least one of: (I) a positive expression value of at least one of the MTHFD2, PSME2, JAK2, INTS12, PSMB7 and RBM7 biomarker/s in the sample, and/or a negative expression value of at least one of the MXI1, ZNF395, XPC and SLC6A8 biomarker/s in the sample indicate(s) that the resistance level is elevated in the subject; and/or (II) a positive expression value of at least one of the MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample indicate(s) that the tolerance level is elevated in the subject. In some embodiments, the level of expression of at least one of the tolerance and/or resistance biomarkers is determined in step (a), may be performed as defined by the present invention. More specifically, in some specific and non-limiting embodiments, step (a) of the disclosed prognostic methods comprises determining in at least one biological sample of the subject the expression level of: (i) the biomarkers of resistance to obtain an expression value for each of the biomarker/s, more specifically, for each of the following biomarkers of resistance MXI1, ZNF395, XPC, MTHFD2, PSME2, JAK2, INTS12, PSMB7, RBM7 and SLC6A8; and (ii) the biomarkers of tolerance to obtain an expression value for each of the biomarker/s, more specifically, for each of the following biomarkers of tolerance are SERINC1, ARLI, COPS2, CRBN, MAP2K2, ARHGDIA, GRINA, STXBP2, RBM7 and SLC6A8.

In some embodiments, the step of determining the level of expression of at least one of the biomarker/s of resistance and/or at least one the biomarker/s of tolerance is performed in the prognostic methods disclosed herein by the step of contacting at least one detecting molecule or any combination or mixture of plurality of detecting molecules with a biological sample of the subject, or with any nucleic acid or protein product obtained therefrom. It should be understood that each of the detecting molecules is specific for one of the biomarkers.

In some specific embodiments, the at least one detecting molecule used for determining the level of expression may be any one of nucleic acid-based detecting molecules and/or amino acid-based detecting molecules.

More specifically, in some embodiments, nucleic acid detecting molecule/s useful in the prognostic methods disclosed herein may comprise at least one of: (a) at least one oligonucleotide/s, each oligonucleotide specifically hybridizes to a nucleic acid sequence encoding the at least one biomarker, or any fragments thereof; and/or (b) at least one nucleic acid aptamer/s specific for the at least one of the biomarkers.

In some embodiments, the biological sample analyzed by the disclosed methods may be any biological sample, for example, any body fluid sample (that may either comprise cells or not), and/or any cell sample of the examined subject.

In some specific and non-limiting embodiments, the sample used for the prognostic methods disclosed herein, may be a blood sample. In yet some further embodiments, the sample analyzed by the disclosed methods may be a cell sample obtained from any tissue or organ of the examined subject. In some embodiments, the sample may be a skin cell sample. In yet some further embodiments, the sample may be any sample obtained by a biopsy from any tissue and/or organ. In some embodiments, the sample is a biopsy of a diseased tissue or organ. Still further in some embodiments, the sample may be a tumor biopsy, or any biopsy or sample of diseased, damaged or injured tissue or organ (e.g., sample of injured liver).

In some embodiments, the prognostic methods disclosed herein may be suitable for any pathological disorder, specifically, at least one immune related disorder.

Still further, in some embodiments such disorder may be at least one of an infectious disease caused by at least one pathogen, an inflammatory disorder, an autoimmune disorder, a proliferative disorder, a neurodegenerative disorder (and/or any protein misfolding disorder), a metabolic disorder and a condition involving at least one wound in at least one tissue and/or organ of the subject.

In some embodiments, the prognostic methods discussed herein may be applicable for infectious disease. In some embodiments, such infectious disease is caused by at least one pathogen. In yet some further embodiments, the pathogen may be any pathogen, for example, at least one of a viral pathogen, a viroid pathogen, a protozoan pathogen, a prion, a bacterial pathogen, a fungal pathogen and a parasite.

In some embodiments, a pathologic disorder caused by at least one pathogen may be a septic shock. In yet some particular and non-limiting embodiments, the prognostic methods disclosed herein may be applicable for viral pathogen such as Influenza A virus (IAV), Ebola virus, Severe acute respiratory syndrome coronavirus 2 (SARS-COV2), Respiratory Syncytial Virus (RSV), and/or Human parainfluenza virus type 3 (HPIV3).

It should be understood that all pathogens disclosed in the present disclosure in connection with other aspects of the present invention are also applicable in the present aspect.

According to such embodiments, a reduced susceptibility and/or a positive outcome of the disorder is characterized by an elevated level of resistance and/or a low level of tolerance. Thus, if the subject tested by the prognostic methods disclosed herein suffers from an infectious disease caused by a viral pathogen and displays elevated level of resistance and/or a low level of tolerance, such subject is determined by the prognostic methods disclosed herein, as a subject having a reduced susceptibility and/or a positive outcome of the viral infectious disease.

In yet some further embodiments, the prognostic method of the present disclosure may be applicable for subjects suffering from an immune related disorder such as an inflammatory or autoimmune disorder.

In yet some further specific and non-limiting embodiments, such inflammatory or autoimmune disorder is any one of Systemic Lupus Erythematosus (SLE) or Rheumatoid Arthritis (RA).

Thus, for a subject suffering from an inflammatory or autoimmune disorder such as SLE or RA, elevated level of resistance and/or a low level of tolerance reflect and indicate susceptibility and/or a negative outcome of the disorder.

In yet some further embodiments, the prognostic methods disclosed herein may be applicable for any proliferative disorder. In some particular and non-limiting embodiments, such proliferative disorder may be a neoplastic disorder, specifically, cancer.

In some embodiments for cancers such as glioma or breast cancer, at least one of: (i) the susceptibility and/or negative outcome of these particular cancers is characterized by an elevated level of resistance and or reduced level of tolerance; and (ii) the reduced susceptibility and/or positive outcome of these cancers is characterized by an elevated level of tolerance and/or a reduced level of resistance.

Thus, if subject that suffers from cancers such as glioma or breast cancer, displays elevated level of resistance and/or reduced level of tolerance, this subject is determined by the prognostic methods disclosed herein as having increased susceptibility and/or negative outcome. In case however, the subject displays an elevated level of tolerance and/or a reduced level of resistance, such subject is determined by the prognostic methods disclosed herein as having reduced susceptibility and/or positive outcome.

In yet some further embodiments, the subject prognosed by the methods disclosed herein is suffering from a cancer such as Leukemia (CLL). In some embodiments at least one of: (i) a reduced susceptibility and/or positive outcome of the cancer is characterized by elevated level of tolerance; and (ii) susceptibility and/or negative outcome of the cancer is characterized by a reduced level of tolerance. Thus, if the CLL subject displays elevated level of tolerance, the subject is classified by the prognostic methods as having a reduced susceptibility and/or positive outcome. If however the prognosed CLL subject displays a reduced level of tolerance, this subject is classified as having increased susceptibility and/or negative outcome.

Still further, in some embodiments, the prognosed subject is suffering from a cancer such as Multiple Myeloma (MM). In some embodiments, at least one of: (i) the susceptibility and/or negative outcome of the cancer is characterized by an elevated level of tolerance; and (ii) the reduced susceptibility and/or positive outcome of the cancer is characterized by a reduced level of tolerance. Thus, MM subjects displaying an elevated level of tolerance is prognosed as having susceptibility and/or negative outcome of the MM cancer. If however, a subject suffering from MM display reduced level of tolerance, such subject is prognosed by the prognostic methods disclosed herein as having reduced susceptibility and/or expected to display a positive outcome.

In yet some further embodiments, the prognostic methods disclosed herein may prognose a subject suffering from a cancer such as any one of Lung adenocarcinoma, Neuroblastoma and Astrocytoma. It should be noted that these cancers are characterized by at least one of: (i) the susceptibility and/or negative outcome of the cancer is characterized by an elevated level of resistance; and (ii) reduced susceptibility and/or positive outcome of the cancer is characterized by a reduced level of resistance. Thus, a subject displaying an elevated level of resistance is classified as having increased susceptibility and/or negative outcome of the cancer. If however, the subject displays a reduced level of resistance, this subject is prognosed as having reduced susceptibility and/or positive outcome of the cancer.

Still further, in some embodiments of the prognostic methods disclosed herein, the condition is at least one wound (either acute, or chronic) in at least one tissue and/or organ of the subject. It should be noted that a positive outcome of wound healing is characterized by an elevated level of tolerance. Thus, subjects that display elevated level of tolerance are prognosed as having an increased chance for positive outcome and successful healing of the wound.

As shown by Example 9, chronic wound in liver injury may display a clear dependency on tolerance markers. Thus, in some embodiments, high baseline tolerance predicts a positive outcome of the disease. However, in chronic liver injury, a subject that display elevated tolerance is prognosed as having increased probability of negative outcome.

The prognostic methods disclosed herein may be used for determining a particular and personalized treatment regimen to the prognosed subjects and such, in some embodiments, the methods disclosed herein may further comprise the step of administering to the prognosed subject an effective amount of at least one therapeutic compound that modifies the tolerance and/or resistance in the subject. In more specific embodiments, such therapeutic compound is any one of: (a) a compound that elevates resistance and/or reduces tolerance, may be applicable for a disorder where a responsiveness and/or positive outcome is characterized with elevated resistance and/or reduced tolerance; and (b) a compound that reduces resistance and/or elevates tolerance, is applicable in a disorder where a responsiveness and/or positive outcome in a subject is characterized with reduced resistance and/or elevated tolerance. Thus, if a subject display reduced resistance in a disorder where a responsiveness and/or positive outcome is characterized with elevated resistance, this subject will be administered with (a) a compound that elevates resistance and/or reduces tolerance. If the subject that suffers from a disorder where a responsiveness and/or positive outcome in a subject is characterized with reduced resistance and/or elevated tolerance, displays an elevated resistance and/or reduced tolerance, such subject will be administered with a compound (b) that reduces resistance and/or elevates tolerance. Non-limiting examples of disorders that display positive outcome when tolerance is elevated and resistance is reduced, include RA, SLE, Breast cancer and glioma. Reduced tolerance and increased resistance are associated with positive outcome in viral infections Reduced tolerance is beneficial min MM, elevated tolerance display positive outcome in wound healing, and reduced resistance is associated with positive outcome in neuroblastoma and astrocytoma.

A further aspect of the present disclosure relates to a prognostic method for predicting and assessing responsiveness of a subject suffering from a pathologic disorder, to at least one compound or to a treatment regimen comprising this specific compound. Optionally, or additionally, the disclosed method may be also applicable for monitoring disease progression. In some embodiments, the method disclosed herein may comprise the following steps. First in step (a), determining the levels of resistance and/or tolerance of the subject, in at least one sample of the subject. The next step (b), involves classifying the subject as: (I) a responder to the at least one compound or a treatment regimen comprising the compound, if at least one sample obtained after the initiation of the treatment regimen and/or a sample of the subject contacted with the compound displays at least one of: (i) an elevated resistance and/or reduced tolerance, in a disorder where responsiveness and/or positive outcome in a subject is characterized with elevated resistance and/or reduced tolerance; and (ii) reduced resistance and/or elevated tolerance, in a disorder where responsiveness and/or positive outcome is characterized with reduced resistance and/or elevated tolerance. Alternatively, the subject may be classified as (II), a non-responder to the at least one compound or a treatment regimen comprising the compound, if at least one sample obtained after the initiation of the treatment regimen and/or a sample of the subject contacted with the compound displays at least one of: (i) elevated resistance and/or reduced tolerance, in a disorder where a responsiveness and/or positive outcome is characterized with reduced resistance and/or elevated tolerance; and (ii) reduced resistance and/or elevated tolerance, in a disorder where a responsiveness and/or positive outcome in a subject is characterized with elevated resistance and/or reduced tolerance. The method thereby enables predicting and assessing responsiveness of the subject to the treatment regimen.

In some embodiments, positive or negative outcome in a subject is determined by determining for each disorder the standard levels of resistance and/or tolerance in a (control) population of subjects having developed a negative outcome of the pathological disorder and/or the standard levels of resistance and/or tolerance in a (control) population of subjects having developed a positive outcome from the disorder.

In some embodiments, step (a), of the disclosed prognostic method, that concerns determination of the resistance and/or tolerance in the subject is performed by the method comprising the steps of: First (a), determining in at least one biological sample of the subject the expression level of at least one of: (i) at least one biomarker of the resistance, to obtain an expression value for each of the at least one biomarker/s, wherein the at least one biomarker is at least one of MXI1, ZNF395, XPC, MTHFD2, PSME2, JAK2, INTS12, PSMB7, RBM7, SLC6A8 or any combination thereof; and
(ii) at least one biomarker of the tolerance, to obtain an expression value for each of the at least one biomarker/s, wherein the at least one biomarker is at least one of SERINC1, ARLI, COPS2, CRBN, MAP2K2, ARHGDIA, GRINA, STXBP2, RBM7, SLC6A8 or any combination thereof.

The next step (b), concerns determining if the expression values obtained in steps (a) (i) and/or (a) (ii) for each of the at least one biomarker/s, is positive or negative with respect to a predetermined standard expression value or to an expression value of the biomarker/s in at least one control sample. It should be noted that at least one of: In some embodiments (I), a positive expression value of at least one of the MTHFD2, PSME2, JAK2, INTS12, PSMB7 and RBM7 biomarker/s in the sample, and/or a negative expression value of at least one of the MXII, ZNF395, XPC and SLC6A8 biomarker/s in the sample indicate(s) that the resistance level is elevated in the subject. In some alternative or additional embodiments (II), a positive expression value of at least one of the MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample, indicate(s) that the tolerance level is elevated in the subject.

In some embodiments, step (a), of the disclosed prognostic method, that concerns determination of the resistance and/or tolerance in the subject is performed by the method comprising the steps of: First (a), determining in at least one biological sample of the subject the expression level of at least three biomarkers of at least one of: (i) at least one biomarker of the resistance, to obtain an expression value for each of the at least one biomarker/s, wherein the at least one biomarker is at least one of MXI1, ZNF395, XPC, MTHFD2, PSME2, JAK2, INTS12, PSMB7, RBM7, SLC6A8 or any combination thereof; and
(ii) at least one biomarker of the tolerance, to obtain an expression value for each of the at least one biomarker/s, wherein the at least one biomarker is at least one of SERINC1, ARLI, COPS2, CRBN, MAP2K2, ARHGDIA, GRINA, STXBP2, RBM7, SLC6A8 or any combination thereof.

The next step (b), concerns determining if the expression values obtained in steps (a) (i) and/or (a) (ii) for each of the at least three biomarkers, is positive or negative with respect to a predetermined standard expression value or to an expression value of the biomarker/s in at least one control sample. It should be noted that at least one of: In some embodiments (I), a positive expression value of at least one of the MTHFD2, PSME2, JAK2, INTS12, PSMB7 and RBM7 biomarker/s in the sample, and/or a negative expression value of at least one of the MXII, ZNF395, XPC and SLC6A8 biomarker/s in the sample indicate(s) that the resistance level is elevated in the subject. In some alternative or additional embodiments (II), a positive expression value of at least one of the MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample, indicate(s) that the tolerance level is elevated in the subject.

It should be understood that in some embodiments, determination of the resistance and/or tolerance in the subject, by the prognostic method disclosed herein is based on the determination of the expression level/s of at least one, at least two, at least three, at least four, at least five, at least six, at least seven, at least eight, at least nine biomarkers, at least ten, at least eleven, at least twelve, at least thirteen, at least fourteen, at least fifteen, at least sixteen, at least seventeen, at least eighteen of the disclosed biomarkers, and/or of any of the biomarkers disclosed by the present disclosure, specifically, any one of the biomarkers disclosed in Tables 1A, 1B, 4 and 5. It should be further understood that any additional biomarker and/or control protein/gene may be determined by the disclosed methods.

In some embodiments, the level of expression of at least one of the tolerance and/or resistance biomarkers is determined in step (a), as defined by the present disclosure in connection with other aspects of the invention. More specifically, in some embodiments, the step of determining the level of expression of at least one of the biomarker/s of resistance and/or at least one the biomarker/s of tolerance is performed in the prognostic methods disclosed herein by the step of contacting at least one detecting molecule or any combination or mixture of plurality of detecting molecules with a biological sample of the subject, or with any nucleic acid or protein product obtained therefrom. It should be understood that each of the detecting molecules is specific for one of the biomarkers. In some specific embodiments, the at least one detecting molecule used for determining the level of expression may be any one of nucleic acid-based detecting molecules and/or amino acid-based detecting molecules.

More specifically, in some embodiments, nucleic acid detecting molecule/s useful in the prognostic methods disclosed herein may comprise at least one of: (a) at least one oligonucleotide/s, each oligonucleotide specifically hybridizes to a nucleic acid sequence encoding the at least one biomarker; and/or (b) at least one nucleic acid aptamer/s specific for the at least one of the biomarkers.

In some embodiments, the biological sample analyzed by the disclosed methods may be any biological sample, for example, any body fluid sample (that may either comprise cells or not), and/or any cell sample of the examined subject.

In some specific and non-limiting embodiments, the sample used for the prognostic methods disclosed herein, may be a blood sample. In yet some further embodiments, the sample analyzed by the disclosed methods may be a cell sample obtained from any tissue or organ of the examined subject. In some embodiments, the sample may be a skin cell sample. In yet some further embodiments, the sample may be any sample obtained by a biopsy from any tissue and/or organ. In some embodiments, the sample is a biopsy of a diseased tissue or organ. Still further in some embodiments, the sample may be a tumor biopsy.

As indicated above, the prognostic methods discussed herein in the present aspect, may further provide a tool for monitoring disease progression. In some embodiments, monitoring disease progression may provide an important tool for predicting and determining disease relapse and assessing a remission interval. Thus, in some embodiments, the prognostic method disclosed herein may further comprises the steps of: (c) repeating step (a) to determine the levels of resistance and/or tolerance in at least one more temporally-separated sample of the subject, specifically, to a sample obtained from the subject in at least one additional time point. The next and (d), concerns predicting and/or determining disease relapse in the subject, if at least one temporally separated sample obtained after the initiation of the treatment regimen displays at least one of: (i) elevated resistance and/or reduced tolerance, in a disorder where a responsiveness and/or positive outcome is characterized with reduced resistance and/or elevated tolerance; and (ii) reduced resistance and/or elevated tolerance, in a disorder where a responsiveness and/or positive outcome in a subject is characterized with elevated resistance and/or reduced tolerance.

In some embodiments, the pathological disorder applicable in the preset prognostic method is at least one immune related disorder.

In yet some further embodiments, the immune related disorder is at least one of an infectious disease caused by at least one pathogen, an inflammatory disorder, an autoimmune disorder, a proliferative disorder, a neurodegenerative disorder, a metabolic disorder and a condition involving at least one wound in at least one tissue and/or organ of said subject.

As discussed herein above in connection with other aspects of the present disclosure, the prognostic methods discussed herein further provide a powerful therapeutic tool allowing tailored treatment for the prognosed subject. Moreover, by monitoring the subject using the methods disclosed herein, a continuous tailored treatment regimen is offered for each stage of the disease and recovery. Therefore, in some embodiments, the methods disclosed herein may further comprises the step of administering to the prognosed and/or monitored subject an effective amount of at least one therapeutic compound that modifies the tolerance and/or resistance in the subject. It should be noted that a suitable therapeutic compound is any one of: (i) a compound that elevates resistance and/or reduces tolerance, in a disorder where a responsiveness and/or positive outcome is characterized with elevated resistance and/or reduced tolerance; and (ii) a compound that reduces resistance and/or elevates tolerance, in a disorder where a responsiveness and/or positive outcome in a subject is characterized with reduced resistance and/or elevated tolerance.

As demonstrated by Example 10, compounds that modulate the levels of at least one of the disclosed biomarkers may be used to modulate the tolerance and/or resistance of the subject. For example, a compound that reduces the expression levels of the tolerance biomarker ARHGDIA (e.g., gRNAs specifically directing CRISPR-Cas system to the ARHGDIA encoding sequence), reduced the tolerance of the subject and led to a positive outcome of a virally infected subject.

A further aspect of the present disclosure relates to a method for determining a personalized treatment regimen for a subject suffering from a pathologic disorder. The therapeutic method disclosed herein is personally adapted for each patient and may further provide a continuous and monitored treatment regimen. This therapeutic method therefore combines diagnostic steps for determining the immunological state of the treated subject, specifically, the resistance and/or tolerance levels of the treated subject. More specifically, in some embodiments, the method comprising the steps of: First in step (a), determining the level/s of resistance and/or tolerance of the subject.

The next step (b), involves selecting a treatment regimen determined as modifying the levels of resistance and/or tolerance in the subject. More specifically, where reduction of resistance and/or elevation of tolerance is required, an appropriate treatment regimen selected is a treatment that reduces resistance and/or elevates tolerance. In some other embodiments, where elevation of resistance and/or reduction of tolerance is required, an appropriate treatment regimen selected is a treatment that elevates resistance and/or reduces tolerance. It should be further understood that an appropriate treatment regimen may affect only one of, resistance or tolerance.

Thus, in some embodiments, a treatment regimen is selected if at least one of: (i) the treatment regimen elevates resistance and/or reduces tolerance, in at least one sample of the subject, wherein the subject is suffering from a disorder where a positive outcome is characterized with elevated resistance and/or reduced tolerance; and (ii) the treatment regimen reduces resistance and/or elevated tolerance, in at least one sample of the subject. In some embodiments, the subject is suffering from a disorder where positive outcome is characterized with reduced resistance and/or elevated tolerance. As discussed above, in some embodiments, the standard levels of resistance and/or tolerance are determined in a (control) population of responders and/or the standard levels of resistance and/or tolerance in a (control) population of non-responders.

In some specific embodiments, the diagnostic step (a) of the therapeutic methods discussed herein may be performed by the method comprising the steps of: (a) determining in at least one biological sample of the subject the expression level of at least one of: (i) at least one biomarker of the resistance, to obtain an expression value for each of the at least one biomarker/s, wherein the at least one biomarker is at least one of MXII, ZNF395, XPC, MTHFD2, PSME2, JAK2, INTS12, PSMB7, RBM7, SLC6A8 or any combination thereof; and (ii) at least one biomarker of the tolerance, to obtain an expression value for each of the at least one biomarker/s, wherein the at least one biomarker is at least one of SERINC1, ARLI, COPS2, CRBN, MAP2K2, ARHGDIA, GRINA, STXBP2, RBM7, SLC6A8 or any combination thereof. The next step (b) involves determining if the expression values obtained in steps (a) (i) and/or (a) (ii) for each of the at least one biomarker/s is positive or negative with respect to a predetermined standard expression value or to an expression value of the biomarker/s in at least one control sample. It should be noted that at least one of: (I) a positive expression value of at least one of the MTHFD2, PSME2, JAK2, INTS12, PSMB7 and RBM7 biomarker/s in the sample, and/or a negative expression value of at least one of said MXI1, ZNF395, XPC and SLC6A8 biomarker/s in the sample, indicate(s) that the resistance level is elevated in the subject; and/or (II) a positive expression value of at least one of the MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample, indicate(s) that the tolerance level is elevated in the subject.

In yet some further embodiments, the diagnostic step (a) of the therapeutic methods discussed herein may be performed by the method comprising the steps of: (a) determining in at least one biological sample of the subject the expression level of at least three biomarkers of at least one of: (i) at least one biomarker of the resistance, to obtain an expression value for each of the at least one biomarker/s, wherein the at least one biomarker is at least one of MXI1, ZNF395, XPC, MTHFD2, PSME2, JAK2, INTS12, PSMB7, RBM7, SLC6A8 or any combination thereof; and (ii) at least one biomarker of the tolerance, to obtain an expression value for each of the at least one biomarker/s, wherein the at least one biomarker is at least one of SERINC1, ARLI, COPS2, CRBN, MAP2K2, ARHGDIA, GRINA, STXBP2, RBM7, SLC6A8 or any combination thereof. The next step (b) involves determining if the expression values obtained in steps (a) (i) and/or (a) (ii) for each of the at least three biomarkers is positive or negative with respect to a predetermined standard expression value or to an expression value of the biomarker/s in at least one control sample. It should be noted that at least one of: (I) a positive expression value of at least one of the MTHFD2, PSME2, JAK2, INTS12, PSMB7 and RBM7 biomarker/s in the sample, and/or a negative expression value of at least one of said MXI1, ZNF395, XPC and SLC6A8 biomarker/s in the sample, indicate(s) that the resistance level is elevated in the subject; and/or (II) a positive expression value of at least one of the MAP2K2, ARHGDIA, GRINA, STXBP2 and SLC6A8 biomarker/s in the sample, and/or a negative expression value of at least one of the SERINC1, ARLI, COPS2, CRBN and RBM7 biomarker/s in the sample, indicate(s) that the tolerance level is elevated in the subject. It should be understood the at least three biomarkers may be in some embodiments, at least three or more of the resistance biomarkers (i), in some other embodiments, at least three or more of the tolerance biomarkers (ii), or in some other embodiments, at least three or more of the resistance (i), and the tolerance biomarkers (ii).

In some embodiments, the level of expression of at least one of tolerance and/or resistance biomarkers is determined in step (a) of the disclosed therapeutic method as defined by the present invention herein above. In some embodiments, the step of determining the level of expression of at least one of the biomarker/s of resistance and/or at least one the biomarker/s of tolerance is performed in the therapeutic methods disclosed herein by the step of contacting at least one detecting molecule or any combination or mixture of plurality of detecting molecules with a biological sample of the subject, or with any nucleic acid or protein product obtained therefrom. It should be understood that each of the detecting molecules is specific for one of the biomarkers.

In some specific embodiments, the at least one detecting molecule used for determining the level of expression may be any one of nucleic acid-based detecting molecules and/or amino acid-based detecting molecules. More specifically, in some embodiments, nucleic acid detecting molecule/s useful in the prognostic methods disclosed herein may comprise at least one of: (a) at least one oligonucleotide/s, each oligonucleotide specifically hybridizes to a nucleic acid sequence encoding the at least one biomarker; and/or (b) at least one nucleic acid aptamer/s specific for the at least one of the biomarkers.

In some embodiments, the biological sample analyzed by the disclosed methods may be any biological sample, for example, any body fluid sample (that may ither comprise cells or not), and/or any cell sample of the examined subject. In some specific and non-limiting embodiments, the sample used for the therapeutic methods disclosed herein, may be a blood sample. In yet some further embodiments, the sample analyzed by the disclosed methods may be a cell sample obtained from any tissue or organ of the examined subject. In some embodiments, the sample may be a skin cell sample. In yet some further embodiments, the sample may be any sample obtained by a biopsy from any tissue and/or organ. In some embodiments, the sample is a biopsy of a diseased tissue or organ. Still further in some embodiments, the sample may be a tumor biopsy.

In some embodiments, the methods disclosed herein are suitable for determining a treatment regimen for a pathological disorder that may be at least one immune related disorder.

In some embodiments, the immune related disorder is at least one of an infectious disease caused by at least one pathogen, an inflammatory disorder, an autoimmune disorder, a proliferative disorder, a neurodegenerative disorder, a metabolic disorder and a condition involving at least one wound in at least one tissue and/or organ of the subject. It should be noted that the disclosed conditions may be congenital or acquired conditions.

In yet some further embodiments, the methods disclosed herein may further comprise the step of administering to the subject an effective amount of at least one therapeutic compound that modifies the tolerance and/or resistance in the subject. More specifically, an appropriate therapeutic compound is any one of: (i) a compound that elevates resistance and/or reduces tolerance, in a disorder where a responsiveness and/or positive outcome is characterized with elevated resistance and/or reduced tolerance; and (ii) a compound that reduces resistance and/or elevates tolerance, in a disorder where a responsiveness and/or positive outcome in a subject is characterized with reduced resistance and/or elevated tolerance. In some particular embodiments, such compounds may be any compound that specifically modulates the expression of the at least one biomarker/s of resistance and/o the at least one biomarker/s of tolerance. For example, any nucleic acid-based compound, e.g., siRNA, shRNA, anti-sense oligonucleotide, and CRISPR-Cas or any other gene editing system, miRNA, lncRNA, or any molecule that directly or indirectly modulate the expression of at least one of the resistance and/or tolerance biomarkers disclosed herein.

In some embodiments, the method of the invention is directed at diagnosing, prognosing and treating a pathologic disorder. In more specific embodiments, such disorder may be at least one of a proliferative disorder, an inflammatory disorder, an infectious disease caused by a pathogen, an autoimmune-disease as well as CVDs and metabolic conditions. Thus, in some specific embodiments, the subject treated by the method of the invention may be a subject suffering of an immune-related disorder. An “Immune-related disorder” or “Immune-mediated disorder”, as used herein encompasses any condition that is associated with the immune system of a subject, more specifically through inhibition of the immune system, or that can be treated, prevented or ameliorated by reducing degradation of a certain component of the immune response in a subject, such as the adaptive or innate immune response. An immune-related disorder may include infectious condition (e.g., by a pathogen, specifically, viral, bacterial or fungal infections), inflammatory disease, autoimmune disorders, metabolic disorders and proliferative disorders, specifically, cancer. In some specific embodiments wherein the immune-related disorder or condition may be a primary or a secondary immunodeficiency.

As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions and kits of the present invention may be applicable for a patient suffering from any one of non-solid and solid tumors.

Malignancy, as contemplated in the present invention may be any one of carcinomas, melanomas, lymphomas, leukemia, myeloma and sarcomas. Therefore, in some embodiments any of the methods of the invention (specifically, therapeutic, prognostic and non-therapeutic methods), kits and compositions disclosed herein, may be applicable for any of the malignancies disclosed by the present disclosure.

More specifically, carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges. Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges. Particular and non-limiting embodiments of tumors applicable in the methods, compositions and kit of the present disclosure may include but are not limited to at least one of ovarian cancer, liver carcinoma, colorectal carcinoma, breast cancer, pancreatic cancer, brain tumors and any related conditions, as well as any metastatic condition, tissue or organ thereof.

In some other embodiments, the methods, compositions and kits of the invention are relevant to colorectal carcinoma, or any malignancy that may affect all organs in the peritoneal cavity, such as liposarcoma for example. In some further embodiments, the method of the invention may be relevant to tumors present in the pleural cavity (mesothelioma, invading lung) the urinary bladder, and tumors of the brain meninges.

It should be understood that the methods, compositions and kits of the present disclosure are applicable for any type and/or stage and/or grade of any of the malignant disorders discussed herein or any metastasis thereof. Still further, it must be appreciated that the methods, compositions and kits of the invention may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, compositions and kits of the present disclosure are applicable for any type and/or stage and/or grade of any metastasis, metastatic cancer or status of any of the cancerous conditions disclosed herein.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. Further malignancies that may find utility in the present invention can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer,

Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma;

Vaginal cancer; Visual pathway and hypothalamic glioma, childhood; Vulvar cancer; Waldenstrom macroglobulinemia and Wilms tumor (kidney cancer).

Thus, according to some embodiments, the method of the invention may be used for the treatment of a patient suffering from any autoimmune disorder. In some specific embodiments, the methods of the invention may be used for treating an autoimmune disease such as for example, but not limited to Systemic Lupus Erythematosus (SLE), Rheumatoid Arthritis (RA), inflammatory bowel disease (IBD), ulcerative colitis, Crohn's disease, fatty liver disease, Lymphocytic colitis, Ischaemic colitis, Diversion colitis, Behçet's syndrome, Indeterminate colitis, Graft versus Host Disease (GvHD), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

Still further, as shown by Example 8, the disclosed T and R states determined by the methods of the present disclosure may be applicable for any injury, for example, any organ injury, specifically, liver injury. Thus, in some embodiments, the disclosed methods may be applicable for liver injury. More specifically, according to the present disclosure, liver injury encompasses acute or chronic liver disease, cirrhosis and any disease or complication associated therewith. For example, at least one of hepatic encephalopathy, spontancous bacterial peritonitis (SBP), ascites, variceal bleeding, cirrhosis associated hyperdynamic circulation, hepatorenal syndrome, hepatopulmonary syndrome, portopulmonary hypertension and variceal bleeding, and even hepatic carcinoma. In some further embodiments. hepatic injury discussed herein may result from any type of insult, for example, a viral pathogen, including HCV. HBV, CMV, and EBV, alcoholism and/or fatty liver disease. The term “cirrhosis” as used herein refers to the final common histological outcome of a wide verity of chronic liver diseases, characterized by the replacement of liver tissue by fibrous scar tissue and regeneration of nodules, leading to progressive loss of liver function. Cirrhosis is usually caused by Hepatitis B and C viruses, alcoholism and fatty liver disease. The term “ascites”, as used herein describes the condition of pathologie fluid accumulation within the abdominal cavity, most commonly due to cirrhosis and sever liver disease.

In some further embodiments, the methods of the invention may be applicable for immune-related disorder or condition that may be a pathologic condition caused by at least one pathogen. In yet some specific embodiments, the prognostic and therapeutic methods of the invention, as well as the kits and compositions may be also applicable for treating a subject suffering from an infectious disease. It should be appreciated that an infectious disease as used herein also encompasses any infectious disease caused by a pathogenic agent, specifically, a pathogen. More specifically, such infectious disease may be any pathological disorder caused by a pathogen. As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode, as well as toxins and venoms.

In yet some further embodiments, the methods of the invention may be applicable for any infectious disorders caused by a viral pathogen or a virus. The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis. It should be noted that the term “viruses” is used in its broadest sense to include viruses of the families Flaviviruses, Alphaviruses, Togaviruses, Coronaviruses, Hepatitis D, Orthomyxoviruses, Paramyxoviruses, Rhabdovirus. Still further, more specific embodiments relate to Influenza viruses A and B, coronaviruses (e.g. SARS-COV2), Ebola viruses, adenoviruses, papovaviruses, herpesviruses: simplex, varicella-zoster, Epstein-Barr (EBV), Cowpox viruses, Cytomegalo virus (CMV), pox viruses: smallpox, vaccinia, hepatitis B (HBV), rhinoviruses, hepatitis A (HBA), poliovirus, respiratory syncytial virus (RSV), Middle East Respiratory Syndrome (MERS), rubella virus, hepatitis C (HBC), arboviruses, rabies virus, measles virus, mumps virus, human deficiency virus (HIV), HTLV I and II, flaviviruses such as Dengue virus, west nile virus, yellow fever virus, and Zika virus.

In yet some other specific embodiments, the methods and composition of the invention may be applicable for prognosing, monitoring and treating an infectious disease caused by bacterial pathogens. More specifically, a prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Mycoplasma pulmonis, Salmonella typhimurium, Treponema pallidum, Borrelia burgdorferi, Neisseria gonorrhea, Neisseria meningitidis, Legionella pneumophila, Bordetella pertussis, Escherichia coli, Salmonella typhi, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes. A lower eukaryotic organism includes a yeast or fungus such as but not limited to Candida albicans, Pneumocystis carinii, Aspergillus, Histoplasma capsulatum, Blastomyces dermatitidis, Cryptococcus neoformans, Trichophyton and Microsporum, are also encompassed by the invention. A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania. More specifically, in certain embodiments the methods and compositions of the invention may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for the methods, kits and compositions for the treatment, prognosis and monitoring of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species. As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In yet some further specific embodiments, the present invention provides compositions and methods for use in the treatment, prevention, amelioration or delay the onset of a pathological disorder, wherein said pathological disorder is a result of a prion. As used herein, the term “prion” refers to an infectious agent composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue and all are currently untreatable and universally fatal. It should be appreciated that an infectious disease as used herein also encompasses any pathologic condition caused by toxins and venoms.

In yet some further embodiments, immune-related disorder as used herein may further encompass in some embodiments, any neurodegenerative disorders or diseases. Neurodegeneration is the umbrella term for the progressive loss of structure or function of neurons, including synaptic dysfunction and death of neurons. Many neurodegenerative diseases including Parkinson's and Alzheimer's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present invention. More specifically, the term “neurodegenerative diseases” is the general term for the progressive loss of structure or function of neurons, leading to their death. The major risk factor for neurodegenerative diseases is aging. Mitochondrial DNA mutations as well as oxidative stress both contribute to aging. Many of these diseases are late-onset, meaning there is some factor that change as a person ages, for each disease. One constant factor is that in each disease, neurons gradually lose function as the disease progresses with age. Still further, it should be appreciated that in certain embodiments, the methods disclosed herein may be further applicable for disorders characterized by beta-amyloid protein aggregation. A group of disorders associated with beta-amyloid protein aggregation include Alzheimer's disease (AD), where deposits of a protein precursor called beta-amyloid build up (termed plaques) in the spaces between nerve cells and twisted fibers of tau protein build up (termed tangles) inside the cells. More specifically, “Beta-amyloid protein aggregations” as used herein relates to cerebral plaques laden with β-amyloid peptide (Aβ) and dystrophic neurites in neocortical terminal fields as well as prominent neurofibrillary tangles in medial temporal-lobe structures, which are important pathological features of Alzheimer's disease. Subsequently, loss of neurons and white matter, congophilic (amyloid) angiopathy are also present.

In addition to neurodegenerative diseases disclosed herein, the present methods may be also applicable for metabolic disorders and/or as well as vascular conditions that may include in some embodiments, atherosclerosis and peripheral vascular diseases, as well as cardiovascular diseases such as coronary artery diseases (CAD). Of particular interest in connection with metabolic disorders are conditions associated with obesity, hypertension, elevated cholesterol (combined hyperlipidemia), such conditions often termed metabolic syndrome (it is also known as Syndrome X, Reavan's syndrome, or CHAOS).

Still further, as providing a tool for modulating the balance between resistance and tolerance, the methods of the invention may offer a promising therapeutic modality for a variety of any immune-related disorder. In some embodiments, such immune-related disorders may be any disorder associated with immunodeficiency. For example, innate and acquired immunodeficiencies caused by immunosuppressive treatments (chemo- and radiotherapy), pathogenic infections, cancer and HSCT. More specifically, Immunodeficiency (or immune deficiency) is a state in which the immune system's ability to fight infectious disease and cancer is compromised or entirely absent. Most cases of immunodeficiency are acquired (“secondary”) due to extrinsic factors that affect the patient's immune system. Examples of these extrinsic factors include viral infection, specifically, HIV, extremes of age, and environmental factors, such as nutrition. In the clinical setting, the immunosuppression by some drugs, such as steroids, can be either an adverse effect or the intended purpose of the treatment. Examples of such use are in organ transplant surgery as an anti-rejection measure and in patients suffering from an overactive immune system, as in autoimmune diseases. Immunodeficiency also decreases cancer immuno-surveillance, in which the immune system scans the cells and kills neoplastic ones. Still further, Primary immunodeficiencies (PID), also termed innate immunodeficiencies, are disorders in which part of the organism immune system is missing or does not function normally. To be considered a primary immunodeficiency, the cause of the immune deficiency must not be caused by other disease, drug treatment, or environmental exposure to toxins). Most primary immune-deficiencies are genetic disorders; the majority is diagnosed in children under the age of one, although milder forms may not be recognized until adulthood. While there are over 100 recognized PIDs, most are very rare. There are several types of immunodeficiency that include, Humoral immune deficiency (including B cell deficiency or dysfunction), which generally includes symptoms of hypogammaglobulinemia (decrease of one or more types of antibodies) with presentations including repeated mild respiratory infections, and/or agammaglobulinemia (lack of all or most antibody production) and results in frequent severe infections (mostly fatal); T cell deficiency, often causes secondary disorders such as acquired immune deficiency syndrome (AIDS); Granulocyte deficiency, including decreased numbers of granulocytes (called as granulocytopenia or, if absent, agranulocytosis) such as of neutrophil granulocytes (termed neutropenia); granulocyte deficiencies also include decreased function of individual granulocytes, such as in chronic granulomatous disease; Asplenia, where there is no function of the spleen; and Complement deficiency in which the function of the complement system is deficient. Secondary immunodeficiencies occur when the immune system is compromised due to environmental factors. Such factors include but are not limited to radiotherapy as well as chemotherapy. While often used as fundamental anti-cancer treatments, these modalities are known to suppress immune function, leaving patients with an increased risk of infection; indeed, infections were found to be a leading cause of patient death during cancer treatment. Neutropenia was specifically associated with vulnerability to life-threatening infections following chemotherapy and radiotherapy. In more specific embodiments, such secondary immunodeficiency may be caused by at least one of chemotherapy, radiotherapy, biological therapy, bone marrow transplantation, gene therapy, adoptive cell transfer or any combinations thereof.

The invention provides prognostic methods for assessing responsiveness of a subject for a specific treatment regimen, for monitoring a disease progression and for predicting relapse of the disease in a subject. It should be noted that “Prognosis”, is defined as a forecast of the future course of a disease or disorder, based on medical knowledge. This highlights the major advantage of the invention, namely, the ability to assess responsiveness or drug-resistance and thereby predict progression of the disease, based on the biomarker levels indicating the resistance and tolerance levels of the prognosed subject. The term “relapse”, as used herein, relates to the re-occurrence of a condition, disease or disorder that affected a person in the past. Specifically, the term relates to the re-occurrence of a disease being treated a regimen.

The term “response” or “responsiveness” to a certain treatment, refers to an improvement in at least one relevant clinical parameter as compared to an untreated subject diagnosed with the same pathology (e.g., the same type, stage, degree and/or classification of the pathology), or as compared to the clinical parameters of the same subject prior to interferon treatment with said medicament. The term “non responder” or “drug resistance” to treatment with a specific medicament, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament.

In some embodiments, the at least one more temporally-separated sample may be obtained after the initiation of at least one treatment regimen. It should be understood that in some particular embodiments, at least one sample may be obtained prior to initiation of the treatment. However, in some embodiments, the methods disclosed herein may be applied to subjects already treated by a treatment regimen. Such monitoring may therefore provide a powerful therapeutic tool used for improving and personalizing the treatment regimen offered to the treated subject.

As indicated above, in accordance with some embodiments of the invention, in order to assess the patient condition, or monitor the disease progression, as well as responsiveness to a certain treatment, at least two “temporally-separated” test samples must be collected from the examined patient and compared thereafter, in order to determine if there is any change or difference in the levels of resistance and/or tolerance between the samples. Such change may reflect a change in the responsiveness of the subject. In practice, to detect a change having more accurate predictive value, at least two “temporally-separated” test samples and preferably more, must be collected from the patient.

The resistance and/or tolerance levels are determined using the method disclosed herein, applied for each sample. As detailed above, the change in resistance and/or tolerance levels is calculated by determining the change in resistance and/or tolerance levels between at least two samples obtained from the same patient in different time-points or time intervals. This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least one week, at least two weeks, at least three weeks, at least one month, at least two months, at least three months, at least four months, at least five months, at least six months, at least one year, or even more.

The number of samples collected and used for evaluation and classification of the subject either as a responder or alternatively, as a drug resistant or as a subject that may experience relapse of the disease, may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the disease progression according to the present disclosure, it is understood that the change in resistance and/or tolerance levels, may be calculated as an average change over at least three samples taken in different time points, or the change may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more.

A further aspect of the present disclosure relates to a method for treating, preventing, inhibiting, reducing, eliminating, protecting or delaying the onset at least one pathological disorder in a subject in need thereof. The therapeutic methods disclosed herein provide tailored and monitored treatment as discussed above, by combining a diagnostic step that allows determination of the specific state of the subject and evaluation of the effect of a particular therapeutic compound on each treated subject. In some embodiments, the method comprises the following steps: First in the diagnostic step (a), determining the levels of resistance and/or tolerance of the subject.

The next step (b), involves classifying the subject as a responder or non-responder to a candidate compound or a treatment regimen comprising the compound, and/or selecting a treatment regimen determined as modifying the levels of resistance and/or tolerance in the subject.

The next step (c) concerns administering a specific compound or subjecting the subject to a treatment regime comprising the compound, if at least one of: (i) the compound or a treatment regimen comprising the compound elevates resistance and/or reduces tolerance, in at least one sample of the subject. In case the subject is suffering from a disorder where a positive outcome is characterized with elevated resistance and/or reduced tolerance; and (ii) the compound or a treatment regimen comprising the compound reduces resistance and/or elevated tolerance, in at least one sample of the subject. In case the subject is suffering from a disorder where positive outcome is characterized with reduced resistance and/or elevated tolerance.

As indicated above, in some embodiments, positive or negative outcome in a subject is determined by determining for each disorder the standard levels of resistance and/or tolerance in a (control) population of subjects having developed a negative outcome of the pathological disorder and/or the standard levels of resistance and/or tolerance in a (control) population of subjects having developed a positive outcome from the disorder.

In some embodiments, the diagnostic step (a) of the therapeutic methods disclosed herein is performed by the method comprising the following steps. First in step (a), determining in at least one biological sample of the subject the expression level of at least one of: (i) at least one biomarker of said resistance, to obtain an expression value for each of the at least one biomarker/s, wherein said at least one biomarker is at least one of MXI1, ZNF395, XPC, MTHFD2, PSME2, JAK2, INTS12, PSMB7, RBM7, SLC6A8 or any combination thereof; and (ii) at least one biomarker of the tolerance, to obtain an expression value for each of the at least one biomarker/s, wherein the at least one biomarker is at least one of SERINC1, ARLI, COPS2, CRBN, MAP2K2, ARHGDIA, GRINA, STXBP2, RBM7, SLC6A8 or any combination thereof.

In the next step (b), determining if the expression values obtained in steps (a) (i) and/or (a) (ii) for each of the at least one biomarker/s is positive or negative with respect to a predetermined standard expression value or to an expression value of the biomarker/s in at least one control sample.

It should be noted that at least one of: (I) a positive expression value of at least one of the MTHFD2, PSME2, JAK2, INTS12, PSMB7 and RBM7 biomarker/s in the sample, and/or a negative expression value of at least one of said MXI1, ZNF395, XPC and SLC6A8 biomarker/s in the sample, indicate(s) that the resistance level is elevated in the subject; and/or (II) a positive expression value of at least one of the MAP2K2, ARHGDIA, GRINA, STXBP2 and SLC6A8 biomarker/s in the sample, and/or a negative expression value of at least one of the SERINCI, ARLI, COPS2, CRBN and RBM7 biomarker/s in the sample, indicate(s) that the tolerance level is elevated in the subject.

In some further embodiments, the diagnostic step (a) of the therapeutic methods disclosed herein is performed by the method comprising the following steps. First in step (a), determining in at least one biological sample of the subject the expression level of at least three biomarkers of at least one of: (i) at least one biomarker of said resistance, to obtain an expression value for each of the at least one biomarker/s, wherein said at least one biomarker is at least one of MXI1, ZNF395, XPC, MTHFD2, PSME2, JAK2, INTS12, PSMB7, RBM7, SLC6A8 or any combination thereof; and (ii) at least one biomarker of the tolerance, to obtain an expression value for each of the at least one biomarker/s, wherein the at least one biomarker is at least one of SERINCI, ARLI, COPS2, CRBN, MAP2K2, ARHGDIA, GRINA, STXBP2, RBM7, SLC6A8 or any combination thereof. It should be understood the at least three biomarkers may be in some embodiments, at least three or more of the resistance biomarkers (i), in some other embodiments, at least three or more of the tolerance biomarkers (ii), or in some other embodiments, at least three or more of the resistance (i), and the tolerance biomarkers (ii). In the next step (b), determining if the expression values obtained in steps (a) (i) and/or (a) (ii) for each of the at least three biomarkers is positive or negative with respect to a predetermined standard expression value or to an expression value of the biomarker/s in at least one control sample.

It should be noted that at least one of: (I) a positive expression value of at least one of the MTHFD2, PSME2, JAK2, INTS12, PSMB7 and RBM7 biomarker/s in the sample, and/or a negative expression value of at least one of said MXI1, ZNF395, XPC and SLC6A8 biomarker/s in the sample, indicate(s) that the resistance level is elevated in the subject; and/or (II) a positive expression value of at least one of the MAP2K2, ARHGDIA, GRINA, STXBP2 and SLC6A8 biomarker/s in the sample, and/or a negative expression value of at least one of the SERINCI, ARLI, COPS2, CRBN and RBM7 biomarker/s in the sample, indicate(s) that the tolerance level is elevated in the subject. In some embodiments, the level of expression of at least one of tolerance and/or resistance biomarkers is determined in the diagnostic step (a), as defined by the present disclosure herein above.

More specifically, in some embodiments, the step of determining the level of expression of at least one of the biomarker/s of resistance and/or at least one the biomarker/s of tolerance is performed in the therapeutic methods disclosed herein by the step of contacting at least one detecting molecule or any combination or mixture of plurality of detecting molecules with a biological sample of the subject, or with any nucleic acid or protein product obtained therefrom. It should be understood that each of the detecting molecules is specific for one of the biomarkers.

In some specific embodiments, the at least one detecting molecule used for determining the level of expression may be any one of nucleic acid-based detecting molecules and/or amino acid-based detecting molecules. More specifically, in some embodiments, nucleic acid detecting molecule/s useful in the therapeutic methods disclosed herein may comprise at least one of: (a) at least one oligonucleotide/s, each oligonucleotide specifically hybridizes to a nucleic acid sequence encoding the at least one biomarker; and/or (b) at least one nucleic acid aptamer/s specific for the at least one of the biomarkers.

In some embodiments, the biological sample analyzed by the disclosed methods may be any biological sample, for example, any body fluid sample (that may ither comprise cells or not), and/or any cell sample of the examined subject. In some specific and non-limiting embodiments, the sample used for the therapeutic methods disclosed herein, may be a blood sample. In yet some further embodiments, the sample analyzed by the disclosed methods may be a cell sample obtained from any tissue or organ of the examined subject. In some embodiments, the sample may be a skin cell sample. In yet some further embodiments, the sample may be any sample obtained by a biopsy from any tissue and/or organ. In some embodiments, the sample is a biopsy of a diseased tissue or organ. Still further in some embodiments, the sample may be a tumor biopsy.

In some embodiments, the therapeutic methods disclose herein may be applicable for any pathological disorder, for example, at least one immune related disorder.

In yet some further embodiments, the therapeutic methods may be applicable for immune related disorder such as at least one of an infectious disease caused by at least one pathogen, an inflammatory disorder, an autoimmune disorder, a proliferative disorder, a neurodegenerative disorder, a metabolic disorder and a condition involving at least one wound in at least one tissue and/or organ of the subject. In yet some particular and non-limiting embodiments, the therapeutic methods disclosed herein may be applicable for treating viral pathogen such as Influenza A virus (IAV), Ebola virus, SARS-COV2, RSV, and/or HPIV3. According to such embodiments, the subject may be treated with a compound and/or a treatment regimen that increases the resistance levels and/or reduces the tolerance levels. In yet some further embodiments, the therapeutic methods of the present disclosure may be applicable for subjects suffering from an immune related disorder such as an inflammatory or autoimmune disorder. In yet some further specific and non-limiting embodiments, such inflammatory or autoimmune disorder is any one of Systemic Lupus Erythematosus (SLE) or Rheumatoid Arthritis (RA). Still further, for a subject suffering from an inflammatory or autoimmune disorder such as SLE or RA, increasing the tolerance and/or reducing the resistance may lead to a positive outcome. Therefore, in some embodiments, the subject is administered with a compound that reduces resistance and/or increases tolerance.

In yet some further embodiments, the therapeutic methods disclosed herein may be applicable for any proliferative disorder. In some particular and non-limiting embodiments, such proliferative disorder may be a neoplastic disorder, specifically, cancer.

In some embodiments for cancers such as glioma or breast cancer, reduced susceptibility and/or positive outcome of the cancers is characterized by an elevated level of tolerance and/or reduced level of resistance. Thus, in some embodiments, a subject suffering from glioma and/or breast cancer may be administered with a compound or treatment regimen that increase tolerance and/or reduce resistance.

In yet some further embodiments, the subject treated by the methods disclosed herein is suffering from a cancer such as Leukemia (CLL). In some embodiments a reduced susceptibility and/or positive outcome of this cancer is characterized by elevated levels of tolerance. Therefore, in some embodiments, CLL patients may be treated with a compound or treatment regimen that elevates tolerance.

Still further, in some embodiments, the treated subject is suffering from a cancer such as MM. In some embodiments, at least one of: (i) the susceptibility and/or negative outcome of the cancer is characterized by an elevated level of tolerance; and (ii) the reduced susceptibility and/or positive outcome of the cancer is characterized by a reduced level of tolerance. Thus, MM subjects may be administered with a compound and/or treatment regimen that reduce tolerance.

In yet some further embodiments, the therapeutic methods disclosed herein may be applicable for treating a subject suffering from a cancer such as any one of Lung adenocarcinoma, Neuroblastoma and Astrocytoma. It should be noted that these cancers are characterized by at least one of: (i) increased susceptibility and/or negative outcome of the cancer is characterized by an elevated level of resistance; and (ii) reduced susceptibility and/or positive outcome of the cancer is characterized by a reduced level of resistance.

Thus, subjects suffering from these disorders may be administered with a compound or treatment regimen that reduces resistance.

Still further, in some embodiments of the therapeutic methods disclosed herein, the condition is at least one wound (either acute, or chronic) in at least one tissue and/or organ of the subject. It should be noted that a positive outcome of wound healing is characterized by an elevated level of tolerance. Thus, to enhance wound healing, these subjects may be treated with a compound and/or treatment regimen that elevates tolerance. It should be understood that the present disclosure refers to any acute or chronic wounds caused by any one of: a metabolic disease (e.g., diabetes ulcers), a pathogenic infectious disease (e.g., viral or bacterial), exposure to any stress, either chemical stress, temperature (burns), and any physical injury.

It should be understood that any of the disclosed disorders, diseases and pathologic condition disclosed in the present disclosure are applicable for any aspect disclosed herein.

The inventors also demonstrate the robustness of the disclosed framework through experimental validations of Arhgdia as a key regulator of the cellular T program. Arhgdia was co-expressed with program T in all cell types and under various conditions. Arhgdia is known as a regulator of Rho proteins and its activity improves survival of stem cells [Riggs, M. J., Sheridan, S. D. & Rao, R. R. Stem Cells Dev 30, 705-713 (2021).] and kidney functions [Gupta, I. R. et al.] Med Genet 50, 330-338 (2013)]. However, its more general role in control of disease tolerance has not been reported. Consistent with the disclosed predictions, the inventors identified a novel role for Arhgdia in regulation of the cellular T state. The approach presented here, particularly the specific scores (and markers) of T and R levels, is a generally applicable framework. The approach can therefore be used to identify novel regulators and therapies that specifically target the balance between disease tolerance and resistance states at the molecular level.

Thus, the present disclosure provides in an additional aspect thereof, a method for manipulating the immunological state of a subject suffering from a pathologic condition by modulating the levels of resistance and/or tolerance of the subject. In some embodiments, the method comprising administering to the subject a therapeutically effective amount of at least one of:

In some embodiments (a), at least one compound or a procedure that leads to an increase in the level of at least one of MTHFD2, PSME2, INTS12, PSMB7 and RBM7, and optionally, JAK2, biomarker/s and/or at least one compound or a procedure that leads to a decrease in the level of at least one of MXI1, ZNF395, XPC and SLC6A8 biomarker/s, and/or at least one compound or a procedure that leads to a decrease in the level of at least one of MAP2K2, ARHGDIA, GRINA and STXBP2 biomarker/s and/or at least one compound or a procedure that leads to an increase in the level of at least one of SERINC1, ARL1, COPS2 and CRBN biomarker/s, for a subject suffering from a disorder where a positive outcome is characterized with elevated resistance and/or reduced tolerance.

In yet some further embodiments (b), at least one compound or a procedure that leads to a decrease in the level of at least one of MTHFD2, PSME2, INTS12, PSMB7 and RBM7, and optionally, JAK2, biomarker/s and/or at least one compound or a procedure that leads to an increase in the level of at least one of MXI1, ZNF395, XPC and SLC6A8 biomarker/s, and/or at least one compound or a procedure that leads to an increase in the level of at least one of MAP2K2, ARHGDIA, GRINA and STXBP2 biomarker/s and/or at least one compound or a procedure that leads to a decrease in the level of at least one of SERINC1, ARLI, COPS2 and CRBN biomarker/s, for a subject suffering from a disorder where positive outcome is characterized with reduced resistance and/or elevated tolerance.

It should be understood that the various Tolerance and Resistance biomarkers of the present disclosure are used herein as targets and means for manipulating the tolerance and the resistance state of a given subject, specifically, a subject suffering from a pathologic disorder or conditions as specified by the present disclosure. It should be understood that although referring to a modulatory compound that affects the levels of the specified biomarkers, the present disclosure further encompasses the option of subjecting or exposing the subject to any procedure or treatment that leads to modulation of the levels of the discussed biomarkers. Compounds or procedures that specifically lead, either directly or indirectly to modulation of the levels of the specific biomarker, are compounds and/or procedures that affect, either directly or indirectly, the expression, the level, the stability and/or the activity of a specific biomarker as disclosed by the invention. More specifically, the term “modulating the level” thus relates to any compounds or procedures that lead to either an increase or alternatively, to a decrease in the level/s of a specific biomarker, specifically, the expression, the stability and/or the activity of a specific biomarker.

As used herein, the term “modulating the expression” includes altering or modifying gene expression by increasing or upregulating gene expression, or alternatively, by decreasing or downregulating gene expression.

More specifically, the terms “inhibition”, “moderation”, “reduction” or “attenuation” as referred to herein, relate to the retardation, restraining or reduction of the expression, levels, stability and/or activity of at least one of the biomarkers of the present disclosure by any one of about 1% to 99.9%, as will be specified herein after. Alternatively, the terms “enhancement”, “increase”, “elevation” or “enlargement” as referred to herein, relate to the enhancement, increase and elevation of the expression, levels, stability and/or activity of at least one of the biomarkers of the present disclosure by any one of about 1% to 99.9%. Specifically, 1% to 99.9% as indicated herein refers to about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. It should be appreciated that 10%, 50%, 120%, 500%, etc., are interchangeable with “fold change” values, i.e., 0.1, 0.5, 1.2, 5, etc., respectively. 10%, 50%, 120%, 500%, etc., are interchangeable with “fold change” values, i.e., 0.1, 0.5, 1.2, 5, etc., respectively. Therefore, the term inhibit, or decrease or alternatively, induce and enhance refers to an inhibition or alternatively an increase of about 2, 3, 4, 5, 6, 7, 8, 9, 10, 20, 30, 40, 50, 60, 70, 80, 90,100,200, 300, 400, 500, 600, 700, 800, 900, 1000 folds or more.

Thus, in some embodiments, where a positive outcome of the subject suffering from a pathologic disorder is characterized with, caused or enhanced by, an increased resistance and/or reduced tolerance, any compound or procedure that may lead directly or indirectly to increased resistance and/or reduced tolerance may be effectively used in the disclosed therapeutic methods. For example, any compound that may lead to an increase in the expression, the level, the stability and/or the activity of any one of MTHFD2, PSME2, INTS12, PSMB7, RBM7, and optionally, JAK2, may elevate the levels of resistance. In yet some further embodiments, compounds and/or procedures that lead either directly or indirectly to decrease in the expression, the level, the stability and/or the activity of any one of MXI1, ZNF395, XPC and SLC6A8, may elevate the levels of resistance. Such compounds or procedures that elevate the levels of resistance in a subject may be useful in pathologies and conditions such as infectious diseases caused by a pathogen, for example, viral infections. Alternatively, or additionally, in such conditions it may be also useful to reduce the levels of tolerance in a subject, for example by administering to the subject and/or exposing the subject to a procedure that leads to a decrease in the tolerance. Such procedure or compound involve any compound or procedure that leads to a decrease in the expression, the level, the stability and/or the activity of any one of MAP2K2, ARHGDIA, GRINA and STXBP2 biomarker/s, that may lead to reduction in the tolerance. In yet some further alternative or additional embodiments, compounds and/or procedures that lead either directly or indirectly to increase in the expression, the level, the stability and/or the activity of any one of SERINCI, ARLI, COPS2, RBM7 and CRBN, may reduce the levels of tolerance.

Reducing the levels of tolerance, for example by reducing the levels of one of the specifying biomarkers thereof, for example, ARHGDIA, as exemplified in Example 9, may be useful in various disorders, for example in infectious diseases caused by a pathogen. Non-limiting embodiments for such disorders include viral infections, cancers such as multiple myeloma, and as shown by Example 8, also chronic injury.

Still further, in some embodiments, elevating the levels of tolerance is desired to get a positive outcome in the treated subject. Thus, where a positive outcome of the subject suffering from a pathologic disorder is characterized with, caused or enhanced by, an increased tolerance and/or reduced resistance, any compound or procedure that may lead directly or indirectly to increased tolerance and/or reduced resistance may be effectively used in the disclosed therapeutic methods. For example, any compound that may lead to an increase in the expression, the level, the stability and/or the activity of any one of MAP2K2, ARHGDIA, GRINA and STXBP2 may elevate the levels of tolerance. In yet some further embodiments, compounds and/or procedures that lead either directly or indirectly to decrease in the expression, the level, the stability and/or the activity of any one of SERINC1, ARL1, COPS2, RBM7 and CRBN, may elevate the levels of tolerance. Such compounds or procedures that elevate the levels of tolerance in a subject may be useful in pathologies and conditions such as autoimmune disorders such as RA, SLE, in acute wounds, where wound healing is desired, in cancers such as breast cancer, glioma and CLL, as well as in healthy conditions such as pregnancy.

Alternatively, or additionally, in such conditions it may be also useful to reduce the levels of resistance in a subject, for example by administering to the subject and/or exposing the subject to a procedure that leads to a decrease in the resistance. Such procedure or compound involve any compound or procedure that leads to a decrease in the expression, the level, the stability and/or the activity of any one of MTHFD2, PSME2, INTS12, PSMB7, RBM7, and optionally, JAK2 biomarker/s, that may lead to reduction in the resistance. In yet some further alternative or additional embodiments, compounds and/or procedures that lead either directly or indirectly to increase in the expression, the level, the stability and/or the activity of any one of MXI1, ZNF395, XPC and SLC6A8, may reduce the levels of resistance.

Reducing the levels of resistance, may be useful in various disorders, for example in autoimmune disorders such as RA, SLE, in cancers such as breast cancer, glioma neuroblastoma and astrocytoma, as well as in healthy conditions such as pregnancy. Compounds that modulate the expression, stability, activity and the level of a specific biomarker as discussed herein may affect the expression, processing, cellular compartmentalization, post translational modifications and stability of any of the disclosed biomarkers. In some specific embodiments, such compounds may be nucleic acid-based compounds that affect the expression of a specific biomarker.

In some embodiments, the compound may enhance the expression of the specific biomarker, may include any vector that comprise nucleic acid sequence encoding the specific biomarker and at least one inducible and/or non-inducible regulatory elements (e.g., inducible or constitutive promotors, enhancers, repressors, or post translationally regulatory elements such as for example degrons). In yet some further embodiments when reducing the levels of a specific biomarker is desired, the modulatory compound used herein may be a nucleic acid-based molecule.

In some specific embodiments, such modulatory compound that reduces the levels of at least one of the disclosed biomarkers, may be or may comprise a nucleic acid molecule for example, a ribonucleic acid (RNA) molecule or any nucleic acid sequence encoding said RNA molecule. In more specific embodiments, such RNA molecule may be at least one of a double-stranded RNA (dsRNA), an antisense RNA, a single-stranded RNA (ssRNA), gRNAs and a Ribozyme.

Thus, in some embodiments, the modulatory compound that reduces the levels of at least one of the disclosed biomarkers may be nucleic acid molecules that may comprise at least one of a small interfering RNA (siRNA), a short hairpin RNA (shRNA), microRNA (miRNA), antisense oligonucleotide (ASO), locked nucleic acid (LNA), as well as other nucleic acids derivatives.

In some embodiments, the modulatory compound that reduces the levels of at least one of the disclosed biomarkers may be dsRNA molecules participating in RNA interference. More specifically, the dsRNA encompassed by the invention may be selected from the group consisting of small interfering RNA (siRNA), MicroRNA (miRNA), short hairpin RNA (shRNA), PIWI interacting RNAs (piRNAs). RNA interference (RNAi) is a general conserved eukaryotic pathway which down regulates gene expression in a sequence specific manner. It is the process of sequence-specific, post-transcriptional gene silencing in animals and plants, initiated by siRNA that is homologous in its duplex region to the sequence of the silenced gene. Gene silencing is induced and maintained by the formation of partly or perfectly double-stranded RNA (dsRNA) between the target RNA and the siRNA/shRNA derived ‘guide” RNA strand. The expression of the gene is either completely or partially inhibited. As known in the art RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs/RNAs containing a sequence complementary to one of the siRNA strands. One of the strands of the double-stranded siRNA molecule (the “guide” strand) comprises a nucleotide sequence that is complementary to a nucleotide sequence of the target gene, or a portion thereof, and the second strand of the double-stranded siRNA molecule (the passenger” strand) comprises a nucleotide sequence substantially similar to the nucleotide sequence of the target gene, or a portion thereof. After binding to RISC, the guide strand is directed to the target mRNA cleaved between bases 10 and 11 relative to the 5′ end of the siRNA guide strand by the cleavage enzyme Argonaute-2 (AGO2). Thus, the process of mRNA translation can be interrupted by siRNA.

In yet some further embodiments, the modulatory compound that reduces the levels of at least one of the disclosed biomarkers used by the methods of the present disclosure, may be based on any gene editing system, specifically programmable system, that is specifically directed against nucleic acid sequences comprised within the nucleic acid sequence encoding at least one of the disclosed biomarkers. According to such embodiments, the modulatory compound that reduces the levels of at least one of the disclosed biomarkers, may comprise at least one nucleic acid sequence that targets a modifier protein, for example, a nuclease or any fusion proteins thereof, to a target sequence within the nucleic acid sequence encoding at least one of the disclosed biomarkers. Targeting the nucleic acid modifier to a specific target sequence, by a targeting molecule (such as a specific guide RNA), leads to specific binding to the target sequence and targeted manipulation (e.g., cleavage or any other modification), that leads to reduction in the expression, stability and/or activity of at least one of the disclosed biomarkers. Still further, in some embodiments the modulatory compound that reduces the levels of at least one of the disclosed biomarkers is at least one guide RNA that guides at least one programmable engineered nucleases (PEN) to the target nucleic acid sequence as specified herein. In some embodiments, the PEN comprises at least one clustered regulatory interspaced short palindromic repeat (CRISPR)/CRISPR associated (cas) protein. Thus, according to some embodiments, modulatory compound that reduces the levels of at least one of the disclosed biomarkers comprises: first (a), at least one nucleic acid sequence comprising at least one gRNA, or any nucleic acid sequence encoding the gRNA; or any kit, composition, vector or vehicle comprising the gRNA or nucleic acid sequence encoding the gRNA. Optionally, modulatory compound that reduces the levels of at least one of the disclosed biomarkers may further comprise (b), at least one CRISPR/cas protein, or any nucleic acid molecule encoding the Cas protein, or any kit, composition, vector or vehicle comprising the CRISPR/cas protein or nucleic acid sequence encoding the CRISPR/cas protein, or any nucleic acid sequence encoding the gRNA; or any kit, composition or vehicle comprising at least one of (a) and (b).

Thus, in some embodiments, the Cas protein and the specific gRNA may be provided to and/or contacted with the target cell, or administered to the treated subject, either as a protein and gRNA, or alternatively, as nucleic acid sequences encoding these two elements, either in two separate nucleic acid molecules (e.g., two separate constructs), or in one nucleic acid molecule.

The term “programmable engineered nucleases (PEN)” as used herein also known as “molecular DNA scissors”, refers to enzymes either synthetic or natural, and used to replace, eliminate or modify target sequences in a highly targeted way. PEN target and cut specific genomic sequences (recognition sequences) such as DNA sequences. The at least one PEN may be derived from natural occurring nucleases or may be an artificial enzyme, all involved in DNA repair of double strand DNA lesions and enabling direct genome editing. In some alternative or additional embodiments the modulatory compound that reduces the levels of at least one of the disclosed biomarkers according with the present disclosure encompasses also any nucleic acid molecule comprising at least one nucleic acid sequence encoding the PEN or any kit, composition or vehicle comprising the at least one PEN, or any nucleic acid sequence encoding the PEN.

In yet some further specific embodiments, such nucleases may include RNA guided nucleases such as CRISPR-Cas. However, it should be understood that in some alternative embodiments, other nucleases such as ZFN, TALEN, Homing endonuclease, Meganuclease, Mega-TALEN may be used by the methods of the invention for targeting at least one target nucleic acid sequence comprised within the nucleic acid sequence that encodes at least one of the disclosed biomarkers.

More specifically, in some embodiments, the at least one PEN may be at least one of a mega nuclease, a zinc finger nuclease (ZFN), a transcription activator-like effector-based nuclease (TALEN), or a clustered regularly interspaced short palindromic repeats (CRISPR/Cas) system.

In some embodiments, the at least one PEN may be a mega nuclease. Mega nucleases are endodeoxyribonucleases characterized by a large recognition site (double-stranded DNA sequences of 12 to 40 base pairs); such that this site generally occurs only once in any given genome. Meganucleases are specific naturally occurring restriction enzymes and include among others, the LAGLIDADG family of homing endonucleases, mostly found in the mitochondria and chloroplasts of eukaryotic unicellular organisms.

In some embodiments, the at least one PEN may be a megaTAL. MegaTALs are fusion proteins that combine homing endonucleases, such as LAGLIDADG family, with the modular DNA binding domains of TALENs.

In some alternative embodiments, the at least one PEN may be a zinc finger nuclease (ZFN). ZFNs are artificial restriction enzymes generated by fusing a zinc finger DNA-binding domain to a DNA-cleavage domain. Zinc finger domains can be engineered to target specific desired DNA sequences, enabling ZFN to target the target sequences within the target transcripts of the biomarkers specified by the invention, thereby inhibiting the expression, activity and/or stability of at least one of the disclosed biomarkers.

In yet some other embodiments, the at least one PEN may be a transcription activator-like effector-based nuclease (TALEN). TALEN are restriction enzymes that can be engineered to cut specific sequences of DNA. TALEN are made by fusing a TAL effector DNA-binding domain to a DNA cleavage domain (a nuclease which cuts DNA strands).

In some specific embodiments, the targeting of the target nucleic acid sequence that is comprised within the nucleic acid sequence that encodes at least one of the disclosed biomarkers, may be mediated by a PEN that may comprise at least one clustered regulatory interspaced short palindromic repeat (CRISPR)/CRISPR associated (cas) protein system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI. Thus, in some embodiments, the Cas protein may be a member of at least one of CRISPR-associated system of Class 1 and Class 2. In some embodiments, the cas protein may be a member of at least one of CRISPR-associated system of any one of type II, type I, type III, type IV, type V and type VI from E. coli, Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly any of the CRISPR systems disclosed herein. The CRISPR-Cas system, targets DNA molecules based on short homologous DNA sequences, called spacers that exist between repeats. These spacers guide CRISPR-associated (Cas) proteins to matching sequences within the target DNA, called proto-spacers, which are subsequently cleaved. The spacers can be rationally designed to form guide RNAs (gRNAs) that target any target DNA sequence, for example, the target sequence within the nucleic acid sequence that encodes at least one of the disclosed biomarkers. It should be noted that the modulatory compound that reduces the levels of at least one of the disclosed biomarkers may comprise in some embodiments at least one gRNA targeted against at least one nucleic acid target that is comprised within at least one nucleic acid sequence that encodes at least one of the disclosed biomarkers. Alternatively, the modulatory compound that reduces the levels of at least one of the disclosed biomarkers may comprise any nucleic acid sequence encoding such gRNA.

In some specific embodiment, the RNA guided DNA binding protein nuclease used by the invention may be a CRISPR Class 2 system. In yet some further particular embodiments, such class 2 system may be a CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage. It should be appreciated that any type II CRISPR-Cas systems may be applicable in the present invention, specifically, any one of type II-A. typeII-B or typeII-C. In more particular embodiments, at least one cas protein of type II CRISPR system used by the invention may be the cas9 protein, or any fragments, mutants, fusion proteins, variants or derivatives thereof (e.g., Cas9/Cpf1/CTc (1/2/3), SpCas9, SaCas9, engineered Cas9, and any mutants or fusion proteins thereof, for example, dCas9-Fok1, and the like). The CRISPR-associated protein Cas9 is an RNA-guided DNA endonuclease that uses RNA: DNA complementarity to a target site (proto-spacer). After recognition between Cas9 and the target sequence double stranded

DNA (dsDNA) cleavage occur, creating the double strand brakes (DSBs). Still further, CRISPR type

II system as used herein requires the inclusion of two essential components: a “guide” RNA (gRNA), that is comprised within the modulatory compound that reduces the levels of at least one of the disclosed biomarkers disclosed and used by the methods of the present disclosure, and a non-specific CRISPR-associated endonuclease (Cas9). Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”. In some embodiments, the gRNA of the invention may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers, or gRNA may comprise between about 20-35 nucleotides.

Non-limiting embodiments for such modulatory compound that reduces the levels of at least one of the disclosed biomarkers, may be the sgRNA used for inhibiting the expression of ARHGDIA. In some embodiments, the disclosed modulatory compound that reduces the levels of at least one of the disclosed biomarkers, may be at least one sgRNA directed against at least one protospacer in the ARHGDIA encoding sequence. In some particular embodiments such sgRNA may comprise the nucleic acid sequence as denoted by SEQ ID NO: 37, or the nucleic acid sequence as denoted by SEQ ID NO: 38, or any variants and homologs thereof. It should be noted that in some embodiments, sgRNA directed against any target sequence within the ARHGDIA encoding sequence may be useful in the present disclosure. In some embodiments, sgRNA directed against any sequence comprised within the ARHGDIA encoding sequence as denoted by SEQ ID NO: 32, or any homologs, variants and orthologs thereof. Thus, in some embodiments, the methods of the present disclosure may comprise the step of administering to a subject suffering from a viral infection, specifically, infection by IAV, at least one compound that leads directly or indirectly to reduction of the levels of ARHGDIA. According to some embodiments, administration of such compound lead to reduction of the expression of ARHGDIA, and thus to reduction of the tolerance in the treated subject. This reduction leads to a positive outcome of the subject that is further reflected in some embodiments in reduced viral load.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein. The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention.

As indicated above, the methods and compositions provided by the present invention may be used for the treatment of a “pathological disorder”, specifically, immune-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

A further aspect of the present disclosure relates to a screening method for identifying (and or evaluating) at least one therapeutic compound for the treatment of a pathologic disorder. It should be noted that a selected compound modifies the level of resistance and/or tolerance in at least one subject suffering from the pathologic disorder. In some embodiments, the method comprising the steps of: First (a), determining the levels of resistance and/or tolerance of at least one biological sample contacted with the candidate compound. The sample is of a subject suffering from the specific pathologic disorder. The next step (b), involves determining that the candidate compound is a therapeutic compound for the disorder if: (i) the candidate compound elevates resistance and/or reduces tolerance, in a sample of the subject contacted with the candidate compound, as compared to a control sample, in case the subject is suffering from a disorder where a positive outcome is characterized with elevated resistance and/or reduced tolerance; and (ii) the compound reduces resistance and/or elevates tolerance, in a sample of the subject contacted with the candidate compound, as compared to a control sample, in case subject is suffering from a disorder where positive outcome is characterized with reduced resistance and/or elevated tolerance.

Still further, in some embodiments, step (a) is performed by the method comprising the steps of: First in step (a), determining in at least one biological sample of the subject the expression level of at least one of: (i) at least one biomarker of resistance, to obtain an expression value for each of the at least one biomarker/s. It should be noted that the at least one biomarker is at least one of MXI1,
ZNF395, XPC, MTHFD2, PSME2, JAK2, INTS12, PSMB7, RBM7, SLC6A8 or any combination thereof; and (ii) at least one biomarker of said tolerance, to obtain an expression value for each of the at least one biomarker/s. The at least one biomarker is at least one of SERINC1, ARLI, COPS2, CRBN, MAP2K2, ARHGDIA, GRINA, STXBP2, RBM7, SLC6A8 or any combination thereof.

The next step (b), involves determining if the expression values obtained in steps (a) (i) and/or (a) (ii) for each of the at least one biomarker/s is positive or negative with respect to a predetermined standard expression value or to an expression value of the biomarker/s in at least one control sample.

It should be noted that at least one of:
(I) a positive expression value of at least one of the MTHFD2, PSME2, JAK2, INTS12, PSMB7 and RBM7 biomarker/s in the sample, and/or a negative expression value of at least one of the MXI1, ZNF395, XPC and SLC6A8 biomarker/s in the sample, indicate(s) that the resistance level is elevated in the subject; and/or
(II) a positive expression value of at least one of the MAP2K2, ARHGDIA, GRINA, STXBP2 and SLC6A8 biomarker/s in the sample, and/or a negative expression value of at least one of the SERINC1, ARL1, COPS2, CRBN and RBM7 biomarker/s in the sample, indicate(s) that the tolerance level is elevated in the subject.

In yet some further embodiments, step (a) is performed by the method comprising the steps of: First in step (a), determining in at least one biological sample of the subject the expression level of at least three biomarkers of at least one of: (i) at least one biomarker of resistance, to obtain an expression value for each of the at least one biomarker/s. It should be noted that the at least one biomarker is at least one of MXI1, ZNF395, XPC, MTHFD2, PSME2, JAK2, INTS12, PSMB7, RBM7, SLC6A8 or any combination thereof; and (ii) at least one biomarker of said tolerance, to obtain an expression value for each of the at least one biomarker/s. The at least one biomarker is at least one of SERINC1, ARLI, COPS2, CRBN, MAP2K2, ARHGDIA, GRINA, STXBP2, RBM7, SLC6A8 or any combination thereof. It should be understood the at least three biomarkers may be in some embodiments, at least three or more of the resistance biomarkers (i), in some other embodiments, at least three or more of the tolerance biomarkers (ii), or in some other embodiments, at least three or more of the resistance (i), and the tolerance biomarkers (ii).

The next step (b), involves determining if the expression values obtained in steps (a) (i) and/or (a) (ii) for each of the at least three biomarkers is positive or negative with respect to a predetermined standard expression value or to an expression value of the biomarker/s in at least one control sample. It should be noted that at least one of:
(I) a positive expression value of at least one of the MTHFD2, PSME2, JAK2, INTS12, PSMB7 and RBM7 biomarker/s in the sample, and/or a negative expression value of at least one of the MXI1, ZNF395, XPC and SLC6A8 biomarker/s in the sample, indicate(s) that the resistance level is elevated in the subject; and/or
(II) a positive expression value of at least one of the MAP2K2, ARHGDIA, GRINA, STXBP2 and SLC6A8 biomarker/s in the sample, and/or a negative expression value of at least one of the SERINC1, ARL1, COPS2, CRBN and RBM7 biomarker/s in the sample, indicate(s) that the tolerance level is elevated in the subject.

In some embodiments, the candidate molecule is a therapeutic agent/drug. More specifically, a compound to be tested by the disclosed screening methods may be referred to as a test compound or a candidate compound. The candidate compounds may be any known used for a specific disorder, or any unknown drug or compound that is screened herein based on its effect on the T and/or R levels, and thus, as a candidate compound that may modulate the immunological state of a given subject. Any compound may be used as a test compound in various embodiments. In some embodiments a library of FDA approved compounds that can be used by humans may be used. Compound libraries are commercially available from a number of companies including but not limited to Maybridge Chemical Co. (Trevillet, Cornwall, UK), Comgenex (Princeton, NJ), Microsource (New Milford, CT), Aldrich (Milwaukee, WI), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, CA), ChemDiv, (San Diego, CA), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, NJ), Interbio screen (Moscow, Russia), Interchim (Montlucon, France), Life Chemicals, Inc. (Orange, CT), Microchemistry Ltd. (Moscow, Russia), Otava, (Toronto, ON), PharmEx Ltd. (Moscow, Russia), Princeton Biomolecular (Monmouth Junction, NJ), Scientific Exchange (Center Ossipee, NH), Specs (Delft, Netherlands), TimTec (Newark, DE), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds. It will be understood that the agents to be screened could also be derived or synthesized from chemical compositions or man-made compounds. In some embodiments a library useful in the present invention may comprise at least 10,000 compounds, at least 50,000 compounds, at least 100,000 compounds, at least 250,000 compounds, or more.

In yet some further embodiments, the candidate compound may be at least one of a small molecule, aptamer, a peptide, a nucleic acid molecule and an immunological agent, and any combinations thereof. In some specific embodiments, the compound used by the screening methods of the present disclosure, that specifically modulate the T and/or R levels in a subject, may be a small molecule. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the candidate therapeutic compound is a known drug used for the treatment of said particular disorder. In such case, the method disclosed herein is used to evaluate if the particular drug is suitable and/or optimal for treating the particular disorder in the specific subject, thereby providing a personalized therapeutic tool.

Still further, the methods disclosed herein may provide screening of compounds suitable for a group of patients suffering from a disease characterized by a particular resistance and tolerance state. In such case, the candidate compounds are screened using cells of various subjects suffering from the same disease. Alternatively, the screening method may be used to screen a particular therapeutic agent for a particular patient.

A further aspect of the present disclosure relates to a diagnostic composition comprising at least one detecting molecule or any combination or mixture of plurality of detecting molecules specific for determining the level of expression of at least one of: (i) at least one biomarker of resistance, such at least one biomarker is at least one of MXII, ZNF395, XPC, MTHFD2, PSME2, JAK2, INTS12, PSMB7, RBM7, SLC6A8 or any combination thereof; and (ii) at least one biomarker of tolerance, such at least one biomarker is at least one of SERINCI, ARLI, COPS2, CRBN, MAP2K2, ARHGDIA, GRINA, STXBP2, RBM7, SLC6A8 or any combination thereof. It should be noted that each of the detecting molecules is specific for one of the biomarker/s.

A further aspect of the present disclosure relates to a diagnostic composition comprising at least three detecting molecules or any combination or mixture of plurality of detecting molecules specific for determining the level of expression of at least three biomarkers of at least one of: (i) at least one biomarker of resistance, such at least one biomarker is at least one of MXII, ZNF395, XPC, MTHFD2, PSME2, JAK2, INTS12, PSMB7, RBM7, SLC6A8 or any combination thereof; and (ii) at least one biomarker of tolerance, such at least one biomarker is at least one of SERINC1, ARLI, COPS2, CRBN, MAP2K2, ARHGDIA, GRINA, STXBP2, RBM7, SLC6A8 or any combination thereof. It should be noted that each of the detecting molecules is specific for one of the biomarker/s. The disclosed composition comprises any of the detecting molecules disclosed by the present disclosure and any combinations thereof, as specified in connection with other aspects of the invention.

A further aspect of the present disclosure relates to a kit comprising:

(a) at least one detecting molecule specific for determining the level of expression of at least one of: (i) at least one biomarker of resistance, the at least one biomarker is at least one of MXI1, ZNF395, XPC, MTHFD2, PSME2, JAK2, INTS12, PSMB7, RBM7, SLC6A8 or any combination thereof; and
(ii) at least one biomarker of tolerance, the at least one biomarker is at least one of SERINC1, ARL1, COPS2, CRBN, MAP2K2, ARHGDIA, GRINA, STXBP2, RBM7, SLC6A8 or any combination thereof in a biological sample. It should be noted that each of the detecting molecule/s is specific for one of the biomarkers. In some embodiment the kit may optionally further comprises at least one of:

(b) pre-determined calibration curve/s or predetermined standard/s providing standard expression values of said at least one biomarker/s; and (c) at least one control sample.

In some specific embodiments, the present disclosure comprises a kit comprising: (a) at least three detecting molecules specific for determining the level of expression of at least three biomarkers of at least one of:
(i) at least one biomarker of resistance, the at least one biomarker is at least one of MXI1, ZNF395, XPC, MTHFD2, PSME2, JAK2, INTS12, PSMB7, RBM7, SLC6A8 or any combination thereof; and
(ii) at least one biomarker of tolerance, the at least one biomarker is at least one of SERINC1, ARLI, COPS2, CRBN, MAP2K2, ARHGDIA, GRINA, STXBP2, RBM7, SLC6A8 or any combination thereof in a biological sample. It should be noted that each of the detecting molecule/s is specific for one of the biomarkers. In some embodiment the kit may optionally further comprises at least one of: (b) pre-determined calibration curve/s or predetermined standard/s providing standard expression values of said at least three biomarker/s; and (c) at least one control sample. It should be understood that any of the detecting molecules disclosed by the present disclosure and any combinations thereof, as specified in connection with other aspects of the invention, are also applicable in the present aspect. Still further, the kit and/or compositions disclosed herein may comprise at least one detecting molecule specific for each of the at least 1, 2, 3, 4, 5, 6, 7, 8, 9, 10, 11, 12, 13, 14, 15, 16, 17, or 18 biomarkers disclosed herein. Each of the detecting molecules is specific for one of the biomarkers, however, a plurality of detection molecules can be used for each of the biomarkers, specifically, at least 1, 2, 3, 4, 5, 6, 7, 8, 9., 10, 15, 20, 25, 30, 40, 50, 60, 70, 80, 90, 100 or more. Still further, the inventors consider the kit of the invention in compartmental form. It should be therefore noted that in certain embodiments the detecting molecules used for detecting the expression levels of the biomarkers may be provided in a kit attached to an array. As defined herein, a “detecting molecule array” refers to a plurality of detection molecules that may be nucleic acids based or protein based detecting molecules, optionally attached to a support where each of the detecting molecules is attached to a support in a unique pre-selected and defined region.

For example, an array may contain different detecting molecules, such as specific antibodies, labeled or tagged proteins, peptides, aptamers, probes and/or primers or any combinations thereof. As indicated herein before, in case a combined detection of the biomarker expression level, the different detecting molecules for each target may be spatially arranged in a predetermined and separated location in an array. For example, an array may be a plurality of vessels (test tubes), plates, micro-wells in a micro-plate, each containing different detecting molecules, specifically, aptamers, primers and antibodies, specific for each marker protein used by the invention. An array may also be any solid support holding in distinct regions (dots, lines, columns) different and known, predetermined detecting molecules.

As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin™ or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above.

In certain embodiments, the detecting molecule/s used in the diagnostic compositions and kits of the invention may be provided in a mixture. In some alternative embodiments, the detecting molecules may be provided as molecules that are not attached to any solid support. In some embodiments, the non-attached detecting molecules may be provided in separate containers, wells, tube vessels and the like. In some alternative embodiments, the attached or non-attached detecting molecules may be provided in a mixture that contains at least two detecting molecules specific for at least two biomarker/s of the invention.

It should be appreciated that the components in the kit may depend on the method of detection and are not limited to any method. Some embodiments of the present disclosure concern a kit that further comprises at least one reagent for conducting a nucleic acid amplification-based assay, for example, a Real-Time PCR, micro arrays, PCR, in situ Hybridization and Comparative Genomic Hybridization.

According to some specific embodiments, the kit of the invention may be specifically suitable for determining the T and the R levels, and thereby the immunological state of a subject, for example a subject suffering from a pathologic disorder.

In some embodiments, the polynucleotide-based detection molecules used by the disclosed methods, compositions and kits may be in the form of nucleic acid probes which can be spotted onto an array to measure RNA from the sample of a subject to be diagnosed. As defined herein, a “nucleic acid array” refers to a plurality of nucleic acids (or “nucleic acid members”), optionally attached to a support where each of the nucleic acid members is attached to a support in a unique pre-selected and defined region. These nucleic acid sequences are used herein as detecting nucleic acid molecules. In one embodiment, the nucleic acid member attached to the surface of the support is DNA. In a preferred embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, a “nucleic acid array” refers to a plurality of unique nucleic acid detecting molecules attached to nitrocellulose or other membranes used in Southern and/or Northern blotting techniques. For oligonucleotide-based arrays, the selection of oligonucleotides corresponding to the gene of interest which are useful as probes is well understood in the art.

As indicated above, assay based on micro array or RT-PCR may involve attaching or spotting of the probes in a solid support. As used herein, the terms “attaching” and “spotting” refer to a process of depositing a nucleic acid onto a substrate to form a nucleic acid array such that the nucleic acid is stably bound to the substrate via covalent bonds, hydrogen bonds or ionic interactions.

As used herein, “stably associated” or “stably bound” refers to a nucleic acid that is stably bound to a solid substrate to form an array via covalent bonds, hydrogen bonds or ionic interactions such that the nucleic acid retains its unique pre-selected position relative to all other nucleic acids that are stably associated with an array, or to all other pre-selected regions on the solid substrate under conditions in which an array is typically analyzed (i.e., during one or more steps of hybridization, washes, and/or scanning, etc.).

In some embodiments, the kit of the invention further comprising at least one reagent for conducting an immunological assay selected from protein microarray analysis, ELISA, RIA, slot blot, dot blot, FACS, western blot, immunohistochemical assay, immunofluorescent assay and a radio-imaging assay. Accordingly, such kit may comprise antibodies, labeling material, in some embodiments reagents substrates and enzymes required to perform colorimetric or electrochemical reaction, optionally, secondary antibodies, filters, beads and any required solid support. In some embodiments, the kit of the invention may further comprise at least one reagent for conducting a mass spectrometry assay. Such reagents may include trypsin, buffers, filters and the like, for peptide purification. In further embodiments, the kit of disclosed herein may further comprise at least one device, means or any reagent for obtaining a biological sample, from a subject, for example any cell, tissue or body fluid sample (needles, aspirators and the like).

As indicated in Example 14, determination of tolerance and resistance and thereby evaluating the immunological state of a particular subject may further provide a tool for improving diagnostic biomarkers and providing stronger diagnostic tools to distinguish a specific group of subjects affected by a specific pathologic disorder (or characterized by a particular physiological process), from a control group of subjects, in a manner that is independent of various covariant parameters. The present disclosure thus relates to a method for selecting improved biomarker/s for a pathologic disorder. The method comprising the step of (a) providing at least one candidate biomarker that display a differential expression in a specific group of subjects affected by the pathologic disorder, as compared to a control group of subject (a biomarker that display a high ‘association score’); and (b) selecting a biomarker that distinguish between both groups independently of at least one of the following covariant parameters: (i) resistance and/or tolerance level/s (b) age: (c) gender; (d) ethnic origin; (e) BMI; (f) smoking (display a low ‘association score’ with said at least one covariant/s); thereby selecting an improved biomarker for the pathologic disorder. The framework disclosed herein can be applied to prioritize particular disease-associated factors in which the association is independent of variation in the resistance and tolerance states. Such prioritization strategy has potential to guide development of effective clinical diagnostics and selection of drug targets.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to +10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental procedures

### Mice

The CC cohort includes female mice aged 7-10 weeks from the Tel Aviv University (TAU) collection of Collaborative Cross recombinant inbred mice [Welsh, C. E. et al. Mamm Genome 23, 706-712 (2012)], as well as the C57BL/6J strain from Envigo, Israel. The mice were raised at the Animal Facility at the Sackler Faculty of Medicine of TAU. All experimental procedures were approved by the Institutional Animal Care and Use Committee (IACUC) of TAU (approval number 04-14-049) and adhere to the Israeli guidelines and the US NIH animal care and use protocols. Mice were held in individually ventilated cages and housed on hardwood chip bedding under a 12h light/dark cycle, humidity-controlled and temperature-controlled conditions. Mice were given tap water and standard rodent chow diet ad libitum from their weaning day until the end of the experiment.

In vivo IAV infection

Mouse-adapted H1N1 influenza A/PR/8/34 virus was grown in allantoic fluid of 10-day-old embryonated chicken eggs at 37° C. for 72 h. Allantoic fluid was harvested and viral titers were determined by standard plaque assay in Madin-Darby canine kidney (MDCK). All mice were first anesthetized (intraperitoneally) with 7 mg ml-1 ketamine and 1.4 mg ml-1 xylazine at 0.1 ml per 10g body weight. Next, animals were infected intranasally with PR8 (4.8×10³ pfu in 40 μl PBS). The data consist of one mouse from each strain and each time point. Over the time course for 33 CC strains, data were missing for only a few individuals. For additional six strains, only one or two individuals were evaluated at late time points of infection.

Measurement of organismal phenotypes

Whole-body weight and lungs' functions were measured daily. Respiratory parameters were measured using an unrestrained whole-body plethysmography system (DSI, MN, USA). A non-anesthetized mouse was placed in a plethysmography chamber and acclimated for 20 min. Data were then collected for an additional 5 min, and average values were used for the enhanced pause (Penh) parameter to assess airway dysfunction as previously described (Menachery, V. D., et al (2015; PLOS One 10, e0131451-e0131451). Over the time course for 33 CC strains, data were missing for only a few individuals. For additional six strains, only one or two individuals were evaluated at late time points of infection.

Measurement of gene expression

Lung tissues were collected into RNAlater (Qiagen). Lysis was performed with QIAzol (Qiagen). RNA isolation was performed according to miRNeasy kit protocol (Qiagen). mRNA quality was checked using the Agilent 2100 Bioanalyzer according to the manufacturer's instructions. All RNA Integrity Numbers (RINs) were higher than 8. cDNA libraries were prepared from 2 μg of isolated RNA using the SENSE mRNA-Seq Library Prep Kit V2 for Illumina (Lexogen). Each sample had its own index primer. DNA size and quality were checked using the Agilent 2100 Bioanalyzer. Libraries were quantified using the Qubit DNA HS Assay kit (Invitrogen). The amplified libraries were pooled at a total concentration of 2 nM and sequenced using the Illumina HiSeq platform at the Technion Genome Center (Israel). The entire dataset is deposited in the GEO database (GSE174253).

Quantification and statistical analysis

Pre-processing of gene expression. A joint alignment of sequencing reads was applied for both the mouse genome and the influenza virus genome using the Bowtie2 software and then applied a joint quantification of both the virus and mouse transcripts. For this quantification, an FPKM-normalization was applied using the RSEM software, and then In-transformed the data. Additional standardization of expression levels (centered and divided by standard deviation) was applied based on the data collected before infection. Unless stated otherwise, the ‘expression levels’ refer to these standardized log-transformed gene expression levels.

Disease severity phenotypes. Disease severity was quantified by analyses of viral burden, weight loss, breathing dysfunction, Ifnbl and Ccl2 expression, the quantity of immune cells, and tissue damage. The ‘viral burden’ phenotype was defined as the averaged expression level of viral transcripts NC_002016, NC_002017, NC_002018, NC_002019, NC_002020, NC_002021, NC_002022, and NC_002023; values were In-transformed but were not standardized. ‘Weight loss’ is reported as the percentage of reduction in the whole-body weight compared to the weight immediately prior to infection. ‘Breathing dysfunction’ is defined by the Penh metric (Menachery, et al. (2015). PLOS One 10, e0131451-e0131451). The expression levels of Ifnbl and Ccl2 were obtained from the transcriptome data (In-transformed, with standardization).

The mean expression levels of CD45+-specific genes were used as a signature for the quantity of immune cells, and the negation of mean expression levels of genes specific to CD45-cells was used as a signature for tissue damage. To define the sets of CD45+−specific and CD45--specific genes, the inventors first used Seuret v3 (Stuart, T., et al. (2019). Cell 177, 1888-1902.e21) to annotate each single cell from the Tabula Muris dataset (Schaum, N., et al. (2018). Nature 562, 367-372), and then selected lung cells that did or did not express CD45. The CD45+−specific genes were defined as the genes at the top 25% of average expression in the CD45+ cells and at the bottom 25% of average expression across the CD45-cells. Similarly, the CD45--specific genes were defined as a set of genes at the top 25% of average expression across the CD45-cells and at the bottom 25% of average expression across the CD45+ cells.

Additionally, as an independent signature for tissue damage, the inventors used, first, the negation of the average of cilium genes (termed a ‘signature of ciliary damage’). Second, the inventors used a signature of ‘cytokine storm’, which was the average expression of the set of cytokines typically dysregulated during cytokine storm syndrome (Tisoncik, J. R., et al. (2012). Microbiol Mol Biol Rev 76, 16-32). Third, the ‘relative tissue damage’ of each CC strain was calculated as the slope of a regression line in which the explained variable is the tissue damage, and the explaining variable is the viral burden [7]. Fourth, the Mx1 genetics of each strain was determined as previously described (Ferris, M.T., et al. (2013). PLOS Pathog 9, e1003196-e1003196). Finally, to calculate the heritability (inherited variation) measure for each phenotype, each phenotype was quantified in two or more individuals from each CC strain at the same time point.

Assessment of inherited variation. To calculate the heritability measure (variation due to genetic differences) for each phenotype, we quantified two mice per strain on day 4 post IAV infection (12 CC strains). Measurements of gene expression were collected and pre-processes as previously described [Altboum, Z. et al. Mol Syst Biol 10, 720-720 (2014)]. As the main longitudinal CC dataset was generated using a different technology (see above), this limited 4-days dataset was only used for the assessment of inherited variation. This dataset is deposited in the GEO database (GSE184278). A p-value of heritability was calculated using comparison to a null distribution of the heritability score, which was calculated for each phenotype using permutation of individuals between strains.

Assessment of the ‘relative tissue damage’

The ‘relative tissue damage’ phenotype is generally defined as the level of disease severity relative to the pathogen burden [Raberg, L., Graham, A. L. & Read, A. F. Philos Trans R Soc Lond B Biol Sci 364, 37-49 (2009)]. At the broad sense, this phenotype is calculated based on the entire physiological path in the health space [Ayres, J. S. Cell 181, 250-269 (2020)]. However, most datasets do not contain the entire course of infection but only one or a few time points (e.g., in our case, the data includes the incubation and acute phases but not the outcome phase [, a limitation imposed by ethics considerations) and therefore it is impossible to analyze the entire health continuum.

An alternative approach, commonly used in the relevant literature, relies on reaction norms [Medzhitov, R., Schneider, D. S. & Soares, M. P. Science 335, 936-941 (2012); Ayres, J. S. Cell 181, 250-269 (2020); Råberg, L., Graham, A. L. & Read, A. F. Philos Trans R Soc Lond B Biol Sci 364, 37-49 (2009). Reaction norms plot the level of disease severity for an individual at each pathogen burden. The “relative tissue damage” is defined as the slope of the severity-to-pathogen regression in this plot, such that a shallower slope indicates a better ability to tolerate the pathogen. The analysis of relative tissue damage compares the slope between groups of individuals. To calculate the slope for a given group, the group must contain a wide spectrum of pathogen burden and disease-severity levels. To address this, previous studies commonly used multiple genetically distinct individuals in the same time point [Råberg Lars, Sim Derek, & Read Andrew F. Science 318, 812-814 (2007)], or alternatively, multiple individuals from the same strain in several time points [Sanchez, K. K. et al. Cell 175, 146-158.e15 (2018); Sorci, G., Léchenault-Bergerot, C. & Faivre, B. Infection, Genetics and Evolution 88, 104698 (2021)] (it is also common to use multiple individuals from the same strain and the same time point, but only when the pathogen burden is constant [Talmi-Frank, D. et al. Cell Host & Microbe 20, 458-470 (2016)]).

Here, the ‘relative tissue damage phenotype’ was calculated by the slope of each CC strain using a group of multiple infected individuals from the same strain. The viral mRNA level was used as the measure of pathogen burden and used the tissue-damage signature as the measure of disease severity. The tissue damage signature relies on either CD45-cells or ciliated cells.

Construction and analysis of programs R and T

The inventors aimed at constructing a model representing the co-regulation of genes during the course of IAV infection. The analysis consists of two steps. First, construction of a map, which allowed us to define the R and T programs, and second, calculation of tissue-immune states based on programs R and T.

Construction of a map. The inventors' goals were twofold: first, to integrate multiple time points and strains into one representative model, and second, to construct a model relevant to the severity of IAV infection. The map was constructed in three steps. In step 1, the inventors selected genes that have high relevance to disease severity and that provide a uniform coverage of behaviors across time points and strains as follows: (i) the Pearson's correlation was calculated between the weight loss phenotype at 96h p.i. and the expression of each gene across mouse samples (with a separate calculation at each time point). The output is a matrix of correlations for each gene at each time point. (ii) The inventors selected genes with relevance to disease severity as those with absolute correlation with the weight loss higher than 0.15 and with the same direction of correlation in two consecutive time points. Of 24,500 genes, 5075 genes were retained in the correlation matrix. (iii) Hierarchical clustering was applied on the correlation matrix resulting in 15 main clusters of genes. (iv) For each cluster and each time point, a ‘core’ group of co-regulated genes was identified. Each core is a set of 30 genes whose average correlation with the remaining genes in the same cluster is the highest. There were 75 cores, one for each cluster at each time point. (v) For each individual mouse, the average expression was calculated across the 30 genes in each core at the relevant time point, resulting in a vector of 75 values for each individual mouse, which represents the main co-regulation patterns across all time points.

In the second step, the inventors constructed a core-centered representation in which each gene was represented by its interrelations with the 75 core signatures. Specifically, the correlation between each core and each of the 5075 genes across individuals was calculated (using gene expression at 96h p.i.).

In the third step, a map was constructed based on this core-centered representation of genes. Using the core-centered representation of genes as input, an autoencoder neural network was trained with a two-dimensional bottleneck layer and a sigmoid activation function. The inventors focused on a two-dimensional map, since the third dimension has a limited contribution to the explained variation (FIG. 4H). The bottleneck layer was used as the two-dimensional representation, referred to herein as the ‘map’. The map construction was performed in two steps: first, the encoder was trained using data from the 5075 selected genes, and then the genes that were filtered out in step 1 were projected onto the map using the trained network. Of note, the entire construction was applied without pre-standardization of expression levels. To avoid a bias toward early time points (in which the selected time intervals were shorter), the data at 3h p.i. was omitted from the construction of the map.

Definition of the main programs and their levels

The two-dimensional map of the host response consists of two axes that are referred to as “axis T” and “axis R”. The inventors decomposed the observed gradient of each individual (FIG. 3A-II) into two gradients along these axes. Decomposition was performed through a deconvolution-based approach by solving the following regression model:

gᵢⱼ=Tᵢxⱼ+Rᵢyⱼ+bᵢ

where gᵢⱼ, xⱼ, and yⱼ are given as input and the Tᵢ and Rᵢ values are the output. Specifically, gᵢⱼ is the (standardized) expression level of gene j in sample i, and xⱼ and yⱼ are the input positions of gene j in the map. The values of Tᵢ and Rᵢ are the output “levels” of the gradients along the T and R axes in sample i, respectively (bᵢ is a sample-specific constant).

Given that expression levels are largely orchestrated along each of the axes, the inventors reasoned that each axis represents a certain program of regulation. In accordance, the T and R axes are referred to as programs and the inferred levels of gradients along the T and R axes (i.e., the Tᵢ, Rᵢ values) are referred to as the levels of programs T and R (in short, the “T level” and “R level”), respectively. Given the observed increase in T and R levels during infection, positive levels are referred to as ‘activated’ programs, negative levels are referred to as ‘inactivated’ programs, and a zero-level of a program corresponds to an intermediate level of activation. The R and T programs/levels are also referred to as ‘resistance’ and ‘tolerance’ programs/levels, respectively.

As R levels in bulk transcriptomes involve cell intrinsic and cell composition components, this program likely reflects a high-order functional unit. To quantify the cell-intrinsic component of the R program, a pre-defined set was used. A set of 101 genes that are consistently upregulated at the cell autonomous level in nine major cell types during in vivo IAV infection (identified based on single-cell data of the lung tissue in a previous study (Steuerman, Y., et al. (2018). Cell Systems 6, 679-691.e4). This set of genes reflects a cell intrinsic R-response because it relies on a single-cell experiment at a time point in which the R program is activated in the lung tissue (C57BL6/j strain at 48h p.i.). To ensure specificity for the activation of program R, the 50 top R-correlated genes were selected from the 101 genes. The average across these 50 genes was used as the cell-autonomous R level.

Validation data

The inventors used several gene expression studies for additional validation. In each dataset, pre-processing was applied as described in its original publication. For each dataset, gene expression values were centered using the ‘healthy’ samples of the dataset, and then T and R levels were calculated for each sample. The following datasets were evaluated:

(1) In vitro response to PR8 virus stimulation of primary human bronchial epithelial cells in 10 time points (Shapira, S.D., et al. (2009). Cell 139, 1255-1267) (GEO accession GSE19392; FIG. 3C).

(2) A human IAV-infection cohort, consisting of 21 individuals (12 females, 9 males), infected with IAV during the 2010-2011 influenza season (Zhai, Y., et al. (2015). PLOS Pathog 11, e1004869-e1004869). The inventors analyzed, for each individual, blood transcription profiles that were taken before the influenza season and at 0, 2, 4, 6 and 21 days after symptom onset. As clinical phenotypes are not available, the R level at 2 days after symptom onset was used as the marker of disease severity (GSE68310; FIGS. 3D, 11A, 11B, 15C, 15D). Fisher's combined p-values reported in FIG. 15 were calculated across the Pearson's p-values of the markers.

(3) Lung samples of the C57BL/6J mouse strain at 10 time points following in vivo IAV infection (Altboum, Z., et al. (2014). Mol Syst Biol 10, 720-720) (GEO accession GSE49934, FIG. 3E).

(4) In vitro infection of human epithelial cells with various viruses (Daamen, A.R., et al. (2021). Sci Rep 11, 7052-7052) (GEO accession GSE147507, FIG. 6D) and responses of murine dendritic cells (DCs) to the presence of microbiome (Schaupp, L., et al. (2020). Cell 181, 1080-1096.e19) (ArrayExpress accession E-MTAB-5190, FIG. 6D). In these cases, as the numbers of control samples were low, the inventors only calculated differential expression for each gene (using the DESeq2 R library). Specifically, for the latter case, transcriptomes of wild-type C57BL/6J mice that were grown in specific pathogen free (SPF) conditions, were compared to mice grown in a germ-free (GF) facility, which enabled us to compare data from DCs in the presence of microbiome to data from DCs in the absence of microbiome. Positive/negative differential expression scores refer to a level in an SPF facility that is higher/lower than the level in a GF facility, respectively.

(5) Skin samples of the ICR mouse strain at 8 time points (0-192 h) post injury (St. Laurent, et al. (2017). Frontiers in Molecular Biosciences 4, 57) (FIG. 3F, FIG. 6C).

(6) Microarray data for LPS-treated and control primary macrophages of BXD strains (Orozco, L. D., et al. (2012). Cell 151, 658-670) (GEO accession GSE38705, FIGS. 11C-11D, 15, 6C).

Phenotypes across these strains were downloaded from the GeneNetwork database (Mulligan, M. K., et al. (2017). Methods Mol Biol 1488, 75-120).

(7) Transcriptional profiles from liver across CC mice, including Ebola-infected mice and controls (Price, A., et al. (2020). Cell Reports 30, 1702-1713.e6) (GEO accession GSE130629, FIGS. 11C-11D, 6C).

(8) A human cohort of whole blood samples from children with septic shock (n=82) and healthy controls (n=21). Samples taken within 24 hours of admission to the pediatric intensive care unit (Wynn, J. L., et al. (2011). Mol Med 17, 1146-1156) (GEO accession GSE26378, e.g., FIGS. 11C-11D, 6C).

(9) A human cohort of whole blood samples from patients with community-acquired pneumonia (CAP) by SARS-COV-2 within the first 24 h of hospital admission (Aschenbrenner, A. C., et al. (2021). Genome Medicine 13, 7) (FIG. 11C-11D).

(10) Isolated cells from 26 cell types derived from blood samples across 63 healthy subjects, 60 SLE patients, 45 SSc patients, 19 RA patients, 42 IIM patients and 19 MCTD patients [Ota, M. et al. Cell 184, 3006-3021.e17 (2021)].

(11) A mouse cohort of lung samples from IAV (H3N2 strain)-infected CC mice (9 MX1-deficient strains, 3 mice per strains, profiled at 3 and 5 days post infection) [Noll, K. E. et al. Cell Rep 31, 107587-107587 (2020)] (GEO accession GSE136748).

Evolutionary conserved differentially expressed genes was further used from stimulated (dsRNA) fibroblasts across four species (human, macaque, rat, and mouse) (Hagai, T., et al. (2018). Nature 563, 197-202) (FIG. 6E).

Estimation of tolerance and resistance from reduced marker sets

The inventors aimed to identify a small set of gene markers that could be used to assess the R and T levels, thereby allowing practical evaluation of the combined resistance-tolerance state. To that end, the Pearson's correlation coefficient was calculated of each candidate gene with the level of each program across samples (a ‘gene-to-program correlation’ score). For a given dataset, the gene-to-program correlations were calculated using all samples (i.e., all strains and time points from the same dataset). This analysis was applied independently to three gene expression datasets: (i) all time points and strains in the CC cohort (bulk data from the lungs); (ii) all individuals in the human IAV-infection cohort (blood samples from GSE68310), and (iii) all samples of primary bronchial epithelial cells (in vitro infection, data from GSE19392). Encouraged by the consistency of gene-to-program correlations between the three datasets (FIG. 7A, 7B, 7C, 7E), the inventors selected the marker genes in two steps: first, the inventors took the top-associated genes based on the CC cohort (Pearson's absolute r>0.75 for tolerance, r>0.65 for resistance), and then filtered out genes with either opposite directions of correlation or low correlations (absolute r<0.4) in at least one of the two other datasets. Only markers with linear relationships to the state were retained. Overall, 51 markers for tolerance and 18 markers for resistance were identified (Table 1, discloses biomarkers of Tolerance (A) and Resistance (B). As expected, the averaged expression of these genes was closely linked to the overall T and R levels calculated for each of the three datasets (FIG. 7D).

Table 1A-1B: Markers of disease-tolerance and resistance levels. The selected marker genes indicated in column 1, of tolerance (Table 1A) and resistance (Table 1B). For a marker, the table provides its group (column 2), various identifiers (columns 3-8), and the correlation coefficients (Pearson's r) of the markers with tolerance levels (columns 9-11) and resistance levels (columns 12-14) based on data in the CC cohort (columns 9, 12), human epithelial cells (columns 10, 13) and the human blood dataset (columns 11, 14).

TABLE 1A

| | | | | | | | | | Gene-to-T | | | Gene-to-T | |
|---|---|---|---|---|---|---|---|---|---|---|---|---|---|
| | | | | | | | | | correlation, | Gene-to-T | | correlation, | Gene-to-T |
| | | Human | Human | Mouse | | Entrez | | Gene-to-T | Human | correlation, | Gene-to-T | Human | correlation, |
| | Marker | gene stable | gene stable | gene stable | Human | gene | Ensembl | correlation, | epithelial | Human | correlation, | epithelial | Human |
| Symbol | group | ID | ID version | ID | gene name | id | gene id | CC, lungs | cells | blood | CC, lungs | cells | blood |
| Pcnp | T− | ENSG000000 | ENSG0000008 | ENSMUSG0000 | PCNP | 57092 | ENSG000000 | 0.768613769 | −0.635167972 | −0.788779333 | −0.000308133 | 0.29775855 | 0.301131741 |
| | | 81154 | 1154.12 | 0071533 | | | 81154 | | | | | | |
| Vbp1 | T− | ENSG000001 | ENSG0000015 | ENSMUSG0000 | VBP1 | 7411 | ENSG000001 | −0.864101293 | −0.630954504 | −0.75237244 | 0.181666302 | 0.535741787 | 0.399265368 |
| | | 55959 | 5959.12 | 0031197 | | | 55959 | | | | | | |
| Abce1 | T− | ENSG000001 | ENSG0000016 | ENSMUSG0000 | ABCE1 | 6059 | ENSG000001 | −0.848242949 | −0.557388165 | −0.752027826 | 0.347736725 | 0.109281729 | 0.207202182 |
| | | 64163 | 4163.11 | 0058355 | | | 64163 | | | | | | |
| Tfb2m | T− | ENSG000001 | ENSG0000016 | ENSMUSG0000 | TFB2M | 64216 | ENSG000001 | −0.785195179 | −0.402199593 | −0.723747788 | −0.097072169 | 0.014400231 | 0.201276474 |
| | | 62851 | 2851.8 | 0026492 | | | 62851 | | | | | | |
| Csnk1g3 | T− | ENSG000001 | ENSG0000015 | ENSMUSG0000 | CSNK1G3 | 1456 | ENSG000001 | −0.839106797 | −0.702875894 | −0.696336461 | −0.02562088 | 0.479882217 | 0.362775511 |
| | | 51292 | 1292.17 | 0073563 | | | 51292 | | | | | | |
| Rb1cc1 | T− | ENSG000000 | ENSG0000002 | ENSMUSG0000 | RB1CC1 | 9821 | ENSG000000 | −0.860175688 | −0.492066575 | −0.676949235 | 0.028572417 | −0.214611109 | 0.32896904 |
| | | 23287 | 3287.13 | 0025907 | | | 23287 | | | | | | |
| Gmfb | T− | ENSG000001 | ENSG0000019 | ENSMUSG0000 | GMFB | 2764 | ENSG000001 | 0.874967679 | −0.705953867 | −0.671212744 | 0.165694035 | 0.637296374 | 0.351466962 |
| | | 97045 | 7045.13 | 0062014 | | | 97045 | | | | | | |
| Asf1a | T− | ENSG000001 | ENSG0000011 | ENSMUSG0000 | ASF1A | 25842 | ENSG000001 | −0.78297893 | −0.466229785 | −0.668841816 | 0.144753864 | 0.279492256 | 0.084848694 |
| | | 11875 | 1875.8 | 0019857 | | | 11875 | | | | | | |
| Senp6 | T− | ENSG000001 | ENSG0000011 | ENSMUSG0000 | SENP6 | 26054 | ENSG000001 | −0.809207236 | −0.424135712 | −0.6658152 | 0.036403411 | −0.150437578 | 0.130226361 |
| | | 12701 | 2701.18 | 0034252 | | | 12701 | | | | | | |
| Fbxo3 | T− | ENSG000001 | ENSG0000011 | ENSMUSG0000 | FBXO3 | 26273 | ENSG000001 | −0.857510231 | −0.51951937 | −0.656515423 | 0.05452526 | −0.07238112 | 0.380873302 |
| | | 10429 | 0429.14 | 0027180 | | | 10429 | | | | | | |
| Sucla2 | T− | ENSG000001 | ENSG0000013 | ENSMUSG0000 | SUCLA2 | 8803 | ENSG000001 | −0.783640052 | −0.55365828 | 0.647048053 | 0.276373564 | 0.22917828 | 0.50387239 |
| | | 36143 | 6143.16 | 0022110 | | | 36143 | | | | | | |
| Ube2v2 | T− | ENSG000001 | ENSG0000016 | ENSMUSG0000 | UBE2V2 | 7336 | ENSG000001 | −0.76909476 | −0.442346487 | −0.645494595 | 0.160682073 | 0.498285608 | 0.260401821 |
| | | 69139 | 9139.12 | 0022674 | | | 69139 | | | | | | |
| Dld | T− | ENSG000000 | ENSG0000009 | ENSMUSG0000 | DLD | 1738 | ENSG000000 | −0.815092057 | −0.587181857 | −0.643340526 | 0.300232824 | 0.282421961 | 0.477641042 |
| | | 91140 | 1140.14 | 0020664 | | | 91140 | | | | | | |
| Uchl5 | T− | ENSG000001 | ENSG0000011 | ENSMUSG0000 | UCHL57 | 5137 | ENSG000001 | −0.754708783 | −0.562105695 | −0.623617265 | 0.328087198 | 0.056413901 | 0.515025823 |
| | | 16750 | 6750.14 | 0018189 | | | 16750 | | | | | | |
| Prpf4b | T− | ENSG000001 | ENSG0000011 | ENSMUSG0000 | PRPF4B | 8899 | ENSG000001 | −0.810870208 | −0.504297457 | −0.6147224 | −0.042078328 | 0.292980965 | 0.15484937 |
| | | 12739 | 2739.17 | 0021413 | | | 12739 | | | | | | |
| Eif3m | T− | ENSG000001 | ENSG0000014 | ENSMUSG0000 | EIF3M | 10480 | ENSG000001 | −0.784048397 | −0.538826558 | −0.604297139 | 0.464122803 | 0.455161198 | 0.040925333 |
| | | 49100 | 9100.13 | 0027170 | | | 49100 | | | | | | |
| Armc1 | T− | ENSG000001 | ENSG0000010 | ENSMUSGO000 | ARMC1 | 55156 | ENSG000001 | −0.816721478 | −0.69470331 | −0.598145276 | 0.268803392 | 0.22281981 | −0.065358063 |
| | | 04442 | 4442.10 | 0027599 | | | 04442 | | | | | | |
| Tmco1 | T− | ENSG000001 | ENSG0000014 | ENSMUSG0000 | TMCO1 | 54499 | ENSG000001 | −0.823807455 | −0.463682996 | −0.580470636 | −0.041754154 | 0.636584139 | 0.575941389 |
| | | 43183 | 3183.18 | 0052428 | | | 43183 | | | | | | |
| Vps26a | T− | ENSG000001 | ENSG0000012 | ENSMUSGO000 | VPS26A | 9559 | ENSG000001 | −0.756897971 | −0.538237332 | −0.57140374 | 0.333664013 | 0.170962966 | 0.347513053 |
| | | 22958 | 2958.15 | 0020078 | | | 22958 | | | | | | |
| Rsbn1 | T− | ENSG000000 | ENSG0000008 | ENSMUSG0000 | RSBN1 | 54665 | ENSG000000 | −0.815369296 | −0.548341086 | −0.566830329 | −0.014745713 | 0.143767059 | 0.1514815 |
| | | 81019 | 1019.13 | 0044098 | | | | | | | | | |
| Copb1 | T− | ENSG000001 | ENSG0000012 | ENSMUSG0000 | COPB1 | 1315 | ENSG000001 | −0.816955975 | −0.579479075 | −0.553344175 | 0.396138406 | 0.000765997 | 0.339865107 |
| | | 29083 | 9083.13 | 0030754 | | | 29083 | | | | | | |
| Ggps1 | T− | ENSG000001 | ENSG0000015 | ENSMUSG0000 | GGPS1 | 9453 | ENSG000001 | −0.79529167 | −0.438222714 | −0.535133422 | 0.204714203 | 0.389700679 | 0.463719648 |
| | | 52904 | 2904.11 | 0021302 | | | 52904 | | | | | | |
| Nxt2 | T− | ENSG000001 | ENSG0000010 | ENSMUSG0000 | NXT2 | 55916 | ENSG000001 | −0.817343005 | −0.456121342 | −0.529619851 | −0.026396044 | 0.610538553 | 0.328990126 |
| | | 01888 | 1888.12 | 0042271 | | | 01888 | | | | | | |
| Lin7c | T− | ENSG000001 | ENSG0000014 | ENSMUSG0000 | LIN7C | 55327 | ENSG000001 | −0.76299793 | −0.57660625 | −0.528793618 | −0.183608303 | 0.520533611 | 0.181029011 |
| | | 48943 | 8943.12 | 0027162 | | | 48943 | | | | | | |
| Chmp 2b | T− | ENSG000000 | ENSG0000008 | ENSMUSG0000 | CHMP 2B | 25978 | ENSG000000 | −0.820323799 | −0.420940941 | −0.520484215 | 0.065993037 | 0.321758633 | 0.663168394 |
| | | 83937 | 3937.10 | 0004843 | | | 83937 | | | | | | |
| Ppp2r 3c | T− | ENSG000000 | ENSG0000009 | ENSMUSG0000 | PPP2R 3C | 55012 | ENSG000000 | −0.756887068 | −0.544877394 | −0.505097354 | 0.488212215 | 0.512483932 | 0.531073374 |
| | | 92020 | 2020.10 | 0021022 | | | 92020 | | | | | | |
| Zc3h1 4 | T− | ENSG000001 | ENSG0000010 | ENSMUSG0000 | ZC3H1 4 | 79882 | ENSG000001 | −0.834051528 | −0.484512515 | −0.49960538 | −0.015144052 | −0.261915349 | 0.164495221 |
| | | 00722 | 0722.20 | 0021012 | | | 00722 | | | | | | |
| Rab21 | T− | ENSG000000 | ENSG0000008 | ENSMUSG0000 | RAB21 | 23011 | ENSG000000 | −0.753680368 | −0.565005924 | −0.492752771 | 0.344769263 | 0.268901705 | 0.359172621 |
| | | 80371 | 0371.6 | 0020132 | | | 80371 | | | | | | |
| Ppp3c b | T− | ENSG000001 | ENSG0000010 | ENSMUSG0000 | PPP3C B | 5532 | ENSG000001 | −0.811361407 | −0.425105728 | −0.470349492 | 0.077533559 | 0.146568435 | 0.207613281 |
| | | 07758 | 7758.16 | 0021816 | | | 07758 | | | | | | |
| Agps | T− | ENSG000000 | ENSG0000001 | ENSMUSG0000 | AGPS | 8540 | ENSG000000 | −0.810783112 | −0.5108468 | −0.435572276 | 0.124777787 | 0.269844667 | 0.673398248 |
| | | 18510 | 8510.17 | 0042410 | | | 18510 | | | | | | |
| Uba3 | T− | ENSG000001 | ENSG0000014 | ENSMUSG0000 | UBA3 | 9039 | ENSG000001 | −0.816353328 | −0.428776893 | −0.421895495 | 0.393727911 | 0.112536047 | 0.626197816 |
| | | 44744 | 4744.17 | 0030061 | | | 44744 | | | | | | |
| Eps15 | T− | ENSG000000 | ENSG0000008 | ENSMUSG0000 | EPS15 | 2060 | ENSG000000 | −0.842449408 | −0.606224667 | −0.418587747 | 0.008122612 | 0.278836361 | 0.568982415 |
| | | 85832 | 5832.17 | 0028552 | | | 85832 | | | | | | |
| Arl1 | T− | ENSG000001 | ENSG0000012 | ENSMUSG0000 | ARL1 | 400 | ENSG000001 | −0.813967799 | −0.42284644 | −0.403975682 | 0.221900391 | 0.605074995 | 0.336426686 |
| | | 20805 | 0805.14 | 0060904 | | | 20805 | | | | | | |
| Rhbdf2 | T+ | ENSG000001 | ENSG0000012 | ENSMUSG0000 | RHBDF2 | 79651 | ENSG000001 | 0.781739143 | 0.479322964 | 0.401682759 | 0.185953835 | −0.425853233 | 0.39246697 |
| | | 29667 | 9667.13 | 0020806 | | | 29667 | | | | | | |
| Ercc1 | T+ | ENSG000000 | ENSG0000001 | ENSMUSG0000 | ERCC1 | 2067 | ENSG000000 | 0.790018563 | 0.455739478 | 0.408065349 | −0.012552639 | 0.093927882 | −0.155477466 |
| | | 12061 | 2061.16 | 0003549 | | | 12061 | | | | | | |
| Tmem | T+ | ENSG000001 | ENSG0000011 | ENSMUSG0000 | TMEM214 | 54867 | ENSG000001 | 0.821547425 | 0.609723743 | 0.415685669 | −0.161890254 | −0.061641169 | −0.073547691 |
| 214 | | 19777 | 9777.20 | 0038828 | | | 19777 | | | | | | |
| Ccnd3 | T+ | ENSG000001 | ENSG0000011 | ENSMUSG0000 | CCND3 | 896 | ENSG000001 | 0.77739194 | 0.453525972 | 0.42460916 | 0.117193763 | 0.156432344 | 0.236225135 |
| | | 12576 | 2576.13 | 0034165 | | | 12576 | | | | | | |
| Lrp10 | T+ | ENSG000001 | ENSG0000019 | ENSMUSG0000 | LRP10 | 26020 | ENSG000001 | 0.864838433 | 0.565212607 | 0.425236289 | −0.1829607 | −0.665974215 | −0.011397294 |
| | | 97324 | 7324.9 | 0022175 | | | 97324 | | | | | | |
| Mboat7 | T+ | ENSG000001 | ENSG0000012 | ENSMUSG0000 | MBOAT7 | 79143 | ENSG000001 | 0.760210219 | 0.527378315 | 0.431201816 | −0.23700286 | −0.58029022 | −0.083546217 |
| | | 25505 | 5505.17 | 0035596 | | | 25505 | | | | | | |
| Grina | T+ | ENSG000001 | ENSG0000017 | ENSMUSGO000 | GRINA | 2907 | ENSG000001 | 0.771664164 | 0.54527754 | 0.451311786 | 0.299237669 | −0.361263402 | −0.217543682 |
| | | 78719 | 8719.17 | 0022564 | | | 78719 | | | | | | |
| Zyx | T+ | ENSG000001 | ENSG0000015 | ENSMUSG0000 | ZYX | 7791 | ENSG000001 | 0.837657765 | 0.433487802 | 0.460062392 | −0.058771669 | −0.20725023 | 0.069831693 |
| | | 59840 | 984 0.16 | 0029860 | | | 59840 | | | | | | |
| Tnfrsf1a | T+ | ENSG000000 | ENSG0000006 | ENSMUSG0000 | TNFRSF1A | 7132 | ENSG000000 | 0.785215637 | 0.479867055 | 0.461557583 | 0.161127011 | −0.48119086 | 0.152230543 |
| | | 67182 | 7182.8 | 0030341 | | | 67182 | | | | | | |
| Carm1 | T+ | ENSG000001 | ENSG0000014 | ENSMUSG0000 | CARM1 | 10498 | ENSG000001 | 0.779066217 | 0.521429274 | 0.487774028 | −0.241755566 | −0.236793732 | −0.444108818 |
| | | 42453 | 2453.12 | 0032185 | | | 42453 | | | | | | |
| Nucb1 | T+ | ENSG000001 | ENSG0000010 | ENSMUSG0000 | NUCB1 | 4924 | ENSG000001 | 0.804099634 | 0.678382479 | 0.497091307 | −0.388308011 | −0.444910235 | 0.140857779 |
| | | 04805 | 4805.16 | 0030824 | | | 04805 | | | | | | |
| Capn1 | T+ | ENSG000000 | ENSG0000001 | ENSMUSG0000 | CAPN1 | 823 | ENSG000000 | 0.759155909 | 0.45674056 | 0.502163452 | −0.164224483 | −0.526097183 | −0.249230825 |
| | | 14216 | 4216.16 | 0024942 | | | 14216 | | | | | | |
| Cant1 | T+ | ENSG000001 | ENSG0000017 | ENSMUSG0000 | CANT1 | 124583 | ENSG000001 | 0.775769418 | 0.459059139 | 0.511680879 | −0.217648771 | −0.519966125 | −0.223253096 |
| | | 71302 | 1302.17 | 0025575 | | | 71302 | | | | | | |
| Med1 6 | T+ | ENSG000001 | ENSG0000017 | ENSMUSG0000 | MED1 6 | 10025 | ENSG000001 | 0.789795589 | 0.571633938 | 0.516113734 | −0.422865779 | −0.226098392 | −0.399994261 |
| | | 75221 | 5221.15 | 0013833 | | | 75221 | | | | | | |
| Vat1 | T+ | ENSG000001 | ENSG0000010 | ENSMUSG0000 | VAT1 | 10493 | ENSG000001 | 0.753147707 | 0.450254284 | 0.541443864 | −0.400031329 | −0.42403693 | −0.422926818 |
| | | 08828 | 8828.16 | 0034993 | | | 08828 | | | | | | |
| Ctsa | T+ | ENSG000000 | ENSG0000006 | ENSMUSG0000 | CTSA | 5476 | ENSG000000 | 0.758639472 | 0.498059314 | 0.582371237 | 0.015871337 | −0.547122152 | 0.076843787 |
| | | 64601 | 4601.20 | 0017760 | | | 64601 | | | | | | |
| Hgs | T+ | ENSG000001 | ENSG0000018 | ENSMUSG0000 | HGS | 9146 | ENSG000001 | 0.872132153 | 0.68351648 | 0.597170641 | −0.25378847 | −0.48563196 | −0.147844314 |
| | | 85359 | 5359.14 | 0025793 | | | 85359 | | | | | | |
| Rhog | T+ | ENSG000001 | ENSG0000017 | ENSMUSG0000 | RHOG | 391 | ENSG000001 | 0.796046805 | 0.401528684 | 0.61480042 | 0.27694249 | −0.220917355 | 0.12663924 |
| | | 77105 | 7105.10 | 0073982 | | | 77105 | | | | | | |

TABLE 1B

| | | | | | | | | | Gene-to-T | | | Gene-to-T | |
|---|---|---|---|---|---|---|---|---|---|---|---|---|---|
| | | | | | | | | | correlation, | Gene-to-T | | correlation, | Gene-to-T |
| | | Human | Human | Mouse | | Entrez | | Gene-to-T | Human | correlation, | Gene-to-T | Human | correlation, |
| | Marker | gene stable | gene stable | gene stable | Human | gene | Ensembl | correlation, | epithelial | Human | correlation, | epithelial | Human |
| Symbol | group | ID | ID version | ID | gene name | id | gene id | CC, lungs | cells | blood | CC, lungs | cells | blood |
| Zfp395 | R− | ENSG000001 | ENSG00000186 | ENSMUSG00000 | ZNF395 | 55893 | ENSG0000 | 0.274240757 | 0.349171485 | 0.013930939 | −0.742118755 | −0.75798956 | −0.527590521 |
| | | 86918 | 918.14 | 034522 | | | 0186918 | | | | | | |
| Wdr 6 | R− | ENSG000001 | ENSG00000178 | ENSMUSG00000 | WDR 6 | 11180 | ENSG0000 | 0.274535849 | 0.27404459 | 0.162771665 | −0.739460788 | −0.435007897 | −0.436293988 |
| | | 78252 | 252.18 | 066357 | | | 0178252 | | | | | | |
| Prkcz | R− | ENSG000000 | ENSG00000067 | ENSMUSG00000 | PRKC Z | 5590 | ENSG0000 | 0.143213906 | 0.402404461 | 0.322376023 | −0.695997643 | −0.569490662 | −0.437975714 |
| | | 67606 | 606.17 | 029053 | | | 0067606 | | | | | | |
| Lgals8 | R+ | ENSG000001 | ENSG00000116 | ENSMUSG00000 | LGALS8 | 3964 | ENSG0000 | −0.211027542 | −0.051776136 | 0.039525938 | 0.674572125 | 0.421918807 | 0.541322307 |
| | | 16977 | 977.19 | 057554 | | | 0116977 | | | | | | |
| Ripk2 | R+ | ENSG000001 | ENSG00000104 | ENSMUSG00000 | RIPK2 | 8767 | ENSG0000 | −0.041040263 | 0.022236059 | 0.079315361 | 0.678183641 | 0.480246915 | 0.517883096 |
| | | 04312 | 312.8 | 041135 | | | 0104312 | | | | | | |
| B2m | R+ | ENSG000001 | ENSG00000166 | ENSMUSG00000 | B2M | 567 | ENSG0000 | −0.061112198 | −0.23614226 | −0.333566322 | 0.719836713 | 0.639704517 | 0.607648595 |
| | | 66710 | 71 0.21 | 060802 | | | 0166710 | | | | | | |
| Psme1 | R+ | ENSG000000 | ENSG00000092 | ENSMUSG00000 | PSME1 | 5720 | ENSG0000 | 0.079810807 | 0.070931227 | 0.254893674 | 0.727217938 | 0.416518022 | 0.580545665 |
| | | 92010 | 01 0.15 | 022216 | | | 0092010 | | | | | | |
| Psma4 | R+ | ENSG000000 | ENSG00000041 | ENSMUSG00000 | PSMA4 | 5685 | ENSG0000 | −0.365274966 | −0.265664918 | −0.375176803 | 0.730883041 | 0.481695926 | 0.660134755 |
| | | 41357 | 357.16 | 032301 | | | 0041357 | | | | | | |
| Mthfd2 | R+ | ENSG000000 | ENSG00000065 | ENSMUSG00000 | MTHF | 10797 | ENSG0000 | 0.133802301 | −0.432512969 | −0.188235886 | 0.734418457 | 0.55972593 | 0.805843577 |
| | | 65911 | 911.13 | 005667 | D2 | | 0065911 | | | | | | |
| Lap3 | R+ | ENSG000000 | ENSG00000002 | ENSMUSG00000 | LAP3 | 51056 | ENSG0000 | −0.238184841 | −0.043194898 | 0.139878891 | 0.735326717 | 0.495183365 | 0.626924457 |
| | | 02549 | 549.12 | 039682 | | | 0002549 | | | | | | |
| Clic1 | R+ | ENSG000002 | ENSG00000213 | ENSMUSG00000 | CLIC1 | 1192 | ENSG0000 | 0.183657018 | −0.085590367 | −0.023846649 | 0.74599201 | 0.474918307 | 0.693550632 |
| | | 13719 | 719.8 | 007041 | | | 0213719 | | | | | | |
| Eif2ak2 | R+ | ENSG000000 | ENSG00000055 | ENSMUSG00000 | EIF2AK2 | 5610 | ENSG0000 | −0.253205042 | −0.026818797 | 0.173654124 | 0.747263307 | 0.447641916 | 0.592475419 |
| | | 55332 | 332.18 | 024079 | | | 0055332 | | | | | | |
| Casp3 | R+ | ENSG000001 | ENSG00000164 | ENSMUSG00000 | CASP3 | 836 | ENSG0000 | −0.354054911 | −0.465570858 | −0.54213965 | 0.760260609 | 0.521676267 | 0.528899526 |
| | | 64305 | 305.19 | 031628 | | | 0164305 | | | | | | |
| Rnf114 | R+ | ENSG000001 | ENSG00000124 | ENSMUSG00000 | RNF114 | 55905 | ENSG0000 | 0.241365893 | −0.002696209 | 0.14377658 | 0.766789806 | 0.580113368 | 0.44055243 |
| | | 24226 | 226.11 | 006418 | | | 0124226 | | | | | | |
| Psma6 | R+ | ENSG000001 | ENSG00000100 | ENSMUSG00000 | PSMA6 | 5687 | ENSG0000 | −0.474369028 | −0.061251882 | −0.537980141 | 0.768975912 | 0.544623446 | 0.594707064 |
| | | 00902 | 902.11 | 021024 | | | 0100902 | | | | | | |
| Tdrd7 | R+ | ENSG000001 | ENSG00000196 | ENSMUSG00000 | TDRD7 | 23424 | ENSG0000 | −0.111118676 | 0.087237535 | 0.100259783 | 0.785873225 | 0.468374585 | 0.6007937 |
| | | 96116 | 116.8 | 035517 | | | 0196116 | | | | | | |
| Gch1 | R+ | ENSG000001 | ENSG00000131 | ENSMUSG00000 | GCH1 | 2643 | ENSG0000 | 0.124186538 | −0.080245181 | 0.046971735 | 0.800916302 | 0.500638794 | 0.613511935 |
| | | 31979 | 979.20 | 037580 | | | 0131979 | | | | | | |
| Glrx | R+ | ENSG000001 | ENSG00000173 | ENSMUSG00000 | GLRX | 2745 | ENSG0000 | −0.063259228 | −0.319930756 | −0.249240182 | 0.839814335 | 0.666864865 | 0.72051088 |
| | | 73221 | 221.14 | 021591 | | | 0173221 | | | | | | |

Functional organization of the map

Functional analysis proceeded in two steps. First, the inventors calculated the correlation of each gene to the levels of each program (a gene-to-program correlation score, as defined above). Then, for each gene set and each program, the inventors calculated the bias of the correlations of genes within the gene sets compared to the remaining genes (a Wilcoxon rank-sum test). The resulting score is called the ‘geneset-to-program association’ and is defined as the log of the Wilcoxon p-value (FDR-corrected), signed by the direction of bias, such that positive and negative signs indicate correlations that are higher and lower than the expected distribution. This functional analysis was applied systematically across several public repositories of gene expression datasets. For clarity, the gene sets were organized into six collections of distinct biological interpretations.

(1) Genesets of biological functions and processes. The entire collection of gene sets from MSigDB's ‘Hallmark’ and ‘Reactome’ repositories (n=1568) was used and several hallmark resistance and tolerance functions that were missing in these repositories were added. The geneset-to-program associations are therefore referred to as ‘function-to-program associations’ (e.g., FIGS. 8A, 14C). In the classification of functions as either resistance or tolerance functions, functions with unknown roles in resistance and tolerance or with known roles in both resistance and tolerance were omitted.

(2) Sets of genes responding to a wide range of stress conditions. The inventors used all genesets from the GO database with annotations that included the term ‘response to’ or ‘cellular response to’ (356 genesets). These geneset-to-program associations are referred to as ‘stress-to-program associations’ (FIG. 8C).

(3) All genesets from the C2 and TFT sections of the MsigDB database (1783 genesets). As each geneset in these sections is a set of gene targets for certain transcription regulators, this collection forms a comprehensive regulator-target network. These geneset-to-program association scores are termed ‘regulator-to-program associations’ (FIG. 13D-13E).

(4) Genesets related to NFκB and interferon signaling, collected manually from the Ingenuity knowledge base, Reactome, and the MsigDB's hallmarks, C2, TFT, and C7 collections (32 genesets, Table 2). The geneset-to-program associations are referred to as ‘function-to-program association’ (e.g., FIG. 14A, 14B).

(5) Positive and negative regulators of various biological functions. Included were genesets from the GO repositories with annotations that start with either ‘positive regulation of’ or ‘negative regulation of’. Overall, n=625 pairs of genesets were used, where each pair includes one geneset of positive regulators and one geneset of negative regulators for the same function. The geneset-to-program associations are referred to as ‘function-to-program association’ (FIG. 14D). The analysis excluded genes that encoded factors that were dual positive and negative regulators of the same function.

(6) All genesets from the C7 section of MsigDB repository, which have immunological functions (n=4872). Each geneact includes the top differentially expressed genes (DBGs) between two immunological settings. Association scores are referred to as ‘DBG-to-program associations’.

TABLE 2
Association of resistance (R) and disease-tolerance (T) with key signaling pathways.

| | | | Function-to- | Function-to- |
|---|---|---|---|---|
| | | | T | R |
| | | | associ- | associ- |
| | | Catego- | ation | ation |
| | | rization | (signed | (signed |
| | Source of | of | log q- | log q- |
| Geneset | geneset | geneset | value) | value) |
| HALLMARK_INTERFERON_GAMMA_RESPONSE | Hallmarks | IFN | 2.27471888 | 43.79588002 |
| | MSigDB | | | |
| HALLMARK_INTERFERON_ALPHA_RESPONSE | Hallmarks | IFN | 1.067883898 | 40.47885161 |
| | MSigDB | | | |
| REACTOME_INTERFERON_SIGNALING | Reactome | IFN | 1.661941036 | 32.1370853 |
| REACTOME_INTERFERON_ALPHA_BETA_SIGNALING | Reactome | IFN | 3.199099739 | 21.72492047 |
| REACTOME_INTERFERON_GAMMA_SIGNALING | Reactome | IFN | 2.997899293 | 15.98452143 |
| Interferon Signaling | Ingenuity | | | |
| | knowledge | IFN | 1.326608685 | 12.81797632 |
| | base | | | |
| REACTOME_DDX58_IFIH1_MEDIATED_INDUCTION_OF_ | Reactome | IFN | 1.074014615 | 7.995872548 |
| INTERFERON_ALPHA_BETA | | | | |
| Role of RIG1-like Receptors in Antiviral Innate Immunity | Ingenuity | IFN | 2.240017753 | 6.252253051 |
| | knowledge | | | |
| | base | | | |
| Role of JAK1, JAK2 and TYK2 in Interferon Signaling | Ingenuity | IFN | 1.050837103 | 5.940762699 |
| | knowledge | | | |
| | base | | | |
| GRANDVAUX_IRF3_TARGETS_UP | C2, MSigDB | IFN | 0.027358922 | 4.677754915 |
| IRF_Q6 | TFT, MSigDB | IFN | 2.57198097 | 3.827035936 |
| REACTOME_IRF3_MEDIATED_ACTIVATION_ | Reactome | IFN | 0.49166684 | 1.0411946 |
| OF_TYPE_1_IFN | | | | |
| HALLMARK_TNFA_SIGNALING_VIA_NFKB | Hallmarks | NFKB | 11.8540849 | 36.86203763 |
| | MSigDB | | | |
| REACTOME_ACTIVATION_OF_NF_KAPPAB_IN_B_CELLS | Reactome | NFkB | −0.533253683 | 23.42634124 |
| NFKBIA_TARGET_GENES | TFT, MSigDB | NFKB | 13.79588002 | −14.44909253 |
| GSE7219_WT_VS_NIK_NFKB2_KO_DC_UP | C7, MSigDB | NFKB | 4.251780427 | 11.52376668 |
| HINATA_NFKB_TARGETS_KERATINOCYTE_UP | C2, MSigDB | NFKB | 3.184855781 | 11.49521638 |
| NF-KB Signaling | Ingenuity | NFKB | 2.427320659 | 8.058602854 |
| | knowledge | | | |
| | base | | | |
| RASHI_NFKB1_TARGETS | C2, MSigDB | NFKB | 3.006805235 | 6.903715392 |
| HINATA_NFKB_TARGETS_FIBROBLAST_UP | C2, MSigDB | NFKB | 1.253993141 | 5.900321553 |
| REACTOME_RIP_MEDIATED_NFKB_ACTIVATION_ | Reactome | NFKB | 0.934276015 | 5.220880636 |
| VIA_ZBP1 | | | | |
| REACTOME_TAK1_ACTIVATES_NFKB_ | Reactome | NFKB | 1.048744133 | 2.634841478 |
| BY_PHOSPHORYLATION_AND_ | | | | |
| ACTIVATION_OF_IKKS_COMPLEX | | | | |
| GGGNNTTTCC_NFKB_Q6_01 | TFT, MSigDB | NFKB | 8.012983192 | 2.427197076 |
| GSE7219_WT_VS_NIK_NFKB2_KO_DC_DN | C7, MSigDB | NFKB | −0.483951174 | 2.287043265 |
| NFKAPPAB_01 | TFT, MSigDB | NFKB | 10.62836746 | 2.241218346 |
| NFkB non-canonical pathway | Ingenuity | NFKB | 3.301229164 | 2.220098423 |
| | knowledge | | | |
| | base | | | |
| NFkB (complex) | Ingenuity | NFKB | 3.6698359 | 2.124768777 |
| | knowledge | | | |
| | base | | | |
| GSE7219_WT_VS_NIK_NFKB2_KO_LPS_AND_ANTI_ | C7, MSigDB | NFKB | 0.886478376 | 1.492775733 |
| CD40_STIM_DC_DN | | | | |
| NFkB canonical pathway | Ingenuity | NFKB | 0.178180729 | 1.2610569 |
| | knowledge | | | |
| | base | | | |
| NFKAPPAB65_01 | TFT, MSigDB | NFKB | 7.378799714 | 0.83221124 |
| NFKB_Q6 | TFT, MSigDB | NFKB | 7.954095223 | 0.327750936 |
| NFKB_C | TFT, MSigDB | NFKB | 8.331744621 | 0.013917328 |

Co-expression with R and T levels in human data

The analysis relied on the SEEK algorithm (Zhu, Q., et al. (2015). Nature Methods 12, 211-214), which takes as input a query gene set and calculates a score for the co-expression of each human gene with the query set across a large collection of 3405 human transcriptome datasets. As input query sets, the inventors used the groups of 100 genes from the two negative extremities of the T and the R axes (referred to as N-T and N-R, FIG. 12A). The inventors define the “co-expression with T” score of each gene as the SEEK's co-expression scores when using T-specific genes as the query geneset (specifically, using the N-T group as the query set, see FIG. 12A), and define the “co-expression with R” score of each gene as the SEEK's co-expression scores when using R-specific genes as the query geneset (using the N-R group as the query set, see FIG. 12A). Co-expression scores were calculated using the entire set of 3405 human transcriptome datasets (all cell types, FIG. 12A) or using human datasets of each specific cell type independently, providing cell-type-specific co-expression scores (FIG. 12C).

### Analysis of Arhgdia

Cell lines

Immortalized mouse Lung Epithelial Type I (LET1) (BEI Resources, NIAID, NIH, NR-42941), Madin-Darby canine kidney (MDCK), 293 FT and mouse lung type II epithelial (MLE-12) cell lines were maintained in Dulbecco's modified Eagle's medium (DMEM, high glucose) supplemented with 10% fetal bovine serum (FBS), 2 mM L-glutamine, 1% penicillin/streptomycin (all from Biological Industries, Kibbutz Beit-Haemek, Israel). All cells were grown at 37° C. in a humidified atmosphere containing 5% CO₂.

### Viruses

Influenza A virus strain A/PR/8/34 (H1N1) and recombinant PR8-mNeonGreen (a clone named “PB2/PA PTV1 mNeonGreen”, which harbors two copies of the gene of the mNeonGreen fluorescence marker, fused to the PB2 and PA polymerase subunits, via a porcine teschovirus-1 2A peptide sequence), were propagated by allantoic inoculation of 10-days-old chicken embryonated eggs (Kimron Veterinary Institute) and stored at-80° C. Viral titers were determined using MDCK cells and expressed as median tissue culture infectious dose (TCID50). For infection experiments, 24-well plates were seeded with LET1 (50000 cells/well) or MLE-12 (100000 cells/well) cells. After 24h, cells were washed with PBS and infected with IAV (MOI=5), in a serum-free medium for Ihr with gentle rocking. Following infection, the inoculum was removed, and cells were incubated in OptiMEM I reduced medium (Thermo Fisher Scientific) for the indicated time. All experiments were carried out in triplicate. For overexpression experiments, cells were induced for 72 hr before IAV infection, under the presence of doxycycline (2 μg/ml, Sigma; #D9891). Flow cytometry data were acquired from 10,000 gated events using $100EXi flow cytometer (Stratedigm) and analyzed using FlowJo version v10 software (FlowJo, LLC).

Generation of stable cell lines

Lenti vectors harboring the CRISPR/Cas9 system for Arhgdia targeting were produced by co-transfection of 293 FT cells with psPAX2 (Addgene, #12260), pMD2.G (Addgene, #12259), and LentiCRISPRv2 (Addgene, #52961) plasmids, using Lipofectamine 3000 (Thermo Fisher Scientific). Single-guide RNA (sgRNA) included sg-Arhgdia- #1 (5′-CACCGTGAGTTCCTGACACCCATGG-3′, also denoted by SEQ ID NO: 37), and sg-Arhgdia- #2 (5′-CACCGTGAGTTCCTGACACCCATGG-3′, also denoted by SEQ ID NO: 38), targeting the murine Arhgdia gene, or a non-targeting sgRNA (‘control’, 5′-CACCGACGGAGGCTAAGCGTCGCAA-3′, also denoted by SEQ ID NO: 39). After 48 hr, supernatants containing virus-like particles (VLPs) were collected, filtered (0.45 μm), and used to transduce LET1 and MLE-12 cells in the presence of polybrene (8 μg/ml). After 24h, cells were placed under puromycin (2 μg/ml; Sigma #P8833) selection. LET1 resistant cells were seeded into a 96-well dish (1 cell/well) for the expansion of single clones. Transduced LET1 colonies and transduced pooled MLE-12 cells were selected and verified for the loss of Arhgdia expression by immunoblotting. Stable and inducible expression of Arhgdia in naïve LET1 cell line was achieved by lentivirus transduction of a pCW-Arhgdia lentivector expressing the Arhgdia construct. This construct was generated by replacing the Cas9 gene from pCW-Cas9 plasmid (Addgene #50661), residing between the Nhel and BamHI restriction sites, with the murine Arhgdia cDNA (NM_133796.8) using the same cloning sites. For the addback experiment, Arhgdia-depleted (sgRNA #1) cell line was transduced with pCW-Arhgdia lentivector expressing the Arhgdia sequence with silent mutation in the sgRNA #1 target, rendering the resulting sequence resistant to CRISPR/Cas9 cleavage.

Immunoblot analysis

Immunoblot analysis was performed with the following primary antibodies and dilutions: anti-Arhgdia (Santa Cruz Biotechnology, #sc-373723, 1:1000), anti-β-actin clone C4 (MP Biomedicals, #0869100, 1:10000), anti-Influenza A nucleoprotein (NovusBio, #NBP2-16965, 1:1000). Secondary antibodies and dilutions included the Goat anti-mouse horseradish peroxidase-conjugated (Jackson ImmunoResearch, #115-035-062, 1:10000), Goat anti-Rabbit horseradish peroxidase-conjugated (Jackson ImmunoResearch, #111-035-045, 1:10000).

Cell viability

Cell viability was determined with Live/Dead fixable red stain kit (Thermo Fisher, #L34972).

RNA profiling

Library construction, sequencing and pre-processing of data were performed as described above, with the following differences: Total RNA was extracted from cells using the Direct-zol RNA Miniprep kit (ZYMO Research). Sequencing of the RNA samples (0.5 μg) was performed using the Illumina NextSeq 550 platform (Technion Genome Center, Israel) and alignment of IAV and mouse (GRCm39.104) chimeric genome was performed using STAR 2.7.9 aligner. Data were deposited in the GEO database (GSE193160).

### Quantitative PCR

To measure viral RNA levels in the relevant samples, total RNA (1 μg) was reverse transcribed using the SuperScript kit (BioRad), and real-time quantitative reverse transcription polymerase chain reaction assay (qRT-PCR) was performed using the Fast SYBR Green Master Mix (Applied Biosystems). Viral RNA was measured by qRT-PCR using primer sequences for the influenza virus M2 gene (forward: 5′-CATGGAATGGCTAAAGACAAGACC-3′, also denoted by SEQ ID NO: 40 reverse: 5′-CCATTAAGGGCATTTTGGACA-3′, also denoted by SEQ ID NO: 41, and normalized to the level of GAPDH (forward: 5′-GGCAAATTCAACGGCACAGT-3′, also denoted by SEQ ID NO: 42, reverse: 5′-AGATGGTGATGGGCTTCCC-3′, also denoted by SEQ ID NO: 43). qRT-PCR was conducted using a StepOnePlus Real-Time PCR System (Applied Biosystems).

### Example 1

Longitudinal study of IAV infection across genetically distinct mouse strains

To characterize transcriptome alterations upon activation of the host defense system, the inventors collected longitudinal data during IAV infection across genetically distinct mouse strains (FIG. 1A). The inventors recorded lung transcriptomes of 33 mouse strains of the collaborative cross (CC) cohort [Noll et al., Cell Host & Microbe 25, 484-498 (2019),], both in steady state (before infection) and at five time points during the course of IA V infection (3h to 96h p.i.). For each gene, expression levels that were centered around the mean of that gene in steady state, were used. In addition, several phenotypes were recorded for these mice, including viral burden by quantification of viral mRNA levels in lungs, whole-body weight loss, breathing dysfunction, expression levels of central antiviral and inflammatory mediators Ifnβ1 and Cel2, and expression signatures for tissue damage and the quantity of immune cells. The selected time interval encompasses the initial incubation period between exposure to the virus and the onset of systemic symptoms (3-24h p.i), and the acute stage that is characterized by exponential increase in viral burden, pronounced symptoms, and a strong immune response (24-96h p.i., FIG. 1B, FIG. 2A). Due to ethical restrictions, this in vivo study did not include later time points.

The data show substantial diversity in disease severity (FIG. 1B, FIG. 1C, FIG. 2B). For instance, some mice lost around 20% of their whole-body weight, whereas others gained weight during the four days of infection. Two lines of evidence showed that this diversity was not simply dominated by noise. First, phenotypes of disease severity correlated with the viral burden, as expected (e.g., Pearson's correlation r=0.85, p<10⁻⁴⁸ for comparison between type 1 interferon and viral burden; FIG. 1C, FIG. 2C). Second, much of this diversity was inherited as variation in disease severity among strains was significantly larger than the variation within mice of the same strain (FIG. 2D). For example, 83% of the variation in weight loss at 96h p.i. was due to genetic differences. Encouraged by these observations, the inventors used the wide diversity to identify central transcriptional programs that underlie the host response, as detailed below.

### Example 2

Gene-expression states during the course of infection are largely captured by two central programs

The inventors constructed a model of the host response using lung transcriptomes across the CC cohort. The analysis consisted of two main steps. In the first step, a deep learning approach was used to reduce the multi-dimensionality of the data (i.e., multiple genes, mouse strains, and time points before and during IAV infection) into a two-dimensional space (FIG. 3A-I). In this arrangement, referred to as a “map”, each gene transcript is embedded at a certain coordinate within the two-dimensional space. The construction of the map relied on a ‘similarity rule’: The closer two genes are to each other in the map, the higher the similarity of their transcriptional responses in all measured strains and at all time points (FIG. 4A). As shown in this study, the horizontal and vertical dimensions of the map are linked to tolerance and resistance functions (see subsequent sections); accordingly, these axes are referred to as “axis T” and “axis R”, respectively.

In the next step, the inventors used the map to describe the gene-expression state of each specific individual. It was found that in many individuals, there is a gradual change in expression levels over the map (see color coding of several individuals in FIG. 3A-II), with a nearly linear change in the expression of genes along the gradient (FIG. 4B). Different individuals showed distinct directions and distinct rates of change along their gradients (FIG. 3A-II), indicating that the overall state of each individual could be represented well by its gradient. As any gradient can be decomposed into two gradients that run along the two axes of the map, the expression state of each individual is represented by two scores: one score for the gradient along axis T (the “level of T”) and one score for the gradient along axis R (the “level of R”). Positive and negative levels indicate increasing and decreasing gradients along the axes, respectively, and a zero level indicates an absence of a gradient. A two-dimensional representation of individual states by their R and T levels is presented in FIG. 3A-III. For instance, for an individual with a global bottom-left to top-right gradient, its overall expression state is described by positive T and R levels (e.g., strain 5000A, 96h p.i., in FIG. 3A-II and 3A-III). The inventors confirmed the R and T scores are robust, reliable, and successfully explain a large percentage of the variation (FIG. 4C-4F). Thus, the analysis suggests two central programs that together underlie the overall gene-expression state: one program that underlies the R-levels (“program R”), and another program that underlies the T-levels (“program T”).

What is the biological meaning of this model? The R and T levels are primarily increasing during infection (FIG. 3B), suggesting that positive R and T levels describe the activation of the two programs. This implies that the levels of each program scale from inactivation (negative values) to activation (positive values; FIG. 3A-III). The baseline R and T levels (before infection) are centered around zero (FIG. 3B), and nearly-zero levels are highly prevalent (FIG. 4G), suggesting that the steady state of the lung tissue commonly exists in a certain tonic (or intermediate) level of activation. As shown in FIG. 3A-IV, the expression of genes can be visualized along with the two-dimensional representation of individual states from FIG. 3A-III. This visualization demonstrates that each individual state, described by a certain combination of R and T levels, is marked by a different signature of genes. For example, individuals with an R-positive/T-positive state are marked by high expression of Itga5, and individuals with a T-negative state are marked by high expression of Rockl (FIG. 3A-IV and FIG. 5A). Importantly, a closer examination reveals that the state-specificity of individual genes (FIG. 3A-IV) corresponds to their positions in the map (FIG. 3A-I)—for instance, high IL6 levels exist in the R-positive state and this gene resides near the positive end of the R axis; high Ecml levels exist in the T-positive state and the gene resides near the positive end of the Taxis (FIG. 5B, 5C for systematic analysis of these relations). Thus, the map can be viewed as a faithful representation of the modularity of the relationship between gene expression and the R/T programs. Taken together, the analysis suggests a combination of two decoupled programs-R and T—that together capture the global gene-expression state of the lung tissue during the course of IA V infection. Although both programs are generally activated during infection, each program often exhibits distinct responses that vary substantially between individuals. Of note, decoupling does not imply complete independence and it is possible that the two programs are interrelated (A cross-talk between the two programs below is demonstrated).

### Example 3

The two programs define a generic cell autonomous response to IAV infection in human epithelial and blood cells

To study the generality of the model, several mouse and human datasets were used, including transcription profiles from (i) in vitro IAV infection of primary human bronchial epithelial cells (Shapira, S.D., et al. (2009). Cell 139, 1255-1267), (ii) blood samples from IAV-infected human subjects (Zhai, Y., et al. (2015). PLOS Pathog 11, e1004869-e1004869), (iii) longitudinal data of IAV infection in C57BL/6J mice (Altboum, et al. (2014). Mol Syst Biol 10, 720-720), (iv) in vitro response of macrophages (MFs) to LPS stimulations across the BXD mouse strains (Orozco, L. D., et al. (2012). Cell 151, 658-670), (v) liver samples from Ebola-infected CC mice (Price, A., et al. (2020). Cell Reports 30, 1702-1713.e6), (vi) human blood samples of septic shock (Wynn, J. L., et al. (2011). Mol Med 17, 1146-1156); (vii) blood samples from SARS-COV-2-infected human subjects (Aschenbrenner, et al. (2021). Genome Medicine 13, 7), and (viii) wound healing of murine skin cells (St. Laurent, G., et al. (2017). Frontiers in Molecular Biosciences 4, 57). The inventors relied on the map that was constructed using IAV-infected CC mice (FIG. 3A-I) for the calculation of R and T levels for each of these samples.

Analysis of these datasets provide evidence for the generality of the R/T model. First, multiple different directions of global gradients were found for individuals in these datasets, with substantial inter-individual variation of gradients within each of these datasets (FIG. 6A-6C; see time-series datasets in FIGS. 3C-3F)-indicating that R and T define generic programs. Second, association of genes with the levels of programs R and T are consistent between in vivo and in vitro infections, between human and mouse data, between immune and non-immune cell types, and between viral and bacterial infections (FIG. 7A). As additional evidence for the generality of the model, it was found that different combinations of R and T levels are observed in responses of human epithelial cells to various pathogenic and commensal microbes (FIG. 6D) and that both programs are conserved over relatively long evolutionary times (FIG. 6E).

Overall, these analyses indicate that the R/T model initially developed based on IAV-infected mice, is generally relevant to a variety of normal and inflammatory conditions, in both mouse and human. The R/T model is applicable to in vitro response of MFs and epithelial cells, suggesting that the R and T programs exist at the cell autonomous level, in both immune and non-immune cell types. The fact that different individuals have different R/T dynamics and that the R/T dynamics is pathogen dependent (e.g., FIGS. 3B-3F, FIG. 4E, FIG. 6D) emphasizes the importance of genetics, environmental factors and the microbes in driving the R/T state.

### Example 4

The two programs are the molecular underpinning of resistance and disease tolerance

Immune defense mechanisms have been classified into two broad categories: resistance mechanisms, which sense and react to the invading pathogen, and tolerance mechanism, which maintain vital homeostasis by limiting stress and tissue damage [1-3, 6-7]. Comparisons with established hallmarks of resistance and tolerance-including (i) molecular functions, (ii) transcriptional responses, and (iii) physiological signatures-indicated that the R and T programs underlie resistance and tolerance, respectively. For each of these aspects, the inventors first describe the methodology and then consider the results.

(i) Functional properties. The analysis relies on the calculation of correlations between the expression of each gene and the levels of each program (termed ‘gene-to-program correlation’; positive and negative correlations indicate genes induced or repressed, respectively, during activation of the program, FIGS. 5A, FIG. 3A-IV). The inventors scored the association between a given program and a given functional gene set based on the bias of these genes toward specific correlations with that program (a ‘function-to-program association’, defined as a two-sided Wilcoxon rank-sum test on the correlation of the genes with the program; a positive/negative sign indicates an association between increasing activation of the program and induction/repression of the functional set of genes, respectively;). Previous investigations have annotated specific functions as either resistance functions or tolerance functions based on their biological role (e.g., ‘wound healing’ in tolerance [1]). Annotations were collected and utilized in the analyses.

It was found that activation of program R was primarily associated with induction of resistance functions (T: p>0.1, R: p<10-43, t-test) and activation of program T was associated with induction of tolerance functions (T: p<10-71, R: p>0.1, t-test) (FIG. 8A). For example, the molecular function of wound healing, which has a known tolerance role [1], tended to be induced during activation of program T but not program R (function-to-T association q<10-12, function-to-R association q>0.05, FIG. 8B-I, top). Indeed, Furin and Pdgfb, which have a known role in wound healing (Siegfried, G., et al. (2005). Oncogene 24, 6925-6935), are positively correlated with program T (Pearson's r>0.56) but not program R (absolute Pearson's r<0.12). Another example is ‘cytokine storm syndrome’, which is considered as an uncontrolled resistance response (Tisoncik, J. R., et al. (2012). Microbiol Mol Biol Rev 76, 16-32), and in agreement, it was found that cytokine storm genes are induced during activation of program R (q<10⁻⁶) but not program T (q>0.05, FIG. 8B-II, middle e.g., Illb and Tnf, with Pearson's r>0.72 for R and r<0.24 for T). Consistent with the notion that both resistance and tolerance are activated during infection, general immune activation functions are associated with both programs (e.g., ‘cytokine signaling in immune system’, with function-to-T q<109, function-to-R q<10-42; FIG. 8B-III, bottom). Collectively, this analysis suggests that R and T are resistance and tolerance programs, respectively.

(ii) Response patterns. The resistance-tolerance paradigm holds that resistance primarily responds to biotic stress and that tolerance is activated in response to both biotic and abiotic stress (Martins et al., 2019; Soares et al., 2017, 2014). To test this, associations of transcriptome programs was calculated with all stress-response categories in the GO annotation (termed ‘stress-to-program associations’; 325 abiotic stress categories and 30 biotic stress categories). As shown in FIG. 8C, program T was activated in response to any stress or damage, both biotic (t-test p<0.003) and abiotic (t-test p<0.001), whereas program R was activated in response to biotic (t-test p<10-64) but not abiotic (t-test p>0.05) stress. For example, ‘response to oxygen-containing compound’ genes are correlated with activation of program T but not program R (q<10-22, 0.05, respectively), and ‘response to biotic stimulus’ genes are correlated with both programs (e.g., q<10-47, 10-5 for R and T, respectively) (FIG. 8C). Thus, the analysis of response patterns supports the hypothesis that R is a resistance program and T is a tolerance program.

(iii) Physiological phenotypes. The previously described resistance-tolerance model postulates that resistance is linked to disease severity, whereas tolerance is linked to the level of fitness relative to the pathogen burden [7]. The inventors found that the levels of program R are significantly correlated with disease symptoms (Pearson's r>0.68 for all symptoms), whereas levels of program T are not (Pearson's r<0.21 for all symptoms; FIGS. 8D, 9A), supporting the notion that R is a resistance program. In support of this conclusion, similar relations appear in two additional comparisons: when considering a broad categorization of mouse strains into symptomatic and asymptomatic groups (FIG. 10A-10G) and when using a signature of a cell-autonomous definition for the R program (an analysis that is independent of cell composition; FIG. 9B). Additionally, tissue damage relative to the IAV burden (in short, ‘relative tissue damage’) was evaluated, focusing on symptomatic individuals that are pertinent to tissue damage. Interestingly, although program T was not correlated with disease symptoms, it was tightly linked with the relative tissue damage (Pearson's r=0.4, p<0.05; FIG. 8E)
supporting the notion that T is a tolerance program.

Collectively, these analyses suggest that R and T represent molecular programs underpinning resistance and tolerance, respectively (FIG. 8F) and imply that the cellular state is shaped by the combination of these two programs. The “resistance-tolerance hypothesis” [1, 2, 7] points to a large collection of distinct mechanisms that are shown to confer either resistance or tolerance, operating at different time points during infection. The findings show that many of these mechanisms stem from the same two programs. Therefore, subsequently the R/T and the resistance/tolerance notations were used interchangeably.

### Example 5

The resistance-tolerance interplay is a central aspect of ‘healthy’ and ‘responding’ states

Given that programs R and T are the molecular underpinnings of resistance and tolerance, it was next asked how the combination of these programs shapes the tissue-immune state. Interestingly, it was found that resistance and tolerance are anti-correlated in both normal and inflammatory conditions, with a stronger antagonistic effect in healthy subjects (FIG. 11A-11C). The same pattern appears in mouse and human samples, in in vivo and in vitro settings, and under exposure to various pathogens. It was therefore speculated that the tissue-immune state reflects a mixture of two effects: an antagonistic crosstalk between the programs and a distinct response to stimulation of each program. Indeed, systematic analysis of thousands of transcriptome datasets confirmed that resistance and tolerance are generally anti-correlated in their levels but are uncoupled in their change of levels following stimulations (all cell types: FIG. 12A, 12B; specific cell types: FIGS. 12C, 12D). These findings coincide with the general understanding that the two programs serve multiple fundamentally distinct roles, including a central role of tolerance in suppression of resistance [1]. These observations have two additional implications. First, the fact that resistance and tolerance vary substantially within the healthy and infected groups emphasizes that the two programs are central to both normal and inflammatory states. Second, whereas R levels are primarily high during inflammatory conditions, T levels are highly heterogeneous-within the same disease (e.g., SARS-CoV-2 infection, septic shock, and MF activation) and between inflammatory conditions (e.g., high T in Ebola infection, p<0.05; low T in human IAV infection, p<10⁻³; t tests; FIG. 11D). As shown below, the wide spectrum of R/T states, both under normal and inflammatory conditions, has important functional and clinical implications.

### Example 6 Novel annotations expand the known functional properties of resistance and disease tolerance Given that programs R and T capture the known properties of resistance and tolerance (FIG. 8), the inventors next highlight novel functionalities of these programs. Examples include genes implicated in the epithelial-mesenchymal transition that are primarily associated with activation of tolerance (function-to-T q<10⁻⁷; e.g., Anpep, Pmepal, Flna, Slc6a8, FIG. 13A) and ligand-dependent nuclear receptors that are negatively associated with activation of resistance (function-to-R q<105; e.g., Ahr, Rxra, Nrldl, Thrb, Nr3c2, FIGS. 3A-IV, FIG. 13B). Indeed, previous studies have shown that Ahr impairs resistance functions and leads to increased morbidity and mortality in IAV-infected mice [11]. It was also found that estrogen signaling genes are associated with activation of tolerance (function-to-T q<10-11; FIG. 13C), consistent with known roles of estrogen signaling in tissue repair [12]. Interestingly, the association of estrogen signaling with resistance is weak (q<10⁻²), despite its documented negative effect on the induction of resistance mechanisms such as interferon signaling (Kovats, S. (2015). Cell Immunol 294, 63-69). This coincides with the notion that induction of tolerance limits the activity of resistance (FIG. 11B). An additional example comes from the analysis of the regulator-target network: the inventors found 955 transcription regulators with targets that showed significant associations with R and T levels (q<0.01, FIG. 13D-13E). These included upstream regulators known to function in resistance and tolerance (e.g., Hifla and P53) [1], and others with uncharacterized functions in these processes (e.g., Hmgal, Znf436)

The interferon and NFKB pathways are clear examples of pathways with distinct roles in resistance and tolerance. The inventors found that these two pathways have strikingly different patterns. Only R levels, but not T levels, are associated with interferon signaling (e.g., the functional category ‘hallmark interferon alpha response’, q<10⁻²¹, FIG. 14A)—for instance, Dhx58, Irf7, and Isg15 are specifically correlated with the level of resistance (FIGS. 14A, 3A-IV). In contrast, NFKB signaling is associated with the activation of either resistance, tolerance, or both (e.g., q<10⁻⁸ for ‘activation of NFKB signaling in B cells’, ‘targets of the NFKB complex’, and ‘TNF-mediated NFkB signaling’;

FIG. 14A). Thus, the antiviral response through the interferon pathway is mainly a resistance response, whereas the NFKB-mediated inflammatory response has roles in both resistance and tolerance. Remarkably, all genes in the non-canonical NFKB pathway are positively correlated with both resistance and tolerance (e.g., Nfkb2, Relb, Traf2. Pearson's r>0.45 for both R and T), unlike those in the canonical NFKB pathway (FIG. 14B), raising the possibility that the non-canonical NFKB pathway is a predominant mechanism of coordination between resistance and tolerance.

The alterations in cellular metabolism that occur during infection are particularly interesting. The various functions of protein expression (e.g., functional categories of transcription, translation, and RNA/protein processing) are correlated with activation of resistance and inactivation of tolerance (p<10-10. 109, respectively; FIG. 14C). For instance, ‘Rrna processing’ and ‘eukaryotic translation initiation’ are positively associated with resistance and negatively associated with tolerance (FIG. 14C). One plausible explanation is that the global up-regulation of protein expression is required for a rapid resistance against invading pathogens, whereas the opposite effect of tolerance on protein expression restricts the level of resistance. Importantly, other metabolic functions also demonstrate opposite relations with resistance and tolerance levels. For example, low resistance and high tolerance levels are related to ‘carbohydrate homeostasis’ functions (q<10⁻⁶,10⁻⁵, respectively; FIG. 14C). Thus, these antagonistic relationships with cellular metabolism may be a general principle by which tolerance counteracts the resistance during the course of infection. The inventors speculate that the observation that in each state there is a certain restriction on metabolic resources (e.g., restricted lipids in high-resistance/low-tolerance state; restricted protein production in low-resistance/high-tolerance state; FIG. 14C) ensures that the environment is suppressive for viral replication. The inventors note that resistance and tolerance are critical not only in fighting infection but also in health —e.g., the high-resistance/low-tolerance baseline state is associated with induction of genes associated with protein production and repression of those involved in carbohydrate metabolism, whereas the low-resistance/high-tolerance baseline state has opposite patterns.

The inventors further evaluated 625 biological processes whose positive and negative regulators were systematically curated as two distinct gene sets (genes that operate as dual positive and negative factors of the same process were excluded). Interestingly, it was found that positive and negative regulators of the same biological process shared similar associations with the resistance program (Pearson's r=0.39) and the tolerance program (Pearson's r=0.62; FIG. 14D). For example, both the positive and negative factors of interferon production are positively associated with resistance levels (p<10⁻⁷, p<10⁻⁵). Another example is cell differentiation, for which both the positive and negative regulatory factors are positively associated with tolerance (p<10-13. 10-7, respectively). The inventors suggest that the observed coordination of positive and negative regulators for each given process is a mechanism that maintains a balanced immune response.

### Example 7

Resistance and disease tolerance are central to autoimmunity, infectious diseases and cancer

Given the wide inter-individual variation in R/T states (FIG. 11B-11C), the inventors now face the question of how the R/T state affects immune-related diseases. To address this question, the R/T levels were used to predict immune-related phenotypes using an independent cohort of BXD mice strains. Particularly, R/T levels in activated MFs of healthy individuals (FIG. 11C) were used to predict 37 traits of susceptibility to various infectious diseases (eight distinct viral, bacterial and fungal infections) and 19 autoimmune and inflammatory markers (such as systemic lupus erythematosus (SLE) and rheumatoid arthritis (RA) markers). Interestingly, susceptibility to inflammatory/autoimmune and infectious diseases have opposite relations with the R/T state: high R and low T levels are associated with lower severity of infections (p<10^{-7, 10}-12, respectively; t-test) and higher levels of inflammation/autoimmunity markers (p<10^{-2. 10}-6, respectively; t-test) (FIG. 15A). Thus, a ‘hit hard, hit quickly’ strategy (high-R/low-T) is beneficial against pathogenic infections but is accompanied by a higher risk of inflammation/autoimmune disease. This implies that the R/T state plays a key role in maintaining a balance between appropriate and disproportionate immune responses.

The baseline R/T state in resting MFs also has an effect on immune-related diseases. First, R/T states in resting and activated MFs have similar associations with diseases (Pearson's p<0.002, p<10-13 for R and T, respectively; FIG. 15B). For instance, the R/T states in both resting and activated MFs show the same relations with the severity of IAV infection (FIG. 15C), consistent with the observations across the CC mice (FIG. 16A-16C). Second, the baseline R and T levels in resting MFs are correlated with the levels of R and T in activated MFs (r=0.4, 0.64 for R and T, respectively, FIG. 15D). These findings suggest that inter-individual variation in immune-related diseases is affected, at least in part, by baseline differences in the R/T state, which precede activation of cells. The next goal was to confirm the utility of resistance and tolerance in a practical setting. To that end. a generic collection of gene biomarkers for resistance and tolerance was first identified (including both positive and negative markers for each program, denoted as R+, R-, T+ and T-markers; Table 1 and FIG. 7) and validated that the levels of these markers persist for at least several weeks in the blood of healthy human subjects (FIG. 16D-16E). In a human IAV-infection cohort, it was found that resistance and tolerance markers in blood (before infection) successfully predict susceptibility to IAV infection (FIGS. 15E left, 15F, e.g., R-markers p<104, Fisher's combined p-values across the Pearson's p-values of these markers). The same pattern appears when using the markers in resting MFs or in lungs before infection (FIG. 15E, middle and right); in all cases, the correlations of positive and negative markers are in opposing directions, as expected. Thus, resistance and tolerance levels can be practically measured in blood samples to predict disease severity.

Given the relevance of the R/T state to immune-related disease, it was further hypothesized that the R/T state within tumors may predict the survival of cancer patients. To test this, the inventors relied on the PRECOG database (Gentles, A. J., et al. (2015). Nat Med 21, 938-945), which provides the prognostic p-values of each gene in multiple malignancies (i.e., for each malignancy, the associations between each gene with tumor survival). It was found that R and T markers are prognostic for five and four tumors, respectively (Fisher combined p<10⁻⁷ and an average prognostic p<0.05; FIG. 15G). In all of these cases, and as expected, the negative and positive markers have an opposite effect on survival (FIG. 15H-151). Importantly, program T has a tumor-specific direction of effect: T markers are an adversely prognostic set for multiple myeloma and a favorable prognostic set for glioma, breast cancer and chronic lymphocytic leukemia (CLL); this is in contrast to program R that is adversely prognostic to all five tumors (FIG. 15H). Compared to gene sets of immune activation that were previously reported to be related to cancer, the inventors found that the R and T markers are the best prognostic sets across multiple human cancers (Table 3). Collectively, the analysis indicates that resistance and tolerance are significant, yet distinct, predictors of survival in cancer patients, emphasizing the critical role of the R/T state in tumor progression and prognosis. Overall, it was found that both resistance and tolerance have significant but distinct contribution to disease. This emphasizes the broad implications of an independent assessment of resistance and tolerance states.

Table 3: Prediction across human cancers. Average prognostic values across human cancers and gene signatures. For each set of genes (column 1), indicated the two opposing subsets that were used (column 2), the original publication (columns 3,4), and the average prognostic values across cancer types (columns 5-14). The average prognostic values were calculated across the log 10 p-values of all genes in the gene set, assuming opposing direction of effects for genes from the two opposing subsets (the p-value of each gene in each human cancer was obtained from the PRECOG database (Gentles et al., 2015). Out of 39 human cancers in PRECOG, included are those cancers in which at least one gene set obtained an average prognostic value that is better than p<0.05 (−log p>1.3); melanoma was excluded from the analysis as many of the compared gene sets were tailored to this particular cancer. Significant prognostic gene sets (using Fisher test, p<10⁻⁷) are indicated in gray or orange. The top prognostic gene set for each cancer is indicated in orange. Column 15 reports the number of human cancers in which a gene set outperforms all other gene sets, highlighting R as the best prognostic gene set (best in 5 of 11 cancer types) and T as a second-best prognostic gene set (best in 2 of 11 cancer types).

TABLE 3

| | | | | | Brain | | | | | | Hematopoietic | Lung | | |
|---|---|---|---|---|---|---|---|---|---|---|---|---|---|---|
| | Two opposing | Reference and | | Brain cancer | cancer | Brain cancer | Breast | Germ cell | Hematopoietic | Hematopoietic | cancer | cancer | Ovarian | Top |
| Gene set | subsets | pubmed ID | | Astrocytoma | Glioma | Neuroblastoma | cancer | tumors | cancer CLL | cancerDLBCL | Multiple myeloma | ADENO | cancer | gene set |
| R markers | R+ and R− subsets | | | 1.72 | 3.29 | 5.81 | 2.43 | 0.77 | 0.89 | 0.82 | 1.26 | 2.76 | 1.19 | 5 |
| T markers | T+ and T− subsets | | | 1.14 | 2.55 | 0.10 | 1.48 | 0.59 | 1.30 | 0.85 | 1.72 | 0.61 | 0.88 | 2 |
| Nivolumab (molecular) | Up-regulated and | Riaz et al., | 290 | 0.86 | 1.32 | 2.21 | 0.83 | 1.35 | 0.80 | 1.03 | 0.56 | 1.62 | 1.03 | 0 |
| resistant melanoma | down-regulated genes | Cell 2017 | 331 | | | | | | | | | | | |
| | | | 30 | | | | | | | | | | | |
| Nivolumab resistant | Up-regulated and down- | Riaz et al., | 290 | 0.72 | 0.78 | 2.16 | 0.95 | 0.35 | 0.59 | 0.83 | 0.63 | 0.59 | 0.98 | 0 |
| melanoma | regulated genes | Cell 2017 | 331 | | | | | | | | | | | |
| | | | 30 | | | | | | | | | | | |
| On Nivolumab | Up-regulated and down- | Riaz et al., | 290 | 0.72 | 1.32 | 3.12 | 0.81 | 1.98 | 1.12 | 1.10 | 0.93 | 0.97 | 2.05 | 2 |
| | regulated genes | Cell 2017 | 331 | | | | | | | | | | | |
| | | | 30 | | | | | | | | | | | |
| Responders on | Up-regulated and down- | Riaz et al., | 290 | 0.85 | 1.42 | 5.46 | 2.26 | 1.43 | 0.65 | 0.97 | 1.10 | 1.47 | 1.20 | 0 |
| Nivolumab | regulated genes | Cell 2017 | 331 | | | | | | | | | | | |
| | | | 30 | | | | | | | | | | | |
| Anti-PD-1 resistant | Up-regulated and down- | Hugo et al., | 269 | 1.27 | 1.89 | 2.03 | 1.05 | 0.64 | 0.69 | 0.88 | 0.64 | 1.28 | 1.30 | 0 |
| melanoma . | regulated genes | Cell 2016 | 974 | | | | | | | | | | | |
| | | | 80 | | | | | | | | | | | |
| MAPKi resistant | Up-regulated and down- | Hugo et al., | 263 | 1.03 | 0.98 | 2.79 | 1.39 | 0.78 | 0.61 | 0.78 | 0.68 | 0.88 | 0.72 | 0 |
| melanoma | regulated genes | Cell 2015 | 599 | | | | | | | | | | | |
| | | | 85 | | | | | | | | | | | |
| Mouse activation of | Top genes from LPS- | Buscher | 287 | 0.83 | 1.39 | 3.10 | 0.99 | 0.80 | 0.75 | 0.66 | 0.66 | 1.00 | 0.67 | 0 |
| MFs | responder and LPS non- | et al., Nat. | 371 | | | | | | | | | | | |
| | responder MFs | Comm 2017 | 75 | | | | | | | | | | | |
| Mouse bone- | LPS + IFNy 24h (M1), | Jablonski | 266 | 0.78 | 1.10 | 1.54 | 0.71 | 0.64 | 0.96 | 1.45 | 0.58 | 0.71 | 0.49 | 1 |
| marrow derived | IL-4 (M2) | KA et al., | 996 | | | | | | | | | | | |
| MFs human CD14+ | | PLOS one | 15 | | | | | | | | | | | |
| monocyte-derived | | 2015 | | | | | | | | | | | | |
| MFs, M-CSF | LPS + IFNy or TNFa | Becker et al., | 263 | 0.67 | 1.02 | 1.42 | 0.84 | 0.43 | 0.80 | 0.90 | 0.62 | 1.40 | 1.62 | 0 |
| polarized | (M1) ,IL-4 or IL-13 (M2) | Sci. Rep. | 028 | | | | | | | | | | | |
| | | 2015 | 99 | | | | | | | | | | | |

### Example 8

The baseline T and R states in peritoneal MFs is associated with pathophysiology in response to infection and injury

The quantitative T/R metrics was next used to test how baseline levels of T and R are linked to future disease. Specifically, the inventors used data from a different cohort [Orozco, L. D. et al. Cell 151, 658-670 (2012)] (the BXD mouse strains [Peirce, J. L., Lu, L., Gu, J., Silver, L. M. & Williams, R. W. BMC Genet 5, 7-7 (2004)]), which allowed us to compare between the basal T/R levels in peritoneal MFs from healthy individuals and phenotypes in these strains following injury (abiotic stimulus) and infection (biotic stimulus).

The inventors first investigated the programs in the context of hepatic injury. Hepatic injury may lead to two different outcomes: mild injury typically leads to tissue repair (wound healing), but a repetitive injury or chronic wound may lead to fibrosis, with important roles of MFs in these processes [Adler, M. et al. iScience 23, 100841-100841 (2020)]. To explore these distinct response states, a collection of tissue-pathophysiology markers following hepatic injury across the BXD strains were analyzed, including phenotypes of an aberrant wound healing (tissue damage) following a mild, transient hepatic injury, as well as fibrosis susceptibility markers following profibrotic/repetitive hepatic injury. As shown by FIGS. 17A, 17B, a high baseline T level of MFs is associated with a beneficial state (lesser tissue damage following a transient, mild injury, p<104, t-test), but is also associated with an unfavorable state in fibrosis following a profibrotic/repetitive injury (p<10⁻⁶, t-test). Thus, the baseline state of program T in peritoneal MFs is associated with tissue pathophysiology following hepatic injury

### Example 9

Assessing the functional roles of Arhgdia

The inventors next directly assessed the predicted role of Arhgdia, one of the T markers, in the regulation of the T program. Therefore, Arhgdia was depleted in two mouse epithelial cells (LET1 and MLE-12) by CRISPR-Cas9 editing system, and then the effect of this depletion was tested on the cellular response to infection. Control (non-targeting sgRNA) and Arhgdia-depleted (Arhgdia-targeted sgRNA) cells, with and without IAV infection, were analyzed. As confirmed in FIG. 18A, the parental LET1 and MLE-12 cells express Arhgdia, while the CRISPR/Cas9-edited cells do not.

To assess the effect of Arhgdia on disease severity, cell damage (using the percentages of cell death) and viral burden (quantified in infected cells by the fluorescent of the mNeonGreen marker, expressed by a recombinant PR8 virus, named ‘PR8-mNeonGreen’), were measured; both recorded by FACS analysis of IAV-infected cells 24 h post infection. Notably, it was found that knockout of Arhgdia in both LET1 and MLE-12 cells was beneficial using sgRNA #1 (also denoted by SEQ ID NO: 37), leading to reduced viral protein expression (FIGS. 18B, 18C, 18D) and reduced cell death (FIG. 18E, 18F). Similar observations (albeit with smaller effects) obtained using a different sgRNA against Arhgdia (Arhgdia sgRNA #2, (also denoted by SEQ ID NO: 38). The independent knock-out cell line of Arhgdia in LET1 cells showed a reduced IAV expression compared to control cells, 24 hr post infection (FIG. 18A, 18C).

To further support Arhgdia function in IAV expression and cell survival, a LET1 cell line with an inducible expression of Arhgdia was next generated. This was done by infecting LET1 cells with a lentivector expressing the Arhgdia cDNA under the control of doxycycline-inducible promoter (named ‘Arhgdia/Tet-on LET1’). An immunoblot analysis of IAV nucleoprotein (NP) expression in lysates of IAV-infected cells, with or without doxycycline addition, showed an increase in NP expression when Arhgdia expression was induced (FIG. 19A). FACS analysis further supported these findings: Arhgdia over-expression resulted in a higher viral load (deduced from the expression of the mNeongreen marker from the PR8-mNeongreen virus) (FIG. 19B). Remarkably, the inventors found that over-expression of Arhgdia induced an extensive cytopathic effect (CPE) upon IAV infection (FIG. 19C).

The inventors next asked whether the reestablishment of Arhgdia expression in knockout cells would affect viral expression (an ‘add-back’ experiment). To this end, a pCW lentivector expressing the murine Arhgdia cDNA under the control of a doxycycline-regulated promoter was created. Moreover, silent mutations were introduced in the sgRNA #1 target sequence (also denoted by SEQ ID NO: 37) that render the resulting Arhgdia cDNA resistant to the recognition of the CRISPR/Cas9 system, guided by this sgRNA. The resulting pCW-Arhgdia lentivector was then transduced into the Let1 cell clone, in which Arhgdia expression was depleted by sgRNA #1, resulting in cells termed ‘Arhgdia sgRNA #1 reversed’. As shown by FIG. 19D, the addback experiment resulted in restoration of Arhgdia expression (in a doxycycline-regulated manner), and in increased viral protein expression (NP) in Arhgdia sgRNA #1 reversed cells, compared to depleted cells. Similar observations obtained using PR8-mNeongreen, it was found that ‘Arhgdia sgRNA #1 reversed’ cells showed increasing IAV levels, compared to Arhgdia-depleted cells at 24h post infection. These results clearly demonstrate that the defects in viral expression were specifically linked to reduced Arhgdia levels

(FIG. 19E). Altogether, knocking-down Arhgdia expression resulted in lower IAV infection levels and reduced cell death, while Arhgdia over-expression resulted in the opposite effects: higher IA V infection and increased cell death.

Next, using transcriptomes, the effect of Arhgdia expression on disease tolerance and resistance programs was next evaluated at early stages of infection (2-6h post infection)-a window of time in which viral RNA is readily detectable (FIG. 20A) but CPE is not evident. The inventors selected this period for analyses to avoid the effects of cellular damage on the overall gene-expression state. The transcriptome analyzes provided several lines of evidence to support a positive effect of Arhgdia on the disease tolerance state (rather than the resistance state). First, Arhgdia's responding genes were enriched with disease tolerance-associated functions such as wound healing, ECM and hepatic fibrosis (hyper-geometric test p<10-11, 106, 10-28, respectively). Second, the effect of Arhgdia on the expression of genes strongly resembled the associations of genes with disease tolerance program (denoted as T) (e.g., Pearson's r=0.53, p<10-230; FIG. 20B) rather than resistance program (denoted as R) (r=−0.1, FIG. 20C). Indeed, depletion of Arhgdia led to a significant, greater-than-normal downregulation of T levels, but the effect on R levels was only weak (p<0.006 for T, p >0.05 for R, paired t-test; FIG. 20D).

Finally, the ‘relative tissue damage’, defined at the cellular level as the slope of cell death against viral burden was calculated. As shown in FIG. 20E, the presence of Arhgdia is linked to a higher relative tissue damage compared to control cells (F test p<0.015;), consistent with the observation that higher baseline T levels are linked to a lower ability to tolerate future in vivo infections (see Example 7). Collectively, these results indicate that the effect of Arhgdia-depletion is beneficial to IAV infection, leading to reduced infection severity and improved ability to tolerate infection. Moreover, these observations provide additional support for the inventor's conclusion that disease tolerance program (and Arhgdia as one of its regulators) is associated with alterations in the phenotype of disease tolerance during IAV infection. Still further, these results clearly establish the feasibility of using the T and/or the R biomarkers of the present disclosure as targets in mediating modulation of the T and/or R state of a subject, thereby manipulating the disease outcome in a subject in need thereof.

### Example 10

Potential regulators of the resistance programs

To identify regulators that may induce disease tolerance and resistance activation, possible markers of these programs were ranked. The ranking was based on consistent associations of regulators with the disease tolerance or resistance programs across multiple cell types and contexts (FIG. 21). Briefly, an unbiased approach was used to rank candidate regulators. The ranking was performed separately for each program. Two selection criteria were first applied: (1) Whether the gene is tightly associated with the program (absolute r >0.8) in at least one cell type using at least one cohort (either healthy individuals or one of five autoimmune-disease cohorts; Ota et al., (2021) Cell 184, 3006-3021.e17). (2) Whether the gene is annotated as a regulator, using annotations of “signaling molecules” “receptor”, “transcription factor” or “transcription regulator” in the Ingenuity Knowledge Base. Next, genes were ranked by their average associations to disease tolerance or resistance across autoimmune-disease cohorts. As expected, validated IAV restriction factors that have an established effect on the virus replication cycle (Villalón-Letelier et al., (2017) Infection. Viruses 9, 376), obtained high ranking in the resistance program (p<0.04, Wilcoxon test; FIG. 21). These findings provide additional support for the hypothesis that the molecular resistance program is linked to resistance functions.

These results therefore confirm that also the resistance biomarkers of the present disclosure can serve as targets for modulation, for manipulating the T and R levels and thereby the disease outcome.

### Example 11

The functions of resistance and disease tolerance in health and disease

Historically, mechanisms of the immune response to infections have been classified by whether they function to eliminate the pathogen, referred to as resistance mechanisms, or by whether they serve to maintain vital homeostasis and tissue recovery even in the presence of infection, the so-called tolerance mechanisms. This classification has been the basis for the idea that an effective immune defense requires the activity of both resistance and tolerance [1, 2, 6, 7]. As the two types of mechanisms operate together in response to infections, it has been challenging to identify distinct molecular programs for resistance and tolerance. Relying on the wide variation among mouse strains, this study reveals two transcriptional programs that have extensive pleiotropic effects on gene expression during IAV infection. Using functional analysis, the inventors propose that one program (R) is a resistance response against infections and the other program (T) underlies the tolerance response. Both programs were indeed activated in response to biotic stimuli and function together in cytokine responses and inflammation, supporting their key roles in immune defense. By integrating data from in vivo and in vitro studies, in both human and mouse, the inventors showed that both programs are robustly detected at the cell-autonomous levels across various cell types. By analyzing responses to infections, wound healing, autoimmunity, cancer and a healthy steady state, it was found that each of these conditions is shaped by the combined contributions of resistance and tolerance-indicating that these two programs are basal constituents of many tissue-immune states. Thus, this study defines, for the first time, the molecular programs that underlie the previously described classification of resistance and tolerance.

This study provides new insights into the organization and functions of resistance and tolerance. It was found that resistance and tolerance are largely antagonistic, under both steady state and inflammatory conditions, and further identified novel functionalities that are linked to resistance, tolerance, or both (FIG. 22). The identified functions shed light on the various possible roles of the R/T state. For instance, focusing on the wide diversity of T levels under inflammatory conditions, the high protein production in low-tolerance inflammation potentially allows a robust host response but at the risk of high levels of pathogen production, hyperinflammation, and associated systemic deleterious effects. In contrast, the low protein production in high-tolerance inflammation has the benefit of reducing viral replication at the risk of a limited host response.

An important finding is that resistance and tolerance programs are predictive and prognostic in validation cohorts, both in mouse and human. Particularly, relations of the baseline resistance and tolerance levels was demonstrated with clinical susceptibility to infectious diseases, autoimmune diseases and cancer survival. Interestingly, it was found that the two programs have distinct (and even antagonistic) contribution to disease-supporting the uncoupling between the two programs, and further indicating that targeting one program while preserving the activity of the other program warrants evaluation as a personalized therapeutic approach.

A striking finding of this study is that the R/T state has an antagonistic effect on autoimmune and infectious diseases: increased resistance and reduced tolerance are linked to a higher level of autoimmune markers and reduced susceptibility to infectious diseases (FIG. 22). It was demonstrated this tradeoff for viral, bacterial and fungal infections, as well as autoimmune markers such as anti-DNA antibodies and rheumatoid factors. It is speculated that the relatively high baseline activity of tolerance interferes with the activity of resistance, thereby reducing the risk of autoimmunity but potentially leading to a limited defense against infectious diseases. According to this logic, the counterbalance between resistance and tolerance is a central component of immune homeostasis in health and disease. Importantly, these findings indicate that individuals with the intermediate baseline state of mid-T/mid-R may benefit from the compromise between opposing forces, consistent with the high prevalence of the intermediate state in healthy subjects.

The correlation between a strong baseline resistance and a mild course of various infections implies a beneficial ‘hit hard, hit quickly’ model of host defense. It has been long argued whether death resulting from dysregulation of inflammation in sepsis is due to a poor initial immune response followed by high pathogen load and secondary hyper-inflammation or, alternatively, it is due to a disproportionately strong initial immune response against a low pathogen load that leads to systemic inadequate responses. These findings argue for the former, in line with studies of bacterial sepsis (Westendorp, R. G., et al. (1997). The Lancet 349, 170-173) and respiratory infections (Bradley, K. C., et al. (2019) Cell Reports 28, 245-256.e4; Graham, J. B., et al. (2021) PLOS Pathog 17, e1009287-e1009287; Lee, J. S., et al. (2020) Nature Reviews Immunology 20, 585-586) and consistent with the efficacy of approaches that boost the baseline/early resistance such as vaccines (O'Neill, L.A.J., et al. (2020) Nat Rev Immunol 20, 335-337; Sánchez-Ramón, et al. (2018). Frontiers in Immunology 9, 2936).

The data imply that a balanced immune response is maintained through four key layers of control. The first involves opposing regulation on the two arms of each program. The second results from the antagonistic relationships between the resistance and tolerance programs. Third, there are antagonistic effects of the resistance and tolerance programs on the same biological process—for instance, opposite directions of relations with infectious diseases, autoimmunity, and lipid-metabolism genes. Finally, for each given process, there is a tight coordination of positive and negative regulators

Overall, this study provides (1) a molecular signature for resistance and tolerance; (2) a framework to understand tissue-immune states, in health and disease, in the context of resistance and tolerance; and demonstrates (3) the clinical usefulness of resistance and tolerance quantification in prediction of complex diseases.

### Example 12

Generic resistance and tolerance markers

Using information extracted from all datasets in the present study (all datasets in Example 3), the inventors searched for generic resistance and tolerance markers. Based on the consistency of markers across datasets (FIG. 7A), 205 markers emerged as valuable markers of (i) resistance (44 positive, 37 negative markers), (ii) tolerance (38 positive, 36 negative markers), (iii) markers for the common combination of resistance and tolerance: 25 marker that are positive for R and negative for T, 25 marker that is negative for resistance and positive for tolerance. The list of markers, together with their relevant program and direction are detailed below in Table 4.

TABLE 4
Generic markers of resistance and disease tolerance.

| Marker | marker | | |
|---|---|---|---|
| (mouse name) | (human name) | program | direction |
| Adcy9 | ADCY9 | R− | Negative |
| Add1 | ADD1 | R− | Negative |
| Adipor1 | ADIPOR1 | R− | Negative |
| Arhgef18 | ARHGEF18 | R− | Negative |
| Brd3 | BRD3 | R− | Negative |
| Ctdsp2 | CTDSP2 | R− | Negative |
| Ctnnbip1 | CTNNBIP1 | R− | Negative |
| Dhrs3 | DHRS3 | R− | Negative |
| Erbb3 | ERBB3 | R− | Negative |
| Hagh | HAGH | R− | Negative |
| Hectd3 | HECTD3 | R− | Negative |
| Ldb1 | LDB1 | R− | Negative |
| Lrp6 | LRP6 | R− | Negative |
| Med25 | MED25 | R− | Negative |
| Mef2d | MEF2D | R− | Negative |
| Mink1 | MINK1 | R− | Negative |
| Mlxip | MLXIP | R− | Negative |
| Mtss1 | MTSS1 | R− | Negative |
| Mxd4 | MXD4 | R− | Negative |
| Mxi1 | MXI1 | R− | Negative |
| Nav2 | NAV2 | R− | Negative |
| Pdxk | PDXK | R− | Negative |
| Pik3r2 | PIK3R2 | R− | Negative |
| Pla2g6 | PLA2G6 | R− | Negative |
| Ralbp1 | RALBP1 | R− | Negative |
| Rxra | RXRA | R− | Negative |
| Sh2b1 | SH2B1 | R− | Negative |
| Sorbs3 | SORBS3 | R− | Negative |
| Ssbp3 | SSBP3 | R− | Negative |
| Tacc2 | TACC2 | R− | Negative |
| Tns1 | TNS1 | R− | Negative |
| Tpcn1 | TPCN1 | R− | Negative |
| Ulk1 | ULK1 | R− | Negative |
| Wdr45 | WDR45 | R− | Negative |
| Xpc | XPC | R− | Negative |
| Zfp395 | ZNF395 | R− | Negative |
| Zmym3 | ZMYM3 | R− | Negative |
| Abhd4 | ABHD4 | R−T+ | R-negative, T-positive |
| Ap2m1 | AP2M1 | R−T+ | R-negative, T-positive |
| Atp13a2 | ATP13A2 | R−T+ | R-negative, T-positive |
| Btbd2 | BTBD2 | R−T+ | R-negative, T-positive |
| Cic | CIC | R−T+ | R-negative, T-positive |
| Ciz1 | CIZ1 | R−T+ | R-negative, T-positive |
| Crat | CRAT | R−T+ | R-negative, T-positive |
| Dyrk1b | DYRK1B | R−T+ | R-negative, T-positive |
| Fkbp8 | FKBP8 | R−T+ | R-negative, T-positive |
| Grina | GRINA | R−T+ | R-negative, T-positive |
| Gsk3a | GSK3A | R−T+ | R-negative, T-positive |
| Irak1 | IRAK1 | R−T+ | R-negative, T-positive |
| Lyl1 | LYL1 | R−T+ | R-negative, T-positive |
| Map2k2 | MAP2K2 | R−T+ | R-negative, T-positive |
| Med25 | MED25 | R−T+ | R-negative, T-positive |
| Mt3 | MT3 | R−T+ | R-negative, T-positive |
| Pigq | PIGQ | R−T+ | R-negative, T-positive |
| Pnpla2 | PNPLA2 | R−T+ | R-negative, T-positive |
| Rusc2 | RUSC2 | R−T+ | R-negative, T-positive |
| Slc6a8 | SLC6A8 | R−T+ | R-negative, T-positive |
| Sorbs3 | SORBS3 | R−T+ | R-negative, T-positive |
| Tbc1d17 | TBC1D17 | R−T+ | R-negative, T-positive |
| Tmcc2 | TMCC2 | R-T+ | R-negative, T-positive |
| Wbp2 | WBP2 | R-T+ | R-negative, T-positive |
| Zfp574 | ZNF574 | R-T+ | R-negative, T-positive |
| Arf4 | ARF4 | R+ | Positive |
| B2m | B2M | R+ | Positive |
| Clic4 | CLIC4 | R+ | Positive |
| Eif4e | EIF4E | R+ | Positive |
| Fndc3a | FNDC3A | R+ | Positive |
| Gch1 | GCH1 | R+ | Positive |
| Glrx | GLRX | R+ | Positive |
| Gnl3 | GNL3 | R+ | Positive |
| Gtf2b | GTF2B | R+ | Positive |
| Hat1 | HAT1 | R+ | Positive |
| Hax1 | HAX1 | R+ | Positive |
| Ifi35 | IFI35 | R+ | Positive |
| Ifih1 | IFIH1 | R+ | Positive |
| Ifit2 | IFIT2 | R+ | Positive |
| Ifit3 | IFIT3 | R+ | Positive |
| Il15 | IL15 | R+ | Positive |
| Il15ra | IL15RA | R+ | Positive |
| Ints12 | INTS12 | R+ | Positive |
| Isg20 | ISG20 | R+ | Positive |
| Jak2 | JAK2 | R+ | Positive |
| Katna1 | KATNA1 | R+ | Positive |
| Mthfd2 | MTHFD2 | R+ | Positive |
| Myd88 | MYD88 | R+ | Positive |
| Nmi | NMI | R+ | Positive |
| Pla2g4a | PLA2G4A | R+ | Positive |
| Pop4 | POP4 | R+ | Positive |
| Ppp2ca | PPP2CA | R+ | Positive |
| Psma5 | PSMA5 | R+ | Positive |
| Psmb7 | PSMB7 | R+ | Positive |
| Psmb8 | PSMB8 | R+ | Positive |
| Psmd12 | PSMD12 | R+ | Positive |
| Psmd14 | PSMD14 | R+ | Positive |
| Psme2 | PSME2 | R+ | Positive |
| Ptpn2 | PTPN2 | R+ | Positive |
| Ptx3 | PTX3 | R+ | Positive |
| Sarla | SARIA | R+ | Positive |
| Sat1 | SAT1 | R+ | Positive |
| Tap1 | TAP1 | R+ | Positive |
| Tdrd7 | TDRD7 | R+ | Positive |
| Timm17a | TIMM17A | R+ | Positive |
| Tlk2 | TLK2 | R+ | Positive |
| Torlaip1 | TOR1AIP1 | R+ | Positive |
| Ube2n | UBE2N | R+ | Positive |
| Upp1 | UPP1 | R+ | Positive |
| Abce1 | ABCE1 | R+T− | R-positive, T-negative |
| Chuk | CHUK | R+T− | R-positive, T-negative |
| Cops5 | COPS5 | R+T− | R-positive, T-negative |
| Dhx15 | DHX15 | R+T− | R-positive, T-negative |
| Dnttip2 | DNTTIP2 | R+T− | R-positive, T-negative |
| Eef1e1 | EEF1E1 | R+T− | R-positive, T-negative |
| Gtf2f2 | GTF2F2 | R+T− | R-positive, T-negative |
| Ibtk | IBTK | R+T− | R-positive, T-negative |
| Magoh | MAGOH | R+T− | R-positive, T-negative |
| Mapk6 | MAPK6 | R+T− | R-positive, T-negative |
| Mbip | MBIP | R+T− | R-positive, T-negative |
| Mrpl3 | MRPL3 | R+T− | R-positive, T-negative |
| Nfyb | NFYB | R+T− | R-positive, T-negative |
| Pnrc2 | PNRC2 | R+T− | R-positive, T-negative |
| Psma2 | PSMA2 | R+T− | R-positive, T-negative |
| Psmc6 | PSMC6 | R+T− | R-positive, T-negative |
| Rbbp8 | RBBP8 | R+T− | R-positive, T-negative |
| Rbm7 | RBM7 | R+T− | R-positive, T-negative |
| Scfd1 | SCFD1 | R+T− | R-positive, T-negative |
| Taf7 | TAF7 | R+T− | R-positive, T-negative |
| Tmem168 | TMEM168 | R+T− | R-positive, T-negative |
| Twf1 | TWF1 | R+T− | R-positive, T-negative |
| Usp16 | USP16 | R+T− | R-positive, T-negative |
| Vps54 | VPS54 | R+T− | R-positive, T-negative |
| Yme1l1 | YME1L1 | R+T− | R-positive, T-negative |
| Actr6 | ACTR6 | T− | Negative |
| Arl1 | ARL1 | T− | Negative |
| Bmi1 | BMI1 | T− | Negative |
| Capn7 | CAPN7 | T− | Negative |
| Cetn3 | CETN3 | T− | Negative |
| Cnot6 | CNOT6 | T− | Negative |
| Copb1 | COPB1 | T− | Negative |
| Cops2 | COPS2 | T− | Negative |
| Crbn | CRBN | T− | Negative |
| Cul3 | CUL3 | T− | Negative |
| Ddx46 | DDX46 | T− | Negative |
| Dek | DEK | T− | Negative |
| Fbxo3 | FBXO3 | T− | Negative |
| Gmfb | GMFB | T− | Negative |
| Hltf | HLTF | T− | Negative |
| Krcc1 | KRCC1 | T− | Negative |
| Lmbrd1 | LMBRD1 | T− | Negative |
| Mtx2 | MTX2 | T− | Negative |
| Paip1 | PAIP1 | T− | Negative |
| Ppp3cb | PPP3CB | T− | Negative |
| Rad17 | RAD17 | T− | Negative |
| Rasa1 | RASA1 | T− | Negative |
| Rbm39 | RBM39 | T− | Negative |
| Rnf138 | RNF138 | T− | Negative |
| Sacm1l | SACMIL | T− | Negative |
| Senp6 | SENP6 | T− | Negative |
| Serinc1 | SERINC1 | T− | Negative |
| Serp1 | SERP1 | T− | Negative |
| Slc35a3 | SLC35A3 | T− | Negative |
| Spast | SPAST | T− | Negative |
| Tbc1d15 | TBC1D15 | T− | Negative |
| Tm9sf3 | TM9SF3 | T− | Negative |
| Tmco1 | TMCO1 | T− | Negative |
| Top2b | TOP2B | T− | Negative |
| Txndc9 | TXNDC9 | T− | Negative |
| Vbp1 | VBP1 | T− | Negative |
| Ap1b1 | AP1B1 | T+ | Positive |
| Ap2s1 | AP2S1 | T+ | Positive |
| Arhgdia | ARHGDIA | T+ | Positive |
| Atp13a1 | ATP13A1 | T+ | Positive |
| Atp6v0d1 | ATP6V0D1 | T+ | Positive |
| Brd4 | BRD4 | T+ | Positive |
| Capns1 | CAPNS1 | T+ | Positive |
| Carm1 | CARM1 | T+ | Positive |
| Ciz1 | CIZ1 | T+ | Positive |
| Clcn7 | CLCN7 | T+ | Positive |
| Clptm1 | CLPTM1 | T+ | Positive |
| Dpm2 | DPM2 | T+ | Positive |
| Flot2 | FLOT2 | T+ | Positive |
| Gnb2 | GNB2 | T+ | Positive |
| Hgs | HGS | T+ | Positive |
| Hspbp1 | HSPBP1 | T+ | Positive |
| Lmna | LMNA | T+ | Positive |
| Ltbr | LTBR | T+ | Positive |
| Map3k11 | MAP3K11 | T+ | Positive |
| Maz | MAZ | T+ | Positive |
| Mbd3 | MBD3 | T+ | Positive |
| Mfsd5 | MFSD5 | T+ | Positive |
| Myo9b | MYO9B | T+ | Positive |
| Nucb1 | NUCB1 | T+ | Positive |
| Pld3 | PLD3 | T+ | Positive |
| Prkcsh | PRKCSH | T+ | Positive |
| Ranbp3 | RANBP3 | T+ | Positive |
| Scamp2 | SCAMP2 | T+ | Positive |
| Scand1 | SCAND1 | T+ | Positive |
| Sgta | SGTA | T+ | Positive |
| Slc4a2 | SLC4A2 | T+ | Positive |
| Sphk2 | SPHK2 | T+ | Positive |
| Strn4 | STRN4 | T+ | Positive |
| Stxbp2 | STXBP2 | T+ | Positive |
| Tgfb1 | TGFB1 | T+ | Positive |
| Vac14 | VAC14 | T+ | Positive |
| Yipf3 | YIPF3 | T+ | Positive |
| Zyx | ZYX | T+ | Positive |

Top generic resistance and tolerance markers

Using information from all datasets in the present study, 18 markers emerge as the top markers of (i) resistance (5 positive, 3 negative markers), (ii) tolerance (4 positive, 4 negative markers), (iii) markers for the common combination of resistance and tolerance: a marker that is positive for R and negative for T, and a marker that is negative for resistance and positive for tolerance.

The list of markers, together with their relevant program and direction are detailed below in Table 5.

TABLE 5
Top generic markers of resistance and disease tolerance.

| Marker | marker (human | | |
|---|---|---|---|
| (mouse name) | name) | program | direction |
| Serinc 1 | SERINC1 | T− | Negative |
| Arl1 | ARL1 | T− | Negative |
| Cops2 | COPS2 | T− | Negative |
| Crbn | CRBN | T− | Negative |
| Map2k2 | MAP2K2 | T+ | Positive |
| Arhgdia | ARHGDIA | T+ | Positive |
| Grina | GRINA | T+ | Positive |
| Stxbp2 | STXBP2 | T+ | Positive |
| Mxi1 | MXI1 | R− | Negative |
| Zfp395 | ZNF395 | R− | Negative |
| Xpc | XPC | R− | Negative |
| Mthfd2 | MTHFD2 | R+ | Positive |
| Psme2 | PSME2 | R+ | Positive |
| Jak2 | JAK2 | R+ | Positive |
| Ints12 | INTS12 | R+ | Positive |
| Psmb7 | PSMB7 | R+ | Positive |
| Rbm7 | RBM7 | R+T− | R-positive, T-negative |
| Slc6a8 | SLC6A8 | R−T+ | R-negative, T-positive |

### Example 13

Considering resistance and tolerance in prioritization of drugs

Using the drug screen of the connectivity map [Aravind Subramanian et al., Cell 171 (6): 1437-1452 (2017)], the effect of each drug on resistance and tolerance was next analyzed in at least 6 cell lines of different tissues. Identification of multiple drugs that display an effect on resistance/tolerance-related diseases allows (i) repurposing of drugs, and (ii) personalized prioritization of drugs. For instance, the screen tested three P glycoprotein inhibitors: zosuquidar, elacridar, and dofequidar. Interestingly, the three drugs led to increased tolerance in all tissues under study, but different drugs have opposing effect on resistance. Particularly, zosuquidar leads to reduced resistance whereas elacridar and dofequidar lead to increasing resistance (Table 6). Thus, for specific disease in which P glycoprotein inhibitor is required, it is possible to prioritize drugs for each patient based on its baseline combination of resistance and tolerance state.

This example therefore establishes the feasibility of using the disclosed methods for personalized medicine.

TABLE 6
Effect of P glycoprotein inhibitors on resistance and disease
tolerance across tissues. Shown are the effect of each drug (column 1),
in each tissue (column 2), on the levels of resistance and
tolerance (column 3, signed −log p-values, t-test). Positive/negative
signs of −log p-values indicate increasing/decreasing levels of resistance
or tolerance following the drug treatment. Phase 3:
zosuquidar and dofequidar. Phase 1: elacridar.

| | | Effect on resistance | Effect on |
|---|---|---|---|
| Drug | Tissue | level | tolerance level |
| zosuquidar | breast | 0.13 | 11.19 |
| zosuquidar | prostate | −2.05 | 5.97 |
| zosuquidar | large | −7.65 | 5.89 |
| | intestine | | |
| zosuquidar | pancreas | −2.82 | 5.81 |
| zosuquidar | kidney | −2.54 | 5.15 |
| zosuquidar | skin | −6.00 | 2.91 |
| elacridar | large | 10.56 | 13.47 |
| | intestine | | |
| elacridar | kidney | 6.87 | 8.48 |
| elacridar | skin | 4.66 | 2.77 |
| elacridar | breast | 7.26 | 1.84 |
| elacridar | pancreas | 2.33 | −0.10 |
| elacridar | prostate | 4.37 | −1.43 |
| dofequidar | breast | 0.89 | 6.29 |
| dofequidar | large | 9.14 | 6.03 |
| | intestine | | |
| dofequidar | pancreas | 5.84 | 2.60 |
| dofequidar | kidney | 7.33 | 2.40 |
| dofequidar | prostate | 3.47 | 1.54 |
| dofequidar | skin | 9.17 | 0.89 |

### Example 14

Prioritization of disease-specific mechanisms whose effect on disease is independent of the resistance/disease-tolerance state

The framework can be used to identify biomarkers for disease that are independent of variation in resistance and tolerance. Specifically, a marker for a certain disease typically has a high ‘association score’ based on comparison of disease to healthy subjects (using computational models such as multivariate regression and linear mixed models). As the healthy and disease groups do not necessarily have matching characteristics, it is important to consider potential confounding factors (e.g., age, gender, BMI, smoking, ethnicity) as covariates within the calculation of the association scores. In a standard association score, the association score accounts for standard covariates such as gender, age, and BMI (a ‘conventional association score’). The framework disclosed by the present invention allows the use of resistance and tolerance states as additional covariates, thereby revealing disease biomarkers for which the associations are independent of the resistance and tolerance state (a ‘resistance/tolerance-independent association scores’). Thus, the framework can be applied to prioritize particular disease-associated factors in which the association is independent of variation in the resistance and tolerance states. Such prioritization strategy has potential to guide development of effective clinical diagnostics and selection of drug targets.

1-51. (canceled)

52. A method of treating and prognosing a subject by determining the susceptibility of a subject to at least one pathologic disorder, and/or predicting the outcome of said at least one pathological disorder in said subject, the method comprising the steps of:
(a) determining the level/s of resistance and/or tolerance of said subject;
(b) classifying said subject as a subject susceptible to said pathologic disorder and/or to develop a negative outcome of said pathological disorder, if the level of resistance and/or tolerance determined in step (a) is at least one of:
(i) elevated resistance and/or reduced tolerance, in a disorder where a reduced susceptibility and/or positive outcome in a subject is characterized with reduced resistance and/or elevated tolerance; and
(ii) reduced resistance and/or elevated tolerance, in a disorder where a reduced susceptibility and/or positive outcome in a subject is characterized with elevated resistance and/or reduced tolerance;
thereby determining the susceptibility of said subject and/or predicting the outcome of said pathological disorder in said subject; and
(c) administering to said subject an effective amount of at least one therapeutic compound that modifies the tolerance and/or resistance in said subject, wherein said therapeutic compound is any one of:
(i) a compound that elevates resistance and/or reduces tolerance, in a disorder where a responsiveness and/or positive outcome is characterized with elevated resistance and/or reduced tolerance; and
(ii) a compound that reduces resistance and/or elevates tolerance, in a disorder where a responsiveness and/or positive outcome in a subject is characterized with reduced resistance and/or elevated tolerance.

53. The method according to claim 52, wherein said step (a) is performed by a method comprising the steps of:
(a) determining in at least one biological sample of said subject the expression level of at least three biomarkers of at least one of:
(i) at least one biomarker of said resistance, to obtain an expression value for each of said at least one biomarker/s, wherein said at least one biomarker is at least one of MAX Interactor 1 (MXI1), Zinc Finger Protein 395 (ZNF395), Xeroderma Pigmentosum, Complementation group C (XPC), Methylenetetrahydrofolate Dehydrogenase 2 (MTHFD2), Proteasome Activator Subunit 2 (PSME2), Integrator Complex Subunit 12 (INTS12), Proteasome 20S Subunit Beta 7 (PSMB7), RNA Binding Motif Protein 7 (RBM7), Solute Carrier Family 6 Member 8 (SLC6A8), and optionally, Janus Kinase 2 (JAK2), or any combination thereof; and
(ii) at least one biomarker of said tolerance, to obtain an expression value for each of said at least one biomarker/s, wherein said at least one biomarker is at least one of Serine Incorporator 1 (SERINC1), ADP Ribosylation Factor Like GTPase 1 (ARL1), COP9 Signalosome Subunit 2 (COPS2), Cereblon (CRBN), Mitogen-Activated Protein Kinase Kinase 2 (MAP2K2), Rho GDP Dissociation Inhibitor Alpha (ARHGDIA), Glutamate Ionotropic Receptor NMDA Type Subunit Associated Protein 1 (GRINA), Syntaxin Binding Protein 2 (STXBP2), RNA Binding Motif Protein 7 (RBM7), Solute Carrier Family 6 Member 8 (SLC6A8) or any combination thereof; and
(b) determining if the expression values obtained in steps (a) (i) and/or (a) (ii) for each of said at least three biomarkers is positive or negative with respect to a predetermined standard expression value or to an expression value of said biomarker/s in at least one control sample;
wherein at least one of:
(I) a positive expression value of at least one of said MTHFD2, PSME2, INTS12, PSMB7, RBM7, and JAK2, biomarker/s in said sample, and/or a negative expression value of at least one of said MXI1, ZNF395, XPC and SLC6A8 biomarker/s in said sample indicate(s) that the resistance level is elevated in said subject; and/or
(II) a positive expression value of at least one of said MAP2K2, ARHGDIA, GRINA, STXBP2 and SLC6A8 biomarker/s in said sample, and/or a negative expression value of at least one of said SERINC1, ARL1, COPS2, CRBN and RBM7 biomarker/s in said sample indicate(s) that the tolerance level is elevated in said subject.

54. The method according to claim 53, wherein determining the level of expression of at least one said biomarker/s of resistance and/or at least one said biomarker/s of tolerance is performed by the step of contacting at least one detecting molecule or any combination or mixture of plurality of detecting molecules with a biological sample of said subject, or with any nucleic acid or protein product obtained therefrom, wherein each of said detecting molecules is specific for one of said biomarkers; optionally, wherein at least one of:
(i) said at least one detecting molecule is selected from nucleic acid detecting molecules and amino acid detecting molecules; and wherein said nucleic acid detecting molecule/s comprise at least one of: (a) at least one oligonucleotide/s, each oligonucleotide specifically hybridizes to a nucleic acid sequence encoding said at least one biomarker; and (b) at least one nucleic acid aptamer/s specific for said at least one of said biomarkers;
(ii) said biological sample is at least one of a body fluid sample and a cell sample.

55. The method according to claim 52, wherein said pathological disorder is at least one immune related disorder, wherein said immune related disorder is at least one of an infectious disease caused by at least one pathogen, an inflammatory disorder, an autoimmune disorder, a proliferative disorder a neurodegenerative disorder, a metabolic disorder and a condition involving at least one wound in at least one tissue and/or organ of said subject.

56. The method according to claim 55, wherein at least one of:
(a) said infectious disease is caused by at least one pathogen and wherein said pathogen is at least one of a viral pathogen, a bacterial pathogen, a fungal pathogen and a parasite, optionally, at least one of:
(i) said viral pathogen is at least one of Influenza A virus (IAV), Ebola virus, Severe acute respiratory syndrome coronavirus 2 (SARS-COV2), Respiratory Syncytial Virus (RSV), and/or Human parainfluenza virus type 3 (HPIV3);
(ii) said reduced susceptibility and/or a positive outcome of said disorder is characterized by an elevated level of resistance and/or a low level of tolerance;
(b) said immune related disorder is an inflammatory or autoimmune disorder, optionally, at least one of:
(i) said inflammatory or autoimmune disorder is any one of Systemic Lupus Erythematosus (SLE) or Rheumatoid Arthritis (RA); and
(ii) said susceptibility and/or a negative outcome of said disorder is characterized by an elevated level of resistance and/or a low level of tolerance; and
(c) said condition is at least one wound in at least one tissue and/or organ of said subject and wherein a positive outcome of wound healing is characterized by an elevated level of tolerance.

57. The method according to claim 55, wherein said immune related disorder is a proliferative disorder, and wherein said proliferative disorder is cancer; optionally, wherein at least one of:
(a) wherein said cancer is glioma or breast cancer, and wherein at least one of:
(i) said susceptibility and/or negative outcome of said cancer is characterized by an elevated level of resistance and or reduced level of tolerance; and
(ii) said reduced susceptibility and/or positive outcome of said cancer is characterized by an elevated level of tolerance and/or a reduced level of resistance;
(b) wherein said cancer is Leukemia (CLL) and wherein at least one of:
(i) said reduced susceptibility and/or positive outcome of said cancer is characterized by a elevated level of tolerance; and
(ii) said susceptibility and/or negative outcome of said cancer is characterized by a reduced level of tolerance;
(c) wherein said cancer is Multiple Myeloma (MM) cancer, and
wherein at least one of:
(i) said susceptibility and/or negative outcome of said cancer is characterized by an elevated level of tolerance; and
(ii) said reduced susceptibility and/or positive outcome of said cancer is characterized by a reduced level of tolerance; and
(d) wherein said cancer is any one of Lung adenocarcinoma, Neuroblastoma and Astrocytoma, and wherein at least one of:
(i) said susceptibility and/or negative outcome of said cancer is characterized by an elevated level of resistance; and
(ii) said reduced susceptibility and/or positive outcome of said cancer is characterized by a reduced level of resistance.

58. A method for treating, preventing, inhibiting, reducing,
eliminating, protecting or delaying the onset at least one pathological disorder in a subject in need thereof, and for predicting and assessing responsiveness of said subject to at least one compound or a treatment regimen comprising said compound, and optionally for monitoring disease progression, the method comprising the steps of:
(a) determining the levels of resistance and/or tolerance of said subject;
(b) classifying said subject as:
(I) a responder to said at least one compound or a treatment regimen comprising said compound, if at least one sample obtained after the initiation of said treatment regimen and/or a sample of said subject contacted with said compound displays at least one of:
(i) elevated resistance and/or reduced tolerance, in a disorder where responsiveness and/or positive outcome in a subject is characterized with elevated resistance and/or reduced tolerance; and
(ii) reduced resistance and/or elevated tolerance, in a disorder where responsiveness and/or positive outcome is characterized with reduced resistance and/or elevated tolerance; or
(II) a non-responder to said at least one compound or a treatment regimen comprising said compound, if at least one sample obtained after the initiation of said treatment regimen and/or a sample of said subject contacted with said compound displays at least one of:
(i) elevated resistance and/or reduced tolerance, in a disorder where a responsiveness and/or positive outcome is characterized with reduced resistance and/or elevated tolerance; and
(ii) reduced resistance and/or elevated tolerance, in a disorder where a responsiveness and/or positive outcome in a subject is characterized with elevated resistance and/or reduced tolerance;
thereby predicting and assessing responsiveness of said subject to said treatment regimen; and
(c) administering said compound or subjecting said subject to a treatment regime comprising said compound, if at least one of:
(i) said compound or a treatment regimen comprising said compound elevates resistance and/or reduces tolerance, in at least one sample of said subject, wherein said subject is suffering from a disorder where a positive outcome is characterized with elevated resistance and/or reduced tolerance; and
(ii) said compound or a treatment regimen comprising said compound reduces resistance and/or elevated tolerance, in at least one sample of said subject, wherein said subject is suffering from a disorder where positive outcome is characterized with reduced resistance and/or elevated tolerance.

59. The method according to claim 58, wherein said step (a) is performed by the method comprising the steps of:
(a) determining in at least one biological sample of said subject the expression level of at least three biomarkers of at least one of:
(i) at least one biomarker of said resistance, to obtain an expression value for each of said at least one biomarker/s, wherein said at least one biomarker is at least one of MXI1, ZNF395, XPC, MTHFD2, PSME2, INTS12, PSMB7, RBM7, SLC6A8, and optionally, JAK2, or any combination thereof; and
(ii) at least one biomarker of said tolerance, to obtain an expression value for each of said at least one biomarker/s, wherein said at least one biomarker is at least one of SERINC1, ARLI, COPS2, CRBN, MAP2K2, ARHGDIA, GRINA, STXBP2, RBM7, SLC6A8 or any combination thereof; and
(b) determining if the expression values obtained in steps (a) (i) and/or (a) (ii) for each of said at least three biomarkers, is positive or negative with respect to a predetermined standard expression value or to an expression value of said biomarker/s in at least one control sample;
wherein at least one of:
(I) a positive expression value of at least one of said MTHFD2, PSME2, INTS12, PSMB7, RBM7, and JAK2, biomarker/s in said sample, and/or a negative expression value of at least one of said MXI1, ZNF395, XPC and SLC6A8 biomarker/s in said sample indicate(s) that the resistance level is elevated in said subject; and/or
(II) a positive expression value of at least one of said MAP2K2, ARHGDIA, GRINA, STXBP2 and SLC6A8 biomarker/s in said sample, and/or a negative expression value of at least one of said SERINC1, ARL1, COPS2, CRBN and RBM7 biomarker/s in said sample indicate(s) that the tolerance level is elevated in said subject.

60. The method according to claim 58, wherein determining the level of expression of at least one said biomarker/s of resistance and/or at least one said biomarker/s of tolerance is performed by the step of contacting at least one detecting molecule or any combination or mixture of plurality of detecting molecules with a biological sample of said subject, or with any nucleic acid or protein product obtained therefrom, wherein each of said detecting molecules is specific for one of said biomarkers; optionally, wherein at least one of:
(i) said at least one detecting molecule is selected from nucleic acid detecting molecules and amino acid detecting molecules; and wherein said nucleic acid detecting molecule/s comprise at least one of: (a) at least one oligonucleotide/s, each oligonucleotide specifically hybridizes to a nucleic acid sequence encoding said at least one biomarker; and (b) at least one nucleic acid aptamer/s specific for said at least one of said biomarkers;
(ii) said biological sample is at least one of a body fluid sample and a cell sample.

61. The method according to claim 58, further comprising monitoring disease progression comprises at least one of predicting and determining disease relapse and assessing a remission interval, and wherein said method further comprises the steps of:
(d) repeating step (a) to determine the levels of resistance and/or tolerance in at least one more temporally-separated sample of said subject; and
(e) predicting and/or determining disease relapse in said subject, if at least one temporally separated sample obtained after the initiation of said treatment regimen displays at least one of:
(i) elevated resistance and/or reduced tolerance, in a disorder where a responsiveness and/or positive outcome is characterized with reduced resistance and/or elevated tolerance; and
(ii) reduced resistance and/or elevated tolerance, in a disorder where a responsiveness and/or positive outcome in a subject is characterized with elevated resistance and/or reduced tolerance.

62. The method according to claim 58, wherein said pathological disorder is at least one immune related disorder, and wherein said immune related disorder is at least one of an infectious disease caused by at least one pathogen, an inflammatory disorder, an autoimmune disorder, a proliferative disorder, a neurodegenerative disorder, a metabolic disorder and a condition involving at least one wound in at least one tissue and/or organ of said subject.

63. The method according to claim 62, wherein at least one of:
(a) said infectious disease is caused by at least one pathogen and wherein said pathogen is at least one of a viral pathogen, a bacterial pathogen, a fungal pathogen and a parasite, optionally, said viral pathogen is at least one of Influenza A virus (IAV), Ebola virus, Severe acute respiratory syndrome coronavirus 2 (SARS-COV2), Respiratory Syncytial Virus (RSV), and/or Human parainfluenza virus type 3 (HPIV3);
(b) said immune related disorder is an inflammatory or autoimmune disorder, wherein said inflammatory or autoimmune disorder is any one of Systemic Lupus Erythematosus (SLE) or Rheumatoid Arthritis (RA); and
(c) said condition is at least one wound in at least one tissue and/or organ of said subject.

64. The method according to claim 62, wherein said immune related disorder is a proliferative disorder, and wherein said proliferative disorder is cancer; optionally, wherein at least one of:
(a) wherein said cancer is glioma or breast cancer;
(b) wherein said cancer is Leukemia (CLL);
(c) wherein said cancer is Multiple Myeloma (MM) cancer;
(d) wherein said cancer is any one of Lung adenocarcinoma, Neuroblastoma and Astrocytoma

65. A method for manipulating the immunological state of a subject suffering from a pathologic condition by modulating the levels of resistance and/or tolerance of said subject, the method comprising evaluating the immunological state in a subject by determining the levels of resistance and/or tolerance of said subject, the method comprising:
(a) determining in at least one biological sample of said subject the expression level of at least three biomarkers of at least one of:
(i) at least one biomarker of said resistance, to obtain an expression value for each of said at least one biomarker/s, wherein said at least one biomarker/s is at least one of: MAX Interactor 1 (MXI1), Zinc Finger Protein 395 (ZNF395), Xeroderma Pigmentosum, Complementation group C (XPC), Methylenetetrahydrofolate Dehydrogenase 2 (MTHFD2), Proteasome Activator Subunit 2 (PSME2), Integrator Complex Subunit 12 (INTS 12), Proteasome 20S Subunit Beta 7 (PSMB7), RNA Binding Motif Protein 7 (RBM7), Solute Carrier Family 6 Member 8 (SLC6A8), and optionally, Janus Kinase 2 (JAK2), or any combination thereof; and
(ii) at least one biomarker of said tolerance, to obtain an expression value for each of said at least one biomarker/s, wherein said at least one biomarker/s is at last one of: Serine Incorporator 1 (SERINC1), ADP Ribosylation Factor Like GTPase 1 (ARL1), COP9 Signalosome Subunit 2 (COPS2), Cereblon (CRBN), Mitogen-Activated Protein Kinase Kinase 2 (MAP2K2), Rho GDP Dissociation Inhibitor Alpha (ARHGDIA), Glutamate Ionotropic Receptor NMDA Type Subunit Associated Protein 1 (GRINA), Syntaxin Binding Protein 2 (STXBP2), RNA Binding Motif Protein 7 (RBM7), Solute Carrier Family 6 Member 8 (SLC6A8) or any combination thereof;
(b) determining if the expression values obtained in steps (a) (i) and/or (a) (ii) for each of said at least three biomarkers is positive or negative with respect to a predetermined standard expression value or to an expression value of said biomarker/s in at least one control sample;
wherein at least one of:
(I) a positive expression value of at least one of said MTHFD2, PSME2, INTS12, PSMB7, RBM7 and JAK2, biomarker/s in said sample, and/or a negative expression value of at least one of said MXI1, ZNF395, XPC and SLC6A8 biomarker/s in said sample, indicate(s) that the resistance level is elevated in said subject; and/or
(II) a positive expression value of at least one of said MAP2K2, ARHGDIA, GRINA, STXBP2 and SLC6A8 biomarker/s in said sample, and/or a negative expression value of at least one of said SERINC1, ARL1, COPS2, CRBN and RBM7 biomarker/s in said sample indicate(s) that the tolerance level is elevated in said subject, thereby determining the immune/immunological state in said subject; and
(c) administering to said subject a therapeutically effective amount of at least one of:
(i) at least one compound that leads to an increase in the level of at least one of MTHFD2, PSME2, INTS12, PSMB7, RBM7 and JAK2, biomarker/s and/or at least one compound that leads to a decrease in the level of at least one of MXI1, ZNF395, XPC and SLC6A8 biomarker/s, and/or at least one compound that leads to a decrease in the level of at least one of MAP2K2, ARHGDIA, GRINA and STXBP2 biomarker/s and/or at least one compound that leads to an increase in the level of at least one of SERINC1, ARL1, COPS2 and CRBN biomarker/s, for a subject suffering from a disorder where a positive outcome is characterized with elevated resistance and/or reduced tolerance; or
(ii) at least one compound or a procedure that leads to a decrease in the level of at least one of MTHFD2, PSME2, INTS12, PSMB7, RBM7 and JAK2, biomarker/s and/or at least one compound that leads to an increase in the level of at least one of MXI1, ZNF395, XPC and SLC6A8 biomarker/s, and/or at least one compound that leads to an increase in the level of at least one of MAP2K2, ARHGDIA, GRINA and STXBP2 biomarker/s and/or at least one compound that leads to a decrease in the level of at least one of SERINC1, ARLI, COPS2 and CRBN biomarker/s, for a subject suffering from a disorder where positive outcome is characterized with reduced resistance and/or elevated tolerance.

66. The method according to claim 65, wherein step (a) comprises determining in at least one biological sample of said subject the expression level of:
(i) biomarkers of resistance to obtain an expression value for each of said biomarker/s, wherein said biomarkers of resistance are MXI1, ZNF395, XPC, MTHFD2, PSME2, INTS12, PSMB7, RBM7, SLC6A8 and optionally, JAK2; and
(ii) biomarker/s of tolerance to obtain an expression value for each of said biomarker/s, wherein said biomarkers of tolerance are SERINC1, ARLI, COPS2, CRBN, MAP2K2, ARHGDIA, GRINA, STXBP2, RBM7 and SLC6A8.

67. The method according to claim 65, wherein determining the level of expression of at least one said biomarker/s of resistance and/or at least one said biomarker/s of tolerance is performed by the step of contacting at least one detecting molecule or any combination or mixture of plurality of detecting molecules with a biological sample of said subject, or with any nucleic acid or protein product obtained therefrom, wherein each of said detecting molecules is specific for one of said biomarkers; optionally, wherein said at least one detecting molecule is selected from nucleic acid detecting molecules and amino acid detecting molecules; and wherein said nucleic acid detecting molecule/s comprise at least one of: (a) at least one oligonucleotide/s, each oligonucleotide specifically hybridizes to a nucleic acid sequence encoding said at least one biomarker; and (b) at least one nucleic acid aptamer/s specific for said at least one of said biomarkers.

68. The method according to claim 65, wherein said biological sample is at least one of a body fluid sample and a cell sample, optionally, wherein said sample is at least one of blood sample, skin cell sample, tumor biopsy.

69. The method according to claim 65, wherein said subject is any one of: (a) a subject displaying healthy-homeostatic conditions, (b) a subject suffering from at least one pathologic disorder, and (c) a subject exposed to at least one biotic and/or at least one abiotic stimulus.

70. A screening method for identifying and/or evaluating at least one therapeutic compound for the treatment of a pathologic disorder, wherein said compound modifies the level of resistance and/or tolerance in at least one subject suffering from said pathologic disorder, the method comprising the steps of:
(A) determining the levels of resistance and/or tolerance of at least one biological sample contacted with said compound, said sample is of a subject suffering from said pathologic disorder;
(B) determining that said candidate compound is a therapeutic compound for said disorder if:
(i) said compound elevates resistance and/or reduces tolerance, in a sample of said subject contacted with said candidate compound, as compared to a control sample, wherein said subject is suffering from a disorder where a positive outcome is characterized with elevated resistance and/or reduced tolerance; and
(ii) said compound reduces resistance and/or elevates tolerance, in a sample of said subject contacted with said candidate compound, as compared to a control sample, wherein said subject is suffering from a disorder where positive outcome is characterized with reduced resistance and/or elevated tolerance;
wherein said step (A) is performed by the method comprising the steps of:
(a) determining in at least one biological sample of said subject the expression level of at least three biomarkers of at least one of:
(i) at least one biomarker of said resistance, to obtain an expression value for each of said at least one biomarker/s, wherein said at least one biomarker is at least one of MXI1, ZNF395, XPC, MTHFD2, PSME2, INTS12, PSMB7, RBM7, SLC6A8, and optionally, JAK2, or any combination thereof; and
(ii) at least one biomarker of said tolerance, to obtain an expression value for each of said at least one biomarker/s, wherein said at least one biomarker is at least one of SERINC1, ARL1, COPS2, CRBN, MAP2K2, ARHGDIA, GRINA, STXBP2, RBM7, SLC6A8 or any combination thereof; and
(b) determining if the expression values obtained in steps (a) (i) and/or (a) (ii) for each of said at least three biomarkers is positive or negative with respect to a predetermined standard expression value or to an expression value of said biomarker/s in at least one control sample;
wherein at least one of:
(I) a positive expression value of at least one of said MTHFD2, PSME2INTS12, PSMB7, RBM7 and JAK2, biomarker/s in said sample,
and/or a negative expression value of at least one of said MXI1, ZNF395, XPC and SLC6A8 biomarker/s in said sample, indicate(s) that the resistance level is elevated in said subject; and/or
(II) a positive expression value of at least one of said MAP2K2, ARHGDIA, GRINA, STXBP2 and SLC6A8 biomarker/s in said sample,
and/or a negative expression value of at least one of said SERINC1, ARL1, COPS2, CRBN and RBM7 biomarker/s in said sample, indicate(s) that the tolerance level is elevated in said subject.

71. A diagnostic composition or kit comprising at least three detecting molecules or any combination or mixture of plurality of detecting molecules specific for determining the level of expression of at least three biomarkers of at least one of:
(i) at least one biomarker of resistance, said at least one biomarker is at least one of MXI1, ZNF395, XPC, MTHFD2, PSME2, INTS12, PSMB7, RBM7, SLC6A8 and optionally, JAK2, or any combination thereof; and
(ii) at least one biomarker of said tolerance, said at least one biomarker is at least one of SERINC1, ARL1, COPS2, CRBN, MAP2K2, ARHGDIA, GRINA, STXBP2, RBM7, SLC6A8 or any combination thereof; wherein each of said detecting molecules is specific for one of said biomarker/s; wherein said kit optionally further comprises at least one of:
b. pre-determined calibration curve/s or predetermined standard/s providing standard expression values of said at least three biomarkers; and
c. at least one control sample, optionally, said composition and/or kit is adapted for performing the method of claim 52.
